SEQUENCE LISTING (391 sequences)

SEQ ID NO: 1 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGAGYVYDSGSVSRYILSEIKPLHNFLTQLQQFLKLKQKQANLVLKIIEQLPSAKESPDKFLEVCTWVDQIAALNDSKTRKTTSETVRAVLDSLSEKKKSSPAAD

SEQ ID NO: 2 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 3 - protein, 167 aa, artificial sequence
  HIV1_1.3 I-CreI variant
MANTKYNKEFLLYLAGFVDGDGSIIAQIKPGQSYKFKHRLSLTFVVTQKTQRRWFLDKLVDEIGVGYVEDRGSVSNYRLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 4 - protein, 167 aa, artificial sequence
  HIV1_1.3 I-CreI variant
MANTKYNKEFLLYLAGFVDGDGSIIAQIKPGQSYKFKHRLSLTFIVTQKTQRRWFLDKLVDEIGVGYVEDRGSVSNYRLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 5 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 6 - protein, 167 aa, artificial sequence
  HIV1_1.3 I-CreI variant
MANTKYNKEFLLYLAGFVDGDGSIIAQIKPNQSCKFKHRLSLTFAVTQKTQRRWFLDKLVDEIGVGYVYDSGSVSRYQLSEIKPLHNFLTQLQPFLKLKQKQANLVLKIIEQLPSAKESPDKFLEVCTWVDQIAALNDSKTRKTTSETVRAVLDNLSEKKKSSPAAD

SEQ ID NO: 7 - protein, 167 aa, artificial sequence
  HIV1_1.4 I-CreI variant
MANTKYNKEFLLYLAGFVDGDGSIIAQIQPNQSSKFKHRLKLTFKVTQKTQRRWFLDKLVDEIGVGYVYDSGSVSEYSLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 8 - protein, 167 aa, artificial sequence
  HIV1_1.4 I-CreI variant
MANTKYNKEFLLYLAGFVDGDGSIIAQIKPNQSCKFKHALSLTFKVTQKTQRRWFLDKLVDEIGVGYVYDSGSVSEYSLSEIKSLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 9 - protein, 167 aa, artificial sequence
  HIV1_1.4 I-CreI variant
MANTKYNKEFLLYLAGFVDGDGSIIAQIKPNQSSKFKHRLNLTFKVTQKTQRRWFLDKLVDEIGVGYVYDSGSVSEYSLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 10 - protein, 167 aa, artificial sequence
  HIV1_1.4 I-CreI variant
MANTKYNKEFLLYLAGFVDGDGSIIAQIKPCQSTKFKHQLRLTFRVTQKTQRRWFLDKLVDEIGVGYVYDSGSVSDYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 11 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 12 - protein, 167 aa, artificial sequence
  HIV1_1.4 I-CreI variant
MANTKYNKEFLLYLAGFVDGDGSIIAQIKPNQSSKFKHQLSLTFRVTQKTQRRWFLDKLVDEIGVGYVSDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 13 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLSEIKPLHNFLTQLQPFLKLKQKQANLVLKIIEQLPSAKESPDKFLEVCTWVDQIAALNDSKTRKTTSETVRAVLDSLSEKKKSPPAAD

SEQ ID NO: 14 - DNA, 51 bp, artificial sequence
  HIV1_1.3 target oligonucleotide
tggcatacaagtttgcagaactacgtacgtagttctgccaatcgtctgtca

SEQ ID NO: 15 - DNA, 51 bp, artificial sequence
  HIV1_1.5 target oligonucleotide
tggcatacaagtttgcagaactacacacgtagttctgccaatcgtctgtca

SEQ ID NO: 16 - DNA, 26 bp, artificial sequence
  Gal10F primer
gcaactttagtgctgacacatacagg

SEQ ID NO: 17 - DNA, 24 bp, artificial sequence
  Gal10R primer
acaaccttgattggagacttgacc

SEQ ID NO: 18 - DNA, 15 bp, artificial sequence
  assF primer
  misc_feature (4)..(6): n is a, t, c, or g
ctannnttgaccttt

SEQ ID NO: 19 - DNA, 15 bp, artificial sequence
  assR primer
  misc_feature (10)..(12): n is a, t, c, or g
aaaggtcaannntag

SEQ ID NO: 20 - DNA, 51 bp, artificial sequence
  HIV1_1.4 target oligonucleotide
tggcatacaagtttcctggccctggtaccagggccaggcaatcgtctgtca

SEQ ID NO: 21 - DNA, 51 bp, artificial sequence
  HIV1_1.6 target oligonucleotide
tggcatacaagtttcctggccctgacaccagggccaggcaatcgtctgtca

SEQ ID NO: 22 - DNA, 51 bp, artificial sequence
  HIV1_1.2 target oligonucleotide
tggcatacaagtttgcagaactacgtaccagggccaggcaatcgtctgtca

SEQ ID NO: 23 - DNA, 51 bp, artificial sequence
  HIV1_1 target oligonucleotide
tggcatacaagtttgcagaactacacaccagggccaggcaatcgtctgtca

SEQ ID NO: 24 - DNA, 59 bp, artificial sequence
  preATGCreFor primer
gcataaattactatacttctatagacacgcaaacacaaatacacagcggccttgccacc

SEQ ID NO: 25 - DNA, 48 bp, artificial sequence
  ICreIpostRev primer
ggctcgaggagctcgtctagaggatcgctcgagttatcagtcggccgc

SEQ ID NO: 26 - protein, 167 aa, artificial sequence
  HIV1_1.3 I-CreI variant
MANTKYNKEFLLYLAGFVDGDGSIIAQIKPGQSYKFKHRLSLTFVVTQKTQRRWLLDKLVDEIGVGYVEDRGSVSNYRLSKTKPLHNFLTQLQPFLKLKQKQANLVLKIIEQLPSAKESPDKFLEVCTWVDQVAALNDSKTRKTTSETVRAVLDSLSEKKKSSRAAD

SEQ ID NO: 27 - protein, 167 aa, artificial sequence
  HIV1_1.3 I-CreI variant
MANTKYNKEFLLYLAGFVDGDGSIIAQIKPGQSYKFKHRLSLTFVVTQKTQRRWLLDKLVDEIGVGYVEDRGSVSNYRLSKIKPLHNFLTQLQPFLKLKRKQANLVLKIIEHLPSAKESPDKFLEVCTWVDQIAALNDSKTRKTTSETVRAVLDSLSEKKKSSPAAD

SEQ ID NO: 28 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLNEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 29 - protein, 167 aa, artificial sequence
  HIV1_1.3 I-CreI variant
MANTKYNKEFLLYLAGFVDGDGSIIAQIKPGQSYKFKHRLSLTFVVTQKTQRRWLLDKLVDEIGVGYVYDSGSVSRYYLSEIKPLHNFLTQLQPFLKLKQNQANLVLKIIEQLPSAKESPDKFLEVCTWVDQIAALNDSKTRKTTSETVRAVLDSLSEKKKSSPAAD

SEQ ID NO: 30 - protein, 167 aa, artificial sequence; identical to SEQ ID NO: 13
  HIV1_1.3 I-CreI variant

SEQ ID NO: 31 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLSEIKPLHNFLTQLQPFLKLKQKQANLVLKIIEQLPSAKESPDKFLEVCTWVDQIAALNDSKTRKTTSETVRAVLDRLSEKKKSSPAAD

SEQ ID NO: 32 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDRLVDEIGVGYVYDSGSVSRYYLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 33 - protein, 167 aa, artificial sequence
  HIV1_1.3 I-CreI variant
MANTKYNKEFLLYLAGFVDGDGSIIAQIKPGQSYKFKHRLSLTFNVTQKTRRRWFLDKLVDEIGAGYVYDSGSVSRYILSEIKPLHNFLTQLQQFLKLKQKQANLVLKIIEQLPSAKESPDKFLEVCTWVDQIAALNDSKTRKTTSETVRAVLDSLSEKKKSSPAAD

SEQ ID NO: 34 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLSEIKPLHNFLTQLQPFLKLKQKQANLVLKIIEQLPSAKESPDKFLEVCTWVDQIAALNDSKTRKTTSETVRAVLDSLSEKRKSSPAAD

SEQ ID NO: 35 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLSEIKPLHNFLTQLQPFLKLKQKQANLVLRIIEQLPSAKESPDKFLEVCTWVDQIAALNDSKTRKTTSETVRAVLDSLSEKKKSPPAAD

SEQ ID NO: 36 - protein, 167 aa, artificial sequence
  HIV1_1.3 I-CreI variant
MANTKYNKEFLLYLAGFVDGDGSIIAQIKPGQSYKFKHRLSLTFVVTQKTQRRWLLDKLVDEIGVGYVEDRGSVSNYRLSKIKPLYNFLTQLQPFLKLKRKQANLVLKIIEHLPSAKESPDKFLEVCTWVDQIAALNDSKTRKTTSETVRAVLDSLSEKKKSSPAAD

SEQ ID NO: 37 - protein, 167 aa, artificial sequence
  HIV1_1.3 I-CreI variant
MASTKYNKEFLLYLAGFVDGDGSIIAQIKPGQSYKFKHRLSLTFVVTQKTQRRWLLDKLVDEIGVGYVEDRGSVSNYRLSKIKPLHNFLAQLQPFLKLKRKQANLVLKIIEHLPSAKESPDKFLEVCTWVDQVAALNDSKTRKTTSETVRAVLDSQSEKKKSSRAAD

SEQ ID NO: 38 - protein, 167 aa, artificial sequence
  HIV1_1.3 I-CreI variant
MANTKYNKEFLLYLAGLVDGDGSIIAQIKPGRSYKFKHRLSLTFVVTQKTQRRWLLDELVDGIGVGYVEDRGSVSNYRLSKTKPLHNFLTQLQPFLKLKQKQANLVLKIIEQLPSAKESPDKFLEVCTWVDQVAALNDSKTRKTTSETVRAVLDSLSEKKKSPPAAD

SEQ ID NO: 39 - protein, 167 aa, artificial sequence
  HIV1_1.3 I-CreI variant
MANTKYNKEFLLYLAGFVDGDGSIIAQIKPGQSYKFKHRLSLTFVVTQKTQRRWLLDKLVDEIGVGYVEDRGSVSNYRLSKIKPLHNFLTQLQPFLKLKRKQANLVLKIIEHLPSAKESPDKFLEVCTWVDQIAALNDSKTRKTTSETVRAVLDSLSEKKKSSRAAD

SEQ ID NO: 40 - protein, 167 aa, artificial sequence
  HIV1_1.3 I-CreI variant
MANTKYNKEFLLYLAGFADGDGSIIAQIKPGQSYKFKHRLSLAFVVTQKTQRRWLLDKLVDEIGAGYVEDRGSVSNYRLSRIKPLHDFLTQLQPFLKLKRKQANLVLKIIEHLPSAKESPDKFLEVCTWVDQIAALNDSKTRKTTSETVRAVLDSLSEKKKSSPAAD

SEQ ID NO: 41 - protein, 167 aa, artificial sequence
  HIV1_1.3 I-CreI variant
MANTKYNKEFLLYLAGFVDGDGSIIAQIKPGQSYKFKHRLSLTFVVTQKTQRRWLLDKLVDEIGVGYVEDRGSVSNYRLSKTKPLHNFLTQLQPFLKLKQKQANLVLKIIEQLPSAKESPDRFLEVCTWVDQVAALNDSKTRKTTSETVRAVLDSLSEKKESSPAAD

SEQ ID NO: 42 - protein, 167 aa, artificial sequence
  HIV1_1.3 I-CreI variant
MANTKYNKEFLLYLAGFVDGDGSIIAQIRPGQSYKFKHRISLTFVVTQKTQRRWLLDKLVDEIGVGYVEDRGSVSNYRLSKTKPLHSFLTQLQPFLKLKQKQANLVLKIIEQLPSAKESPDKFLEVCTWVDQVAALNDSKTRKTTSETVRTVLDSLSEKKKSPPAAD

SEQ ID NO: 43 - protein, 167 aa, artificial sequence
  HIV1_1.3 I-CreI variant
MANTKYNKEFLLYLAGFVDGDGSIIAQIKPGQSYRFKHRLSLTFVVTQKTQRRWLLDKLVDEIGVGYVEDRGSVSNYRLSKTKPLHNFLTQLQPFLKLKQKQANLVLKIIEQLPSAKESPDKFLEVCTWVDQVAALNDSKTRKTTSETVRAVLDSLSEKKKSSRAAD

SEQ ID NO: 44 - protein, 167 aa, artificial sequence
  HIV1_1.3 I-CreI variant
MANTKYNKEFLLYLAGFVDGDGSIIAQIKPGQSYKFKHRLSLTFNVTQKTQRRWFLDKLVDEIGVGYVYDSGSVSRYYLNEIKPLHNFLTQLQPFLKLKQEQANLALKIIEQLPSAKESPDKFLEVCTWVDQIAALNDSKTRKTTSETVRAVLDSLSEKKKSSPAAD

SEQ ID NO: 45 - protein, 167 aa, artificial sequence
  HIV1_1.3 I-CreI variant
MANTKYNKEFLLYLAGLVDGDGSIIAQIKPGQSYKFKHRLSLTFVVTQKTQRRWLLDKLVDEIGVGYVEDRGSVSNYRLSKIKPLHNFLTQLQPFLKLKRKQANLVLKIIEHQPSAKESPDKFLEVCTWVDQIAALNDSKTRKTTSETVRAVLDSLSEKKKSSPAAD

SEQ ID NO: 46 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGVGYVYDSGSVSDYNLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 47 - DNA, 33 bp, artificial sequence
  G19SF primer
gccggctttgtggactctgacggtagcatcatc

SEQ ID NO: 48 - DNA, 33 bp, artificial sequence
  G19SR primer
gatgatgctaccgtcagagtccacaaagccggc

SEQ ID NO: 49 - DNA, 33 bp, artificial sequence
  F54LF primer
acccagcgccgttggctgctggacaaactagtg

SEQ ID NO: 50 - DNA, 33 bp, artificial sequence
  F54LR primer
cactagtttgtccagcagccaacggcgctgggt

SEQ ID NO: 51 - DNA, 33 bp, artificial sequence
  E80KF primer
ttaagcaaaatcaagccgctgcacaacttcctg

SEQ ID NO: 52 - DNA, 33 bp, artificial sequence
  E80KR primer
caggaagttgtgcagcggcttgattttgcttaa

SEQ ID NO: 53 - DNA, 33 bp, artificial sequence
  F87LF primer
aagccgctgcacaacctgctgactcaactgcag

SEQ ID NO: 54 - DNA, 33 bp, artificial sequence
  F87LR primer
ctgcagttgagtcagcaggttgtgcagcggctt

SEQ ID NO: 55 - DNA, 33 bp, artificial sequence
  V105AF primer
aaacaggcaaacctggctctgaaaattatcgaa

SEQ ID NO: 56 - DNA, 33 bp, artificial sequence
  V105AR primer
ttcgataattttcagagccaggtttgcctgttt

SEQ ID NO: 57 - DNA, 33 bp, artificial sequence
  I132VF primer
acctgggtggatcaggttgcagctctgaacgat

SEQ ID NO: 58 - DNA, 33 bp, artificial sequence
  I132VR primer
atcgttcagagctgcaacctgatccacccaggt

SEQ ID NO: 59 - protein, 167 aa, artificial sequence
  HIV1_1.3 I-CreI variant
MANTKYNKEFLLYLAGFVDSDGSIIAQIKPGQSYKFKHRLSLTFVVTQKTQRRWLLDKLVDEIGVGYVEDRGSVSNYRLSKTKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 60 - protein, 167 aa, artificial sequence
  HIV1_1.3 I-CreI variant
MANTKYNKEFLLYLAGFVDSDGSIIAQIKPGQSYKFKHRLSLTFVVTQKTQRRWLLDKLVDEIGVGYVEDRGSVSNYRLSKTKPLHDFLTQLQPFLKLKRKQANLVLKIIEHLPSAKESPDKFLEVCTWVDQVAALNDSKTRKTTSETVRAVLDSLSEKKKSPPAAD

SEQ ID NO: 61 - protein, 167 aa, artificial sequence
  HIV1_1.3 I-CreI variant
MANTKYNKEFLLYLAGFVDSDGSIIAQIKPGRSYKFKHRLSLTFVVTQKTQRRWLLDKLVDEIGAGYVEDRGSVSNYRLSRIKPLHDFLTQLQPFLKLKQKQANLALKIIEQLPSAKESPDKFLEVCTWVDQVAALNDSKTRKTTSETVRAVLDSLSEKKKSSPAAD

SEQ ID NO: 62 - protein, 167 aa, artificial sequence
  HIV1_1.3 I-CreI variant
MANTKYNKEFLLYLAGFVDSDGSIIAQIKPGQSYKFKHRLSLTFVVTQKTQRRWLLDKLVDEIGVGYVEDRGSVSNYRLSKIKPLYNFLTQLQPFLKLKRKQANLVLKIIEHLPSAKESPDKFLEVCTWVDQIAALNDSKTRKTTSETVRAVLDSLSEKKKSPPAAD

SEQ ID NO: 63 - protein, 167 aa, artificial sequence
  HIV1_1.3 I-CreI variant
MANTKYNKEFLLYLAGFVDSDGSIIAQIKPGQSYKFKHRLSLTFVVTQKTQRRWLLDKLVDEIGAGYVEDRGSVSNYRLSRIKPLHDFLTQLQPFLKLKRKQANLVLKIIEHLPSAKESPDKFLEVCTWVDQIAALNDSKTRKTTSETVRAVLDSLSEKKKSSPAAD

SEQ ID NO: 64 - protein, 167 aa, artificial sequence
  HIV1_1.3/1.5 I-CreI variant
MANTKYNKEFLLYLAGLVDGDGSIIAQIKPGRSYKFKHRLSLTFVVTQKTQRRWLLDKLVDEIGVGYVEDRGSVSNYRLSRIKPLHDFLTQLQPFLKLKQKQANLVLKIIEHLPSAKESPDKFLEVCTWVDQVAALNDSKTRKTTSETVRAVLDSLSEKKKSSRAAD

SEQ ID NO: 65 - protein, 167 aa, artificial sequence
  HIV1_1.3/1.5 I-CreI variant
MANTKYNKEFLLYLAGLVDGDGSIIAQIKPGRSYKFKHRLSLTFVVTQKTQRRWLLDKLVDEIGVGYVEDRGSVSNYRLSKTKPLHNLLTQLQPFLKLKQKQANLALKIIEQLPSAKESPDKFLEVCTWVDQVAALNDSKTRKTTSETVRAVLDSLSEKKKSSRAAD

SEQ ID NO: 66 - protein, 167 aa, artificial sequence
  HIV1_1.4 I-CreI variant
MANTKYNKEFLLYLAGFVDGDGSIIAQIKPNQSCKFKHALSLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 67 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGVGYVCDSGSVSDYNLSEIKPLHNFLTQLQPFLKLKQKQANLVLKIIEQLPSAKESPDKFLEVCTWVDQIAALNDSKTRKTTSETVRAVLDSLSEKKRSSPAAD

SEQ ID NO: 68 - protein, 167 aa, artificial sequence; identical to SEQ ID NO: 11
  HIV1_1.4 I-CreI variant

SEQ ID NO: 69 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSKKKKSSPAAD

SEQ ID NO: 70 - protein, 167 aa, artificial sequence
  HIV1_1.4 I-CreI variant
MANTKYNKEFLLYLAGLVDGDGSIIAQIKPNQSTKFKHQLRLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 71 - protein, 167 aa, artificial sequence
  HIV1_1.4 I-CreI variant
MANTKYNKEFLLYLAGFVDGDGSIIAQIKPNQSTKFKHQLKLTLRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 72 - protein, 167 aa, artificial sequence
  HIV1_1.4 I-CreI variant
MANTKYDKEFLLYLAGFVDGDGSIIAQIKPNQSTKFKHQLKLTFRVTQKTQRRWFLDKLVDEIGVGYVYDSGSVSEYSLSEIKSLHNFLTQLQPFLKLKQKQANLVLKIIEQLPSAKESPDKFLEVCTWVDQIAALNDSKTRKTTSETVRAVLDNLSEKKKPSPAAD

SEQ ID NO: 73 - protein, 167 aa, artificial sequence
  HIV1_1.4 I-CreI variant
MANTKYNKEFLLYLAGFVDGDGSIIAQIKPNQSYKFKHRLSLTFRVTQKTQRRWFLDKLVDEIGVGYVRDSGSVSNYILSEIKPLHNFLTQLQPFLKLKQKQANLALKIIEQLPSAKESPDKFLEVCTWVDRIAALNDSKTRKTTSETVRAVLDSLSEKKKSSPAAD

SEQ ID NO: 74 - protein, 167 aa, artificial sequence
  HIV1_1.4 I-CreI variant
MANTKYNEEFLLYLAGFVDGDGSIIAQIKPNQSTKFKHQLKLTFKVTQKTQRRWFLDKLVDEIGVGYVYDSGPVSEYSLSEIKSLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 75 - protein, 167 aa, artificial sequence
  HIV1_1.4 I-CreI variant
MANAKYNKEFLLYLAGFVDGDGSIIAQIKPNQSCKFKHQLKLTFRVTQKTQRRWFLDKLVDEIGVGYVYDSGSVSDYNLSEIKPLHNFLTQLQPFLKLKQKQANLVLKIIEQLPSAKESPDKFLEVCTWADQIAALNDSKTRKTTSETVRAGLDSLSEKKKSSPAAD

SEQ ID NO: 76 - protein, 167 aa, artificial sequence; identical to SEQ ID NO: 69
  HIV1_1.4 I-CreI variant

SEQ ID NO: 77 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGVGYVYDSGSVSDYNLSEIKPLHNFLTQLQPFLKLKQKQANLVLKIIEQLPSAKGSPDKFLEVCTWVDQIAALNDSKTRKTTSETVRAVLDSLSEKKKSSPAAD

SEQ ID NO: 78 - protein, 167 aa, artificial sequence
  HIV1_1.4 I-CreI variant
MANTKYDKEFLLYLAGFVDGDGSIIAQIKPNQSTKFKHQLKLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 79 - protein, 167 aa, artificial sequence; identical to SEQ ID NO: 11
  HIV1_1.4 I-CreI variant

SEQ ID NO: 80 - protein, 167 aa, artificial sequence
  HIV1_1.4 I-CreI variant
MANTKYNKVFLLYLAGFVDGDGSIIAQIKPNQSTKFKHQLKLTFRVTQKTQRRWFLDKLVDEIGAGYVYDSGSVSDYNLSEIKPLHNFLTQLQPFLKLKQKQAKLVLKIIEQLPSARESPDKFLEVCTWVDQVAALNDSKTRKTTSETVRAVLDSLSEKKKSSPAAD

SEQ ID NO: 81 - protein, 167 aa, artificial sequence
  HIV1_1.4 I-CreI variant
MANTKYNKEFLLYLAGLVDGDGSIIAQIKPNQSTKFKHQLKLTLRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 82 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDELVDEIGVGYVNDSGSVSDYNLSEIKPLHNFLTQLQPFLKLKQKQANLVLKIIEQLPSAKESPDRFLEVCTWVDQVAALNDSKTRKTTSETVRAVLDSLSEKKKSSPAAD

SEQ ID NO: 83 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGAGYVYDTGSVSDYNLSEIKPLHNFLTQLQPFLKLKQKQANLVLKIIEQLPSAKESPDKFLEVCTWVDQIAALNDSKTRKTTSETVRAVLGSLSEKKKSSPAAD

SEQ ID NO: 84 - protein, 167 aa, artificial sequence
  HIV1_1.4 I-CreI variant
MANTKYNKEFLLYLAGFVDGDGSIIAQIKPNQSTKFKHQLKLTFRVTQKTQRRWFLDKLVDEIGVGYVYDSGSVSDYNLSEIKPLHNFLTQLQPFLKLKQKQANLLLKIIEQLPSAKESPDKFLEVCTWVDQIAALNDSKTRKTTSETVRAVLDSLNKKKKSSPAAD

SEQ ID NO: 85 - protein, 167 aa, artificial sequence
  HIV1_1.4 I-CreI variant
MANTKYNKEFLLYLAGFVDGDGSIIAQIKPNQSTKFKHQLKLTLRVTQKTQRRWFLDKLVDEIGVGYVYDSGSVSDYNLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 86 - DNA, 51 bp, artificial sequence
  HIV1_3.3 target oligonucleotide
tggcatacaagtttctcagaccctgtacagggtctgagcaatcgtctgtca

SEQ ID NO: 87 - DNA, 51 bp, artificial sequence
  HIV1_3.5 target oligonucleotide
tggcatacaagtttctcagacccttttaagggtctgagcaatcgtctgtca

SEQ ID NO: 88 - protein, 167 aa, artificial sequence
  HIV1_3.3 I-CreI variant
MANTKYNKEFLLYLAGFVDGDGSIIAQIKPNQSNKFKHYLRLTFKVTQKTQRRWFLDKLVDEIGVGYVSDNGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 89 - protein, 167 aa, artificial sequence
  HIV1_3.3 I-CreI variant
MANTKYNKEFLLYLAGFVDGDGSIIAQIKPCQSCKFKHQLSLTFKVTQKTQRRWFLDKLVDEIGVGYVADSGSVSDYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 90 - protein, 167 aa, artificial sequence
  HIV1_3.3 I-CreI variant
MANTKYNKEFLLYLAGFVDGDGSIIAQIKPCQSCKFKHQLSLTFKVTQKTQRRWFLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 91 - protein, 167 aa, artificial sequence
  HIV1_3.3 I-CreI variant
MANTKYNKEFLLYLAGFVDGDGSIIAQIKPNQKAKFKHQLSLTFQVTQKTQRRWFLDKLVDEIGVGYVEDGGSVSNYLLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 92 - protein, 167 aa, artificial sequence
  HIV1_3.3 I-CreI variant
MANTKYNKEFLLYLAGFVDGDGSIIAQIKPNQTCKFKHQLSLTFKVTQKTQRRWFLDKLVDEIGVGYVRDTGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 93 - protein, 167 aa, artificial sequence
  HIV1_3.3 I-CreI variant
MANTKYNKEFLLYLAGFVDGDGSIIAQIKPNQSNKFKHYLSLTFKVTQKTQRRWFLDKLVDEIGVGYVYDSGSVSYYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 94 - protein, 167 aa, artificial sequence
  HIV1_3.3 I-CreI variant
MANTKYNKEFLLYLAGFVDGDGSIIAQIKPSQSQKFKHQLSLTFQVTQKTQRRWFLDKLVDEIGVGYVADSGSVSEYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 95 - DNA, 51 bp, artificial sequence
  HIV1_3.4 target oligonucleotide
tggcatacaagtttccacactgacgtacgtcagtgtggcaatcgtctgtca

SEQ ID NO: 96 - DNA, 51 bp, artificial sequence
  HIV1_3.6 target oligonucleotide
tggcatacaagtttccacactgactttagtcagtgtggcaatcgtctgtca

SEQ ID NO: 97 - protein, 167 aa, artificial sequence
  HIV1_3.4 I-CreI variant
MANTKYNKEFLLYLAGFVDGDGSIIAQIKPNQSYKFKHYLSLTFYVTQKTQRRWFLDKLVDEIGVGYVSDSGSVSRYVLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 98 - protein, 167 aa, artificial sequence
  HIV1_3.4 I-CreI variant
MANTKYNKEFLLYLAGFVDGDGSIIAQIKPRQSYKFKHELRLTFNVTQKTQRRWFLDKLVDEIGVGYVRDSGSVSRYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 99 - protein, 167 aa, artificial sequence
  HIV1_3.4 I-CreI variant
MANTKYNKEFLLYLAGFVDGDGSIIAQIKPRQSYKFKHELSLTFNVTQKTQRRWFLDKLVDEIGVGYVYDSGSVSRYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 100 - protein, 167 aa, artificial sequence
  HIV1_3.4 I-CreI variant
MANTKYNKEFLLYLAGFVDGDGSIIAQIKPNQSYKFKHYLSLTFYVTQKTQRRWFLDKLVDEIGVGYVSDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 101 - protein, 167 aa, artificial sequence
  HIV1_3.4 I-CreI variant
MANTKYNKEFLLYLAGFVDGDGSIIAQIKPNQDYKFKHYLSLTFYVTQKTQRRWFLDKLVDEIGVGYVSDSGSVSRYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 102 - protein, 167 aa, artificial sequence
  HIV1_3.4 I-CreI variant
MANTKYNKEFLLYLAGFVDGDGSIIAQIKPRQDYKFKHQLSLTFYVTQKTQRRWFLDKLVDEIGVGYVRDSGSVSRYELSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 103 - protein, 167 aa, artificial sequence
  HIV1_3.4 I-CreI variant
MANTKYNKEFLLYLAGFVDGDGSIIAQIKPRQDYKFKHYLSLTFNVTQKTQRRWFLDKLVDEIGVGYVRDSGSVSRYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 104 - protein, 167 aa, artificial sequence
  HIV1_3.4 I-CreI variant
MANTKYNKEFLLYLAGFVDGDGSIIAQIKPNQTYKFKHRLSLTFYVTQKTQRRWFLDKLVDEIGVGYVYDSGSVSRYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 105 - protein, 167 aa, artificial sequence
  HIV1_3.3 I-CreI variant
MANTKYNKEFLLYLAGFVDGDGSIIAQIKPNQKAKFKHQLSLTFKVTQKTRRRWFLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 106 - protein, 167 aa, artificial sequence
  HIV1_3.3 I-CreI variant
MANTKYNKEFLLYLAGFVDGDGSIIAQIKPNQKAKFKHQLSLTFKVTQKTQRRWILDKLVGEIGVGYVEDSGSVSNYRLSEIKTLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 107 - protein, 167 aa, artificial sequence
  HIV1_3.3 I-CreI variant
MANTKYNKEFLLYLAGFVDGDGSIIAQIKPNQKAKFKHQLSLTFKVTQKTQRRWLLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 108 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGSVSNYRLSEIKPLHNFLTQLQPFLRLKQKQANLALKIIEQLPSAKESPDKFLEVCTWVDQIAALNDSKTRKTTSETVRSVLDSLSEKKKSSPAAD

SEQ ID NO: 109 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGSVSNYRLSEIKPLHNFLTQLQPFLKLKQKQANLVLKIIEQLPSAKESPDKFLEVCTWVDQNAALNDSKTRKTTSETVRAVLDSLSEKKKSSPAAD

SEQ ID NO: 110 - protein, 167 aa, artificial sequence
  HIV1_3.3 I-CreI variant
MANTKYNKEFLLYLAGFVDGDGSIIAQIKPNQSSKFKHYLRLTFKVTQKTQRRWFLDKLVDEIGVGYVSDNGSVSDYILSEIKPLHNFLTQLQPFLKLKQKQVNLVLKIIEQLPSAKESPDKFLEVCTWVDQIAALNDSKTRKTTSETVRAVLDSLSEKKKSSPAAD

SEQ ID NO: 111 - protein, 167 aa, artificial sequence
  HIV1_3.3 I-CreI variant
MANTKYNKEFLLYLAGFVDGDGSIIAQIKPNQSSKFKHYLRLTFKVTQKTQRRWFLDKLVDEIGVGYVSDNGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 112 - protein, 167 aa, artificial sequence
  HIV1_3.3 I-CreI variant
MANTKYNKEFLLYLAGFVDGDGSIVAQIKPNQKAKYKHQLSLTFKVTQKTQRRWFLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 113 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGSVSNYRLSEVKPLRNFLTQLQPFLKLKQKQANLVLKIIEQLPSAKESPDKFLEVCTWVDQIAALNDSKTRKTTSETVRAVLDCLSEKKKSSPAAD

SEQ ID NO: 114 - protein, 167 aa, artificial sequence
  HIV1_3.3 I-CreI variant
MANTKYNKEFLLYLAGFVDGDGSIIAQIKPCQSCKFKHQLSLTFKVTQKTQRRWLLDKLVDEIGVGYVADSGSVSDYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 115 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGSVSNYRLSEIKPLHNFLTQLQPFLRLKQKQANLALKIIEQLPSAKESPDKFLEVCTWVDQIAALNDSKTRKTTSETVRAVLDRLSEKKKSSPAAD

SEQ ID NO: 116 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGTVSNYRLSEVKPLRNFLTQLQPFLKLKQKQANLVLKIIEQLPSAKESPDKFLEVCTWVDQIAALNDSKTRKTTSETVRAVLDCLSEKKKSSPAAD

SEQ ID NO: 117 - protein, 167 aa, artificial sequence
  HIV1_3.3 I-CreI variant
MANTKYNKEFLLYLAGFVDGDGSIIAQIKPNQKAKFKHQLSLTLKVTQKTQRRWLLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 118 - protein, 167 aa, artificial sequence
  HIV1_3.3 I-CreI variant
MANTKYNKEFLLYLAGFVDGDGSIIAQIKPNQKAKFKHQLSLTFKVTQKAQRRWFLDKLVDEIGVGYVEDSGSVSNYRLSEVKPLRNFLAQLQPFLKLKQKQANLVLKIIEQLPSAKESPDKFLEVCTWADQIAALNDSKTRKTTSETVRAVLDCLSEQKKSSPAAD

SEQ ID NO: 119 - protein, 167 aa, artificial sequence
  HIV1_3.3 I-CreI variant
MANTKYNKEFLLYLAGFVDGDGSIIAQIKPCQSCKFKHQLSLTFKVTQKTQRRWLLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 120 - protein, 167 aa, artificial sequence
  HIV1_3.3 I-CreI variant
MANTKYNKEFLLYLAGLVDGDGSIIAQIKPNQKAKFKHQLSLTLKVTQKTRRRWFLDKLVDEIGVGYVEDSGSVSNYRLSEVKPLHNFLTQLQPFLKLKQKQANLVLKIIEQLPSAKESPDKFLEVCTWVDQIAALNDSKTRKTTSETVRAVLDCPSEKKKSSPAAD

SEQ ID NO: 121 - protein, 167 aa, artificial sequence
  HIV1_3.3 I-CreI variant
MANTKYNKEFLLYLAGFVDGDGSIVAQIKPNQKAKFKHQLSLTFKVTQKTQRRWFLDKLVDEIGVGYVEDSGSVSNYRLSEVKPLRNLLTQLQPFLKLKQKQANLVLKIIEQLPSAKESPDKFLEVCTWVDQIAALNDSKTRKTTSETVRAVLDCLSEEKKSSPAAD

SEQ ID NO: 122 - protein, 167 aa, artificial sequence
  HIV1_3.3 I-CreI variant
MANTKYNEEFLLYLAGFVDGDGSIIAQIKPNQSSKFKHYLRLTFKVTQKTQRRWFLDKLVDEIGVGYVEDSGSVSYYRLSEIKPLHNFLTQLQPFLRLKQKQANLALKIIEQLPSAKESPDKFLEVCTWVDQIAALNDSKTRKTTSETVRAVLDSLSEKKKSSPAAD

SEQ ID NO: 123 - protein, 167 aa, artificial sequence; identical to SEQ ID NO: 105
  HIV1_3.3 I-CreI variant

SEQ ID NO: 124 - protein, 167 aa, artificial sequence
  HIV1_3.3 I-CreI variant
MASTKYNKEFLLYLAGFVDGDGSIIAQIKPNQKAKFKHQLSLTFKVTQKTQRRWFLDKLVDEIGVGYVEDSGSVSNYRLSEIKPLHNFLTQLQPFLKLKQKQANLVLKIIEQLPSAKESPDKFLEVCTWVDQNAALNDSKTRKTTSETVRAVLDSLSEKKKSSPAAD

SEQ ID NO: 125 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSEVKPLHNFLTQLQPFLKLKQKQANLALRIIEQLPSAKESPDKFLEVCTWVDQIAALNDSKTRKTTSETVRAVLDSLSEKKKSSPAAD

SEQ ID NO: 126 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGSVSNYRLSKIKPLHNFLTQLQPFLKLKQKQANLVLKIIEQLPSAKESPDKFLEVCTWVDQIAALNDSKTRKTTSETVRAVLDRLSEKKKSSPAAD

SEQ ID NO: 127 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGTVSNYRLSKIKPLHNFLTQLQPFLKLKQKQANLVLKIIEQLPSAKESPDKFLEVCTWADQIAALNDSKTRKTTSETVRAVLDCLSEQKKSSPAAD

SEQ ID NO: 128 - protein, 167 aa, artificial sequence
  HIV1_3.3 I-CreI variant
MANTKYDKEFLLYLAGFVDSDGSIIAQIKPNQKAKFKHQLSLTFKVTQKAQRRWFLDKLVDEIGVGYVEDSGSVSNYRLSEVKPLRNFLAQLQPFLKLKQKQANLVLKIIEQLPSAKESPDKFLEVCTWADQVAALNDSKTRKTTSETVRAVLDRLSEKKKSSPAAD

SEQ ID NO: 129 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGTVSNYRLSKIKPLHNFLAQLQPFLKLKQKQANLALKIIEQLPSAKESPDKFLEVCTWVDQIAALNDSKTRKTTSETVRAVLDSLSEKKKSSPAAD

SEQ ID NO: 130 - protein, 167 aa, artificial sequence
  HIV1_3.3 I-CreI variant
MANTKYNKEFLLYLAGFVDSDGSIIAQIKPNQKAKFKHQLSLTLKVTQKAQRRWFLDKLVDEIGVGYVEDSGSVSNYRLSEVKPLRNFLAQLQPFLKLKQKQANLVLKIIEQLPSAKESPDKFLEVCTWADQIAALNDSKTRKTTSETVRAVLDCLSEQKKSSPAAD

SEQ ID NO: 131 - protein, 167 aa, artificial sequence
  HIV1_3.3 I-CreI variant
MANTKYNKEFLLYLAGFVDGDGSIIAQIKPNQKAKFKHQLSLTFKVTQKTQRRWFLDKLVDEIGVGYVEDSGSVSNYRLSKIKPLHNFLTQLQPFLRLKQKQANLALKIIEQLPSAKESPDKFLEVCTWVDQVAALNDSKTRKTTSETVRAVLDCLSEKKKSSPAAD

SEQ ID NO: 132 - protein, 167 aa, artificial sequence
  HIV1_3.3 I-CreI variant
MANTKYNKEFLLYLAGFVDSDGSIIAQIKPNQKAKFKHQLSLTFKVTQKTQRRWFLDKLVDEIGVGYVEDSGTISNYRLSEVKPLRNFLTQLQPFLKLKQKQANLALKIIEQLPSAKESPDKFLEVCTWVDQIAALNDSKTRKTTSETVRAVLDSLSEKKKSSPAAD

SEQ ID NO: 133 - protein, 167 aa, artificial sequence
  HIV1_3.3 I-CreI variant
MANTKYNKEFLLYLAGFVDSDGSIIAQIKPCQSCKFKHQLSLTFKVTQKTQRRWLLDKLVDEIGVGYVEDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 134 - protein, 167 aa, artificial sequence
  HIV1_3.3 I-CreI variant
MANTKYNKEFLLYLAGFVDGDGSIIAQIKPNQKAKFKHQLSLTLKVTQKTQRRWLLDKLVDEIGVGYVEDSGSVSNYRLSKIKPLHNFLTQLQPFLKLKQKQANLVLKIIEQLPSAKESPDKFLEVCTWVDQIAALNDSKTRKTTSETVRAVLDCLSEKKKSSPAAD

SEQ ID NO: 135 - protein, 167 aa, artificial sequence
  HIV1_3.3 I-CreI variant
MANTKYNKEFLLYLAGFVDSDGSIIAQIKPNQKAKFKHQLSLTFKVTQKTQRRWFLDKLVDEIGVGYVEDSGTVSNYRLSKIKPLHNFLTQLRPFLRLKQKQANLALKIIEQLPSAKESPDKFLEVCTWVDQIAALNDSKTRKTTSETVRAVLDRLSEKKKSSPAAD

SEQ ID NO: 136 - protein, 167 aa, artificial sequence; identical to SEQ ID NO: 125
  HIV1_3.4 I-CreI variant

SEQ ID NO: 137 - protein, 167 aa, artificial sequence
  HIV1_3.4 I-CreI variant
MANTKYNKEFLLYLAGFVDGDGSIIAQIKPNQSYKFKHYLSLTFYVTQKTQRRWFLDKLVDEIGVGYVSDSGSVSRYVLSEIKPLHNFLTQLQPFLKLKQKQANLALKIIEQLPSAKESPDKFLEVCTWVDQVAALNDSKTRKTTSETVRAVLDSLSEKKKSSPAAD

SEQ ID NO: 138 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 139 - protein, 167 aa, artificial sequence
  HIV1_3.4 I-CreI variant
MANTKYNKEFLLYLAGFVDGDGSIIAQIKPRQSYKFKHELRLTFNVTQKTQRRWFLDKLVDEIGVGYVRDSGSVSRYNLSEIKPLHNFLTQLQPYLKLKQKQANLALKIIEQLPSAKESPDKFLEVCTWVDQIAALNDSKTRKTTSETVRAVLDSLSEKKKSSPAAD

SEQ ID NO: 140 - protein, 167 aa, artificial sequence
  HIV1_3.4 I-CreI variant
MANTKYNKEFLLYLAGFVDGDGSIIAQIKPRQSYKFKHELRLTFNVTQKTQRRWFLDKLVDEIGVGYVRDSGSVSRYNLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 141 - protein, 167 aa, artificial sequence
  HIV1_3.4 I-CreI variant
MANTKYNKEFLLYLAGFVDGDGSIIAQIKPNQSYKFKHYLSLTFYVTQKTQRRWILDKLVDEIGVGYVSDSGSVSRYVLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 142 - protein, 167 aa, artificial sequence
  HIV1_3.4 I-CreI variant
MANTKYNKEFLLYLAGFVDGDGSIIAQIKPNQSYKFKHYLSLTFYVTQKTQRRWFLDKLVDEIGVGYVSDSGSVSRYVLSGIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 143 - protein, 167 aa, artificial sequence
  HIV1_3.4 I-CreI variant
MANTKYNKEFLLYLAGFVDGDGSIIAQIKPRQSYKFKHELRLTFNVTQKTQRRWFLDKLVDEIGVGCVRDSGSVSRYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 144 - protein, 167 aa, artificial sequence
  HIV1_3.4 I-CreI variant
MANTKYNKEFLLYLAGFVDGDGSIIAQIKPNQSYKFKHYLSLTFYVSQKTQRRWFLDKLVDEIGVGYVSDSGSVSRYVLSEIKPLHNFLTQLQPFLRLKQKQANLALKIIEQLPSAKESPDKFLEVCTWVDQIAALNDSKTRKTTSETVRAVLDSLSEKKKSSPAAD

SEQ ID NO: 145 - protein, 167 aa, artificial sequence; identical to SEQ ID NO: 140
  HIV1_3.4 I-CreI variant

SEQ ID NO: 146 - protein, 167 aa, artificial sequence
  HIV1_3.4 I-CreI variant
MANTKYNKEFLLYLAGFVDGDGSIIAQIKPRQSYKFKHELRLTFNVTQKTQRRWFLDKLVDEIGGGYVRDSGSVSRYNLSEIKPLHNFLTQLQPFLKLKQKQANLALKIIEQLPTAKESPDKFLEVCTWVDQIAALNDSKTRKTTSETVRAVLGSLSEKKKSSPAAD

SEQ ID NO: 147 - protein, 167 aa, artificial sequence
  HIV1_3.4 I-CreI variant
MANTKYNKEFLLYLAGFVDGDGSIVAQIKPNQSYKFKHYLSLTLYVTQKTQRRWFLDKLVDEIGVGYVSDSGSVSRYVLSEIKPLHNFLTQLQPFLKLKQKQANLALKIIEQLPSAKESPDKFLEVCTWVDQIAALNDSKTRKTTSETVRAVLGSLSEKKRSSPAAD

SEQ ID NO: 148 - protein, 167 aa, artificial sequence
  HIV1_3.4 I-CreI variant
MANTIYNKEFLLYLAGFVDGDGSIIAQIKPNQSYKFKHYLSLTFYVTQKTQRRWILDKLVDEIGVGYVSDSGSVSRYVLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 149 - protein, 167 aa, artificial sequence
  HIV1_3.4 I-CreI variant
MANTKYNKEFLLYLAGFVDGDGSIIAQIKPNQSYKFKHYLCLTFYVSQKTQRRWFLDKLVDEIGVGYVSDSGSVSRYVLSEVKPLHNFLTQLRPFLRLKQKQANLALKIIEQLPSAKESPDKFLEVCTWVDQIAALNDSKTRKTTSETVRAVLDSLSEKKKSSPAAD

SEQ ID NO: 150 - protein, 167 aa, artificial sequence
  HIV1_3.4 I-CreI variant
MANTKYNKEFLLYLAGFVDGDGSIIAQIKPNQSCKFKHSLSLTFQVTQKTQRRWFLDKLVDEIGVGYVRDSGSVSNYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 151 - protein, 167 aa, artificial sequence
  HIV1_3.4 I-CreI variant
MANTKYNKEFLLYLAGFVDGDGSIIAQIKPRQSYRFKHELRLTFNVTQKTQRRWFLDKLVDEIGVGYVRDSGSVSRYNLSEIKPLHNFLTQLQPYLKLKQKQANLALKIIEQLPSAKESPDKFLEVCTWVDQIAALNDSKTRKTTSETVRAVLDSLSEKKKSSPAAD

SEQ ID NO: 152 - protein, 167 aa, artificial sequence
  HIV1_3.4 I-CreI variant
MANTKYNKEFLLYLAGFVDGDGSIIAQIKPNQSYKFKHYLSLTFYVTQKTQRRWILDKLVDEIGVGYVSDSGSVSRYVLSEIKPLHNFLTQLQPFLKLKQKQANLALKIIEQLPSAKESPDKFLEVCTWVDQIAALNDSKTRKTTSETVRAVLDSLSEKKKSFPAAD

SEQ ID NO: 153 - protein, 167 aa, artificial sequence
  HIV1_3.4 I-CreI variant
MANTKYNREFLLYLAGFVDGDGSIIAQIKPNQSYKFKHYLCLTFYVTQKTQRRWILDKLVDEIGVGYVSVSGSVSRYVLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 154 - protein, 167 aa, artificial sequence
  HIV1_3.4 I-CreI variant
MANTKYNKEFLLYLAGFVDGDGSIIAQIKPNQSYKFKHYLSLTFYVTQKTQRRWILDKLVDEIGVGYVSDSGSVSRYVLSEIKPLHNFLTQLQPFLKLKQKQANLALKIIEQLPSAKESPGKFLEVCTWVDQIAALNDSKTRKTTSETVRAVLDSLSEKKRSSPAAD

SEQ ID NO: 155 - protein, 167 aa, artificial sequence
  HIV1_3.4 I-CreI variant
MANTKYNKEFLLYLAGFVDGDGSIIAQIKPRQSYKFKHELRLTFNVTQKTQRRWFLDKLVDEIGVGYVRDSGSVSRYNLSEIKPLHNFLTQLQPFLKLKQKQANLALKIIEQLPSAKESPDKFLEVCTWVDQIAALNDSKTRKTTSETVRAVLDSLSKKKKSSPAAD

SEQ ID NO: 156 - protein, 167 aa, artificial sequence
  HIV1_3.4 I-CreI variant
MANTKYNKEFLLYLAGFVDGDGSIIAQIKPNQSYKFKHYLSLTFYVTQKTQRRWFLDKLVDEIGVGYVSDSGSVSRYVLSKIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 157 - protein, 167 aa, artificial sequence
  HIV1_3.4 I-CreI variant
MANTKYNKEFLLYLAGFVDGDGSIIAQISPNQSYKFKHQLKLTLLVTQKTQRRWFLDKLVDEIGVGYVRDNGSVSNYILSKIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 158 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSKIKPLHNFLTQLQPFLKLKQKQANLALKIIEQLPSAKESPDKFLEVCTWVDQVAALNDSKTRKTTSETVRAVLDSLSEKKKSSPAAD

SEQ ID NO: 159 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSKIKPLHNFLTQLQPYLKLKQKQANLALKIIEQLPSAKESPDKFLEVCTWVDQIAALNDSKTRKTTSETVRAVLDSLSEKKKSSPAAD

SEQ ID NO: 160 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSKIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 161 - protein, 167 aa, artificial sequence
  HIV1_3.4 I-CreI variant
MANTKYNKEFLLYLAGFVDGDGSIIAQIKPRQSYKFKHELRLTFYVTQKTQRRWFLDKLVDEIGVGYVSDSGSVSRYVLSEIKPLHNFLTQLQPFLKLKQKQANLALKIIEQLPSAKESPDKFLEVCTWVDQVAALNDSKTRKTTSETVRAVLDSLSEKKKSSPAAD

SEQ ID NO: 162 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSKIKPLHNFLTQLQPFLKLKQKQANLALRIIEQLPSAKESPDKFLEVCTWVDQVAALNDSKTRKTTSETVRAVLDSLSEKKKSSPAAD

SEQ ID NO: 163 - protein, 167 aa, artificial sequence
  HIV1_3.4 I-CreI variant
MANTKYNKEFLLYLAGFVDSDGSIIAQIKPNQSYKFKHYLSLTLYVTQKTQRRWFLDKLVDEIGVGYVSDSGSVSRYVLSEIKPLHNFLTQLQPFLKLKQKQANLALKIIEQLPSAKESPDKFLEVCTWVDQVAALNDSKTRKTTSETVRAVLDSLSEKKKSSPAAD

SEQ ID NO: 164 - protein, 167 aa, artificial sequence; identical to SEQ ID NO: 158
  HIV1_3.4 I-CreI variant

SEQ ID NO: 165 - protein, 167 aa, artificial sequence
  HIV1_3.4 I-CreI variant
MANTKYNKEFLLYLAGFVDGDGSIIAQIKPNQSYKFKHYLSLTLYVTQKTQRRWFLDKLVDEIGVGYVSDSGSVSRYVLSEIKPLHNFLTQLQPFLKLKQKQANLALKIIEQLPSAKESPDKFLEVCTWVDQVAALNDSKTRKTTSETVRAVLDSLSEKKKSSPAAD

SEQ ID NO: 166 - DNA, 51 bp, artificial sequence
  HIV1_4.3 target oligonucleotide
tggcatacaagtttccagcattctgtacagaatgctggcaatcgtctgtca

SEQ ID NO: 167 - DNA, 51 bp, artificial sequence
  HIV1_4.5 target oligonucleotide
tggcatacaagtttccagcattctggacagaatgctggcaatcgtctgtca

SEQ ID NO: 168 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVRDDGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 169 - protein, 167 aa, artificial sequence
  HIV1_4.3 I-CreI variant
MANTKYNKEFLLYLAGFVDGDGSIIAQIKPRQDYKFKHQLSLTFKVTQKTQRRWFLDKLVDEIGVGYVTDGGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 170 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 171 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVADSGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 172 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVADAGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 173 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVGDGGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 174 - protein, 167 aa, artificial sequence
  HIV1_4.3 I-CreI variant
MANTKYNKEFLLYLAGFVDGDGSIIAQIKPRQSYKFKHQLSLTFQVTQKTQRRWFLDKLVDEIGVGYVADSGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 175 - DNA, 51 bp, artificial sequence
  HIV1_4.4 target oligonucleotide
tggcatacaagtttcttgtcttatgtacataagacaagcaatcgtctgtca

SEQ ID NO: 176 - DNA, 51 bp, artificial sequence
  HIV1_4.6 target oligonucleotide
tggcatacaagtttcttgtcttatggacataagacaagcaatcgtctgtca

SEQ ID NO: 177 - protein, 167 aa, artificial sequence
  HIV1_4.4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 178 - protein, 167 aa, artificial sequence
  HIV1_4.4 I-CreI variant
MANTKYNKEFLLYLAGFVDGDGSIIAQIKPNQGTKFKHQLSLTFAVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 179 - protein, 167 aa, artificial sequence
  HIV1_4.4 I-CreI variant
MANTKYNKEFLLYLAGFVDGDGSIIAQIKPHQSMKFKHALSLTFAVTQKTQRRWFLDKLVDEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 180 - protein, 167 aa, artificial sequence
  HIV1_4.4 I-CreI variant
MANTKYNKEFLLYLAGFVDGDGSIIAQIKPNQATKFKHQLSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 181 - protein, 167 aa, artificial sequence
  HIV1_4.4 I-CreI variant
MANTKYNKEFLLYLAGFVDGDGSIIAQIKPNQRAKFKHQLSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 182 - protein, 167 aa, artificial sequence
  HIV1_4.4 I-CreI variant
MANTKYNKEFLLYLAGFVDGDGSIIAQIKPNQSTKFKHQLALTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 183 - protein, 167 aa, artificial sequence
  HIV1_4.4 I-CreI variant
MANTKYNKEFLLYLAGFVDGDGSIIAQIKPNQSGKFKHCLSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 184 - protein, 167 aa, artificial sequence
  HIV1_4.4 I-CreI variant
MANTKYNKEFLLYLAGFVDGDGSIIAQIAPNQSSKFKHRLKLTFNVTQKTQRRWFLDKLADEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 185 - protein, 167 aa, artificial sequence
  HIV1_4.4 I-CreI variant
MANTKYNKEFLLYLAGFVDGDGSIIAQIAPNQSSKFKHRLKLTFAVTQKTQRRWFLDKLVDEIGVGYVRDSGSVSYYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 186 - protein, 167 aa, artificial sequence
  HIV1_4.4 I-CreI variant
MANTKYNKEFLLYLAGFVDGDGSIIAQIKPHQSCKFKHQLSLTFAVTQKTQRRWFLDKLVDEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 187 - DNA, 51 bp, artificial sequence
  HIV1_4.2 target oligonucleotide
tggcatacaagtttccagcattctgtacataagacaagcaatcgtctgtca

SEQ ID NO: 188 - DNA, 51 bp, artificial sequence
  HIV1_4 target oligonucleotide
tggcatacaagtttccagcattctggacataagacaagcaatcgtctgtca

SEQ ID NO: 189 - protein, 167 aa, artificial sequence
  HIV1_4.3 I-CreI variant
MANTKYNKEFLLYLAGFVDGDGSIIAQIQPNQSYKFNHRLKLTFKVTQKTQRRWFLDKLVDEIGVGYVTDGGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 190 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGSVSNYILSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 191 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDKLVDEIGVGYVADSGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 192 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDKLVDEIGVGYVADDGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 193 - protein, 167 aa, artificial sequence
  HIV1_4.3 I-CreI variant
MANTKYNKEFLLYLAGFVDGDGSIIAQIQPNQSYKFKHRLKMTFKVTQKTQRRWLLDKLVDEIGVGYVTDGGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 194 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGSVSNYILSKIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 195 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGPVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 196 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVADDGSVSNYILSEIKPLHNFLTQLQPFLKLKQKQANLVLKIIEQLPSAKESPDKFLEVCTWVDQVAALNDSKTRKTTSETVRAVLDSLREKKKSSPAAD

SEQ ID NO: 197 - protein, 167 aa, artificial sequence
  HIV1_4.3 I-CreI variant
MANTKYNKEFLLYLAGFVDGDGSIIAQIQPNQSYKFKHRLKLTLKVTQKTQRRWFLDKLVDEIGVGYVTDGGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 198 - protein, 167 aa, artificial sequence; identical to SEQ ID NO: 170
  HIV1_4.3 I-CreI variant

SEQ ID NO: 199 - protein, 167 aa, artificial sequence
  HIV1_4.4 I-CreI variant
MANTKYNKEFLLYLAGFVDGDGSIIAQIKPHQSMKFKHALSLTFAVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSRSEKKKSSPAAD

SEQ ID NO: 200 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGSVSDYILSKIKPLHNFLTQLQPFLKLKQRQANLVLKIIEQLPTAKESPDKFLEVCTWVDQIAALNDSKTRKTTSETVRAVLDSLSEKKKSSPAAD

SEQ ID NO: 201 - protein, 167 aa, artificial sequence
  HIV1_4.3 I-CreI variant
MANTKYNKEFLLYLAGFVDGDGSIIAQIQPNQSYKFKHRLKMTFKVTQKTQRRWLLDKLVDEIGVGYVTDGGSVSDYILSEIKPLHNFLTQLQPFLKLKQKQANLVLKIIEQLPSAKESPDKFMEVCTWVDQVAALNDSKTRKTTSETVRAVLDSLSEKKKSSPAAD

SEQ ID NO: 202 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDKLVDEIGVGYVADSGSVSDYILSKIKPLHNFLSQLQPFLKLKQKQANLVLKIIERLPSAKESPDKFLEVCTWVDQVAALNDSKTRKTTSETVRAVLDSLSEKKKSSPAAD

SEQ ID NO: 203 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGSVSDYILSKIKPLHNFLTQLQPFLKLKQKQANLVLKIIEQLPTAKESPDKFLEVCTWVDQIAALNDSKTRKTTSETVRAVLDSLSEKKKSPPAAD

SEQ ID NO: 204 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDKLVDEIGVGYVTDGGSVSDYILSKIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 205 - protein, 167 aa, artificial sequence
  HIV1_4.3 I-CreI variant
MANTKYNKEFLLYLAGFVDGDGSIIAQIQPNQSYKFKHRLKLTFKVTQKTQRRWFLDKLVDEIGVGYVTDGGSVSDYILSKIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 206 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDNLVDEIGVGYVTDGGSVSDYILSEIKPLHNFLTQLQPFLKLKQKQANLVLKIIEQLPSAKESPDKFLEVCTWVDQVAALNDSKTRKTTSETVRAVLDSLSEKEKSSPAAD

SEQ ID NO: 207 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDKLVDGIGAGYVADDGSVSDYILSEIKPLHNFLTQLQPFLKLKQKQANLVLKIIEQLPSAKESPDRFLEVCTWVDQVAALNDSKTRKTTSETVRAVLDSLSEKKKSSPAAD

SEQ ID NO: 208 - protein, 167 aa, artificial sequence
  HIV1_4.3 I-CreI variant
MANTKYNKEFLLYLAGFVDGDGSIIAQIQPNQSYKFKHRLKLTFKVTQKTQRRWLLDKLVDEIGVGYVTDGGSVSDYILSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 209 - protein, 167 aa, artificial sequence
  HIV1_4.3 I-CreI variant
MANTKYNKEFLLYLAGFVDGDGSIIAQIQPNQSYKCKHRLKLTYKVTQKTQRRWFLDKLVDEVGVGYITDGGSVSDYILSEIKPLHNFLTQLQPFLKLKRKQANLVLKIIEQLPSAKESPDKFLEVCTWVDQVAALNDSKTRKTTSETVRAVLDSLSEKKKSSPAAD

SEQ ID NO: 210 - skipped

SEQ ID NO: 211 - protein, 167 aa, artificial sequence
  HIV1_4 I-CreI variant
MANTKYNKEFLLYLAGFVDSDGSIIAQIQPNQSYKFKHRLKLTFKVTQKTQRRWFLDKLVDEIGVGYVTDGGSVSNYILSKIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 212 - protein, 167 aa, artificial sequence
  HIV1_4 I-CreI variant
MANTKYNKEFLLYLAGFVDGDGSIIAQIQPNQSYKFKHRLKLTFKVTQKTQRRWLLDKLVDEIGVGYVTDGGSVSNYILSKIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 213 - protein, 167 aa, artificial sequence
  HIV1_4 I-CreI variant
MANTKYNKEFLLYLAGFVDSDGSIIAQIQPNQSYKFKHRLKLTFKVTQKTQRRWLLDKLVDEIGVGYVTDGGSVSNYILSEIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 214 - protein, 167 aa, artificial sequence
  HIV1_4 I-CreI variant
MANTKYNKEFLLYLAGFVDSDGSIIAQIQPNQSYKFKHRLKLTFKVTQKTQRRWFLDKLVDEIGVGYVTDGGSVSNYILSKIKPLHNFLTQLQPFLKLKQKQANLVLKIIEQLPTAKESPDKFLEVCTWVDQIAALNDSKTRKTTSEAVRAVLDSLSEKKKSPPAAD

SEQ ID NO: 215 - protein, 167 aa, artificial sequence
  HIV1_4 I-CreI variant
MANTKYNKEFLLYLAGFVDGDGSIIAQIQPNQSYKFKHRLKLTFKVTQKTQRRWLLDKLVDEIGVGYVTDGGSVSNYILSKIKPLHNFLTQLQPFLKLKQRQANLVLKIIEQLPTAKESPDKFLEVCTWVDQVAALNDSKTRKTTSETVRAVLDSLSEKKKSPPAAD

SEQ ID NO: 216 - protein, 167 aa, artificial sequence
  HIV1_4 I-CreI variant
MANTKYNKEFLLYLAGFVDSDGSIIAQIQPNQSYKFKHRLKMTFKVTQKTQRRWLLDKLVDEIGVGYVTDGGSVSNYILSKIKPLHNFLTQLQPFLKLKQKQANLVLKIIEQLPSAKESPDKFMEVCTWVDQVAALNDSKTRKTTSETVRAVLDSLSEKKKSSPAAD

SEQ ID NO: 217 - protein, 167 aa, artificial sequence
  HIV1_4 I-CreI variant
MANTKYNKEFLLYLAGFVDSDGSIIAQIQPNQSYKFKHRLKLAFKVTQKTQRRWLLDKLVDEIGVGYVTDGGSVSNYILSKIKPLHNFLTQLQPFLKLKQKQANLALKIIEQLPTAKESPDKFLEVCTWVDQIAALNDSKTRKTTSETVRAVLDSLSEKKKSSPAAD

SEQ ID NO: 218 - protein, 167 aa, artificial sequence
  HIV1_4 I-CreI variant
MANTKYNKEFLLYLAGFVDSDGSIIAQIQPNQSYKFKHRLKLSFKVTQKTQRRWLLDKLVDEIGVGYVTDGGSVSNYILSKIKPLHNFLTQLHPYLKLKQKQANLALKIIEQLPSAKESPDKFLEVCTWVDQIAALNDSKTRKTTSETVRAVLDSLSEKKKSSPAAD

SEQ ID NO: 219 - protein, 167 aa, artificial sequence
  HIV1_4 I-CreI variant
MANTKYNKEFLLYLAGFVDSDGSIIAQIQPNQSYKFKHRLKMTFKVTQKTQRRWLLDKLVDEIGVGYVTDGGSVSNYILSKIKPLHNFLTQLQPFLKLKQKQANLVLKIIEQLPTAKESPDKFLEVCTWVDQIAALNDSKTRKTTSETVRAVLDSLSEKKKSSPAAD

SEQ ID NO: 220 - protein, 167 aa, artificial sequence
  HIV1_4 I-CreI variant
MANTKYNKEFLLYLAGFVDSDGSIIAQIQPNQSYKFKHRLKLTFKVTQKTQRRWFLDKLVDEIGVGYVTDGGSVSNYILSEIKPLHNFLTQLQPFLKLKQKQANLVLKIIEQLPSAKESPDKFMEVCTWVDQVAALNDSKTRKTTSETVRAVLDSLSEKKKSSPAAD

SEQ ID NO: 221 - protein, 167 aa, artificial sequence
  HIV1_4.4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKRSSPAAD

SEQ ID NO: 222 - protein, 167 aa, artificial sequence
  HIV1_4.4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLRLKQKQANLVLKIIEQLPSAKESPDKFLEVCTWVDQIAALNDSKTRKTTSETVRAVLDSLSEKKKSSPAAD

SEQ ID NO: 223 - protein, 167 aa, artificial sequence
  HIV1_4.4 I-CreI variant
MANTKYNKEFLLYLAGFVDGDGSIIAQIKPNQATKFKHQLSLTFAVTQKTQRRWFLDKLVDEIGVGYVYDSGSVSYYRLSEIKPLHNFLTQLQPFLKLRQKQANLVLKIIEQLPSAKESPDKFLEVCTWADQIAALNDSKTRKTTSETVRAVLDSLSEMKKSSPAAD

SEQ ID NO: 224 - protein, 167 aa, artificial sequence
  HIV1_4.4 I-CreI variant
MANTKYNKEFLLYLAGFVDGDGSIIAHIKPHQSMKFKHALSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 225 - protein, 167 aa, artificial sequence
  HIV1_4.4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLKLKRKQANLVLKIIEQLPSAKESPDKFLEVCTWVDQIAALNDSKTRKTTSETVRAVLDSLSEKKKSSPAAD

SEQ ID NO: 226 - protein, 167 aa, artificial sequence
  HIV1_4.4 I-CreI variant
MANTKYNKEFLLYLAGFVDGDGSIIAQIKPNQATKFKHQLSLTFAVTQKTQRRWFLDRLVDEIGVGYVYDSGSVSYYRLSEIKPLHNFLTQLQPFLKLKQKQANLVLKIIEQLPSAKESPDKFLEACTWVDQVAALNDSKTRKTTSETVRAVLDSLSEKKKSSPAAD

SEQ ID NO: 227 - protein, 167 aa, artificial sequence
  HIV1_4.4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSERKKSSPAAD

SEQ ID NO: 228 - protein, 167 aa, artificial sequence
  HIV1_4.4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLKLKQKQANLVLKIIEQLPSARESPDKFLEVCTWVDQIAALNDSKTRKTTSETVRAVLDSLSEKKKSSPAAD

SEQ ID NO: 229 - protein, 167 aa, artificial sequence
  HIV1_4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLRPFLKLKQKQANLVLKIIEQLPSAKESPDKFLEVCTWVDQIAALNDSKTRKTTSETVRAVLDSLSEKKKSSPAAD

SEQ ID NO: 230 - protein, 167 aa, artificial sequence
  HIV1_4 I-CreI variant
MANTKYNKEFLLYQAGFVDSDGSIIAQIKPHQSMKFKHALSLTFAVTQKTQRRWFLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 231 - protein, 167 aa, artificial sequence
  HIV1_4 I-CreI variant
MANTKYNKEFLLYLAGFVDGDGSIIAQIKPHQSMKFKHALSLTFNVTQKTQRRWFLDKLVDEIGVGYVYDSGSVSYYRLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 232 - protein, 167 aa, artificial sequence; identical to SEQ ID NO: 177
  HIV1_4 I-CreI variant

SEQ ID NO: 233 - protein, 167 aa, artificial sequence
  HIV1_4 I-CreI variant
MANTKYNKEFLLYQAGFVDGDGSIIARIKPHQSMKFKHALSLTFAVTQKTQRRWFLDKLVDEIGVGYVYDSGSVSYYKLSEIKPLHNFLTQLRPFLKLKQKQANLVLKIIEQPPSAKESPDKFLEVCTWVDQVAALNDSKTRKTTSETVRAVLDSLSEKKKSSPAAD

SEQ ID NO: 234 - protein, 167 aa, artificial sequence
  HIV1_4 I-CreI variant
MANTKYNKEFLLYQAGFVDGDGSIIARIKPHQSMKFKHALSLTFNVTQKTQRRWFLDKLVDEIGVGYVYDSGSVSYYRLSEIKPLHNLLTQLQPFLKLKQKQANLVLKIIEQLPSAKESPDKFLEVCTWVDQIAALNDSKTRKTTSETVRAVLDSLSEKKKSSPAAD

SEQ ID NO: 235 - protein, 167 aa, artificial sequence
  HIV1_4 I-CreI variant
MANTKYNKEFLLYLAGFVDGDGSIIAQIKPHQSMKFKHALSLTFNVTQKTQRRWLLDKLVDEIGVGYVYDSGSVSYYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 236 - protein, 167 aa, artificial sequence
  HIV1_4 I-CreI variant
MANTKYNKEFLLYLAGFVDGDGSIIAQIKPHQSMKFKHALSLTFNVTQKTQRRWFLDKLVDEIGVGYVYDSGSVSYYKLSEIKPLHNLLTQLRPFLKLKQKQANLVLKIIEQLPSAKESPDKFLEVCTWVDQVAALNDSKTRKTTSETVRAVLDSLSEKKKSSPAAD

SEQ ID NO: 237 - protein, 167 aa, artificial sequence; identical to SEQ ID NO: 212
  HIV1_4 I-CreI variant

SEQ ID NO: 238 - protein, 167 aa, artificial sequence
  HIV1_4 I-CreI variant
MANTKYNKEFLLYQAGFVDGDGSIIARIKPHQSMKFKHALSLTFAVTQKTQRRWFLDKLVDEIGVGYVYDSGSVSYYRLSEIKPLHNFLTQLRPFLKLKQKQANLVLKIIEQLPSAKESPDKFLEVCTWVDQIAALNDSKTRKTTSETVRAVLDSLSEKKRSSPAAD

SEQ ID NO: 239 - DNA, 51 bp, artificial sequence
  HIV1_5.3 target oligonucleotide
tggcatacaagtttgctctattaggtacctaatagagccaatcgtctgtca

SEQ ID NO: 240 - DNA, 51 bp, artificial sequence
  HIV1_5.5 target oligonucleotide
tggcatacaagtttgctctattagatacctaatagagccaatcgtctgtca

SEQ ID NO: 241 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGYVYDQGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 242 - protein, 167 aa, artificial sequence
  HIV1_5.3 -CreI variant
MANTKYNKEFLLYLAGFVDGDGSIIAQIKPNQSCKFKHALSLTFNVTQKTQRRWFLDKLVDEIGVGYVHDSGSVSYYNLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 243 - DNA, 51 bp, artificial sequence
  HIV1_5.4 target oligonucleotide
tggcatacaagtttatctgctcctgtacaggagcagatcaatcgtctgtca

SEQ ID NO: 244 - DNA, 51 bp, artificial sequence
  HIV1_5.6 target oligonucleotide
tggcatacaagtttatctgctcctatacaggagcagatcaatcgtctgtca

SEQ ID NO: 245 - protein, 167 aa, artificial sequence
  HIV1_5.4 -CreI variant
MANTKYNKEFLLYLAGFVDGDGSIIAQIKPAQSSKFKHQLSLTFRVTQKTQRRWFLDKLVDEIGVGYVYDSGSVSNYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 246 - protein, 167 aa, artificial sequence
  HIV1_5.4 -CreI variant
MANTKYNKEFLLYLAGFVDGDGSIIAQIKPQQSGKFKHQLSLTFKVTQKTQRRWFLDKLVDEIGVGYVYDSGSVSNYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 247 - protein, 167 aa, artificial sequence
  HIV1_5.4 -CreI variant
MANTKYNKEFLLYLAGFVDGDGSIIAQIKPQQSTKFKHQLSLTFKVTQKTQRRWFLDKLVDEIGVGYVYDSGSVSNYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 248 - protein, 167 aa, artificial sequence
  HIV1_5.4 -CreI variant
MANTKYNKEFLLYLAGFVDGDGSIIAQIKPSQSNKFKHQLSLTFKVTQKTQRRWFLDKLVDEIGVGYVTDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 249 - protein, 167 aa, artificial sequence
  HIV1_5.4 -CreI variant
MANTKYNKEFLLYLAGFVDGDGSIIAQIKPSQSNKFKHQLSLTFRVTQKTQRRWFLDKLVDEIGVGYVYDSGSVSNYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 250 - protein, 167 aa, artificial sequence
  HIV1_5.4 -CreI variant
MANTKYNKEFLLYLAGFVDGDGSIIAQIKPSQSNKFKHQLSLTFRVTQKTQRRWFLDKLVDEIGVGYVYDSGSVSNYNLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 251 - protein, 167 aa, artificial sequence
  HIV1_5.4 -CreI variant
MANTKYNKEFLLYLAGFVDGDGSIIAQIKPSQSTKFKHQLSLTFKVTQKTQRRWLLDKLVDEIGVGYVYDSGSVSNYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 252 - protein, 167 aa, artificial sequence
  HIV1_5.4 -CreI variant
MANTKYNKEFLLYLAGFVDGDGSIIAQIKPTQSGKFKHQLSLTFKVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAALDSLSEKKKSSPAAD

SEQ ID NO: 253 - protein, 167 aa, artificial sequence
  HIV1_5.4 -CreI variant
MANTKYNKEFLLYLAGFVDGDGSIIAQIKPNQSSKFKHQLSLTFKVTQKTQRRWFLDKLVDEIGVGYVYDSGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 254 - DNA, 51 bp, artificial sequence
  HIV1_5.2 target oligonucleotide
tggcatacaagtttgctctattaggtacaggagcagatcaatcgtctgtca

SEQ ID NO: 255 - DNA, 51 bp, artificial sequence
  HIV1_5 target oligonucleotide
tggcatacaagtttgctctattagatacaggagcagatcaatcgtctgtca

SEQ ID NO: 256 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGYVYDQGSVSNYILSEIKPLHNFLAQLQPFLKLKQKQANLVLKIIEQLPSAKESPDKFLEVCTWVDQIAALNDSKTRKTTSETVRAVLDSLSEKKKSSPAAD

SEQ ID NO: 257 - protein, 167 aa, artificial sequence
  HIV1_5.3 -CreI variant
MANTKYNKEFLLYLAGFVDGDGSIIAQIKPNQSCKFKHALSLTFNVTQKTQRRWFLDKLVDEIGVGYVHDSGSVSYYNLSKIKPLHNFLTQLQPFLKLKQKQASLVLKIIEQLPSAKESPDKFLEVCTWVDQIAALNDSKTRKTTSETVRAVLDSLSEKKKSSPAAD

SEQ ID NO: 258 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVEEIGVGYVYDQGSVSNYILSEIKPLHNFLTQLQPFLKLKQKQADLVLKIIEQLPSAKESPDKFLEVCTWVDQIAALNDSKTRKTTSETVRAVLDSLSEKKKSSPAAD

SEQ ID NO: 259 - protein, 167 aa, artificial sequence; identical to SEQ ID NO: 242
  HIV1_5.3 -CreI variant

SEQ ID NO: 260 - protein, 167 aa, artificial sequence
  HIV1_5.3 -CreI variant
MANTKYNKEFLLYLAGFVDGDGSIIAQIKPNQSCKFKHALSLTFNVTQKTQRRWFLDKLADEIGVGYVHDSGSVSYYNLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 261 - protein, 167 aa, artificial sequence
  HIV1_5.3 -CreI variant
MANTKYNKEFLLYLAGFVDGDGSIIAQIKPNQSCKFKHYLSLTLAVTQKTQRRWFLDKLVDEIGVGYVYDQGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 262 - protein, 167 aa, artificial sequence
  HIV1_5.3 -CreI variant
MANTKYNKEFLLYLAGFVDGDGSIIAQIKPNQSCKFKHYLSLTFAVTQKTQRRWLLDKLVDEIGVGYVYDQGSVSNYILSEIKPLHNFLTQLQPFLKLKQKQANLVLKIIEQLPSAKGSPDKFLEVCTWVDQIAALNDSKTRKTTSETVRAVLDSLSEKKKSSPAAD

SEQ ID NO: 263 - protein, 167 aa, artificial sequence; identical to SEQ ID NO: 241
  HIV1_5.3 -CreI variant

SEQ ID NO: 264 - protein, 167 aa, artificial sequence
  HIV1_5.3 -CreI variant
MANTKYSKEFLLYLAGFVDGDGSIIAQIKPSQSCKFKHYLSLTFAVTQKTQRRWFLDKLVDEIGVGYVHDSGSVSYYNLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 265 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGYVYDQGYVSNYILSEIKPLHNFLTQLQPFLKLKQKQANLVLRIIEQLPSAKESPDKFLEVCTWVDQIAALNDSKTRKTTSETVRAVLDSLSEKKKSSPAAD

SEQ ID NO: 266 - protein, 167 aa, artificial sequence
  HIV1_5.3 -CreI variant
MANTKYNKEFLLYLAGFVDGDGSIIAQIKPNQSCKFKHYLSLTFAVTQKTRRRWFLDKLVEEIGVGYVYDQGSVSNYILTEIKPLRNFLTQLQPFLKLKQKQADLVLKIIEQLPSAKESPDKFLEVCTWVDQIAALNDSKTRKTTSETVRAVLDSLSEKKKSSPAAD

SEQ ID NO: 267 - protein, 167 aa, artificial sequence
  HIV1_5.3 -CreI variant
MANTKYNKEFLLYLAGFVDGDGSIIAQIKPNQSCKFKHALSLTFNVTQKTQRRWFLDKLVDEIGVGYVHDSGSVSYYNLSKIKPLHNFLTQLQPFLKLKQKQANLVLKIIEQLPSAKESPDKFLEVCTWVDQIAALNDSKTRKTTSETVRAVLDSLSEKKKPSPAAD

SEQ ID NO: 268 - protein, 167 aa, artificial sequence
  HIV1_5.3 -CreI variant
MANTKYNKEFLLYLAGFVDGDGSIFAQIKPNQSCKFKHYLSLTFAVTQKTQRRWFLDKLVDEIGVGYVYDQGYVSNYILSEIKPLHNFLTQLQPFLKLKQKQANLVLRIIEQLPSAKESPDKFLEVCTWVDQIAALNDSKTRKTTSETVRAVLYSLSEKKKSSGGAD

SEQ ID NO: 269 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGHVYDQGYVSNYILSEIKPLHNFLTQLQPFLKLKQKQANLVLKVIEQLPSAKESPDKFLEVCTWVDQIAALNDSKTRKTTSETVRAVLDSLSEKKKSSPAAD

SEQ ID NO: 270 - protein, 167 aa, artificial sequence
  HIV1_5.3 -CreI variant
MANTKYSKEFLQYLAGFVDGDGSIIAQIKPSQSCKFKHYLSLTFAVTQKTQRRWFLDKLVDEIGVGYVYDQGSVSNYILSEIKPLHNFLAQLQPFLKLKQKQANLVLKIIEQLPSAKESPDKFLEVCTWVDQIAALNDSKTRKTTSETVRAVLDSLSEKKKSSPAAD

SEQ ID NO: 271 - protein, 167 aa, artificial sequence
  HIV1_5.3 -CreI variant
MANTKYNKEFLLYLAGFVDGDGSIIAQIKPNQSCKFKHALSLTFNVTQKTQRRWFLDKLVDEIGVGYVHDSGSVSYYNLSKIKPLHNFLTQLQPFLKLKQKQASLVLKIIEQLPSAKESPDKFLEVCTWVDQVAALNDSKTRKTTSETVRAVLDSLSEKKKSSPAAD

SEQ ID NO: 272 - protein, 167 aa, artificial sequence; identical to SEQ ID NO: 256
  HIV1_5.3 -CreI variant

SEQ ID NO: 273 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGYVYDQGYVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 274 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVEEIGVGYVYDQGSVSNYILSEIKPLHNFLTQLQPFLKLKQKQADLVLKIIEQLPSAKESPDKFLEVCTWVDQIAALNDSKTRKTTSETVRAVLDSLSVKKKSSPAAD

SEQ ID NO: 275 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGYVYDQGSVSNYILSEIKPLHNFLAQLQPFLKLKQKQANLVLKIIEQLPTAKESPDKFLEVCTWVDQIAALNDSKTRKTTSETVRAALDSLSEKKKSSPAAD

SEQ ID NO: 276 - protein, 167 aa, artificial sequence
  HIV1_5.4 -CreI variant
MANTKYNKEFLLYLAGFVDGDGSIIAQIKPSQSNKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQIAALNDSKTRKTTSETVRAVLDSLSEKKKSSPAAD

SEQ ID NO: 277 - protein, 167 aa, artificial sequence
  HIV1_5.3 -CreI variant
MANTKYSKEFLLYLAGFVDGDGSIIAQIKPSQSCKFKHYLSLTFAVTQKTRRRWFLDKLVEEIGVGYVYDQGSVSNYILTEIKPLRNFLTQLQPFLKLKQKQANLVLKVIEQLPSAKESPDKFLEVCTWVDQIAALNDSKTRKTTSETVRAVLDSLSEKKKSSPAAD

SEQ ID NO: 278 - protein, 167 aa, artificial sequence; identical to SEQ ID NO: 266
  HIV1_5.3 -CreI variant

SEQ ID NO: 279 - protein, 167 aa, artificial sequence
  HIV1_5.3 -CreI variant
MANTKYNKEFLLYLAGFVDSDGSIIAQIKPNQSCKFKHYLSLTFAVTQKTRRRWFLDKLVEEIGVGYVYDQGSVSNYILTEIKPLRNFLTQLQPFLKLKQKQANLVLKVIEQLPSAKESPDKFLEVCTWVDQIAALNDSKTRKTTSETVRAVLDSLSEKKKSSPAAD

SEQ ID NO: 280 - protein, 167 aa, artificial sequence
  HIV1_5.3 -CreI variant
MANTKYNKEFLLYLAGFVDSDGSIIAQIKPNQSCKFKHYLSLTFAVTQKTQRRWFLDKLVDEIGVGYVYDQGSVSNYILTEIKPLRNFLTQLQPFLKLKQKQANLALKIIEQLPSAKESPDKFLEVCTWVDQIAALNDSKTRKTTSETVRAVLDSLSEKKKSSPAAD

SEQ ID NO: 281 - protein, 167 aa, artificial sequence
  HIV1_5.3 -CreI variant
MANTKYSKEFLQYLAGFVDSDGSIIAQIKPSQSCKFKHYLSLTFAVTQKTQRRWFLDKLVDEIGVGYVYDQGSVSNYILSEIKPLHNFLAQLQPFLKLKQKQANLALKIIEQLPSAKESPDKFLEVCTWVDQVAALNDSKTRKTTSETVRAVLDSLSEKKKSSPAAD

SEQ ID NO: 282 - protein, 167 aa, artificial sequence
  HIV1_5.3 -CreI variant
MANTKYNKEFLLYLAGFVDGDGSIIAQIKPNQSCKFKHYLSLTFAVTQKTRRRWFLDKLVEEIGVGYVYDQGSVSNYILTEIKPLRNFLTQLQPFLKLKQKQANLALKIIEQLPSAKESPDKFLEVCTWVDQIAALNDSKTRKTTSETVRAVLDSLSEKKKSSPAAD

SEQ ID NO: 283 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGHVYDQGSGSNYILSEIKPLHNFLAQLQPFLKLKQKQANLALKIIEQLPSAKESPDKFLEVCTWVDQIAALNDSKTRKTTSETVRAVLDSLSEKKKSSPAAD

SEQ ID NO: 284 - protein, 167 aa, artificial sequence
  HIV1_5.3 -CreI variant
MANTKYNKEFLLYLAGFVDSDGSIIAQIKPNQSCKFKHYLSLTFAVTQKTRRRWFLDKLVEEIGVGYVYDQGSVSNYILTEIKPLRNFLTQLQPFLKLKQKQADLVLKIIEQLPSAKESPDKFLEVCTWVDQIAALNDSKTRKTTSETVRAVLDSLSEKKKSSPAAD

SEQ ID NO: 285 - protein, 167 aa, artificial sequence
  HIV1_5.3 -CreI variant
MANTKYSKEFLQYLAGFVDSDGSIIAQIKPNQSCKFKHYLSLTFAVTQKTQRRWFLDKLVDEIGVGHVYDQGYVSNYILSEIKPLHNFLTQLQPFLKLKQKQADLVLKVIEQLPSAKESPDKFLEVCTWVDQIAALNDSKTRKTTSETVRAVLDSLSEKKKSSPAAD

SEQ ID NO: 286 - protein, 167 aa, artificial sequence
  HIV1_5.3 -CreI variant
MANTKYNKEFLLYLAGFVDGDGSIIAQIKPNQSCKFKHYLSLTFAVTQKTQRRWFLDKLVDEIGVGYVYDQGSVSNYILTEIKPLRNFLTQLQPFLKLKQKQANLALKIIEQLPSAKESPDKFLEVCTWVDQIAALNDSKTRKTTSETVRAVLDSLSEKKKSSPAAD

SEQ ID NO: 287 - skipped

SEQ ID NO: 288 - protein, 167 aa, artificial sequence
  HIV1_5.4 -CreI variant
MANTKYNKEFLLYLAGFVDGDGSIIAQIKPSQSNKFKHQLSLTFRVTQKTQRRWILDKLVDEIGVGYVYDSGSVSNYQLSEIKPLHNFLTQLQPFLKLKQKQANLVLKIIEQLPSAKESPDKFLVVCTWVDQIAALNDSKTRKTTSETVRAVLDSLSEKKKSSPAAD

SEQ ID NO: 289 - protein, 167 aa, artificial sequence
  HIV1_5.4 -CreI variant
MANTKYNKEFLLYLAGFVDGDGSIIAQIKPAQSSKFKHQLSLTFRVTQKTQRRWFLDKLVDEIGVGHVYDSGSVSNYNLSEIKPLHNFLSQLQPFLKLKQKQANLVLKIIEQLPSAKESPDKFLEVCTWVDQIAALNDSKTRKTTSETVRAVLDSLSEKKKSSPAAD

SEQ ID NO: 290 - protein, 167 aa, artificial sequence
  HIV1_5.4 -CreI variant
MANTKYNKEFLLYLAGFVDGDGSIIAQIKPNQSSKFKHQLSLTLKVTQKTQRRWLLDKLVDEIGVGYVYDSGSVSYYILSEIKPLHNFLTQLRPFLKLKQKQANLVLKIIEQLPSAKESPDKFLEVCTWVDQIAALNDSKTRKTTSETVRAVLDSLSEKKKSSPAAD

SEQ ID NO: 291 - protein, 167 aa, artificial sequence
  HIV1_5.4 -CreI variant
MANTKYNKEFLLYLAGFVDGDGSIIAQIKPSQSNKFKHQLSLTFRVTQKTQRRWFLNKLVDEIGVGYVYDSGSVSNYNLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 292 - protein, 167 aa, artificial sequence
  HIV1_5.4 -CreI variant
MANTKYNKEFLLYLAGFVDGDGSIIAQIKPNQSSKFKHQLSLTLKVTQKTQRRWLLDKLVDEIGVGYVYDSGSVSYYILSEIRPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 293 - protein, 167 aa, artificial sequence
  HIV1_5.4 -CreI variant
MANTKYNKEFLLYLAGFVDGDGSIIAQIKPNQSSKFKHQLSLTFKVTQKTQRRWFLDKLVDEIGVGYVYDSGSVSNYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 294 - protein, 167 aa, artificial sequence
  HIV1_5.4 -CreI variant
MANTKYNKEFLLYLAGFVDGDGSIIAQIKPQQSTKFKHQLSLTFKVTQKTQRRWFLDKLVDEIGVGYVYDSGSVSDYRLSEIKSLHNFLTQLQPFLKLKQKQANLVLKIIEQLPSAKESPDKFLEVCTWVDQIAALNDSKTRKTTSETVRAALDSLSEKRKSSPAAD

SEQ ID NO: 295 - protein, 167 aa, artificial sequence
  HIV1_5.4 -CreI variant
MANTKYNKEFLLYLAGFVDGDGSIIAQIKPTQSGKFKHQLSLTFKVTQKTQRRWFLDKLVDEIGVGFVYDSGSVSDYRLSEIKPLHNFLTQLQPFLKLKQKQANLVLKIIEQLPSAKESPDKFLEVCTWVDQIAALNDSKTRKTTSETVRAALDSLSEKKKSSPAAD

SEQ ID NO: 296 - protein, 167 aa, artificial sequence
  HIV1_5.4 -CreI variant
MANTKYNKEFLLYLAGFVDGDGSIIAQIKPNQSSKFKHQLSLTFKVTQKTQRRWLLDKLVDEIGVGYVYDSGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 297 - protein, 167 aa, artificial sequence
  HIV1_5.4 -CreI variant
MANTKYSKEFLLYLAGFVDGDGSIIAQIKPNQSSKFKHQLSLTFRVTQKTQRRWILDKLVDEIGVGYVYDSGSVSNYQLSEIKPLHNFLTQLQPFLKLKQKQANLVLKIIEQLPSAKESPDKFLVVCTWVDQIAALNDSKTRKTTSETVRAVLDSLSERKKSSTAAD

SEQ ID NO: 298 - protein, 167 aa, artificial sequence; identical to SEQ ID NO: 276
  HIV1_5.4 -CreI variant

SEQ ID NO: 299 - protein, 167 aa, artificial sequence
  HIV1_5.4 -CreI variant
MANTKYNKEFLLYLAGFVDGDGSIIAQIKPSQSNKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQIAALNDSKTRRTTSETVRAVLDSLSEKKESSPAAD

SEQ ID NO: 300 - protein, 167 aa, artificial sequence; identical to SEQ ID NO: 288
  HIV1_5.4 -CreI variant

SEQ ID NO: 301 - protein, 167 aa, artificial sequence
  HIV1_5.4 -CreI variant
MANTKYNKEFLLYLAGFVDGDGSIIAQIKPNQSSKFKHQLSLTLKMTQKTQRRWLLDKLVDEIGVGHVYDSGSVSNYNLSEIKPLHNFLSQLQPFLKLKQKQANLVLKIIEQLPSAKESPDKFLEVCTWVDQIAALNDSKTRKTTSETVRAVLDSLSEKKKSSPAAD

SEQ ID NO: 302 - protein, 167 aa, artificial sequence
  HIV1_5.4 -CreI variant
MAYTKYNKEFLLYLAGLVDGDGSIIAQIKPSQSSKFKHQLSLTFRVTQKTQRRWFLDKLVDEIGVGHVYDSGSVSNYNLSEIEPLHNFLSQLQPFLKLKQKQASLVLKIIEQLPSAKESPDKFLEVCTWVDQIAALNDSKTRKTTSEAVRAVLDSLSEKKKSSPAAD

SEQ ID NO: 303 - protein, 167 aa, artificial sequence
  HIV1_5.4 -CreI variant
MANTKYNKEFLLYLAGFVDGDGSIIAQIKPNQSSKFKHQLSLTLKVTQKTQRRWLLDKLVDEIGVGYVYDSGSVSYYILSEIKPLHNFLTQLRPFLKLKQKQANLVLKIIEQLPYAKESPDKFLEVCTWVDQIAALNDSKTRKTTSETVRAVLDSLSEKKKSSPAAD

SEQ ID NO: 304 - protein, 167 aa, artificial sequence; identical to SEQ ID NO: 290
  HIV1_5.4 -CreI variant

SEQ ID NO: 305 - protein, 167 aa, artificial sequence
  HIV1_5.4 -CreI variant
MANTKYNKEFLLYLAGFVDGDGSIIAQIKPNQSSKFKHQLSLTLKVTQKTQRRWLLDKLVDEIGVGYVYDSGSVSYYILSEIKPLHNFLTQLRPFLKLKQKQANLVLKIIEQLPSAKESPDKFLEVCTWVDQIAALNDSKTRKTTSETVRAVLYSLSEKKKSSPAAD

SEQ ID NO: 306 - protein, 167 aa, artificial sequence
  HIV1_5.4 -CreI variant
MANTKYNKEFLLYLAGFVDGDGSIIAQIKPAQSSKFKHQLSLTFRVTQKTQRRWFLDKLVDEIGAGHVYDSGSVSNYNLSEIKPLHNFLSQLQPFLKLKQKQADLVLKIIEQLPSAKESPDKFLEVCTRVDQIAALNDSKTRKTTSVTVRAALDSLSEKKKSSPAAD

SEQ ID NO: 307 - protein, 167 aa, artificial sequence
  HIV1_5.4 -CreI variant
MANTKYNKEFLLYLAGFVDSDGSIIAQIKPNQSSKFKHQLSLTFRVTQKTQRRWILDKLVDEIGVGYVYDSGSVSNYQLSEIKPLHNFLTQLQPFLKLKQKQANLVLKIIEQLPSAKESPDKFLVVCTWVDQIAALNDSKTRKTTSETVRAVLDSLSERKKSSTAAD

SEQ ID NO: 308 - protein, 167 aa, artificial sequence
  HIV1_5.4 -CreI variant
MANTKYSKEFLLYLAGFVDSDGSIIAQIKPSQSNKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQIAALNDSKTRRTTSETVRAVLDSLSEKKESSPAAD

SEQ ID NO: 309 - protein, 167 aa, artificial sequence
  HIV1_5.4 -CreI variant
MANTKYNKEFLLYLAGFVDSDGSIIAQIKPSQSNKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQVAALNDSKTRRTTSETVRAVLDSLSEKKESSPAAD

SEQ ID NO: 310 - protein, 167 aa, artificial sequence
  HIV1_5.4 -CreI variant
MANTKYNKEFLLYLAGFVDGDGSIIAQIKPSRSSKFKHQLSLTFRVTQKTQRRWILDKLVDEIGVGYVYDSGSVSNYQLSEIKPLHNFLTQLQPFLKLKQKQANLVLKIIEQLPSAKESPDKFLVVCTWVDQIAALNDSKTRKTTSETVRAVLDSLSERKKSSTAAD

SEQ ID NO: 311 - protein, 167 aa, artificial sequence
  HIV1_5.4 -CreI variant
MANTKYNKEFLLYLAGFVDGDGSIIAQIKPSQSNKFKHQLSLTFKVTQKTQRRWILDKLVDEIGVGYVYDSGSVSNYQLSEIKPLHNFLTQLQPFLKLKQKQANLVLKIIEQLPSAKESPDKFLEVCTWVDQIAALNDSKTRKTTSETVRAVLDSLSERKKSSTTAD

SEQ ID NO: 312 - protein, 167 aa, artificial sequence
  HIV1_5.4 -CreI variant
MANTKYSKEFLLYLAGFVDSDGSIIAQIKPSQSNKFKHQLSLTFKVTQKTQRRWFLDKLVDEIGVGYVYDSGSVSDYRLSKIKPLHNFLAQLQPFLKLKQKQATLVLKIIEQLPSAKESPDKFLVVCTWVDQIAALNDSKTRKTTSETVRAVLDSLSERKKSSTAAD

SEQ ID NO: 313 - protein, 167 aa, artificial sequence
  HIV1_5.4 -CreI variant
MANTKYSKEFLLYLAGFVDSDGSIIAQIKPSQSNKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQIAALNDSKTRKTTSETVRAVLDSLSERKKSSTAAD

SEQ ID NO: 314 - protein, 167 aa, artificial sequence
  HIV1_5.4 -CreI variant
MANTKYNKEFLLYLAGFVDGDGSIIAQIKPSQSYKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQIAALNDSKTRKTTSETVRAVLDSLSEKKESSPAAD

SEQ ID NO: 315 - protein, 167 aa, artificial sequence
  HIV1_5.4 -CreI variant
MANTKYNKEFLLYLAGFVDSDGSIIAQIKPSQSNKFKHQLSLTFKVTQKTQRRWFLDKLVDEIGVGYVYDSGSVSDYRLSEIKPLHNFLTQLQPFLKLKQKQATLVLKIIEQLPSAKESPDKFLEVCTWVDQIAALNDSKTRRTTSETVRAVLDSLSEKKESSPAAD

SEQ ID NO: 316 - protein, 167 aa, artificial sequence
  HIV1_5.4 -CreI variant
MANTKYSKEFLLYLAGFVDSDGSIIAQIKPNQSSKFKHQLSLTFRVTQKTQRRWILDKLVDEIGVGYVYDSGSVSNYQLSEIKPLHNFLTQLQPFLKLKQKQANLALKIIEQLPSAKESPDKFLVVCTWVDRIAALNDSKTRKTTSETVRAVLDSLSERKKSSTAAD

SEQ ID NO: 317 - DNA, 51 bp, artificial sequence
  HIV1_3.2 target oligonucleotide
tggcatacaagtttctcagaccctgtacgtcagtgtggcaatcgtctgtca

SEQ ID NO: 318 - DNA, 51 bp, artificial sequence
  HIV1_3 target oligonucleotide
tggcatacaagtttctcagacccttttagtcagtgtggcaatcgtctgtca

SEQ ID NO: 319 - DNA, 22 bp, artificial sequence
  HIV1_1 target
cagaactacacaccagggccag

SEQ ID NO: 320 - DNA, 22 bp, artificial sequence
  HIV1_1.2 target
cagaactacgtaccagggccag

SEQ ID NO: 321 - DNA, 22 bp, artificial sequence
  HIV1_1.3 target
cagaactacgtacgtagttctg

SEQ ID NO: 322 - DNA, 22 bp, artificial sequence
  HIV1_1.4 target
ctggccctggtaccagggccag

SEQ ID NO: 323 - DNA, 22 bp, artificial sequence
  HIV1_1.5 target
cagaactacacacgtagttctg

SEQ ID NO: 324 - DNA, 22 bp, artificial sequence
  HIV1_1.6 target
ctggccctgacaccagggccag

SEQ ID NO: 325 - DNA, 22 bp, artificial sequence
  HIV1_3 target
tcagacccttttagtcagtgtg

SEQ ID NO: 326 - DNA, 22 bp, artificial sequence
  HIV1_3.2 target
tcagaccctgtacgtcagtgtg

SEQ ID NO: 327 - DNA, 22 bp, artificial sequence
  HIV1_3.3 target
tcagaccctgtacagggtctga

SEQ ID NO: 328 - DNA, 22 bp, artificial sequence
  HIV1_3.4 target
cacactgacgtacgtcagtgtg

SEQ ID NO: 329 - DNA, 22 bp, artificial sequence
  HIV1_3.5 target
tcagacccttttaagggtctga

SEQ ID NO: 330 - DNA, 22 bp, artificial sequence
  HIV1_3.6 target
cacactgactttagtcagtgtg

SEQ ID NO: 331 - DNA, 22 bp, artificial sequence
  HIV1_4  target
cagcattctggacataagacaa

SEQ ID NO: 332 - DNA, 22 bp, artificial sequence
  HIV1_4.2  target
cagcattctgtacataagacaa

SEQ ID NO: 333 - DNA, 22 bp, artificial sequence
  HIV1_4.3  target
cagcattctgtacagaatgctg

SEQ ID NO: 334 - DNA, 22 bp, artificial sequence
  HIV1_4.4  target
ttgtcttatgtacataagacaa

SEQ ID NO: 335 - DNA, 22 bp, artificial sequence
  HIV1_4.5  target
cagcattctggacagaatgctg

SEQ ID NO: 336 - DNA, 22 bp, artificial sequence
  HIV1_4.6  target
ttgtcttatggacataagacaa

SEQ ID NO: 337 - DNA, 22 bp, artificial sequence
  HIV1_5 target
ctctattagatacaggagcaga

SEQ ID NO: 338 - DNA, 22 bp, artificial sequence
  HIV1_5.2   target
ctctattaggtacaggagcaga

SEQ ID NO: 339 - DNA, 22 bp, artificial sequence
  HIV1_5.3 target
ctctattaggtacctaatagag

SEQ ID NO: 340 - DNA, 22 bp, artificial sequence
  HIV1_5.4 target
tctgctcctgtacaggagcaga

SEQ ID NO: 341 - DNA, 22 bp, artificial sequence
  HIV1_5.5 target
ctctattagatacctaatagag

SEQ ID NO: 342 - DNA, 22 bp, artificial sequence
  HIV1_5.6 target
tctgctcctatacaggagcaga

SEQ ID NO: 343 - DNA, 24 bp, artificial sequence
  C1221 target
tcaaaacgtcgtacgacgttttga

SEQ ID NO: 344 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 345 - protein, 32 aa, artificial sequence
  RM2 peptide linker
AAGGSDKYNQALSKYNQALSKYNQALSGGGGS

SEQ ID NO: 346 - protein, 354 aa, artificial sequence
  SCOH-HIV1_1-A
MANTKYNEEFLLYLAGFVDGDGSIIAQIKPNQSTKFKHQLKLTFRVTQKTQRRWFLDKLVDEIGVGYVYDSGSVSDYNLSEIKPLHNFLTQLQPFLELKQKQANLVLKIIEQLPSAKGSPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHRLSLTFVVTQKTQRRWLLDKLVDRIGVGYVEDRGSVSNYRLSKTKPLHNFLTQLQPFLKLKQKQANLVLKIIEQLPSAKESPDKFLEVCTWVDQVAALNDSKTRKTTSETVRAVLDSLSEKKKSSP

SEQ ID NO: 347 - protein, 354 aa, artificial sequence
  SCOH-HIV1_1-B
MANTKYNEEFLLYLAGFADGDGSIIAQIKPGQSYKFKHRLSLAFVVTQKTQRRWLLDKLVDEIGAGYVEDRGSVSNYRLSRIKPLHDFLTQLQPFLELKRKQANLVLKIIEHLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSTKFKHQLKLTFRVTQKTQRRWFLDKLVDRIGVGYVYDSGSVSDYNLSEIKPLHNFLTQLQPFLKLKQKQANLVLKIIEQLPSAKGSPDKFLEVCTWVDQVAALNDSKTRKTTSETVRAVLDSLSEKKKSSP

SEQ ID NO: 348 - protein, 354 aa, artificial sequence
  SCOH-HIV1_1-C
MANTKYNEEFLLYLAGFADGDGSIIAQIKPGQSYKFKHRLSLAFVVTQKTQRRWLLDKLVDEIGAGYVEDRGSVSDYRLSRIKPLHDFLTQLQPFLELKRKQANLVLKIIEHLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSTKFKHQLKLTFRVTQKTQRRWFLDKLVDRIGVGYVYDSGSVSDYNLSEIKPLHNFLTQLQPFLKLKQKQANLVLKIIEQLPSAKGSPDKFLEVCTWVDQVAALNDSKTRKTTSETVRAVLDSLSEKKKSSP

SEQ ID NO: 349 - protein, 354 aa, artificial sequence
  SCOH-HIV1_3-A
MANTKYNEEFLLYLAGFVDGDGSIIAQIKPNQKAKFKHQLSLTFKVTQKTQRRWFLDKLVDEIGVGYVEDSGTVSNYRLSKIKPLHNFLTQLQPFLELKQKQANLVLKIIEQLPSAKESPDKFLEVCTWADQVAALNDSKTRKTTSETVRAVLDCLSEQKKSSPAAGGSDKYNQALSKYNQALSKYNQALSGGGGSNKKFLLYLAGFVDSDGSIIAQIKPNQSYKFKHYLSLTLYVTQKTQRRWFLDKLVDRIGVGYVSDSGSVSRYVLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 350 - protein, 354 aa, artificial sequence
  SCOH-HIV1_3-B
MANTKYNEEFLLYLAGFVDGDGSIIAQIKPNQKAKFKHQLSLTFKVTQKTQRRWFLDKLVDEIGVGYVEDSGSVSNYRLSKIKPLHNFLTQLQPFLELKQKQANLVLKIIEQLPSAKESPDKFLEVCTWVDQVAALNDSKTRKTTSETVRAVLDRLSEKKKSSPAAGGSDKYNQALSKYNQALSKYNQALSGGGGSNKKFLLYLAGFVDSDGSIIAQIKPNQSYKFKHYLSLTLYVTQKTQRRWFLDKLVDRIGVGYVSDSGSVSRYVLSKIKPLHNFLTQLQPFLKLKQKQANLALKIIEQLPSAKESPDKFLEVCTWVDQVAALNDSKTRKTTSETVRAVLDSLSEKKKSSP

SEQ ID NO: 351 - protein, 354 aa, artificial sequence
  SCOH-HIV1_3-C
MANTKYNEEFLLYLAGFVDGDGSIIAQIKPNQKAKFKHQLSLTFKVTQKTQRRWFLDKLVDEIGVGYVEDSGTVSNYRLSKIKPLHNFLTQLQPFLELKQKQANLVLKIIEQLPSAKESPDKFLEVCTWADQVAALNDSKTRKTTSETVRAVLDCLSEQKKSSPAAGGSDKYNQALSKYNQALSKYNQALSGGGGSNKKFLLYLAGFVDSDGSIIAQIKPNQSYKFKHYLSLTLYVTQKTQRRWFLDKLVDRIGVGYVSDSGSVSRYVLSEIKPLHNLLTQLQPYLKLKQKQANLALKIIEQLPSAKESPDKFLEVCTWVDQVAALNDSKTRKTTSETVRAVLDSLSEKKKSSP

SEQ ID NO: 352 - protein, 354 aa, artificial sequence
  SCOH-HIV1_3-D
MANTKYNEEFLLYLAGFVDGDGSIIAQIKPNQKAKFKHQLSLTLKVTQKTQRRWLLDKLVDEIGVGYVEDSGSVSNYRLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSYKFKHYLSLTLYVTQKTQRRWFLDKLVDRIGVGYVSDSGSVSRYVLSEIKPLHNLLTQLQPYLKLKQKQANLALKIIEQLPSAKESPDKFLEVCTWVDQVAALNDSKTRKTTSETVRAVLDSLSEKKKSSP

SEQ ID NO: 353 - protein, 354 aa, artificial sequence
  SCOH-HIV1_3-E
MANTKYNEEFLLYLAGFVDGDGSIIAQIKPNQKAKFKHQLSLTFKVTQKTQRRWFLDKLVDEIGVGYVEDSGSVSNYRLSKIKPLHNFLTQLQPFLELKQKQANLVLKIIEQLPSAKESPDKFLEVCTWVDQVAALNDSKTRKTTSETVRAVLDRLSEKKKSSPAAGGSDKYNQALSKYNQALSKYNQALSGGGGSNKKFLLYLAGFVDSDGSIIAQIKPNQSYKFKHYLSLTLYVTQKTQRRWFLDKLVDRIGVGYVSDRGSVSDYVLSKIKPLHNFLTQLQPFLKLKQKQANLALKIIEQLPSAKESPDKFLEVCTWVDQVAALNDSKTRKTTSETVRAVLDSLSEKKKSSP

SEQ ID NO: 354 - protein, 354 aa, artificial sequence
  SCOH-HIV1_4-A
MANTKYNEEFLLYLAGFVDGDGSIIAQIKPHQSMKFKHALSLTFAVTQKTQRRWFLDKLVDEIGVGYVYDSGSVSYYRLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIQPNQSYKFKHRLKLTFKVTQKTQRRWFLDKLVDRIGVGYVTDGGSVSNYILSEIKPLHNFLTQLQPFLKLKQKQANLVLKIIEQLPSAKESPDKFMEVCTWVDQVAALNDSKTRKTTSETVRAVLDSLSEKKKSSP

SEQ ID NO: 355 - protein, 354 aa, artificial sequence
  SCOH-HIV1_5-A
MANTKYSEEFLLYLAGFVDGDGSIIAQIKPSQSCKFKHYLSLTFAVTQKTRRRWFLDKLVEEIGVGYVYDQGSVSNYILTEIKPLRNFLTQLQPFLELKQKQANLVLKVIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPSRSSKFKHQLSLTFRVTQKTQRRWILDKLVDRIGVGYVYDSGSVSNYQLSEIKPLHNFLTQLQPFLKLKQKQANLVLKIIEQLPSAKESPDKFLVVCTWVDQVAALNDSKTRKTTSETVRAVLDSLSERKKSST

SEQ ID NO: 356 - protein, 355 aa, artificial sequence
  SCOH-HIV1_5-B
MANTKYNEEFLLYLAGFVDGDGSIIAQIKPSQSCKFKHYLSLTFAVTQKTQRRWFLDKLVDEIGVGYVYDQGSVSNYILTEIKPLHNFLTQLQPFLELKQKQANLVLKVIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPSQSNKFKHQLSLTFKVTQKTQRRWILDKLVDRIGVGYVYDSGSVSNYQLSEIKPLHNFLTQLQPFLKLKQKQANLVLKIIEQLPSAKESPDKFLEVCTWVDQVAALNDSKTRKTTSETVRAVLDSLSERKKSSTT

SEQ ID NO: 357 - protein, 354 aa, artificial sequence
  SCOH-HIV1_7-A
MANTKYNEEFLLYLAGFVDGDGSIVAQIKPRQSYKFKHQLSLTFKVTQKTQRRWFLDKLVDEIGVGYVYGSGSVSYYSLSEIKPLHNFLTQLQPFLELKQEQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLHLAGFVDSDGSIIAQIKPNQSCKFKHQLQLTFRVTQRTQRRWFLDKLVDRIGVGYVYDSGSVSYYNLSGIKPLHNFLTQLQPFLKLKQKQANLALKIIEQLPSAKESPDKFLEVCTWVDQVAALNDSKTRKTTSETVRAVLDSLSEKKKSSP

SEQ ID NO: 358 - protein, 354 aa, artificial sequence
  SCOH-HIV1_7-B
MANTKYNEEFLLYLAGFVDGDGSIVAQIKPRQTYKFKHQLSLTFKVTQKTQRRWFLDKLVDEIGVGYVYDSGSVSYYSLSEIKPLHNFLIQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLHLAGFVDSDGSIIAQIKPNQSCKFKHQLQLTFRVTQKTQRRWFLDKLVDRIGVGYVYDSGSVSYYNLSEIKPLHNFLTQLQPFLKLKQKQANLALKIIEQLPSAKESPDKFLEVCTWVDQVAALNDSKTRKTTSETVRAVLDSLSEKKKSSP

SEQ ID NO: 359 - protein, 354 aa, artificial sequence
  SCOH-HIV1_8-A
MANTKYNEEFLLYLAGFVDGDGSIIARIKPRQSYKFKHQLSLTFRVGQKTQRRWFLDKLVDEIGVGYVNDSGSMSQYCLSEIKPLHNFLTQLQPFLELKQKQASLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIQPNQSYKFKHRLKLTFTVTQKTRRRWFLDKLVDRIGVGYVRDSGSVSYYILSEIKPLHNFLTQLQPFLKLKQKQANLVLKIIEQLPSAKESPDKFLEVCTWVDQVAALNDSKTRKTTSETVRAVLGSLSEKKKSSP

SEQ ID NO: 360 - protein, 354 aa, artificial sequence
  SCOH-HIV1_8-B
MASTKYNEEFLLYLAGFVDGDGSIIAQIQPNQSYKFKHRLKLTFQVTQKTQRRWFLDKLVDEIGVGYVHDSGSVSYYNLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPRQSYKFKHQLSLTFKVGQKTQRRWFLDKLVDRIGVGYVNDSGSMSQYCLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 361 - protein, 354 aa, artificial sequence
  SCOH-HIV1_8-C
MANTKYNEEFLLYLAGFVDGDGSIIAQIQPNQSYKFKHRLKLTFTVTQKTQRRWFLDKLVDEIGVGYVTDSGSVSYYRLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPRQSYKFKHQLSLTFKVGQKTQRRWFLDKLVDRIGVGYVNDSGSMSQYCLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 362 - protein, 354 aa, artificial sequence
  SCOH-HIV1_8-D
MANTKYNEEFLLYLAGFVDGDGSIIAQIQPNQSYKFKHRLKLTFTVTQKTRRRWFLDKLVDEIGVGYVRDSGSVSYYILSEIKPLHNFLTQLQPFLELKQKQANLVLKIIEQLPSAKESPDKFLEVCTWVDQVAALNDSKTRKTTSETVRAVLGSLSEKKKSSPAAGGSDKYNQALSKYNQALSKYNQALSGGGGSNKKFLLYLAGFVDSDGSIIAQIKPRQSYKFKHQLSLTFKVGQKTQRRWFLDKLVDRIGVGYVNDSGSMSQYCLSEIKPLHNFLTQLQPFLKLKQKQASLVLKIIEQLPSAKESPDKFLEVCTWVDQVAALNDSKTRKTTSETVRAVLDSLSEKKKSSP

SEQ ID NO: 363 - protein, 354 aa, artificial sequence
  SCOH-HIV1_9-A
MANTKYNEEFLLYLAGFVDGDGSIIAQIKPNRSHKFKHQLQLTFKVTQKTQRRWFLDKLVDEIGVGYVYDSGSVSEYVLSEIKPLHNFLTQLQPFLELKQKQANLVLKIIEQLPSAKESPDKFLEVCTWVDQVAALNDSRTRKTTSETVRAVLDNLSEKKKSSPAAGGSDKYNQALSKYNQALSKYNQALSGGGGSNKKFLLYLAGFVDSDGSIVAQIKPNQSYKFKHQLSLTFYVTQKTQRRWLLDKLVDRIGVGYVRDSGSVSQYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 364 - protein, 354 aa, artificial sequence
  SCOH-HIV1_9-B
MANTKYNEEFLLYLAGFVDGDGSIIAQIKPNRSHKFKHQLQLTFKVTQKTQRRWFLDKLVDEIGVGYVYDSGSVSEYVLSEIKPLHNFLTQLQPFLELKQKQANLVLKIIEQLPSAKESPDKFLEVCTWVDQVAALNDSRTRKTTSETVRAVLDNLSEKKKSSPAAGGSDKYNQALSKYNQALSKYNQALSGGGGSNKKFLLYLAGFVDSDGSIVAQIKPNQSYKFKHQLSLTFYVTQKTQRRWFLDKLVDRIGVGYVRDSGSVSQYVLSVIKPLHNLLTQLQPFLKLKQRQANLVLKIIEQLPSAKESPDKFLEVCTWVDQVAALNDSKTRKTTSETVRAVLDSLSEKKKSSP

SEQ ID NO: 365 - protein, 354 aa, artificial sequence
  SCOH-HIV1_9-C
MANTKYNEEFLLYLAGFVDGDGSIIAQIKPNRSHKFKHQLQLTFKVTQKTQRRWFLDKLVDEIGVGYVYDSGSVSEYVLSEIKPLHNFLTQLQPFLELKQKQANLVLKIIEQLPSAKGSPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIVAQIKPNQSYKFKHQLSLTFYVTQKTQRRWFLDKLVDRIGVGYVRDSGSVSQYVLSVIKPLHNLLTQLQPFLKLKQRQANLVLKIIEQLPSAKESPDKFLEVCTWVDQVAALNDSKTRKTTSETVRAVLDSLSEKKKSSP

SEQ ID NO: 366 - DNA, 22 bp, artificial sequence
  HIV1_7 target
ggagccaccccacaagatttaa

SEQ ID NO: 367 - DNA, 22 bp, artificial sequence
  HIV1_8 target
gggcccctaggaaaaagggctg

SEQ ID NO: 368 - DNA, 22 bp, artificial sequence
  HIV1_9 target
agaaatctgttgactcagattg

SEQ ID NO: 369 - DNA, 30 bp, artificial sequence
  HI8f primer
gacccggccataaagcaagagttttggctg

SEQ ID NO: 370 - DNA, 30 bp, artificial sequence
  HI8r primer
aagctctcttctggtggggctgttggctct

SEQ ID NO: 371 - DNA, 30 bp, artificial sequence
  adaptator sequence
ccatctcatccctgcgtgtctccgactcag

SEQ ID NO: 372 - DNA, 30 bp, artificial sequence
  adaptator sequence
cctatcccctgtgtgccttggcagtctcag

SEQ ID NO: 373 - protein, 9 aa, Artificial
  Protein motif
LAGLIDADG

SEQ ID NO: 374 - DNA, 22 bp, artificial sequence
  Description of Artificial Sequence: synthetic 10_CAG_P polynucleotide
ccagacgtcgtacgacgtctgg

SEQ ID NO: 375 - DNA, 22 bp, artificial sequence
  Description of Artificial Sequence: synthetic 10_ACA_P polynucleotide
cacaacgtcgtacgacgttgtg

SEQ ID NO: 376 - DNA, 22 bp, artificial sequence
  Description of Artificial Sequence: synthetic 10_TAG_P polynucleotide
ctagacgtcgtacgacgtctag

SEQ ID NO: 377 - DNA, 22 bp, artificial sequence
  Description of Artificial Sequence: synthetic 10_TCT_P polynucleotide
ctctacgtcgtacgacgtagag

SEQ ID NO: 378 - DNA, 22 bp, artificial sequence
  Description of Artificial Sequence: synthetic 10_CTG_P polynucleotide
cctgacgtcgtacgacgtcagg

SEQ ID NO: 379 - DNA, 22 bp, artificial sequence
  Description of Artificial Sequence: synthetic 10_TGG_P polynucleotide
ctggacgtcgtacgacgtccag

SEQ ID NO: 380 - DNA, 22 bp, artificial sequence
  Description of Artificial Sequence: synthetic 10_GGA_P polynucleotide
cggaacgtcgtacgacgttccg

SEQ ID NO: 381 - DNA, 22 bp, artificial sequence
  Description of Artificial Sequence: synthetic 10_AGA_P polynucleotide
cagaacgtcgtacgacgttctg

SEQ ID NO: 382 - DNA, 22 bp, artificial sequence
  Description of Artificial Sequence: synthetic 10_TGT_P polynucleotide
ctgtacgtcgtacgacgtacag

SEQ ID NO: 383 - DNA, 22 bp, artificial sequence
  Description of Artificial Sequence: synthetic 10_AGC_P polynucleotide
cagcacgtcgtacgacgtgctg

SEQ ID NO: 384 - DNA, 22 bp, artificial sequence
  Description of Artificial Sequence: synthetic 5_CCT_P polynucleotide
caaaaccctgtacagggttttg

SEQ ID NO: 385 - DNA, 22 bp, artificial sequence
  Description of Artificial Sequence: synthetic 5_GAC_P polynucleotide
caaaacgacgtacgtcgttttg

SEQ ID NO: 386 - DNA, 22 bp, artificial sequence
  Description of Artificial Sequence: synthetic 5_TAG_P polynucleotide
caaaactaggtacctagttttg

SEQ ID NO: 387 - DNA, 22 bp, artificial sequence
  Description of Artificial Sequence: synthetic 5_CTG_P polynucleotide
caaaacctggtaccaggttttg

SEQ ID NO: 388 - DNA, 22 bp, artificial sequence
  Description of Artificial Sequence: synthetic 5_TTC_P polynucleotide
caaaacttcgtacgaagttttg

SEQ ID NO: 389 - DNA, 22 bp, artificial sequence
  Description of Artificial Sequence: synthetic 5_TAC_P polynucleotide
caaaactacgtacgtagttttg

SEQ ID NO: 390 - DNA, 22 bp, artificial sequence
  Description of Artificial Sequence: synthetic 5_TCT_P polynucleotide
caaaactctgtacagagttttg

SEQ ID NO: 391 - DNA, 22 bp, artificial sequence
  Description of Artificial Sequence: synthetic 5_TAT_P polynucleotide
caaaactatgtacatagttttg

(19) United States
(12) Patent Application Publication
(10) Pub. No.: US 2012/0260356 A1
(43) Pub. Date: Oct. 11, 2012

(54) MEGANUCLEASE VARIANTS CLEAVING AT LEAST ONE TARGET IN THE GENOME OF A RETROVIRUS AND USES THEREOF

(75) Inventors: André Choulika, Paris (FR); Roman Galetto, Paris (FR)

(73) Assignee: CELLECTIS, Paris (FR)

(21) Appl. No.: 13/265,575

(22) PCT Filed: Apr. 21, 2010
(86) PCT No.: PCT/IB10/51746

§ 371 Date: Feb. 10, 2012

(30) Foreign Application Priority Data
Apr. 21, 2009 (IB) PCT/IB2009/005582

(51) Int. Cl. C12N 9/16; A61P 31/14; C12N 15/55; C12N 1/21; A01K 67/00; C12N 15/87; C12N 1/19; A61K 38/46; C12N 15/63

(52) U.S. Cl. 800/13; 435/196; 424/94.6; 536/23.2; 435/320.1; 435/440; 435/254.21; 435/252.33

(57) ABSTRACT

Meganuclease variants which cleave at least one target in the provirus of a retrovirus and in particular which cleave the genomic insertion of the provirus. The present invention in particular relates to meganuclease variants which cleave the provirus of the Human Immunodeficiency Virus genome following genomic insertion. Vector encoding such variants, as well as to a cell or multi-cellular organism modified by such a vector and use of said meganuclease variants and derived products for genome engineering and for in vivo and ex vivo (gene cell therapy) genome therapy.

The invention relates to the use of meganuclease variants which cleave at least one target in the provirus of a retrovirus and in particular cleave the genomic insertion of an integrating Virus genome and in particular to meganuclease variants which cleave the Human Immunodeficiency Virus genome following genomic insertion, for the treatment of an infection of one or more of these viruses. The present Invention also relates to such variants and to vectors encoding such variants, as well as to a cell or multi-cellular organism modified by such a vector and to the use of said meganuclease variant and derived products for genome engineering and for in vivo and ex vivo (gene cell therapy) genome therapy.

Viral infections of various sorts are a serious and continuing health, agricultural and economic problem worldwide. In particular viruses present specific treatment and control problems as they always comprise an intracellular stage to their life cycle, in which the nucleic acid genome of the virus is inserted into a host cell and normally transported to the nucleus. During this stage of the virus life cycle, the virus genome can enter into a dormant state whilst inside a host cell, during which time the production of new virus particles/proteins/copies of the viral genome ceases. These characteristics present a significant problem as most medicaments and treatments for viral infection consist of compounds which affect aspects of virus biology involved in the active stages of the virus life cycle, such as compounds which target/inactivate a viral enzyme or structural protein. Therefore whilst in a dormant state the viral genome resident in the cytoplasm or nucleus of a host cell can not be affected by most conventional anti-virus medicaments and therefore persists.

One group of viruses presents additional problems as they integrate into the host cell genome. This group, called retroviruses, like other viruses are transmitted via the infection of new host cells by virus particles and can also cause the endemic infection of the progeny cells of a host cell in which they are genomically integrated. This second mode of transmission, particularly when the retrovirus genome is dormant can result in the clonal expansion of the retrovirus containing cells, which in turn can cause significant problems once the retrovirus genomes activate.

The present invention therefore relates to Retroviruses which are contained with the family Retroviridae which comprises in turn seven genera. Alpharetrovirus, Betaretrovirus, Gammaretrovirus, Deltaretrovirus, Epsilonretrovirus, Lentivirus and Spumavirus. These groups of viruses are responsible for several important diseases such as Human T-lymphotrophic virus (Gammaretrovirus), Rous Sarcoma (Alpharetrovirus) and Human Immunodeficiency Virus (Lentivirus).

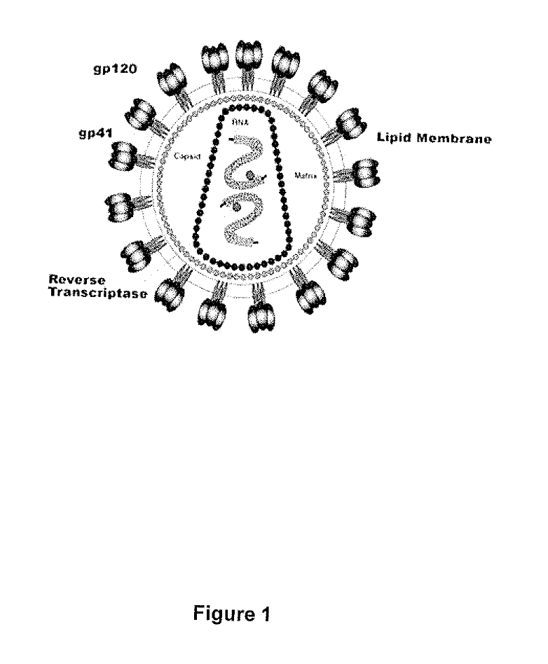

The Human Immunodeficiency Virus (HIV) (FIG. 1) is an example of a Retrovirus which is responsible for a significant and ongoing global medical crisis. HIV viruses persist and continue to replicate for many years in the infected individual before causing overt signs of disease. HIV is the causative agent of the Acquired Immune Deficiency Syndrome (AIDS), which is characterized by a susceptibility to infection with opportunistic pathogens, mainly as a result of a profound decrease in the number of CD4+ T cells. A characteristic feature of the Retroviridae family of viruses is that viral particles contain two copies of an RNA genome. After infection, the genomic RNA is reverse transcribed by a viral enzyme into DNA, which is then permanently integrated into the host genome.

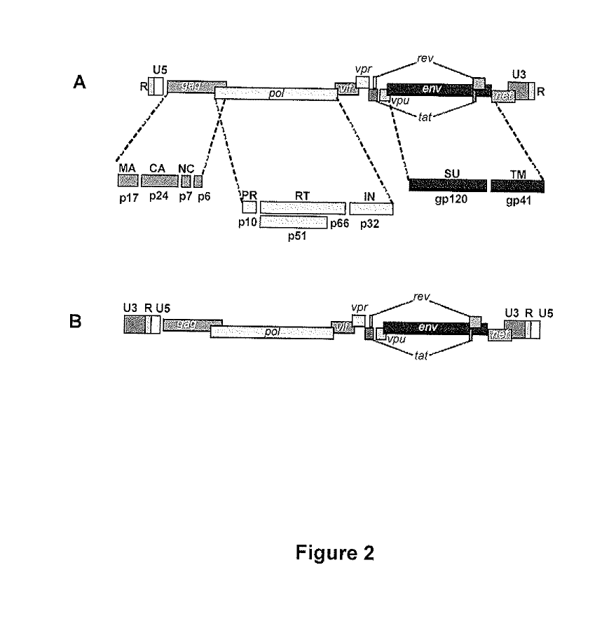

The retroviral genome harbors the sequences coding for the viral enzymatic, structural and regulatory proteins. In addition, the genomic RNA molecule contains a series of non-coding sequences that have important functions in different steps of the viral life cycle (FIG. 2).

The “2007 AIDS epidemic update” report, issued by the UNAIDS (Joint United Nations Programme on HIV/AIDS), indicates that 33.2 million [30.6-36.1 million] people were estimated to be living with HIV, 2.5 million [1.8-4.1 million] people became newly infected with HIV and 2.1 million [1.9-2.4 million] people died of AIDS in 2007.

HIV is characterized by a high genetic variability, due to the rapid viral turnover (10¹⁰-10¹² viral particles produced per day) in an HIV-infected individual, combined with the high mutation rate arising during reverse transcription (10⁻⁴ per nucleotide). Two types of HIV, HIV-1 and HIV-2, which are closely related to each other, have been identified to date (Sharp et al., Philos Trans R Soc Lond B Biol Sci, 2001, 356, 867-76). Most AIDS worldwide is caused by the more virulent HIV-1, while HIV-2 is endemic in West Africa. Both viruses appear to have spread to humans from other primate species and the best evidence from sequence relationships suggests that HIV-1 has passed to humans on at least three independent occasions from the chimpanzee, Pan troglodytes and HIV-2 from the sooty mangabey, Cercocebus atys.

The three zoonotic transmissions that generated the HIV-1 type viruses gave rise to three different viral groups: M, O and N. The M group (for main), represents the substantial majority of worldwide infections. The 0 (for outlier) and N (for non-M/non-O) groups remain essentially restricted to Central Africa (Sharp et al., Philos Trans R Soc Lond B Biol Sci, 2001, 356, 867-76).

HIV is transmitted by direct sexual contact, by blood or blood products, and from an infected mother to infant, either intrapartum, perinatally, or via breast milk. Infection of humans with HIV-1 causes a dramatic decline in the number of CD4+ T lymphocytes. When the number of CD4+ cells is very reduced, opportunistic infections and neoplasms occur (Simon et al., Lancet, 2006, 368, 489-504).

Antiretroviral treatment for HIV infection consists of drugs which work by slowing down the replication of HIV in the body. Currently, there are around 30 antiretroviral drugs approved to treat people infected with HIV in various countries around the world. There are several classes of anti-HIV drugs that attack the virus in different ways and the most common classes of antiretrovirals are nucleoside or nucleotide reverse transcriptase inhibitors, non-nucleoside reverse transcriptase inhibitors, protease inhibitors and entry inhibitors (Flexner C, Nature Reviews Drug Discovery, 2007, 6, 959-966).

People with HIV need to continuously take antiretroviral drugs. Furthermore for antiretroviral treatment to be effective for a long time, it has been found that more than one antiretroviral drug must be taken at a time as single drug treatment regimes invariably lead to HIV resistance to the single drug negating its therapeutic effects.

Combination Therapy, wherein at least two and normally three different medicaments are taken simultaneously prolongs the period of time before resistance develops for one or more of the medicaments. The term Highly Active Antiretroviral Therapy (HAART) is used to describe a combination of three or more anti-HIV drugs. HAART typically combines drugs from at least two different classes of antiretroviral drugs and has been shown to effectively suppress the virus when used properly. Highly active antiretroviral therapy has revolutionalized how people infected with HIV are treated, and reduces the rate at which resistance develops.

Normally when anti-HIV treatment is started, the viral load drops to an undetectable level. When drug resistance develops, the amount of HIV in the blood rises and the risk of the person becoming ill increases and this usually means that the drug regimen needs to be changed (Martinez-Cajas and Wainberg, Drugs, 2008, 68, 43-72).

Currently available HIV treatments have converted HIV infection into a chronic disease, increasing the lifespan of infected individuals. Anti-HIV drugs can reduce the rate of viral replication, retarding therefore the onset of AIDS. Nevertheless, the emergence of strains resistant to these existing treatments, require the continual development of new therapeutic strategies (Rossi et al., Nat. Biotechnol., 2007, 25, 1444-54). Although there are currently no vaccines to prevent or treat HIV, researchers are developing and testing several potential HIV vaccines, either for preventive and/or therapeutic purposes. However, vaccine development encounters the same problem as anti-HIV drugs concerning the rapid viral evolution and the subsequent development of resistance or in the case of a vaccine an evolved HIV strain which no longer comprises the epitope used in the vaccine and hence is not affected by the immune response elicited by the vaccine. At the present time the general consensus in the scientific and medical community is that therapeutic HIV vaccines will not be able to completely eliminate HIV infection, because the virus “hides” in certain cells of the body, where it can last silent for decades meaning that any effect of the vaccine will have been lost.

A new field for the treatment of HIV infection is the development of genetic therapies against HIV. Gene therapy could allow the prevention of progressive HIV infection by persistently blocking viral replication. Gene-targeting strategies are being developed with RNA-based agents such as ribozymes, aptamers and small interfering RNAs and protein-based agents. Among the last group, the use of zinc-finger nucleases against the CCR5 receptor, a protein present on the surface of immune cells that is required to mediate viral entry, is currently in Phase I clinical trials. In this case, the disruption of the CCR5 receptor from the immune cells by the nucleases is proposed to render the patient's cells permanently resistant to CCR5-specific strains of HIV. This approach is based on the fact that people with natural mutations on this receptor are resistant to HIV infection.

To date however the number of effective anti-HIV/retrovirus therapies is very small, due in part to the limited number of target genes/proteins/pathways present in the relatively simple retrovirus genome/life cycle as well as to the rapid creation of ‘escape’ mutants by the retrovirus during replication which allow members of the virus population to evade therapeutic compounds that more slowly evolving pathogens such as bacteria or protozoa would not be able to develop resistance to with the same speed.

In addition due to the existence of dormant intragenomic copies of the provirus which are not affected by any current therapy, the curing of HIV infection (AIDS) is currently simply not possible.

An interesting target that has not been pursued in the fight against the AIDS pandemic and more generally retroviruses is the genomically integrated provirus and/or the reverse transcribed DNA version of the retrovirus genome prior to its integration, since targeting the proviral DNA could lead to the elimination or inactivation of the structure that allows the virus to multiply and the infection to propagate. One novel way to inactivate the provirus which the inventors have decided to investigate is by the use of nucleases that could cleave the integrated form of the virus and generate mutations and/or deletions in the provirus following the action of the cellular DNA repair machinery.

An important point to be considered in this kind of approach is the choice of the target sequences. In a first instance, the target sequences should be located in the coding sequences of essential genes, since the inactivation of an accessory gene may not lead to viral eradication. The viral genome also contains essential regulatory sequences that are located in the long terminal repeats (LTRs) that flank the viral genome in the provirus. Even if mutations in these regions would be expected to have a less drastic effect than a mutation in an essential gene, the fact that they are duplicated sequences could be useful in an approach of “virus clipping”, meaning the excision of long regions of the proviral DNA by the action of a nuclease cleaving twice in the viral sequence. Another important point that should be considered is the degree of sequence variation that is observed in the target sequences among different circulating viral isolates. As discussed above HIV is characterized by a high degree of sequence variability due to the nature of the viral reverse transcriptase. It is therefore essential to check the sequence conservation of the target among the different isolates.

The inventors have developed a new molecular medicine approach based on the inactivation of the retrovirus provirus through the use of tailored meganucleases specifically targeting the proviral DNA, using the HIV-1 provirus in the genome of the infected cell as a model. The principle of this new therapeutic strategy is that the tailored meganucleases against targets in the provirus will generate a double strand break (DSB) at their target sequences, chosen to be located in genes/regulatory sequences/structural sequences that are essential for the virus to replicate or alternatively target sequences which are present in multiple copies in the provirus, for instance in the two flanking LTR regions, so allowing the provirus or a portion thereof to be excised.

The epidemiology of HIV, particularly in sub Saharan Africa, makes research into the HIV virus a major and extremely active area of research. The manipulation of the HIV provirus is one area of research in which to date reagents have not been readily available as workers have instead concentrated on attempting to manipulate the HIV virion per se. Therefore the means to easily engineer the HIV provirus in situ in the genome of an infected cell/organism would likely provide valuable insights into this aspect of HIV biology and potentially open new avenues of attack in combating HIV.

Even if the meganuclease targets have been selected following the criteria mentioned above, namely in essential genes and particularly in sequences showing the highest degree of conservation, the capacity of the virus to generate escape mutants under the selective pressure of a drug/therapy must be considered.

To minimize the effect of drug resistance(s), “Combination Therapy” has already been shown to counter act this feature of HIV biology. In the same way, the possibility of using a combination of meganucleases could help to prevent any resistance that could be generated during viral replication. In addition although HIV shows a very high level of genetic change, not all of the components of the HIV genome are as capable of supporting change as others. Generally speaking it is those portions of the virus which are immunogenic, that is present upon the exterior of the virus particle where they can interact with the components of the hosts immune system, which are most able to support high levels of variability. Whereas the essential internal structural or packaging components of HIV are less able to continue to function following changes in their coding sequences. These differences do not affect the ability of HIV to evolve so as to elude the host immune response, but have proven useful in specifically engineering drugs for which it is more difficult for HIV to develop resistance. The increased levels of conservation of some provirus sequences can also be used to further hone the meganuclease(s) according to the present invention.

In vivo meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffolds to derive novel, highly specific endonucleases.

HEs belong to four major families. The LAGLIDADG family (SEQ ID NO:373), named after a conserved peptide motif involved in the catalytic center, is the most widespread and the best characterized group. Seven structures are now available. Whereas most proteins from this family are monomeric and display two LAGLIDADG motifs (SEQ ID NO:373), a few have only one motif, and thus dimerize to cleave palindromic or pseudo-palindromic target sequences.

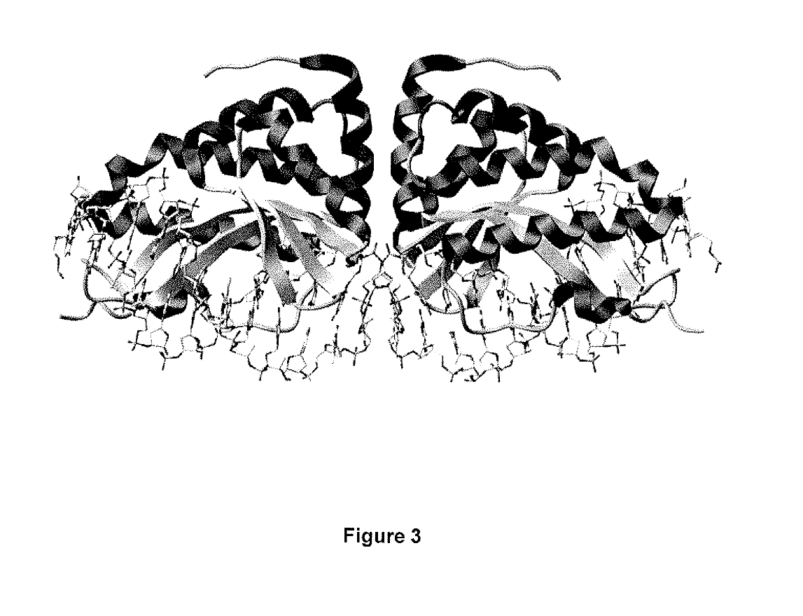

Although the LAGLIDADG peptide (SEQ ID NO:373) is the only conserved region among members of the family, these proteins share a very similar architecture (FIG. 3). The catalytic core is flanked by two DNA-binding domains with a perfect two-fold symmetry for homodimers such as I-CreI (Chevalier, et al., Nat. Struct. Biol., 2001, 8, 312-316), I-MsoI (Chevalier et al., J. Mol. Biol., 2003, 329, 253-269) and I-CeuI (Spiegel et al., Structure, 2006, 14, 869-880) and with a pseudo symmetry for monomers such as I-SceI (Moure et al., J. Mol. Biol., 2003, 334, 685-69, I-DmoI (Silva et al., J. Mol. Biol., 1999, 286, 1123-1136) or I-AniI (Bolduc et al., Genes Dev., 2003, 17, 2875-2888). Both monomers and both domains (for monomeric proteins) contribute to the catalytic core, organized around divalent cations. Just above the catalytic core, the two LAGLIDADG peptides (SEQ ID NO:373) also play an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), whose protein splicing domain is also involved in DNA binding.

The making of functional chimeric meganucleases, by fusing the N-terminal I-DmoI domain with an I-CreI monomer (Chevalier et al., Mol. Cell., 2002, 10, 895-905; Epinat et al., Nucleic Acids Res, 2003, 31, 2952-62; International PCT Applications WO 03/078619 and WO 2004/031346) have demonstrated the plasticity of LAGLIDADG proteins.

Different groups have also used a semi-rational approach to locally alter the specificity of the I-CreI (Seligman et al., Genetics, 1997, 147, 1653-1664; Sussman et al., J. Mol. Biol., 2004, 342, 31-41; International PCT Applications WO 2006/097784, WO 2006/097853, WO 2007/060495 and WO 2007/049156; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and I77 of I-CreI were mutagenized and a collection of variants with altered specificity at positions ±3 to 5 of the DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).
Residues K28, N30 and Q38 or N30, Y33 and Q38 or K28, Y33, Q38 and S40 of I-CreI were mutagenized and a collection of variants with altered specificity at positions ±8 to 10 of the DNA target (10NNN DNA target) were identified by screening (Smith et al., Nucleic Acids Res., 2006, 34, e149; International PCT Applications WO 2007/060495 and WO 2007/049156).

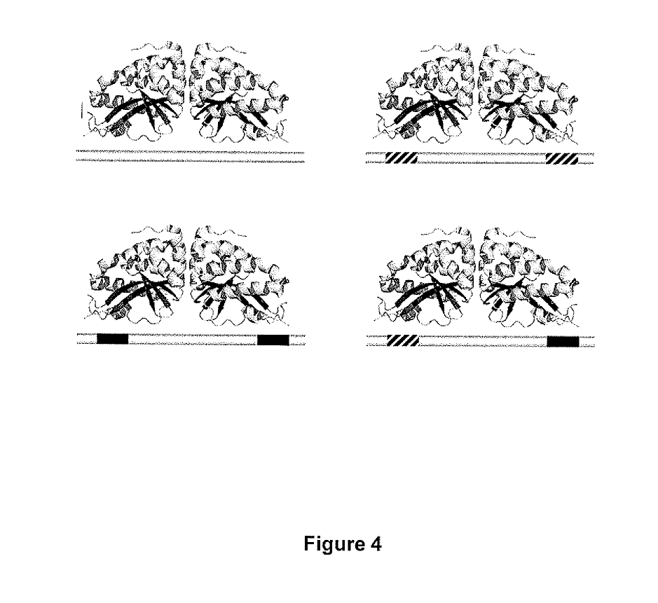

Two different variants were combined and assembled in a functional heterodimeric endonuclease able to cleave a chimeric target resulting from the fusion of two different halves of each variant DNA target sequence (Arnould et al., precited; International PCT Applications WO 2006/097854 and WO 2007/034262), as illustrates in FIG. 4.

Furthermore, residues 28 to 40 and 44 to 77 of I-CreI were shown to form two separable functional subdomains, able to bind distinct parts of a homing endonuclease half-site target sequence (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/049095 and WO 2007/057781).

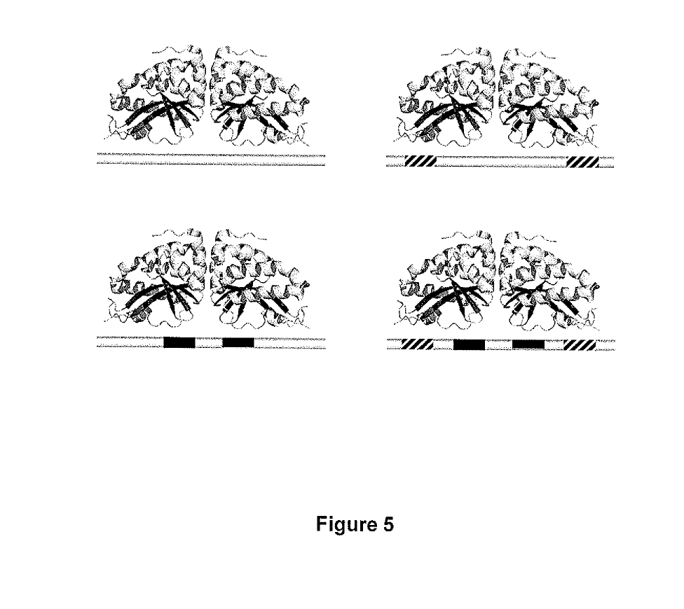

The combination of the two former steps allows a larger combinatorial approach, involving four different subdomains. The different subdomains can be modified separately and combined to obtain an entirely redesigned meganuclease variant (heterodimer or single-chain molecule) with chosen specificity, as illustrated in FIG. 5. In a first step, couples of novel meganucleases are combined in new molecules (“half-meganucleases”) cleaving palindromic targets derived from the target one wants to cleave. Then, the combination of such “half-meganucleases” can result in a heterodimeric species cleaving the target of interest. The assembly of four sets of mutations into heterodimeric endonucleases cleaving a model target sequence or a sequence from different genes has been for instance described in the following patent applications: XPC gene (WO2007093918), RAG gene (WO2008010093), HPRT gene (WO2008059382), beta-2 microglobulin gene (WO2008102274), Rosa26 gene (WO2008152523) and Human hemoglobin beta gene (WO200913622).

The method for producing meganuclease variants and the assays based on cleavage-induced recombination in mammal or yeast cells, which are used for screening variants with altered specificity are described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. These assays result in a functional LacZ reporter gene which can be monitored by standard methods.

These variants can be used to cleave genuine chromosomal sequences and have paved the way for novel perspectives in several fields, including gene therapy.

Even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier, B. S. and B. L. Stoddard, Nucleic Acids Res., 2001, 29, 3757-3774). In vitro selection of cleavable I-CreI targets (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited).

Therefore the inventors seeing the problems associated with retroviruses and in particular HIV, have generated a new class of reagents which can be used to specifically target and manipulate the retroviral provirus. This new class of anti-retroviral molecules can recognize and cleave the integrated provirus either in vitro or in vivo, these reagents can be used for a variety of purposes for instance in research as well as in novel treatment regimes.

According to a first aspect of the present invention there is provided an I-CreI variant which cleaves a target in the provirus of a pathogenic virus, for use in treating an infection of said virus.

The inventors therefore provide a set of I-CreI variants which can recognise and cut targets in a genomically integrated provirus (GIP). Such I-CreI variants provide a new therapeutic route to retrovirus and in particular HIV treatment by HIV provirus inactivation or alteration. This new class of enzymes is also potentially useful in studies into the transcriptional and regulatory behaviour of the provirus.

This new class of anti-HIV medicament can act in a number of ways including by non-homologous end joining, the replacement/removal by homologous recombination with an introduced DNA targeting construct of a portion of the provirus or the removal of the provirus following recombination between chromosome arms. Each of these different mechanisms is discussed in detail below.

In the present Patent Application the genomically integrated provirus (GIP) refers to the DNA sequence present in one or several places in the host cell genome which was inserted following reverse transcription of the RNA virus genome and its integration into the host genome.

In the present Patent Application the terms meganuclease (s) and variant (s) and variant meganuclease (s) will be used interchangeably herein.

The inventors have therefore created a new class of meganuclease based reagents which are useful for the treatment of a retrovirus infection and the most important and potentially useful feature of these enzymes is that instead of acting upon the virion or any component thereof they act upon the genomic insertion of the virus.

Targeting the integrated provirus would allow a clinician to eliminate the structure which leads to the generation of further viral particles, acting at a level that no other anti-viral therapeutic approaches have yet been developed. Conversely, prior art therapies which act upon the different steps of the viral life cycle allow to a clinician to inhibit viral replication, but do not eliminate the source of the virions, which therefore allows for the amplification of the viral infection when the treatment is withdrawn or resistance develops.

These variants also allow the targeting of the DNA version of the virus genome before it has integrated into the host cell genome. By inactivating the virus genome before it can integrate into the host cell genome, the claimed variants can act during the early step of cell infection in a way which no current antiretroviral medicament can.

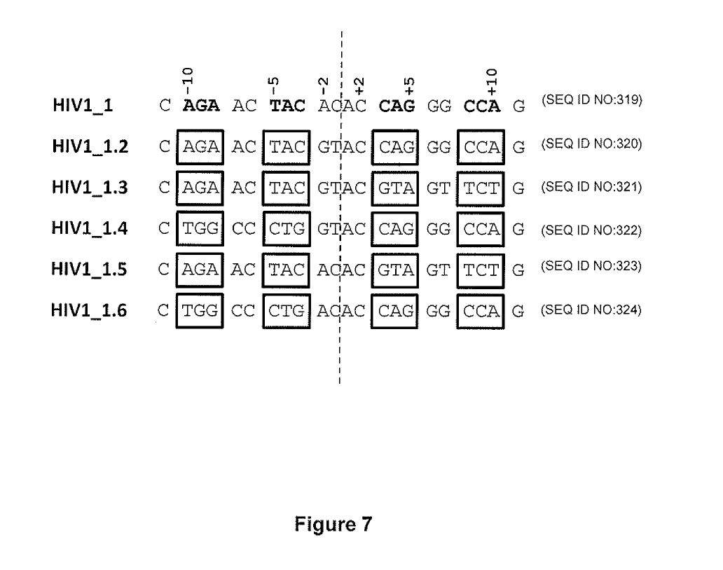

The Inventors have validated this new class of anti-retrovirus reagents by generating meganuclease variants to a series of DNA targets in the genome of the HIV provirus (FIGS. 7, 24, 35 and 48). Seven targets in the HIV provirus were chosen [one in U3 LTR (target HIV1_{—}1 (SEQ ID NO:319)), one in U5 LTR (target HIV1_{—}3 (SEQ ID NO:321)), two in the p24 gene (target HIV1_{—}4 (SEQ ID NO:322)) and (target HIV1_{—}7 (SEQ ID NO:366)), two in the protease gene (target HIV1_{—}5 (SEQ ID NO:323)) and (target HIV1_{—}9 (SEQ ID NO:368)) and one in the p7 gene (target HIV1_{—}8 (SEQ ID NO:367))] and the inventors set out to determine whether it was possible to generate meganucleases capable of cleaving these.

These target sequences are present in the U3 and U5 LTR regions, the coding sequence of the structural gene gag and more specifically in the p7 and p24 proteins therein and in the structural gene pol, specifically in the protease gene. These seven targets were selected based on their therapeutic potential.

As mentioned before, one potential therapeutic approach would be to cleave both LTRs of the integrated provirus which would in turn lead to excision of the viral genome from the infected cells. The inventors have shown that it is possible to generate I-CreI variants which can cleave targets in the U3 (target HIV1_{—}1 (SEQ ID NO:319)) and U5 (target HIV1_{—}3 (SEQ ID NO:321)) LTRs in the present Patent Application.

An alternative therapeutic approach would be to targeting one or more essential genes, the p24 protein is a structural component of the viral capsid and is essential for the virus to replicate. The inventors have shown that it is possible to generate I-CreI variants which can cleave targets in the p24 gene (target HIV1_{—}4 (SEQ ID NO:322)) and (target HIV1_{—}7 (SEQ ID NO:366)) in the present Patent Application. These two targets do not overlap and hence these two enzymes could be used simultaneously so further reducing the chances of resistance developing and/or causing an excision of the portion of p24 situated between the two cleavage sites.

The HIV protease is also an essential protein that is needed for viral particle maturation, without which viral particles remain in an immature state and are not infectious. The inventors have shown that it is possible to generate I-CreI variants which can cleave targets in the protease gene (target HIV1_{—}5 (SEQ ID NO:323)) and (target HIV1_{—}9 (SEQ ID NO:368)) in the present Patent Application. These two targets do not overlap and hence these two enzymes could be used simultaneously so further reducing the chances of resistance developing and/or causing an excision of the portion of protease situated between the two cleavage sites.

The HIV nucleocapsid protein (p7, ou NC) is bound to the single-stranded RNA genome. This protein plays a key role in the HIV life cycle since, being an RNA chaperone, its activity is required for efficient reverse transcription, making it an interesting target for antiviral therapy. The inventors have shown that it is possible to generate I-CreI variants which can cleave targets in the p7 gene (target HIV1_{—}8 (SEQ ID NO:367)).

The inventors have therefore established that meganuclease variants can be generated in both the sequences of essential genes as well as in regulatory non-coding sequences essential for viral replication.

These targets were also selected based on a screen on the “Los Alamos National Laboratory” Sequence database (www.hiv.lan1.gov) to determine their degree of conservation among circulating isolates, which showed a high degree of sequence conservation among the different viral strains for which the complete sequence of their genome is available.

In the present Patent Application essential genes of the GIP provirus are those genes which must remain active in order for the GIP provirus to be converted into further virions which are able to exit the host cell and infect further cells. In addition to essential genes, other types of essential genetic elements can exist such as the regulatory elements of essential genes and/or structural sequence elements of the HIV provirus that are necessary for its packaging and/or insertion into the genome.

According to a further aspect of the present invention the pathogenic virus is from a genus selected from the group consisting of: Alpharetrovirus, Betaretrovirus, Gammaretrovirus, Deltaretrovirus, Epsilonretrovirus, Lentivirus and Spumavirus.

Multiple examples of genomic sequences for viruses of the specified types are available from public databases such as the National Center for Biotechnology Information (http://www.ncbi.nlm.nih.gov/) or the virus genomics and bioinformatics resources centre at University College London (http://www.biochem.ucl.ac.uk/bsm/virus_database/VIDA.html).

In particular the virus is selected from the group consisting of: Human T-lymphotrophic virus, Rous Sarcoma and Human Immunodeficiency Virus.

Most particularly the virus is either Human Immunodeficiency Virus Type 1 (HIV1) or Human Immunodeficiency Virus Type 2 (HIV2).

In particular the DNA target is within a DNA sequence essential for HIV replication, viability, packaging or virulence.

In particular the DNA target is within an essential gene or regulatory element or structural element of the HIV provirus.

In particular the DNA target is within the open reading frame of the HIV provirus encoding a gene or regulatory element of a gene selected from the group: GAG, POL, ENV, TAT and REV.

In particular the target in the HIV1 provirus is selected from the group consisting of the sequences SEQ ID NO: 319 to 342 and SEQ ID NO: 366 to 368.

In particular the variant is selected from one of the sequences SEQ ID NO: 1-13; SEQ ID NO: 26-46; SEQ ID NO: 59-85; SEQ ID NO: 88-94; SEQ ID NO: 97-165; SEQ ID NO: 168-174; SEQ ID NO: 177-186; SEQ ID NO: 189-238; SEQ ID NO: 241-242; SEQ ID NO: 245-253; SEQ ID NO: 256-316; SEQ ID NO: 346-365.

In particular the variant is characterized in that at least one of the two I-CreI monomers has at least two substitutions, one in each of the two functional subdomains of the LAGLIDADG core domain (SEQ ID NO:373) situated from positions; in particular said substitution(s) in the first functional subdomain comprise a substitution in at least one of positions 26, 28, 30, 32, 33, 38 and/or 40 and said substitution(s) in the second functional subdomain comprise a substitution in at least one of positions 44, 68, 70, 75 and/or 77 and being obtainable by a method comprising at least the steps of:

(a) constructing a first series of I-CreI variants having at least one substitution in a first functional subdomain of the LAGLIDADG core domain (SEQ ID NO:373) comprising at least one substitution at a position selected from the group: 26, 28, 30, 32, 33, 38 and/or 40 of I-CreI,

(b) constructing a second series of I-CreI variants having at least one substitution in a second functional subdomain of the LAGLIDADG core domain (SEQ ID NO:373) comprising at least one substitution at a position selected from the group: 44, 68, 70, 75 and/or 77 of I-CreI,

(c) selecting and/or screening the variants from the first series of step (a) which are able to cleave a DNA target sequence selected from the group SEQ ID NO: 319 to 342 and SEQ ID NO: 366 to 368, wherein at least one of (i) the nucleotide triplet in positions −10 to −8 of the I-CreI site has been replaced with the nucleotide triplet which is present in positions −10 to −8 of the selected DNA target sequence from said provirus and (ii) the nucleotide triplet in positions +8 to +10 has been replaced with the reverse complementary sequence of the nucleotide triplet which is present in position −10 to −8 of said DNA target sequence from said provirus,

(d) selecting and/or screening the variants from the second series of step (b) which are able to cleave a mutant I-CreI site wherein at least one of (i) the nucleotide triplet in positions −5 to −3 of the I-CreI site has been replaced with the nucleotide triplet which is present in positions −5 to −3 of said DNA target sequence from said provirus and (ii) the nucleotide triplet in positions +3 to +5 has been replaced with the reverse complementary sequence of the nucleotide triplet which is present in position −5 to −3 of said DNA target sequence from said provirus,

(e) selecting and/or screening the variants from the first series of step (a) which are able to cleave a mutant I-CreI site wherein at least one of (i) the nucleotide triplet in positions +8 to +10 of the I-CreI site has been replaced with the nucleotide triplet which is present in positions +8 to +10 of said DNA target sequence from said provirus and (ii) the nucleotide triplet in positions −10 to −8 has been replaced with the reverse complementary sequence of the nucleotide triplet which is present in positions +8 to +10 of said DNA target sequence from said provirus,

(f) selecting and/or screening the variants from the second series of step (b) which are able to cleave a mutant I-CreI site wherein at least one of (i) the nucleotide triplet in positions +3 to +5 of the I-CreI site has been replaced with the nucleotide triplet which is present in positions +3 to +5 of said DNA target sequence from said provirus and (ii) the nucleotide triplet in positions −5 to −3 has been replaced with the reverse complementary sequence of the nucleotide triplet which is present in positions +3 to +5 of said DNA target sequence from said provirus,

(g) combining in a single variant, the mutation(s) in positions 26, 28, 30, 32, 33, 38 and/or 40 and 44, 68, 70, 75 and/or 77 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said DNA target sequence from said provirus, (ii) the nucleotide triplet in positions +8 to +10 is identical to the reverse complementary sequence of the nucleotide triplet which is present in positions −10 to −8 of said DNA target sequence from said provirus, (iii) the nucleotide triplet in positions −5 to −3 is identical to the nucleotide triplet which is present in positions −5 to −3 of said DNA target sequence from said provirus and (iv) the nucleotide triplet in positions +3 to +5 is identical to the reverse complementary sequence of the nucleotide triplet which is present in positions −5 to −3 of said DNA target sequence from said provirus, and/or

(h) combining in a single variant, the mutation(s) in positions 26, 28, 30, 32, 33, 38 and/or 40, and 44, 68, 70, 75 and/or 77 of two variants from step (e) and step (f), to obtain a novel homodimeric I-CreI variant which cleaves a sequence wherein (i) the nucleotide triplet in positions +8 to +10 of the I-CreI site has been replaced with the nucleotide triplet which is present in positions +8 to +10 of said DNA target sequence from said provirus and (ii) the nucleotide triplet in positions −10 to −8 is identical to the reverse complementary sequence of the nucleotide triplet in positions +8 to +10 of said DNA target sequence from said provirus, (iii) the nucleotide triplet in positions +3 to +5 is identical to the nucleotide triplet which is present in positions +3 to +5 of said DNA target sequence from said provirus, (iv) the nucleotide triplet in positions −5 to −3 is identical to the reverse complementary sequence of the nucleotide triplet which is present in positions +3 to +5 of said DNA target sequence from said provirus,

(i) combining the variants obtained in steps (g) and (h) to form heterodimers, and

(j) selecting and/or screening the heterodimers from step (i) which are able to cleave said DNA target sequence from said provirus.

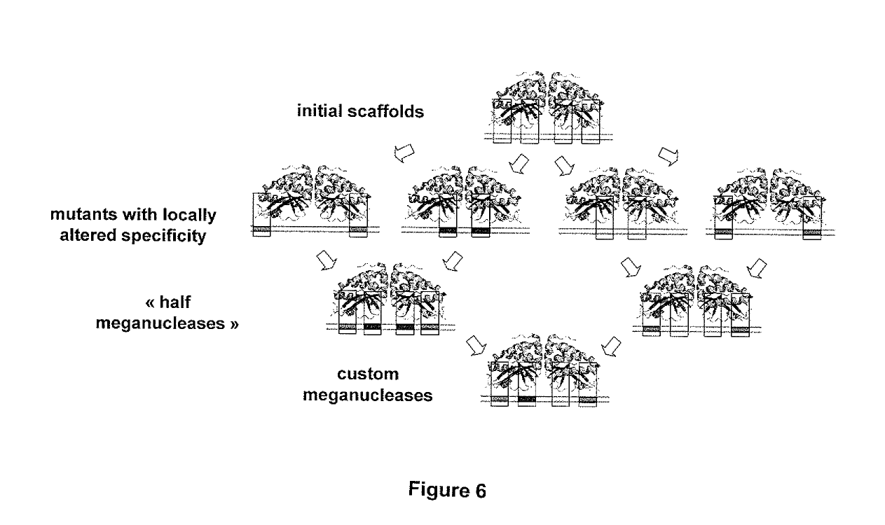

A combinatorial approach, as illustrated schematically in FIG. 6 was used to entirely redesign the DNA binding domain of the I-CreI protein and thereby engineer novel meganucleases with fully engineered specificity.

In particular the heterodimer of step (i) may comprise monomers obtained in steps (g) and (h), with the same DNA target recognition and cleavage activity properties.

Alternatively the heterodimer of step (i) may comprise monomers obtained in steps (g) and (h), with different DNA target recognition and cleavage activity properties.

In particular the first series of I-CreI variants of step (a) are derived from a first parent meganuclease.

In particular the second series of variants of step (b) are derived from a second parent meganuclease.

In particular the first and second parent meganucleases are identical.

Alternatively the first and second parent meganucleases are different.

In particular the variant may be obtained by a method comprising the additional steps of:

(k) selecting heterodimers from step (j) and constructing a third series of variants having at least one substitution in at least one of the monomers of said selected heterodimers,

(l) combining said third series variants of step (k) and screening the resulting heterodimers for enhanced cleavage activity against said DNA target from the GIP.

The inventors have found that although specific meganucleases can be generated to a particular target in the GIP using the above method, that such meganucleases can be improved further by the additional rounds of substitution and selection against the intended target. Meganuclease generated to targets in the GIP using other methods are also comprised within the present Patent Application.

In particular in said step (k) the substitutions in the third series of variants are introduced by site ditected mutagenesis in a DNA molecule encoding said third series of variants, and/or by random mutageneis in a DNA molecule encoding said third series of variants.

In the additional rounds of substitution and selection, the substitution of residues in the meganucleases can be performed randomly, that is wherein the chances of a substitution event occurring are equal chance across all the residues of the meganuclease. Or on a site directed basis wherein the chances of certain residues being subject to a substitution is higher than other residues.

In particular steps (k) and (l) are repeated at least two times and wherein the heterodimers selected in step (k) of each further iteration are selected from heterodimers screened in step (l) of the previous iteration which showed increased cleavage activity against said DNA target from the GIP.

The inventors have found that the meganucleases can be further improved by using multiple iterations of the additional steps (k) and (l).

Through the inventors work they have identified the residues in the first subdomain which when altered have most effect upon altering the I-CreI enzymes specificity.

Through the inventors work they have identified the residues in the second subdomain which when altered have most effect upon altering the I-CreI enzymes specificity.

In particular the variant comprises one or more substitutions in positions 137 to 143 of I-CreI that modify the specificity of the variant towards the nucleotide in positions ±1 to 2, ±6 to 7 and/or ±11 to 12 of the target site in the GIP.

In particular the variant comprises one or more substitutions on the entire I-CreI sequence that improve the binding and/or the cleavage properties of the variant towards said DNA target sequence from the GIP.

As well as specific mutations at the residue identified above, the present invention also encompasses the substitution of any of the residues present in the I-CreI enzyme.

In particular the variant is a heterodimer, resulting from the association of a first and a second monomer having different mutations in positions 26, 28, 30, 32, 33, 38 and/or 40, and 44, 68, 70, 75 and/or 77 of I-CreI, said heterodimer being able to cleave a non-palindromic DNA target sequence from the HIV provirus.

As explained above the I-CreI enzyme acts as a dimer, by ensuring that the variant is a heterodimer this allows a specific combination of two different I-CreI monomers which increases the possible targets cleaved by the variant.

In particular the heterodimeric variant is an obligate heterodimer variant having at least one pair of mutations in corresponding residues of the first and the second monomers which mediate an intermolecular interaction between the two I-CreI monomers, wherein the first mutation of said pair(s) is in the first monomer and the second mutation of said pair(s) is in the second monomer and said pair(s) of mutations impairs the formation of functional homodimers from each monomer without preventing the formation of a functional heterodimer, able to cleave the genomic DNA target from the HIV provirus.

The inventors have previously established a number of residue changes which can ensure an I-CreI monomer is an obligate heterodimer (WO2008/093249).

In particular the monomers have at least one of the following pairs of mutations, respectively for the first and the second monomer:

a) the substitution of the glutamic acid in position 8 with a basic amino acid, preferably an arginine (first monomer) and the substitution of the lysine in position 7 with an acidic amino acid, preferably a glutamic acid (second monomer); the first monomer may further comprise the substitution of at least one of the lysine residues in positions 7 and 96, by an arginine,

b) the substitution of the glutamic acid in position 61 with a basic amino acid, preferably an arginine (first monomer) and the substitution of the lysine in position 96 with an acidic amino acid, preferably a glutamic acid (second monomer); the first monomer may further comprise the substitution of at least one of the lysine residues in positions 7 and 96, by an arginine,

c) the substitution of the leucine in position 97 with an aromatic amino acid, preferably a phenylalanine (first monomer) and the substitution of the phenylalanine in position 54 with a small amino acid, preferably a glycine (second monomer); the first monomer may further comprise the substitution of the phenylalanine in position 54 by a tryptophane and the second monomer may further comprise the substitution of the leucine in position 58 or lysine in position 57, by a methionine, and

d) the substitution of the aspartic acid in position 137 with a basic amino acid, preferably an arginine (first monomer) and the substitution of the arginine in position 51 with an acidic amino acid, preferably a glutamic acid (second monomer).

In particular the variant, which is an obligate heterodimer, wherein the first and the second monomer, respectively, further comprises the D137R mutation and the R51D mutation.

In particular the variant, which is an obligate heterodimer, wherein the first monomer further comprises the K7R, E8R, E61R, K96R and L97F or K7R, E8R, F54W, E61R, K96R and L97F mutations and the second monomer further comprises the K7E, F54G, L58M and K96E or K7E, F54G, K57M and K96E mutations.

According to a further aspect of the present invention there is provided a single-chain chimeric meganuclease which comprises two monomers or core domains of one or two variant(s) according to the first aspect of the present invention, or a combination of both.

An alternative approach to ensuring that the variant consists of a specific combination of monomers is to link the selected monomers for instance using a peptide linker

In particular the single-chain meganuclease comprises a first and a second monomer according to the first aspect of the present invention, connected by a peptidic linker.

According to a further aspect of the present invention the I-CreI variant is combined with other antiretroviral drugs.

Most antiretroviral drugs have at least three names. Sometimes a drug is referred to by its research or chemical name, such as AZT. The second name is the generic name for all drugs with the same chemical structure; for example AZT is also known as zidovudine. The third name is the brand name given by the pharmaceutical company; one of the brand names for zidovudine is Retrovir. Lastly, an abbreviation of the common name might sometimes also be used, such as ZDV, which is the fourth name given to zidovudine.

Lists of drugs approved for use in the USA are provided below:

### Multi-Class Combinations

| Combination | Brand name | Date of FDA approval |
|---|---|---|
| EFV + TDF + FTC | Atripla | 12 Jul. 2006 |
| d4T + 3TC + NVP | — | — |
| AZT + 3TC + NVP | — | — |

### Nucleoside/Nucleotide Reverse Transcriptase Inhibitors (NRTIs)

| Abbreviation | Generic name | Brand name | Date of FDA approval |
|---|---|---|---|
| 3TC | lamivudine | Epivir | 17 Nov. 1995 |
| ABC | abacavir | Ziagen | 17 Dec. 1998 |
| AZT or ZDV | zidovudine | Retrovir | 19 Mar. 1987 |
| d4T | stavudine | Zerit | 24 Jun. 1994 |
| ddI | didanosine | Videx EC | 31 Oct. 2000 |
| FTC | emtricitabine | Emtriva | 02 Jul. 2003 |
| TDF | tenofovir | Viread | 26 Oct. 2001 |

### Combined NRTIs

| Combination | Brand name | Date of FDA approval |
|---|---|---|
| ABC + 3TC | Epzicom (US) | 02 Aug. 2004 |
| | Kivexa (Europe) | |
| ABC + AZT + 3TC | Trizivir | 14 Nov. 2000 |
| AZT + 3TC | Combivir | 27 Sep. 1997 |
| TDF + FTC | Truvada | 02 Aug. 2004 |
| d4T + 3TC | — | — |

### Non-Nucleoside Reverse Transcriptase Inhibitors (NNRTIs)

| Abbreviation | Generic name | Brand name | Date of FDA approval |
|---|---|---|---|
| DLV | delavirdine | Rescriptor | 04 Apr. 1997 |
| EFV | efavirenz | Sustiva (US) | 17 Sep. 1998 |
| | | Stocrin (Europe) | |
| ETR | etravirine | Intelence | 18 Jan. 2008 |
| NVP | nevirapine | Viramune | 21 Jun. 1996 |

### Protease Inhibitors (PIs)

| | | | Date of FDA |
|---|---|---|---|
| Abbreviation | Generic name | Brand name | approval |
| APV | amprenavir | Agenerase | 15 Apr. 1999 |
| FOS-APV | fosamprenavir | Lexiva (US) | 20 Oct. 2003 |
| | | Telzir (Europe) | |
| ATV | atazanavir | Reyataz | 20 Jun. 2003 |
| DRV | darunavir | Prezista | 23 Jun. 2006 |
| IDV | indinavir | Crixivan | 13 Mar. 1996 |
| LPV/RTV | lopinavir + | Kaletra | 15 Sep. 2000 |
| | ritonavir | Aluvia (developing | |
| | | world) | |
| NFV | nelfinavir | Viracept | 14 Mar. 1997 |
| RTV | ritonavir | Norvir | 01 Mar. 1996 |
| SQV | saquinavir | Invirase (hard gel capsule) | 06 Dec. 1995 |
| TPV | tipranavir | Aptivus | 22 Jun. 2005 |

### Fusion or Entry Inhibitors

| Abbreviation | Generic name | Brand Name | Date of FDA approval |
|---|---|---|---|
| T-20 | enfuvirtide | Fuzeon | 13 Mar. 2003 |
| MVC | maraviroc | Celsentri (Europe) | 18 Sep. 2007 |
| | | Selzentry (US) | |

### Integrase Inhibitors

| Abbreviation | Generic name | Brand Name | Date of FDA approval |
|---|---|---|---|
| RAL | raltegravir | Isentress | 12 Oct. 2007 |

Due to the constant evolution of resistance to existing HIV medicaments additional antiretroviral drugs continue to be developed and approved for the treatment of HIV infections.

In accordance with this further aspect of the present invention the I-CreI variant is combined with other antiretroviral agents such as those listed above or with other meganucleases directed against different targets in the HIV provirus.

According to a preferred embodiment of the present invention I-CreI variants according to the present invention are used only once the viral load of an individual has been reduced significantly using antiretroviral drugs. The I-CreI variants are then used to eliminate as many proviruses as possible whilst the HIV virus population is in its enforced dormant state.

Using this strategy it is conceivable that an existent HIV infection could be cured. Perhaps more likely the reduction in the number of active proviruses will lead to a decrease in the number of new virus particles being produced which in turn will reduce the chances of resistant virus particles being generated against any of the medicaments being used to suppress HIV replication. Allowing the use for longer periods of time of the medicaments, so reducing the chances that an individual will ever be infected with HIV particles which are resistant to all anti-HIV medicaments.

In accordance with a further aspect of the present invention there is also provided a kit of parts comprising at least one I-CreI according to the present invention either in the form of a peptide or a nucleotide encoding the variant(s) and one or more other anti-HIV medicaments, together with instructions for the administration of the variant and other anti-HIV medicaments to a patient.

According to the present invention, the meganuclease when used as a polypeptide is associated with:
liposomes, polyethyleneimine (PEI); in such a case said association is administered and therefore introduced into somatic target cells.
membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain meganuclease is fused with the sequence of a membrane translocating peptide (fusion protein).

Alternatively, the meganuclease in the form of a polynucleotide encoding said meganuclease in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

The meganuclease may also comprise a nuclear localization signal (NLS) which is an amino acid sequence which acts like a ‘tag’ on the exposed surface of a protein. The NLS is used to target the protein to the cell nucleus through the Nuclear Pore Complex and to direct a newly synthesized protein into the nucleus via its recognition by cytosolic nuclear transport receptors. Typically, this signal consists of one or more short sequences of positively charged lysines or arginines.

According to a second aspect of the present invention there is provided a polynucleotide fragment encoding the variant according to the first aspect of the present invention or the single-chain chimeric meganuclease according to a second aspect of the present invention.

According to a third aspect of the present invention there is provided an expression vector comprising at least one polynucleotide fragment according to the second aspect of the present invention.

In particular the expression vector, includes a targeting construct comprising a sequence to be introduced flanked by sequences sharing homologies with the regions surrounding said DNA target sequence from the provirus.

One important use of a variant according to the present invention is in increasing the incidence of homologous recombination events at or around the site where the variant cleaves its target. The present invention therefore also relates to a unified genetic construct which encodes the variant under the control of suitable regulatory sequences as well as sequences homologous to portions of the provirus surrounding the variant DNA target site. Following cleavage of the target site by the variant these homologous portions can act as complementary sequences in a homologous recombination reaction with the provirus replacing the existing provirus sequence with a new sequence engineered between the two homologous portions in the unified genetic construct.

Preferably, homologous sequences of at least 50 bp, preferably more than 100 by and more preferably more than 200 by are used. Shared DNA homologies are located in regions flanking upstream and downstream the site of the break and the DNA sequence to be introduced should be located between the two arms.

Therefore, the targeting construct is preferably from 200 by to 6000 bp, more preferably from 1000 by to 2000 bp; it comprises: a sequence which has at least 200 by of homologous sequence flanking the target site, for repairing the cleavage and a sequence for inactivating the provirus and/or a sequence of an exogenous gene of interest which it is intended to insert at the site of the DNA repair event by homologous recombination.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

A vector which can be used in the present invention includes, but is not limited to, a viral vector, a plasmid, a RNA vector or a linear or circular DNA or RNA molecule which may consists of a chromosomal, non chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adeno associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase (HRPT) for eukaryotic cell culture; TRP1 for S. cerevisiae; tetracycline, rifampicin or ampicillin resistance in E. coli.

In particular for the purposes of gene therapy and in accordance with a preferred embodiment of the present invention, the viral vector is selected from the group comprising lentiviruses, Adeno-associated viruses (AAV) and Adenoviruses.

In accordance with another aspect of the present invention the variant and targeting construct may be on different nucleic acid constructs.

In accordance with another aspect of the present invention the variant in the form of a peptide and the targeting construct as a nucleic acid molecule may be used in combination.

In particular, wherein said sequence to be introduced is a sequence which inactivates the HIV provirus.

In particular, wherein the sequence which inactivates the HIV provirus comprises in the 5′ to 3′ orientation: a first transcription termination sequence and a marker cassette including a promoter, the marker open reading frame and a second transcription termination sequence, and said sequence interrupts the transcription of the coding sequence.

In particular, wherein said sequence sharing homologies with the regions surrounding DNA target sequence is from the HIV provirus or a fragment of the HIV provirus comprising sequences upstream and downstream of the cleavage site, so as to allow the deletion of coding sequences flanking the cleavage site.

According to a fourth aspect of the present invention there is provided a host cell which is modified by a polynucleotide according to a second aspect of the present invention or a vector according to a third aspect of the present invention.

A cell according to the present invention may be made according to a method, comprising at least the step of:

(a) introducing into a cell, a meganuclease, as defined above, so as to induce a double stranded cleavage at a site of interest of the GIP comprising a DNA recognition and cleavage site of said meganuclease, and thereby generate a genomically modified cell having repaired the double-strands break, by non-homologous end joining, and

(b) isolating the genomically modified cell of step (a), by any appropriate mean.

The cell which is modified may be any cell of interest. For making transgenic/knock-out animals, the cells are pluripotent precursor cells such as embryoderived stem (ES) cells, which are well-known in the art. For making recombinant cell lines, the cells may advantageously be human cells, for example PerC6 (Fallaux et al., Hum. Gene Ther. 9, 1909-1917, 1998) or HEK293 (ATCC # CRL-1573) cells or an immortal T lymphocyte line such as Jurkat (Schneider et al (1977). Int J Cancer 19 (5): 621-6.). The meganuclease can be provided directly to the cell or through an expression vector comprising the polynucleotide sequence encoding said meganuclease linked to regulatory sequences suitable for directing its expression in the cell used.

Such a modified cell line would have a number of potential uses including the elucidation of aspects of the biology of the modified GIP as well as a model for screening compounds and other substances for therapeutic effects against cells comprising the modified GIP.

According to a fifth aspect of the present invention there is provided a non-human transgenic animal which is modified by a polynucleotide according to a second aspect of the present invention or a vector according to a third aspect of the present invention.

The subject-matter of the present invention is also a method for making an animal which comprises a modified GIP, comprising at least the step of:

(a) introducing into a pluripotent precursor cell or an embryo of an animal, a meganuclease, as defined above, so as to induce a double stranded cleavage at a site of interest of the GIP comprising a DNA recognition and cleavage site of said meganuclease, and thereby generate a genomically modified precursor cell or embryo having repaired the double-strands break by non-homologous end joining,

(b) developing the genomically modified animal precursor cell or embryo of step (a) into a chimeric animal, and

(c) deriving a transgenic animal from a chimeric animal of step (b).

Alternatively, the GIP may be inactivated by insertion of a sequence of interest by homologous recombination between the genome of the animal and a targeting DNA construct according to the present invention.

In particular the targeting DNA is introduced into the cell under conditions appropriate for introduction of the targeting DNA into the site of interest.

In particular, step (b) comprises the introduction of the genomically modified precursor cell obtained in step (a), into blastocysts, so as to generate chimeric animals.

Such a transgenic animal could be used as a multicellular animal model to elucidate aspects of the biology of the GIP, by means of engineering the provirus present in the progenitor cell line. Such transgenic animals also could be used to screen and characterise the effects of for instance novel anti-HIV medicaments.

In particular the targeting DNA construct is inserted in a vector.

For making transgenic animals/recombinant cell lines, including human cell lines expressing an heterologous protein of interest, the targeting DNA comprises the sequence of the exogenous gene encoding the protein of interest, and eventually a marker gene, flanked by sequences upstream and downstream of and essential gene in the HIV provirus, as defined above, so as to generate genomically modified cells (animal precursor cell or embryo/animal or human cell) having replaced the HIV gene by the exogenous gene of interest, by homologous recombination.

The exogenous gene and the marker gene are inserted in an appropriate expression cassette, as defined above, in order to allow expression of the heterologous protein/marker in the transgenic animal/recombinant cell line.

The meganuclease can be used either as a polypeptide or as a polynucleotide construct encoding said polypeptide. It is introduced into somatic cells of an individual, by any convenient means well-known to those in the art, which are appropriate for the particular cell type, alone or in association with either at least an appropriate vehicle or carrier and/or with the targeting DNA.

Once in a cell, the meganuclease and if present, the vector comprising targeting DNA and/or nucleic acid encoding a meganuclease are imported or translocated by the cell from the cytoplasm to the site of action in the nucleus.

According to a sixth aspect of the present invention there is provided a transgenic plant which is modified by a polynucleotide according to a second aspect of the present invention or a vector according to a third aspect of the present invention.

According to a further aspect of the present invention there is provided the use of at least one variant or at least one single-chain chimeric meganuclease as defined above, or at least one vector according to the third aspect of the present invention, for genome engineering for non-therapeutic purposes.

In particular the variant or single-chain chimeric meganuclease or vector is associated with a targeting DNA construct.

In particular the use of the variant is for inducing a double-strand break in a site of interest within the GIP, thereby inducing a DNA recombination event, a DNA loss or cell death.

According to the invention, said double-strand break is for: modifying a specific sequence in the GIP, so as to induce restoration of a GIP function such as replication in studies upon the biology of the virus, or to attenuate or activate the GIP or a gene therein, introducing a mutation into a site of interest of a GIP gene, introducing an exogenous gene or a part thereof, inactivating or deleting the GIP or a part thereof or leaving the DNA unrepaired and degraded.

In particular this present aspect of the present invention relates to the use of a meganuclease variant to treat HIV infection, by inactivating the HIV provirus by therapeutic genome engineering.

According to one aspect of the present invention the use of the meganuclease according to the present invention, comprises at least the following steps:

1) introducing a double-strand break at at least one site of interest in the HIV provirus comprising at least one recognition and cleavage site of said meganuclease, by contacting said cleavage site with said meganuclease;

2) providing a targeting DNA construct comprising the sequence to be introduced flanked by sequences sharing homologies to the targeted locus.

Wherein the meganuclease is provided directly to the cell or through an expression vector comprising the polynucleotide sequence encoding of the meganuclease and is suitable for its expression in the host cell.

This strategy is used to introduce a DNA sequence at the target site, for example to generate a HIV provirus knock-in or knock-out animal model or cell lines that can be used for drug testing or in the case of a cell line, which can be used for administration into a patient from whom it was derived.

According to a further aspect of the present invention the use of the meganuclease, comprises at least the following steps:

1) introducing a double-strand break at a site of interest of the HIV provirus comprising at least one recognition and cleavage site of said meganuclease, by contacting said cleavage site with said meganuclease;

2) maintaining said broken genomic locus under conditions appropriate for homologous recombination with chromosomal DNA sharing homologies to regions surrounding the cleavage site.

As well as inactivating the provirus using a targeting construct, a significant number of inter chromosome arm recombination events are also expected to occur following cleavage of the provirus target. The recombination of chromosome arms occurs most frequently during mitosis, but can also occur as part of the repair mechanism for DNA strand breaks. Such an inter chromosome arm recombination event would either lead to the elimination of the non homologous portions on either side of the break (e.g. the provirus) or more likely cause portions of the provirus to be recombined onto different chromosome arms. In either event this would lead to the inactivation of the provirus.

According to still further aspect of the present invention the use of the meganuclease, comprises at least the following steps:

1) introducing a double-strand break at a site of interest of the HIV provirus comprising at least one recognition and cleavage site of said meganuclease, by contacting said cleavage site with said meganuclease;

2) maintaining said broken genomic locus under conditions appropriate for repair of the double-strands break by non-homologous end joining.

According to a further aspect of the present invention the variant is used for genome therapy to knock-out in animals/cells the GIP, in particular a sequence is introduced which inactivates the HIV provirus.

All HIV proviruses present in the cell have to be targeted in order to totally inactivate the pathogenicity of the virus. In addition, the introduced sequence may also delete the HIV provirus or part thereof, and introduce an exogenous gene or part thereof (knock-in/gene replacement). For making knock-in animals/cells the DNA which repairs the site of interest may comprise the sequence of an exogenous gene of interest, and a selection marker, such as the G418 resistance gene. Alternatively, the sequence to be introduced can be any other sequence used to alter the chromosomal DNA in some specific way including a sequence used to modify a specific sequence, to attenuate or activate the endogenous gene of interest in the HIV provirus or to introduce a mutation into a site of interest in the HIV provirus. Such chromosomal DNA alterations may be used for genome engineering (animal models and recombinant cell lines including human cell lines).

Inactivation of the HIV provirus may occur by insertion of a transcription termination signal that will interrupt the transcription of an essential gene such as GAG, POL and ENV and result in a truncated protein. In this case, the sequence to be introduced comprises, in the 5′ to 3′ orientation: at least a transcription termination sequence (polyA1), preferably said sequence further comprises a marker cassette including a promoter and the marker open reading frame (ORF) and a second transcription termination sequence for the marker gene ORF (polyA2). This strategy can be used with any variant cleaving a target downstream of the relevant gene promoter and upstream of the stop codon.

Inactivation of the HIV provirus may also occur by insertion of a marker gene within an essential gene of HIV, which would disrupt the coding sequence. The insertion can in addition be associated with deletions of ORF sequences flanking the cleavage site and eventually, the insertion of an exogenous gene of interest (gene replacement).

In addition, inactivation of the HIV provirus may also occur by insertion of a sequence that would destabilize the mRNA transcript of an essential gene.

The present invention also provides a composition characterized in that it comprises at least one variant as defined above (variant or single-chain derived chimeric meganuclease) and/or at least one expression vector encoding the variant, as defined above.

The administration of the provirus targeting variant in as both a peptide and nucleotide form allows for the immediate action of the variant as as its persistence in the target cell.

In particular the composition comprises more than one variant, wherein each of the variants is directed towards a different target sequence in the provirus.

In particular the composition comprises a targeting DNA construct comprising a sequence which inactivates the HIV provirus, flanked by sequences sharing homologies with the genomic DNA cleavage site of said variant, as defined above.

Preferably, said targeting DNA construct is either included in a recombinant vector or it is included in an expression vector comprising the polynucleotide(s) encoding the variant according to the invention.

The subject-matter of the present invention is also the use of at least one meganuclease and/or one expression vector, as defined above, for the preparation of a medicament for preventing, improving or curing HIV infection in an individual in need thereof.

The subject-matter of the present invention is also the use of at least one variant and/or one expression vector, as defined above, for the preparation of a medicament for preventing, improving or curing a pathological condition associated with HIV infection in an individual in need thereof.

As discussed above the variants according to the present invention provide a possible means to prevent chromosomal integration of a target cell with the retrovirus genome. The first step of the viral infection following viral entry into the target cell is the reverse transcription (RT) of the viral genomic RNA. During this RT process, a linear double stranded DNA molecule is formed which then enters the nucleus so that it can be integrated in the cellular genome. Meganuclease variants of the present invention are also able to cleave the pre-integration complex (PIC), which is an episomal double stranded DNA molecule, conferring a protective effect during the earliest steps of viral infection, of a cell population.

The use of the meganuclease may comprise at least the step of (a) inducing in somatic tissue(s) of the donor/individual a double stranded cleavage at a site of interest of the HIV provirus comprising at least one recognition and cleavage site of said meganuclease by contacting said cleavage site with said meganuclease, and (b) introducing into said somatic tissue(s) a targeting DNA, wherein said targeting DNA comprises (1) DNA sharing homologies to the region surrounding the cleavage site and (2) DNA which inactivates the HIV provirus upon recombination between the targeting DNA and the chromosomal DNA, as defined above. The targeting DNA is introduced into the somatic tissues(s) under conditions appropriate for introduction of the targeting DNA into the site of interest. The targeting construct may comprise sequences for deleting the HIV provirus or a portion thereof and introducing the sequence of an exogenous gene of interest (gene replacement).

In this case the use of the meganuclease comprises at least the step of: inducing in somatic tissue(s) of the donor/individual a double stranded cleavage at a site of interest of the HIV provirus comprising at least one recognition and cleavage site of the meganuclease by contacting the cleavage site with the meganuclease, and thereby inducing mutagenesis of an open reading frame in the HIV provirus by repair of the double-strands break by non-homologous end joining.

According to the present invention, said double-stranded cleavage may be induced, ex vivo by introduction of said meganuclease into infected cells isolated for instance from the circulatory system of the donor/individual and then transplantation of the modified cells back into the diseased individual.

The subject-matter of the present invention is also a method for preventing, improving or curing HIV infection, in an individual in need thereof, said method comprising at least the step of administering to said individual a composition as defined above, by any means.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted HIV infection.

In particular as far as possible the meganuclease comprising compositions should be non-immunogenic, i.e., engender little or no adverse immunological response. A variety of methods for ameliorating or eliminating deleterious immunological reactions of this sort can be used in accordance with the invention. One means of achieving this is to ensure that the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

In accordance with a further aspect of the present invention, the invention also relates to meganuclease variants, related materials and uses thereof which recognize non-virus retroelements and/or the integrated genomes of viruses which do not have mechanisms to integrate into the host cell genome.

Non-virus retroelements are endogenous genomic DNA elements that include the gene for reverse transcriptase and are also known as class I transposable elements. These retrotransposons, include the long terminal repeat (LTR) retrotransposons, non-LTR retroposons and group II mitochondrial introns. They are though to be derived from partially inactivated retroviruses which have lost the ability to form infective virus particles. These genetic elements however are increasingly becoming associated with various diseases, in particular cancers and immune disorders which result form the integration of the element into a site close to a gene (s) whose misregulation leads to the observed disease phenotype.

The present invention therefore also relates to meganuclease variants which can be used to cleave a genomic retrotransposon either in a specific tissue or cell type or more generally so as to treat the disease phenotype using one or more of the mechanisms described above.

The present invention also relates to meganuclease variants which can recognise and cleave targets in genomic insertions of viruses which do not normally insert into the host cell genome. The non-specific insertion of viral genetic material into the host cell genome is a disease causing mechanism which is currently being investigated. For example in the important virus hepatitis B, chronic infection with this virus is associated with a greatly elevated risk of hepatocellular carcinoma. In the past this association has been explained as a side effect of the episomal hepatitis B genome upon the hepatocyte host cells. Although this is doubtless true, recently the random genomic insertion of copies of the hepatitis B genome into the host cell genome has also been shown to be a causative factor in hepatocyte carcinoma (Goodarzi et al., 2008, Hep. Mon; 8 (2): 129-133).

Hepatocellular carcinoma is one of the most common cancers in the world and hence a treatment for this condition, using a meganuclease variant which can cleave the randomly integrated hepatitis B genome and have a therapeutic affect upon hepatocytes via one or more of mechanisms detailed above is therefore also within the scope of the present invention as are more generally meganuclease variants to genomically integrated copies of virus genetic material which cause a disease phenotype.

### DEFINITIONS

Throughout the present Patent Application a number of terms and features are used to present and describe the present invention, to clarify the meaning of these terms a number of definitions are set out below and wherein a feature or term is not otherwise specifically defined or obvious from its context the following definitions apply.
Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased cleavage activity, refers to an increase in the detected level of meganuclease cleavage activity (see below) against a target DNA sequence by a first meganuclease in comparison to the activity of a second meganuclease against the target DNA sequence. Normally the first meganuclease will be a variant of the second and comprise one or more substituted amino acid residues in comparison to the second meganuclease.
by “beta-hairpin” it is intended two consecutive beta-strands of the antiparallel beta-sheet of a LAGLIDADG (SEQ ID NO:373) homing endonuclease core domain (β₁β₂ or β₃β₄) which are connected by a loop or a turn,
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).
Cleavage activity: the cleavage activity of the variant according to the invention may be measured by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in a yeast or a mammalian expression vector. Usually, the genomic DNA target sequence comprises one different half of each (palindromic or pseudo-palindromic) parent homodimeric meganuclease target sequence. Expression of the heterodimeric variant results in a functional endonuclease which is able to cleave the genomic DNA target sequence. This cleavage induces homologous recombination between the direct repeats, resulting in a functional reporter gene (LacZ, for example), whose expression can be monitored by an appropriate assay. The specificity of the cleavage by the variant may be assessed by comparing the cleavage of the (non-palindromic) DNA target sequence with that of the two palindromic sequences cleaved by the parent homodimeric meganucleases or compared with wild type meganuclease.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “domain” or “core domain” it is intended the “LAGLIDADG (SEQ ID NO:373) homing endonuclease core domain” which is the characteristic α₁β₁β₂α₂β₃β₄α₃ fold of the homing endonucleases of the LAGLIDADG (SEQ ID NO:373) family, corresponding to a sequence of about one hundred amino acid residues. Said domain comprises four beta-strands (β₁β₂β₃β₄) folded in an antiparallel beta-sheet which interacts with one half of the DNA target. This domain is able to associate with another LAGLIDADG (SEQ ID NO:373) homing endonuclease core domain which interacts with the other half of the DNA target to form a functional endonuclease able to cleave said DNA target. For example, in the case of the dimeric homing endonuclease I-CreI (163 amino acids), the LAGLIDADG (SEQ ID NO:373) homing endonuclease core domain corresponds to the residues 6 to 94.
by “DNA target”, “DNA target sequence”, “target sequence”, “target-site”, “target”, “site”; “site of interest”; “recognition site”, “recognition sequence”, “homing recognition site”, “homing site”, “cleavage site” it is intended a 20 to 24 by double-stranded palindromic, partially palindromic (pseudo-palindromic) or non-palindromic polynucleotide sequence that is recognized and cleaved by a LAGLIDADG (SEQ ID NO:373) homing endonuclease such as I-CreI, or a variant, or a single-chain chimeric meganuclease derived from I-CreI These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicated for C1221 (see FIG. 1). Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” it is intended the portion of the DNA target which is bound by each LAGLIDADG (SEQ ID NO:373) homing endonuclease core domain.
by “DNA target sequence from the HIV provirus” it is intended a 20 to 24 by sequence of the HIV provirus which is recognized and cleaved by a meganuclease variant. In particular the DNA target sequence from then HIV provirus is in an essential gene sequence and/or within an essential regulatory sequence and/or within an essential structural sequence of the HIV provirus.
by “first/second/third/n^{th} series of variants” it is intended a collection of variant meganucleases, each of which comprises one or more amino acid substitution in comparison to a parent meganuclease from which all the variants in the series are derived.
by “functional variant” it is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “heterodimer” it is intended to mean a meganuclease comprising two non-identical monomers. In particular the monomers may differ from each other in their peptide sequence and/or in the DNA target half-site which they recognise and cleave.
by “homologous” is intended a sequence with enough identity to another one to lead to a homologous recombination between sequences, more particularly having at least 95% identity, preferably 97% identity and more preferably 99%.
by “I-CreI” it is intended the wild-type I-CreI having the sequence of pdb accession code 1g9y, corresponding to the sequence SEQ ID NO: 344 in the sequence listing.
by “I-CreI variant with novel specificity” it is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present Patent Application all the I-CreI variants described comprise an additional Alanine after the first Methionine of the wild type I-CreI sequence (SEQ ID NO: 344). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present Patent Application, as these references exclusively refer to residues of the wild type I-CreI enzyme (SEQ ID NO: 344) as present in the variant, so for instance residue 2 of I-CreI is in fact residue 3 of a variant which comprises an additional Alanine after the first Methionine.
by “I-CreI site” it is intended a 22 to 24 by double-stranded DNA sequence which is cleaved by I-CreI. I-CreI sites include the wild-type (natural) non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 343), also called C1221.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
by “meganuclease”, it is intended an endonuclease having a double-stranded DNA target sequence of 12 to 45 bp. The meganuclease is either a dimeric enzyme, wherein each domain is on a monomer or a monomeric enzyme comprising the two domains on a single polypeptide.
by “meganuclease domain”, it is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease (natural or variant meganuclease) with a different amino acid.
by “monomer” it is intended to mean a peptide encoded by the open reading frame of the I-CreI gene or a variant thereof, which when allowed to dimerise forms a functional I-CreI enzyme. In particular the monomers dimerise via interactions mediated by the LAGLIDADG motif (SEQ ID NO:373).
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step a. or the meganuclease from which the second series of variants are derived in step b., or the meganuclease from which the third series of variants are derived in step k.
by “peptide linker” it is intended to mean a peptide sequence of at least 10 and preferably at least 17 amino acids which links the C terminal amino acid residue of the first monomer to the N terminal residue of the second monomer and which allows the two variant monomers to adopt the correct conformation for activity and which does not alter the specificity of either of the monomers for their targets.
by “provirus” it is intended to mean a DNA version of a retrovirus genome. In particular the provirus may be the DNA molecule directly resulting from the reverse transcription of the RNA genome of a virus or alternatively it may be the chromosomally integrated version of the virus genome present at one or more sites in one or more chromosomes of the target cell.
by “subdomain” it is intended the region of a LAGLIDADG (SEQ ID NO:373) homing endonuclease core domain which interacts with a distinct part of a homing endonuclease DNA target half-site.
by “single-chain meganuclease”, “single-chain chimeric meganucleave”, “single-chain meganuclease derivative”, “single-chain chimeric meganuclease derivative” or “single-chain derivative” it is intended a meganuclease comprising two LAGLIDADG (SEQ ID NO:373) homing endonuclease domains or core domains linked by a peptidic spacer. The single-chain meganuclease is able to cleave a chimeric DNA target sequence comprising one different half of each parent meganuclease target sequence.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the HIV provirus and the repair matrix, wherein the genomic sequence containing the DNA target is replaced by the third portion of the repair matrix and a variable part of the first and second portions of the repair matrix.
by “vector” is intended a nucleic acid molecule capable of transporting another nucleic acid to which it has been linked into a host cell in vitro, in vivo or ex vivo. For a better understanding of the invention and to show how the same may be carried into effect, there will now be shown by way of example only, specific embodiments, methods and processes according to the present invention with reference to the accompanying drawings in which:

FIG. 1: Schematic representation of an HIV-1 viral particle. The two molecules of genomic RNA are represented, together with the RT, inside the viral capsid. The envelope, derived from the membrane of the infected cells, contains the envelope glycoproteins gp41 and gp120.

FIG. 2: A: Organization of the HIV-1 genomic RNA molecules. Different genes are represented with different shades of grey, and the proteins encoded by these genes are represented in the lower part of the panel. B: Genetic organization of the integrated HIV-1 provirus, showing the structure of the LTRs after duplication of the U3 and U5 sequences during reverse transcription.

FIG. 3: Tridimensional structure of the I-CreI homing endonuclease bound to its DNA target. The catalytic core is surrounded by two αββαββα folds forming a saddle-shaped interaction interface above the DNA major groove.

FIG. 4: Different I-CreI variants binding different sequences derived from the I-CreI target sequence (top right and bottom left) to obtain heterodimers or single chain fusion molecules cleaving non palindromic chimeric targets (bottom right).

FIG. 5: Shows a schematic representation of the smaller independent subunits of the I-CreI meganuclease, i.e., subunit within a single monomer or αββαββα fold (top right and bottom left). These independent subunits allow for the design of novel chimeric molecules (bottom right), by combination of mutations within a same monomer. Such molecules would cleave palindromic chimeric targets (bottom right).

FIG. 6: Shows a schematic representation of a method to combine four different subdomains so as to generate a custom meganuclease which cleaves a selected target.

FIG. 7: The HIV1_{—}1 target sequence (SEQ ID NO:319) and its derivatives. In the HIV1_{—}1.2 target (SEQ ID NO:320), the ACAC sequence in the middle of the target is replaced with GTAC, the bases found in C1221 (SEQ ID NO:343). HIV1_{—}1.3 (SEQ ID NO:321) is the palindromic sequence derived from the left part of HIV1_{—}1.2, (SEQ ID NO:320) and HIV1_{—}1.4 (SEQ ID NO:322) is the palindromic sequence derived from the right part of HIV1_{—}1.2 (SEQ ID NO:320). HIV1_{—}1.5 (SEQ ID NO:323) and HIV1_{—}1.6 (SEQ ID NO:324) are pseudopalindromic targets derived, respectively, from HIV1_{—}1.3 (SEQ ID NO:321) and HIV1_{—}1.4 (SEQ ID NO:322), containing the natural ACAC sequence in the middle of the target. As shown in the Figure, the boxed motives from 10AGA_P (SEQ ID NO:381), 10TGG_P (SEQ ID NO:379), 5TAC_P (SEQ ID NO:389) and 5CTG_P (SEQ ID NO:387) are found in the HIV1_{—}1 series of targets (SEQ ID NO:319 to 324).

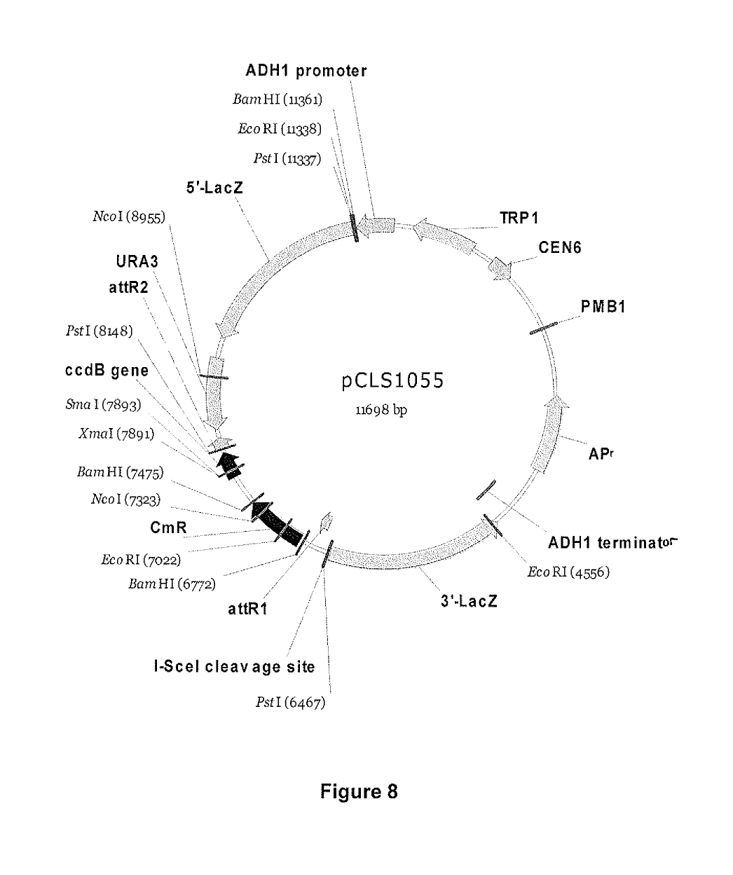

FIG. 8: pCLS1055 plasmid map.

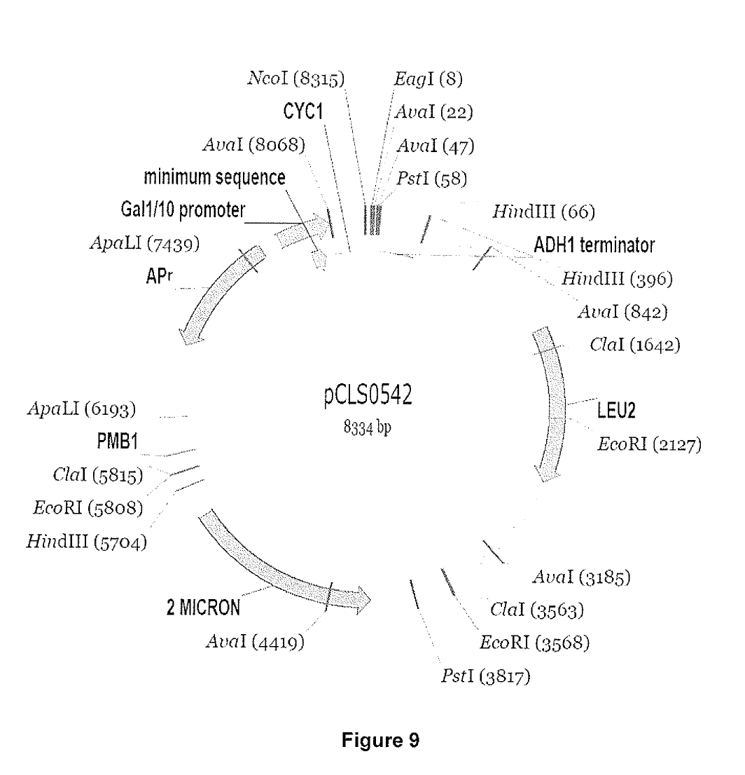

FIG. 9: pCLS0542 plasmid map.

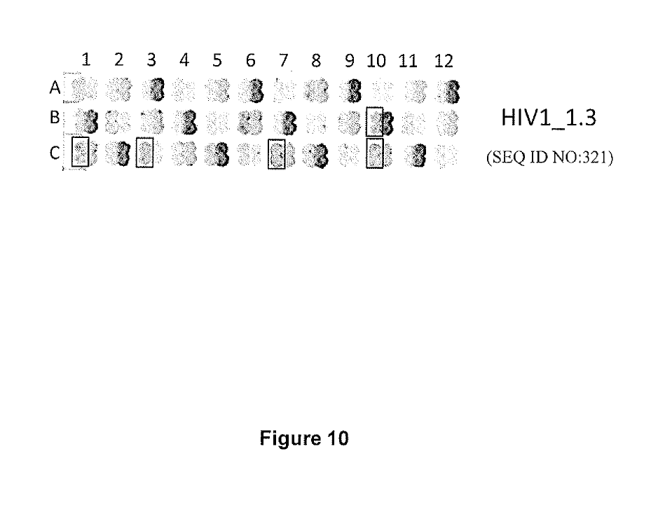

FIG. 10: Cleavage of HIV1_{—}1.3 (SEQ ID NO:321) target by combinatorial variants. The figure displays an example of screening of I-CreI combinatorial variants with the HIV1_{—}1.3 target (SEQ ID NO:321). On the filter, the positive variants correspond to: B10, SEQ ID NO:1; C1, SEQ ID NO:2; C7, SEQ ID NO:3; C10, SEQ ID NO:4; C3, SEQ ID NO:5; all described in Table II. Each cluster contains 4 spots. On the two spots on the left, a yeast strain harboring the HIV1_{—}1.3 target (SEQ ID NO:321) has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

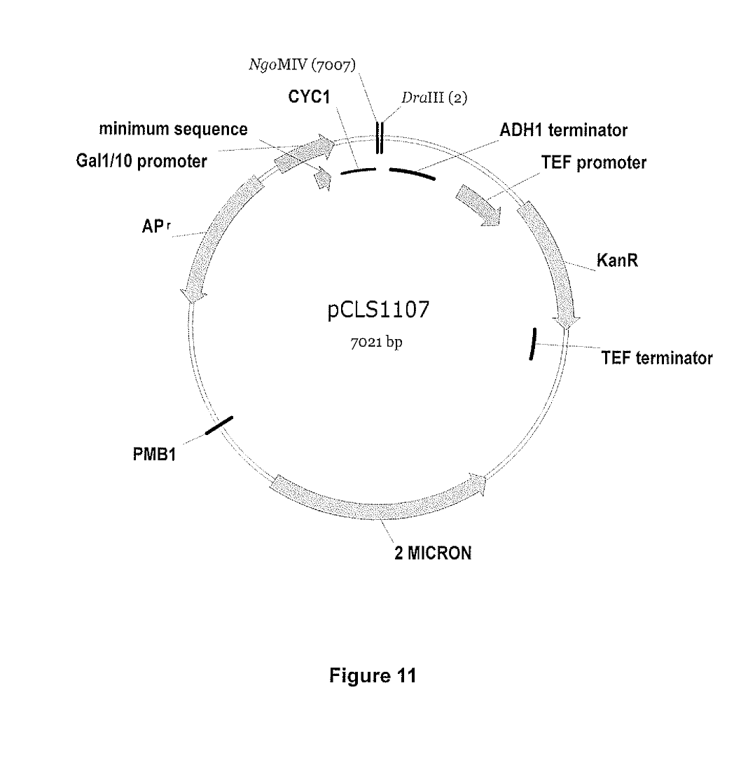

FIG. 11: pCLS1107 plasmid map.

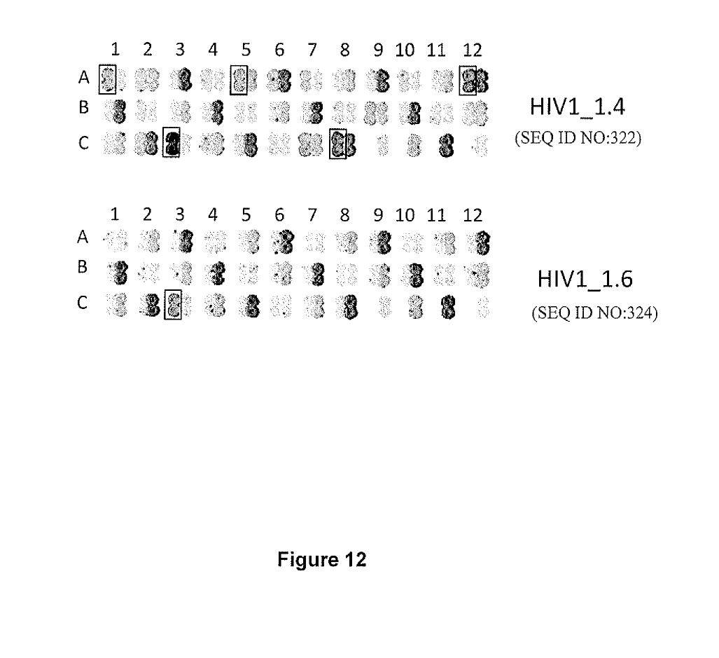

FIG. 12: Cleavage of HIV1_{—}1.4 (SEQ ID NO:322) and HIV1_{—}1.6 (SEQ ID NO:324) targets by combinatorial variants. The figure displays an example of screening of I-CreI combinatorial variants with the HIV1_{—}1.4 (SEQ ID NO:322) and HIV1_{—}1.6 (SEQ ID NO:324) targets. On the filter, the positive variants correspond to: C8, SEQ ID NO:7; A5, SEQ ID NO:8; A1, SEQ ID NO:9; A12, SEQ ID NO:10; C3, SEQ ID NO:11; all described in Table IV. Each cluster contains 4 spots. On the two spots on the left, a yeast strain harboring the HIV1_{—}1.4 (SEQ ID NO:322) or the HIV1_{—}1.6 (SEQ ID NO:324) targets have been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3)

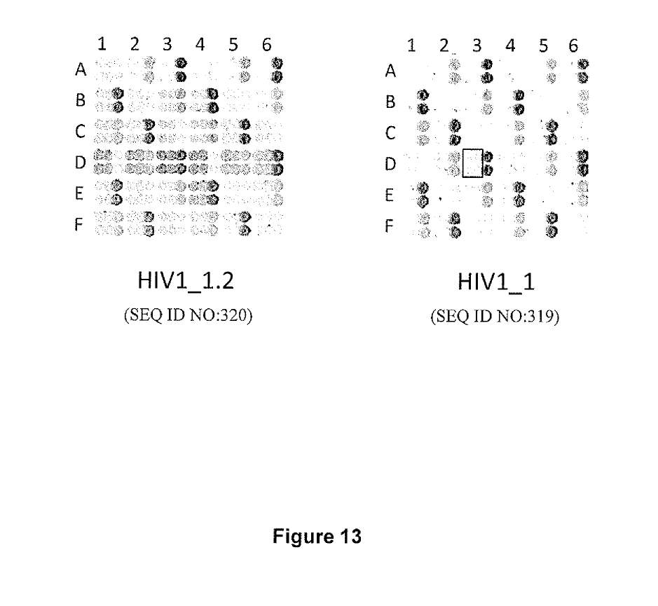

FIG. 13: Cleavage of the HIV1_{—}1.2 (SEQ ID NO:320) and HIV1_{—}1 (SEQ ID NO:319) target sequences by heterodimeric combinatorial variants. Left panel: Example of screening of combinations of I-CreI variants against the HIV1_{—}1.2 target (SEQ ID NO:320). Right panel: Screening of the same combinations of I-CreI variants against the HIV1_{—}1 target (SEQ ID NO:319). Some heterodimers resulted in cleavage of the HIV1_{—}1.2 target (SEQ ID NO:320). The heterodimer displaying a signal with HIV1_{—}1 target (SEQ ID NO:319) is observed at positions D3. On the filter, the position of mutants in certain positions as an example is: line C, SEQ ID NO:10; line D, SEQ ID NO:11; column 2, SEQ ID NO:1; column 3, SEQ ID NO:2; column 4; SEQ ID NO:5. These mutants have been described in Tables II and IV. Each cluster contains 6 spots. On the 4 spots on the left, a yeast strain harboring the HIV1_{—}1 target (SEQ ID NO:319) has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3)

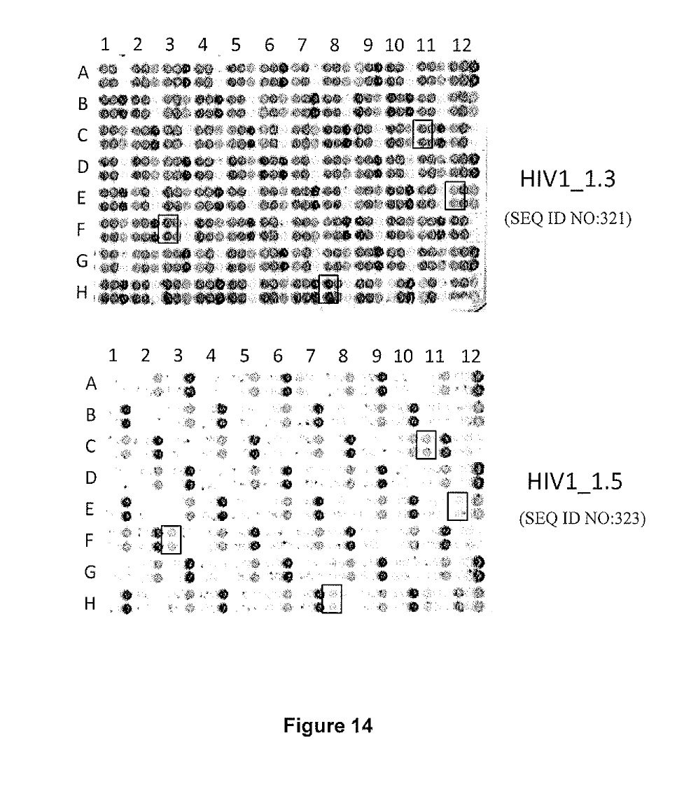

FIG. 14: Cleavage of HIV1_{—}1.3 (SEQ ID NO:321) and HIV1_{—}1.5 (SEQ ID NO:323) targets by meganuclease variants improved by random mutagenesis in example 5. The figure displays an example of screening of I-CreI meganuclease variants with the HIV1_{—}1.3 (SEQ ID NO:321) and HIV1_{—}1.5 (SEQ ID NO:323) targets. On the filter, the positive variants presented correspond to: F3, SEQ ID NO:27; C11, SEQ ID NO:26; H8, SEQ ID NO:28; E12, SEQ ID NO:29; all described in Table VIII. Each cluster contains 6 spots. On the 2 spots on the left, a yeast strain harboring the HIV1_{—}1.3 (SEQ ID NO:321) or the HIV1_{—}1.5 (SEQ ID NO:323) targets have been mated with another yeast strain containing the meganuclease variants. The two spots in the middle contain, as an internal control, a non-improved variant cleaving the HIV1_{—}1.3 target (SEQ ID NO:321). The two spots on the right contain the same negative or positive controls. These controls are serially repeated every three clusters as follows: negative control (i.e. cluster A1), positive control (i.e. cluster A2), and strong positive control (i.e. cluster A3).

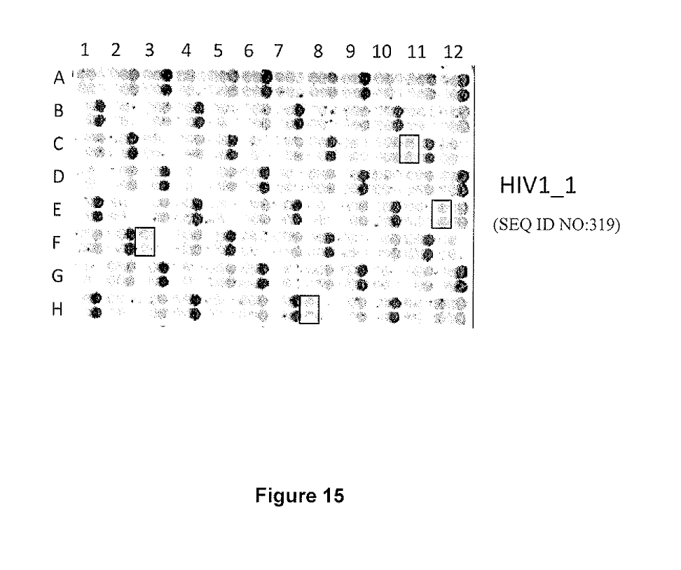

FIG. 15: Cleavage of HIV1_{—}1 target (SEQ ID NO:319) by meganuclease variants improved by random mutagenesis in example 5. The figure displays an example of screening of I-CreI meganuclease variants with the HIV1_{—}1 target, when mated with a meganuclease (SEQ ID NO:46) cleaving the HIV1_{—}1.4 target. On the filter, the positive variants presented correspond to: F3, SEQ ID NO:27; C11, SEQ ID NO:26; H8, SEQ ID NO:28; E12, SEQ ID NO:29; all described in Table VIII. Each cluster contains 6 spots. On the 2 spots on the left, a yeast strain harboring the HIV1_{—}1.4 mutant (SEQ ID NO:46) and the HIV1_{—}1 target (SEQ ID NO:319) have been mated with another yeast strain containing the meganuclease variants. The two spots in the middle contain, as an internal control, a non-improved variant. The two spots on the right contain the same negative or positive controls. These controls are serially repeated every three clusters as follows: negative control (i.e. cluster A1), positive control (i.e. cluster A2), and strong positive control (i.e. cluster A3).

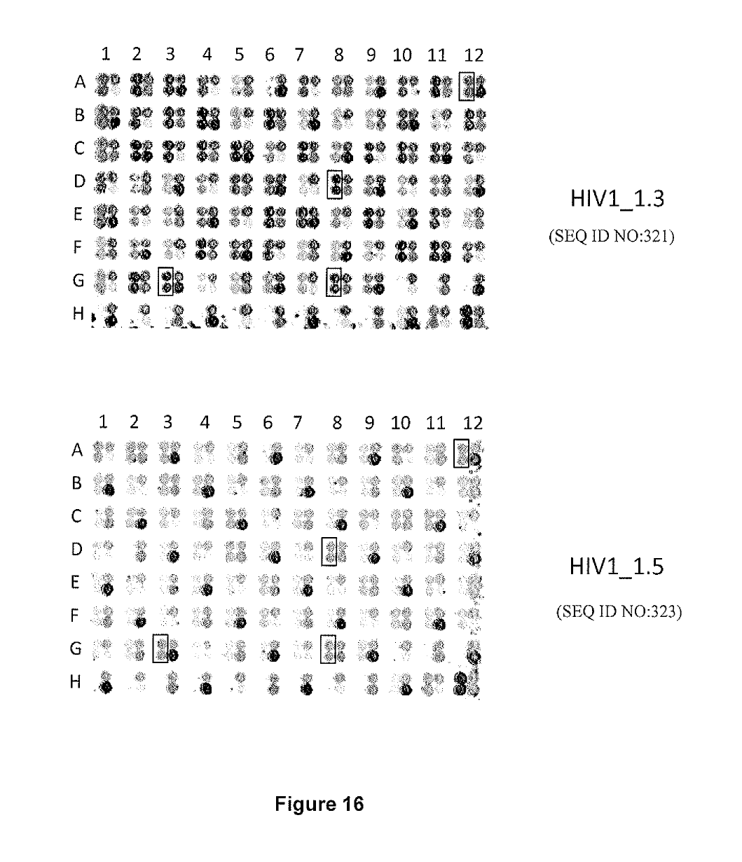

FIG. 16: Cleavage of HIV1_{—}1.3 (SEQ ID NO:321) and HIV1_{—}1.5 (SEQ ID NO:323) targets by meganuclease variants improved by a second round of random mutagenesis in example 5bis. The figure displays an example of screening of I-CreI meganuclease variants with the HIV1_{—}1.3 (SEQ ID NO:321) and HIV1_{—}1.5 (SEQ ID NO:323) targets. On the filter, the positive variants presented correspond to: A12, SEQ ID NO:42; D8, SEQ ID NO:38; G8, SEQ ID NO:36; G3, SEQ ID NO:40; all described in Table IX. Each cluster contains 4 spots. On the 2 spots on the left, a yeast strain harboring the HIV1_{—}1.3 (SEQ ID NO:321) or the HIV1_{—}1.5 (SEQ ID NO:323) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

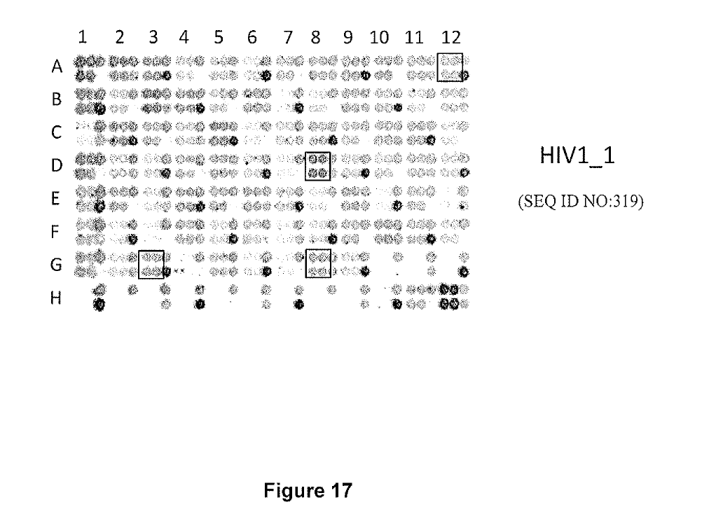

FIG. 17: Cleavage of HIV1_{—}1 (SEQ ID NO:319) target by meganuclease variants improved by a second round of random mutagenesis in example 5bis. The figure displays an example of screening of I-CreI meganuclease variants with the HIV1_{—}1 target (SEQ ID NO:319), when mated with a meganuclease (SEQ ID NO:46) cleaving the HIV1_{—}1.4 target (SEQ ID NO:322). On the filter, the positive variants presented correspond to: A12, SEQ ID NO:42; D8, SEQ ID NO:38; G8, SEQ ID NO:36; G3, SEQ ID NO:40; all described in Table IX. Each cluster contains 6 spots. On the 4 spots on the left, a yeast strain harboring the HIV1_{—}1.4 mutant (SEQ ID NO:46) and the HIV1_{—}1 target (SEQ ID NO:319) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

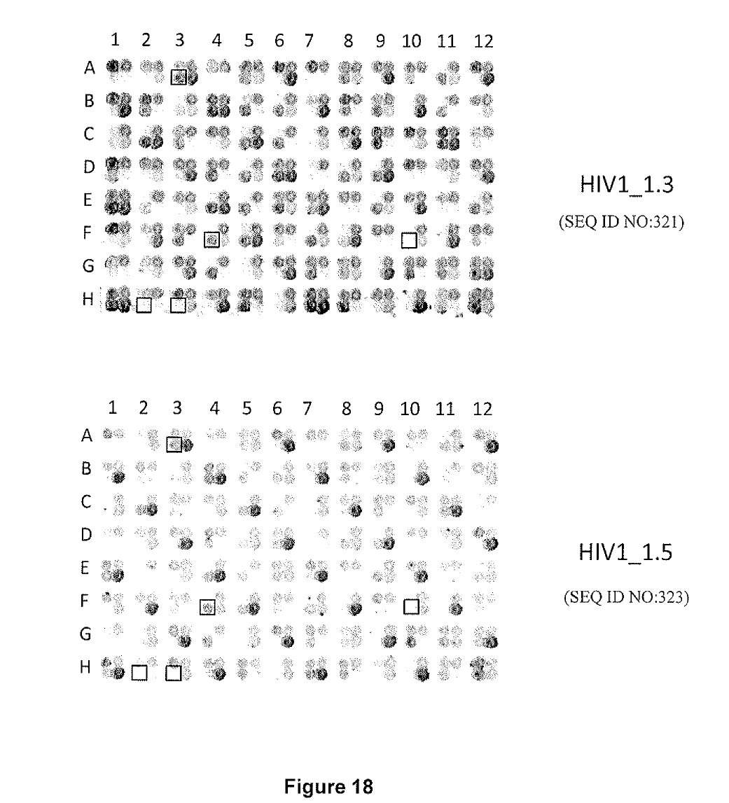

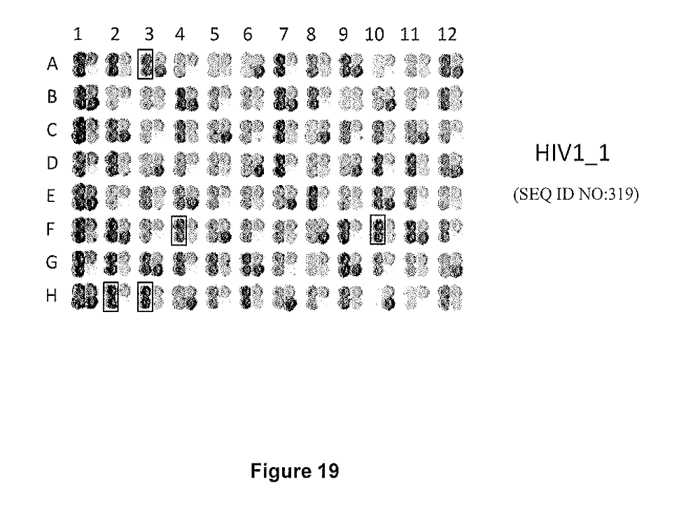

FIG. 18: Cleavage of HIV1_{—}1.3 (SEQ ID NO:321) and HIV1_{—}1.5 (SEQ ID NO:323) targets by meganuclease variants improved by site-directed mutagenesis in example 6. The figure displays an example of screening of I-CreI meganuclease variants with the HIV1_{—}1.3 (SEQ ID NO:321) and HIV1_{—}1.5 (SEQ ID NO:323) targets. On the filter, the positive variants presented correspond to: F10, SEQ ID NO:63; 112, SEQ ID NO:60; H3, SEQ ID NO:59; A3, SEQ ID NO:64; F4, SEQ ID NO:65; some of them described in Table XI. Some of these variants show no cleavage activity as homodimers while they are active as heterodimers on the HIV1_{—}1 target (SEQ ID NO:319) (see FIG. 19). This is due to the presence of the G19S mutation in these variants. Each cluster contains 4 spots. On the 2 spots on the left, a yeast strain harboring the HIV1_{—}1.3 (SEQ ID NO:321) or the HIV1_{—}1.5 (SEQ ID NO:323) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

FIG. 19: Cleavage of HIV1_{—}1 target (SEQ ID NO:319) by meganuclease variants improved by site-directed mutagenesis in example 6. The figure displays an example of screening of I-CreI meganuclease variants with the HIV1_{—}1 target (SEQ ID NO:319), when mated with a meganuclease (SEQ ID NO:46) cleaving the HIV1_{—}1.4 target (SEQ ID NO:322). On the filter, the positive variants presented correspond to: F10, SEQ ID NO:63; H2, SEQ ID NO:60; 113, SEQ ID NO:59; A3, SEQ ID NO:64; F4, SEQ ID NO:65; some of them described in Table XI. Each cluster contains 4 spots. On the 2 spots on the left, a yeast strain harboring the HIV1_{—}1.4 mutant (SEQ ID NO:46) and the HIV1_{—}1 target (SEQ ID NO:319) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

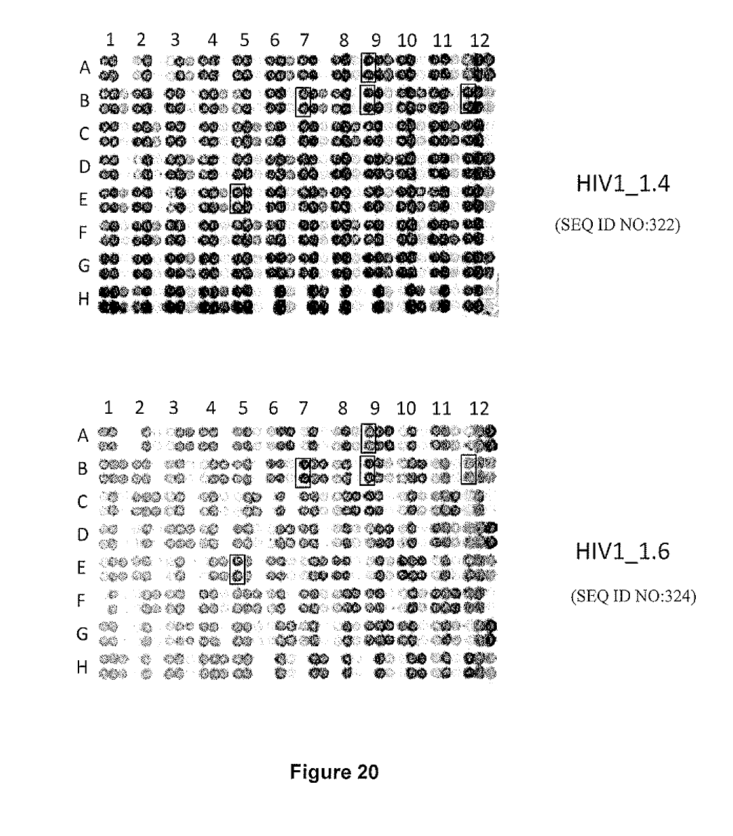

FIG. 20: Cleavage of HIV1_{—}1.4 (SEQ ID NO:322) and HIV1_{—}1.6 (SEQ ID NO:324) targets by meganuclease variants improved by random mutagenesis in example 7. The figure displays an example of screening of I-CreI meganuclease variants with the HIV1_{—}1.4 (SEQ ID NO:322) and HIV1_{—}1.6 (SEQ ID NO:324) targets. On the filter, the positive variants presented correspond to: B7, SEQ ID NO:46; B9, SEQ ID NO:68; B12, SEQ ID NO:69; A9, SEQ ID NO:70; E5, SEQ ID NO:71; all described in Table XIII. Each cluster contains 6 spots. On the 2 spots on the left, a yeast strain harboring the HIV1_{—}1.4 (SEQ ID NO:322) or the HIV1_{—}1.6 (SEQ ID NO:324) targets have been mated with another yeast strain containing the meganuclease variants. The two spots in the middle contain, as an internal control, a non-improved variant cleaving the HIV1_{—}1.4 target (SEQ ID NO:322). The two spots on the right contain the same negative or positive controls. These controls are serially repeated every three clusters as follows: negative control (i.e. cluster A1), positive control (i.e. cluster A2), and strong positive control (i.e. cluster A3).

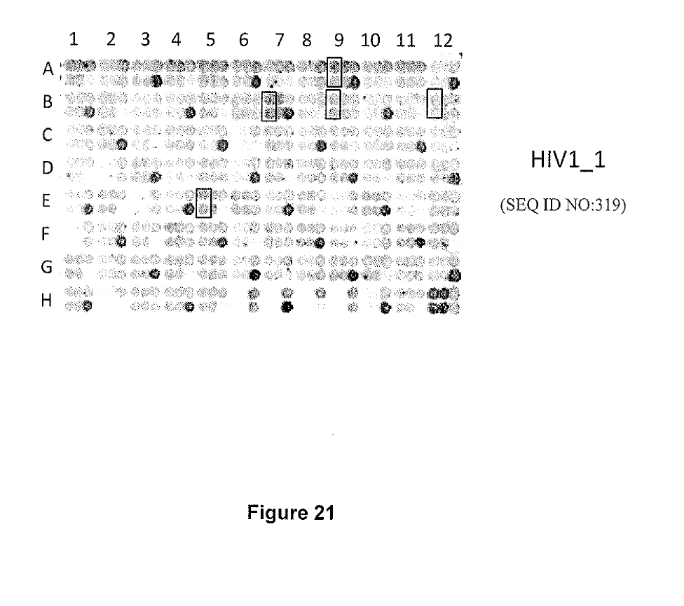

FIG. 21: Cleavage of HIV1_{—}1 target (SEQ ID NO:319) by meganuclease variants improved by random mutagenesis in example 7. The figure displays an example of screening of I-CreI meganuclease variants with the HIV1_{—}1 target (SEQ ID NO:319), when mated with a meganuclease (SEQ ID NO:26) cleaving the HIV1_{—}1.3 target (SEQ ID NO:321). On the filter, the positive variants presented correspond to: B7, SEQ ID NO:46; B9, SEQ ID NO:68; B12, SEQ ID NO:69; A9, SEQ ID NO:70; E5, SEQ ID NO:71; all described in Table XIII. Each cluster contains 6 spots. On the 2 spots on the left, as well as those in the middle, a yeast strain harboring the HIV1_{—}1.3 mutant (SEQ ID NO:26) and the HIV1_{—}1 target (SEQ ID NO:319) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

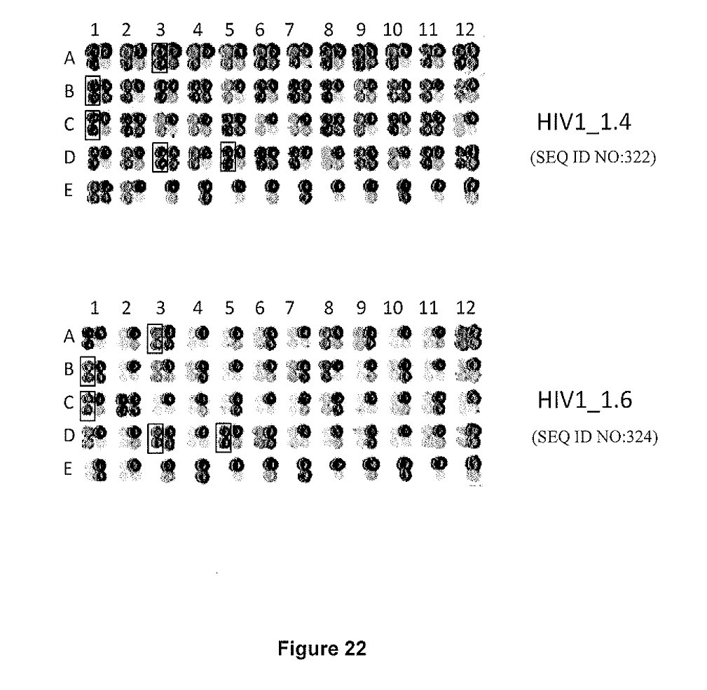

FIG. 22: Cleavage of HIV1_{—}1.4 (SEQ ID NO:322) and HIV1_{—}1.6 (SEQ ID NO:324) targets by meganuclease variants improved by a second round of random mutagenesis in example 7bis. The figure displays an example of screening of I-CreI meganuclease variants with the HIV1_{—}1.4 (SEQ ID NO:322) and HIV1_{—}1.6 (SEQ ID NO:324) targets. On the filter, the positive variants presented correspond to: A3, SEQ ID NO:76; B1, SEQ ID NO:77; C1, SEQ ID NO:78; D3, SEQ ID NO:79; D5, SEQ ID NO:80; all described in Table XIV. Each cluster contains 4 spots. On the 2 spots on the left, a yeast strain harboring the HIV1_{—}1.4 (SEQ ID NO:322) or the HIV1_{—}1.6 (SEQ ID NO:324) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

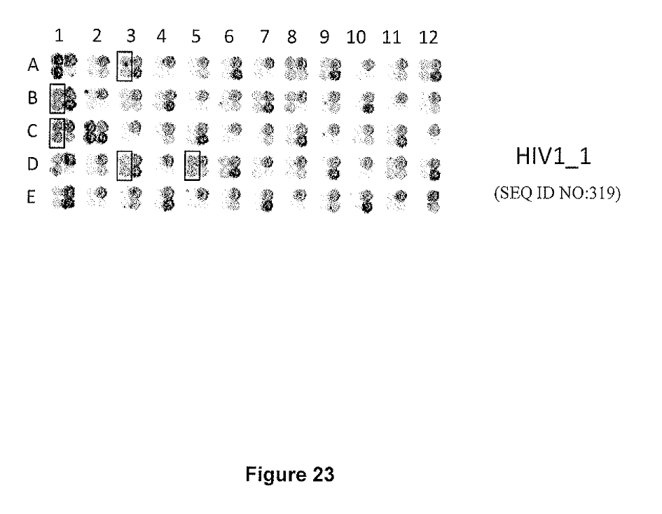

FIG. 23: Cleavage of HIV1_{—}1 target (SEQ ID NO:319) by meganuclease variants improved by a second round of random mutagenesis in example 7bis. The figure displays an example of screening of I-CreI meganuclease variants with the HIV1_{—}1 target (SEQ ID NO:319), when mated with a meganuclease (SEQ ID NO:26) cleaving the HIV1_{—}1.3 target (SEQ ID NO:321). On the filter, the positive variants presented correspond to: A3, SEQ ID NO:76; B1, SEQ ID NO:77; C1, SEQ ID NO:78; D3, SEQ ID NO:79; D5, SEQ ID NO:80; all described in Table XIV. Each cluster contains 4 spots. On the 2 spots on the left, a yeast strain harboring the HIV1_{—}1.3 mutant (SEQ ID NO:26) and the HIV1_{—}1 target (SEQ ID NO:319) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

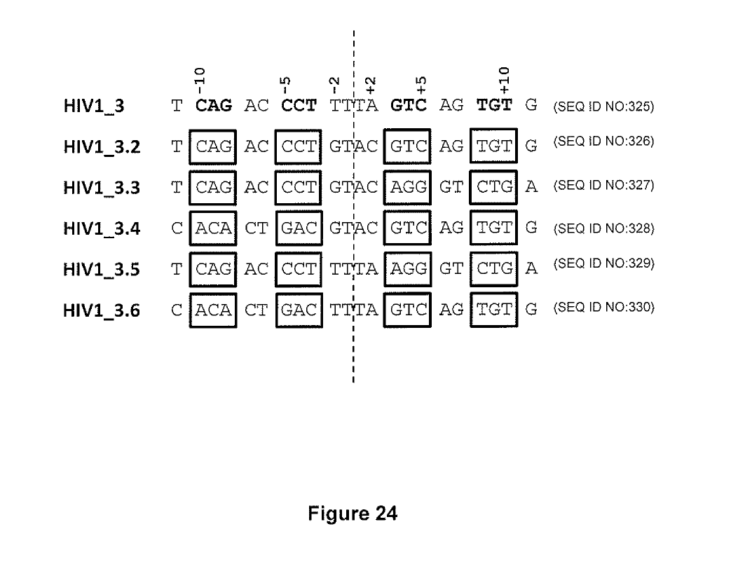

FIG. 24: The HIV1_{—}3 target sequence (SEQ ID NO:325) and its derivatives. In the HIV1_{—}3.2 target (SEQ ID NO:326), the TTTA sequence in the middle of the target is replaced with GTAC, the bases found in C1221 (SEQ ID NO:343). HIV1_{—}3.3 (SEQ ID NO:327) is the palindromic sequence derived from the left part of HIV1_{—}3.2 (SEQ ID NO:326), and HIV1_{—}3.4 (SEQ ID NO:328) is the palindromic sequence derived from the right part of HIV1_{—}3.2 (SEQ ID NO:326). HIV1_{—}3.5 (SEQ ID NO:329) and HIV1_{—}3.6 (SEQ ID NO:330) are pseudopalindromic targets derived, respectively, from HIV1_{—}3.3 (SEQ ID NO:327) and HIV1_{—}3.4 (SEQ ID NO:328), containing the natural TTTA sequence in the middle of the target. As shown in the Figure, the boxed motives from 10CAG_P (SEQ ID NO:374), 10ACA_P (SEQ ID NO:375), 5CCT_P (SEQ ID NO:384) and 5GAC_P (SEQ ID NO:385) are found in the HIV1_{—}3 series of targets (SEQ ID NO:325 to 330).

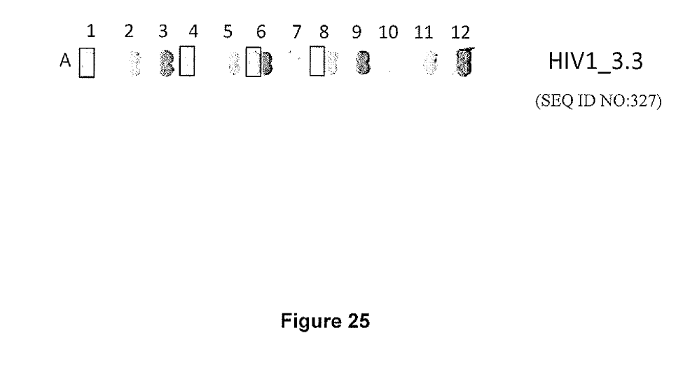

FIG. 25: Cleavage of HIV1_{—}3.3 target (SEQ ID NO:327) by combinatorial variants. The figure displays an example of screening of I-CreI combinatorial variants with the HIV1_{—}3.3 target (SEQ ID NO:327). On the filter, the positive variants correspond to: A6, SEQ ID NO:89; A1, SEQ ID NO:91; A8, SEQ ID NO:90; A4, SEQ ID NO:88; all described in Table XVI. Each cluster contains 4 spots. On the spots on the left, a yeast strain harboring the HIV1_{—}3.3 target (SEQ ID NO:327) has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

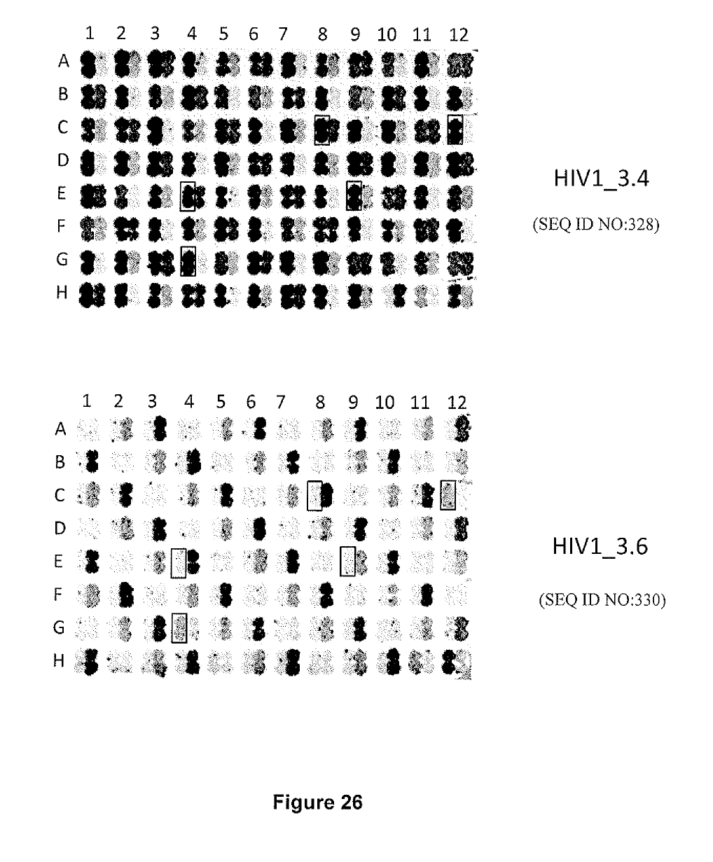

FIG. 26: Cleavage of HIV1_{—}3.4 (SEQ ID NO:328) and HIV1_{—}3.6 (SEQ ID NO:330) targets by combinatorial variants. The figure displays an example of screening of I-CreI combinatorial variants with the HIV1_{—}3.4 (SEQ ID NO:328) and HIV1_{—}3.6 (SEQ ID NO:330) targets. On the filter, the positive variants correspond to: C12, SEQ ID NO:98; C8, SEQ ID NO:99; E4, SEQ ID NO:100; G4, SEQ ID NO:97; E9, SEQ ID NO:101; all described in Table XVIII. Each cluster contains 4 spots. On the spots on the left, a yeast strain harboring the HIV1_{—}3.4 (SEQ ID NO:328) or the HIV1_{—}3.6 (SEQ ID NO:330) targets has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

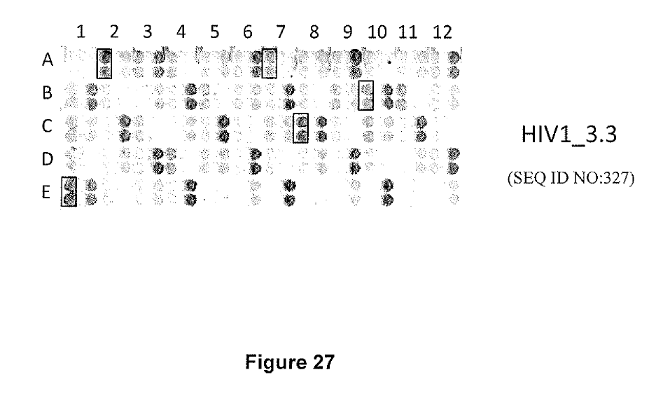

FIG. 27: Cleavage of HIV1_{—}3.3 target (SEQ ID NO:327) by meganuclease variants improved by random mutagenesis in example 12. The figure displays an example of screening of I-CreI meganuclease variants with the HIV1_{—}3.3 target (SEQ ID NO:327). On the filter, the positive variants presented correspond to: E1, SEQ ID NO:105; C8, SEQ ID NO:106; A2, SEQ ID NO:107; A7, SEQ ID NO:108; B10, SEQ ID NO:109; all described in Table XIX. Each cluster contains 6 spots. On the 2 spots on the left, a yeast strain harboring the HIV1_{—}3.3 target (SEQ ID NO:327) has been mated with another yeast strain containing the meganuclease variants. The two spots in the middle contain, as an internal control, a non-improved variant cleaving the HIV1_{—}3.3 target (SEQ ID NO:327). The two spots on the right contain the same negative or positive controls. These controls are serially repeated every three clusters as follows: negative control (i.e. cluster A1), positive control (i.e. cluster A2), and strong positive control (i.e. cluster A3).

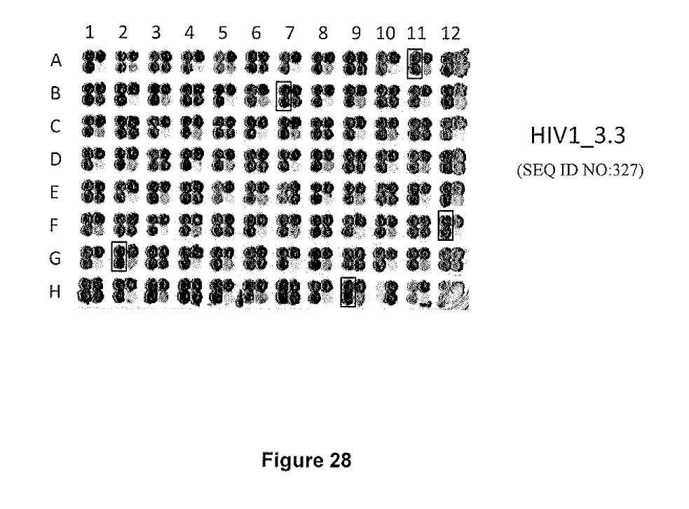

FIG. 28: Cleavage of HIV1_{—}3.3 target (SEQ ID NO:327) by meganuclease variants improved by a second round of random mutagenesis in example 12bis. The figure displays an example of screening of I-CreI meganuclease variants with the HIV1_{—}3.3 target (SEQ ID NO:327). On the filter, the positive variants presented correspond to: A11, SEQ ID NO:115; B7, SEQ ID NO:116; F12, SEQ ID NO:117; G2, SEQ ID NO:118; H9, SEQ ID NO:119; all described in Table XX. Each cluster contains 4 spots. On the 2 spots on the left, a yeast strain harboring the HIV1_{—}3.3 target (SEQ ID NO:327) has been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a variant issued from the first round of improvement.

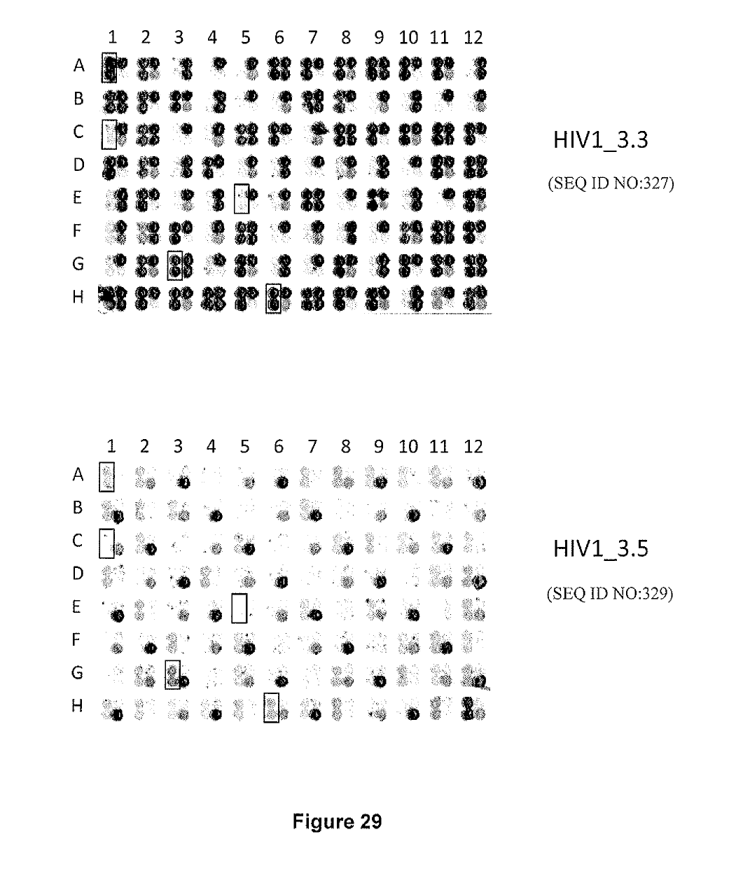

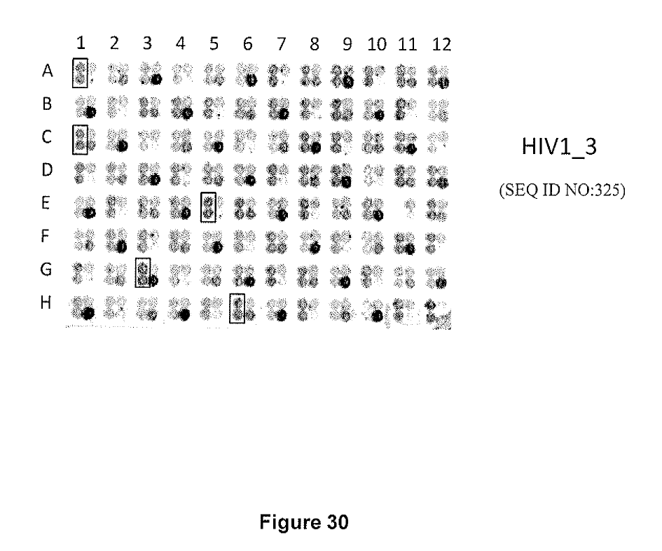

FIG. 29: Cleavage of HIV1_{—}3.3 (SEQ ID NO:327) and HIV1_{—}3.5 (SEQ ID NO:329) targets by meganuclease variants improved by site-directed mutagenesis in example 13. The figure displays an example of screening of I-CreI meganuclease variants with the HIV1_{—}3.3 (SEQ ID NO:327) and HIV1_{—}3.5 (SEQ ID NO:329) targets. On the filter, the positive variants presented correspond to: A1, SEQ ID NO:126; G3, SEQ ID NO:127; C1, SEQ ID NO:128; H6, SEQ ID NO:129; E5, SEQ ID NO:130; described in Table XXI. Some of these variants show no cleavage activity as homodimers while they are active as heterodimers on the HIV1_{—}3 target (SEQ ID NO:325) (see FIG. 30). This is due to the presence of the G19S mutation in these variants. Each cluster contains 4 spots. On the 2 spots on the left, a yeast strain harboring the HIV1_{—}3.3 (SEQ ID NO:327) or the HIV1_{—}3.5 (SEQ ID NO:329) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a previously improved variant.

FIG. 30: Cleavage of HIV1_{—}3 (SEQ ID NO:325) target by meganuclease variants improved by site-directed mutagenesis in example 13. The figure displays an example of screening of I-CreI meganuclease variants with the HIV1_{—}3 target (SEQ ID NO:325), when mated with a meganuclease (SEQ ID NO:125) cleaving the HIV1_{—}3.4 target (SEQ ID NO:328). On the filter, the positive variants presented correspond to: A1, SEQ ID NO:126; G3, SEQ ID NO:127; C1, SEQ ID NO:128; H6, SEQ ID NO:129; E5, SEQ ID NO:130; described in Table XXI. Each cluster contains 4 spots. On the 2 spots on the left, a yeast strain harboring the HIV1_{—}3.4 mutant (SEQ ID NO:125) and the HIV1_{—}3 target (SEQ ID NO:325) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a previously improved variant.

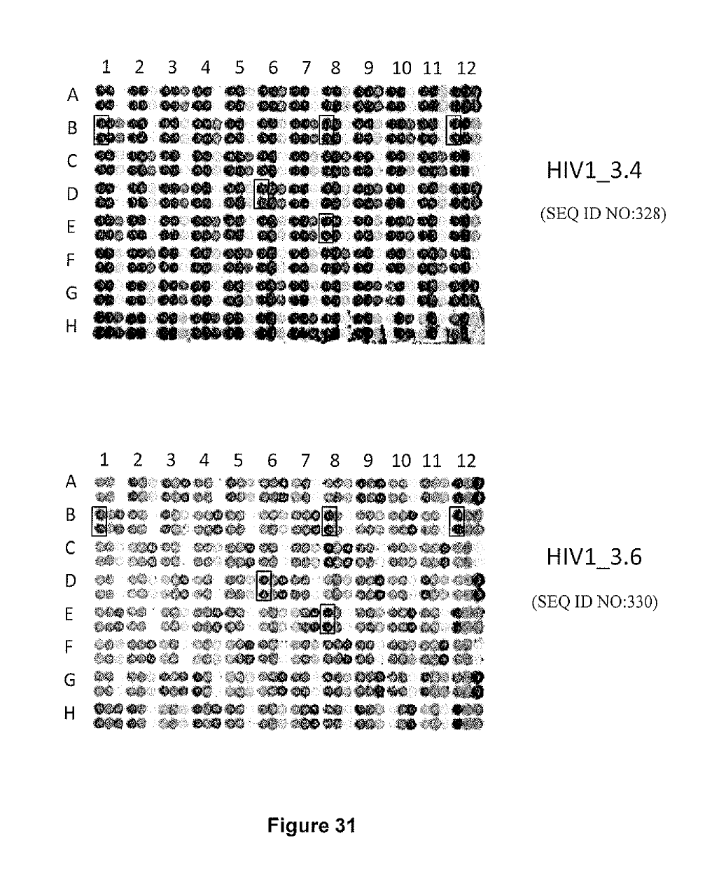

FIG. 31: Cleavage of HIV1_{—}3.4 (SEQ ID NO:328) and HIV1_{—}3.6 (SEQ ID NO:330) targets by meganuclease variants improved by random mutagenesis in example 14. The figure displays an example of screening of I-CreI meganuclease variants with the HIV1_{—}3.4 (SEQ ID NO:328) and HIV1_{—}3.6 (SEQ ID NO:330) targets. On the filter, the positive variants presented correspond to: E8, SEQ ID NO:136; B12, SEQ ID NO:137; B1, SEQ ID NO:138; B8, SEQ ID NO:139; D6, SEQ ID NO:140; all described in Table XXII. Each cluster contains 6 spots. On the 2 spots on the left, a yeast strain harboring the HIV1_{—}3.4 (SEQ ID NO:328) or the HIV1_{—}3.6 (SEQ ID NO:330) targets has been mated with another yeast strain containing the meganuclease variants. The two spots in the middle contain, as an internal control, a non-improved variant cleaving the HIV1_{—}3.4 target (SEQ ID NO:328). The two spots on the right contain the same negative or positive controls. These controls are serially repeated every three clusters as follows: negative control (i.e. cluster A1), positive control (i.e. cluster A2), and strong positive control (i.e. cluster A3).

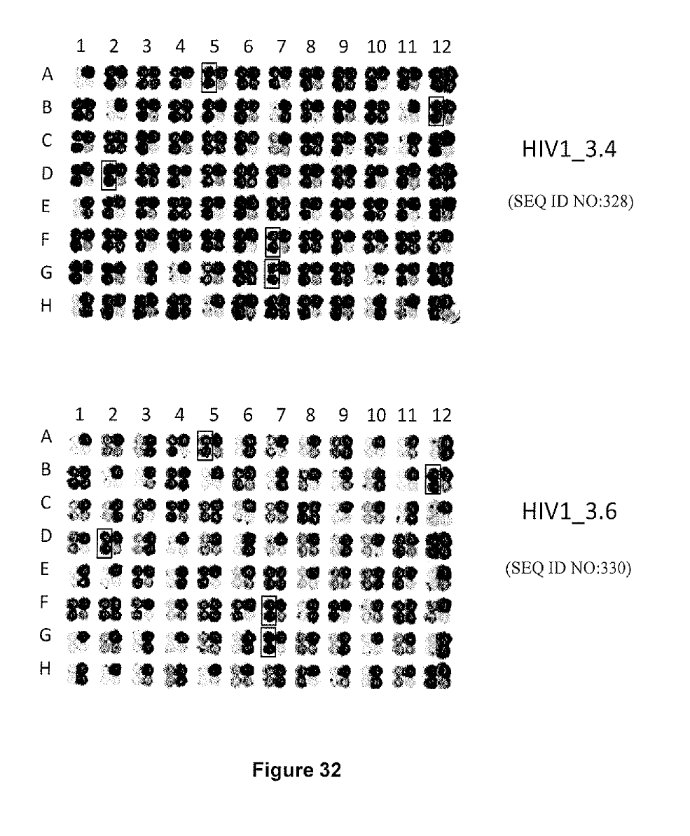

FIG. 32: Cleavage of HIV1_{—}3.4 (SEQ ID NO:328) and HIV1_{—}3.6 (SEQ ID NO:330) targets by meganuclease variants improved by a second round of random mutagenesis in example 14bis. The figure displays an example of screening of I-CreI meganuclease variants with the HIV1_{—}3.4 (SEQ ID NO:328) and HIV1_{—}3.6 (SEQ ID NO:330) targets. On the filter, the positive variants presented correspond to:

F7, SEQ ID NO:146; B12, SEQ ID NO:147; G7, SEQ ID NO:148; D2, SEQ ID NO:149; A5, SEQ ID NO:150; all described in Table XXIII. Each cluster contains 4 spots. On the 2 spots on the left, a yeast strain harboring the HIV1_{—}3.4 (SEQ ID NO:328) or the HIV1_{—}3.6 (SEQ ID NO:330) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a previously improved variant.

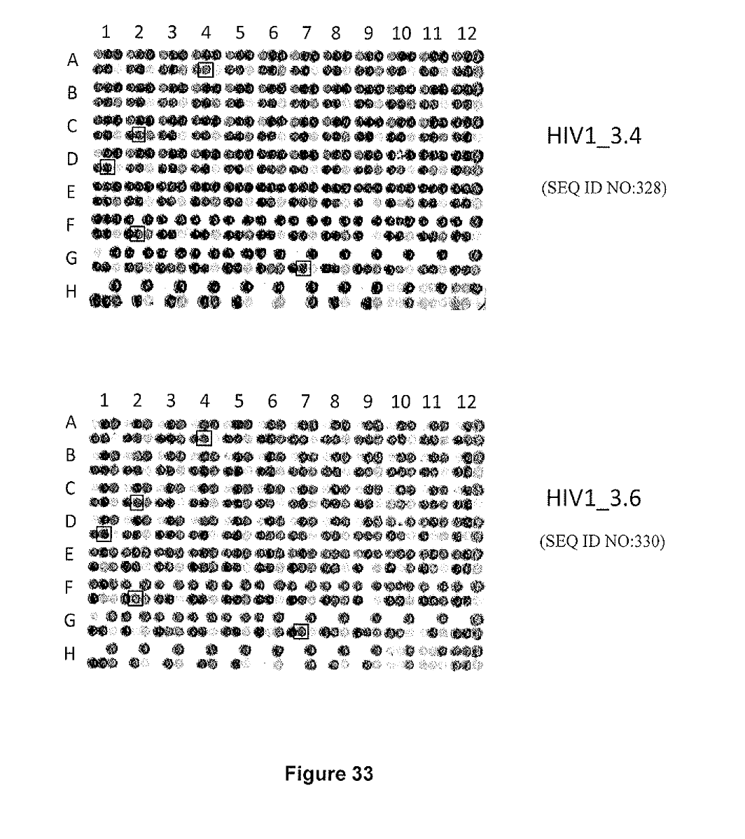

FIG. 33: Cleavage of HIV1_{—}3.4 (SEQ ID NO:328) and HIV1_{—}3.6 (SEQ ID NO:330) targets by meganuclease variants improved by site-directed mutagenesis in example 15. The figure displays an example of screening of I-CreI meganuclease variants with the HIV1_{—}3.4 (SEQ ID NO:328) and HIV1_{—}3.6 (SEQ ID NO:330) targets. On the filter, the positive variants presented correspond to: D1, SEQ ID NO:156; C2, SEQ ID NO:157; F2, SEQ ID NO:158; A4, SEQ ID NO:159; G7, SEQ ID NO:160; described in Table XXIV. Each cluster contains 6 spots. On the 4 spots on the left, a yeast strain harboring the HIV1_{—}3.4 (SEQ ID NO:328) or the HIV1_{—}3.6 (SEQ ID NO:330) targets have been mated with another yeast strain containing 4 different meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a non-improved variant.

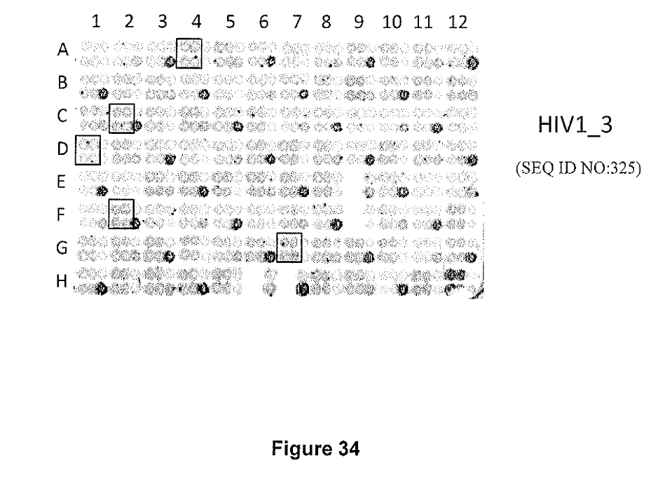

FIG. 34: Cleavage of HIV1_{—}3 target (SEQ ID NO:325) by meganuclease variants improved by site-directed mutagenesis in example 15. The figure displays an example of screening of I-CreI meganuclease variants with the HIV1_{—}3 target (SEQ ID NO:325), when mated with a meganuclease (SEQ ID NO:109) cleaving the HIV1_{—}3.3 target (SEQ ID NO:327). On the filter, the positive variants presented correspond to: D1, SEQ ID NO:156; C2, SEQ ID NO:157; F2, SEQ ID NO:158; A4, SEQ ID NO:159; G7, SEQ ID NO:160; described in Table XXIV. Each cluster contains 6 spots. On the 4 spots on the left, a yeast strain harboring the HIV1_{—}3 target (SEQ ID NO:325) and the HIV1_{—}3.3 mutant (SEQ ID NO:109) has been mated with another yeast strain containing different meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a previously improved variant.

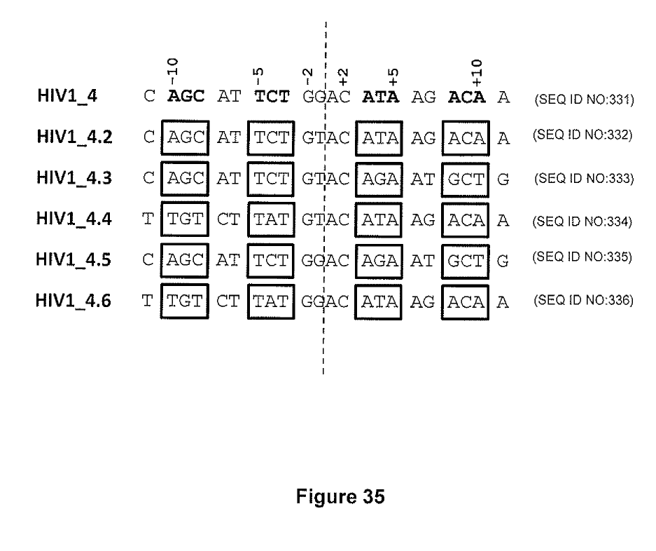

FIG. 35: The HIV1_{—}4 (SEQ ID NO:331) target sequence and its derivatives. In the HIV1_{—}4.2 target (SEQ ID NO:332), the GGAC sequence in the middle of the target is replaced with GTAC, the bases found in C1221 (SEQ ID NO:343). HIV1_{—}4.3 (SEQ ID NO:333) is the palindromic sequence derived from the left part of HIV1_{—}4.2 (SEQ ID NO:332), and HIV1_{—}4.4 (SEQ ID NO:334) is the palindromic sequence derived from the right part of HIV1_{—}4.2 (SEQ ID NO:332). HIV1_{—}4.5 (SEQ ID NO:335) and HIV1_{—}4.6 (SEQ ID NO:336) are pseudopalindromic targets derived, respectively, from HIV1_{—}4.3 (SEQ ID NO:333) and HIV1_{—}4.4 (SEQ ID NO:334), containing the natural GGAC sequence in the middle of the target. As shown in the Figure, the boxed motives from 10AGC_P (SEQ ID NO:383), 10TGT_P (SEQ ID NO:382), 5TCT_P (SEQ ID NO:390) and 5TAT_P (SEQ ID NO:391) are found in the HIV1_{—}4 series of targets (SEQ ID NO:331 to 336).

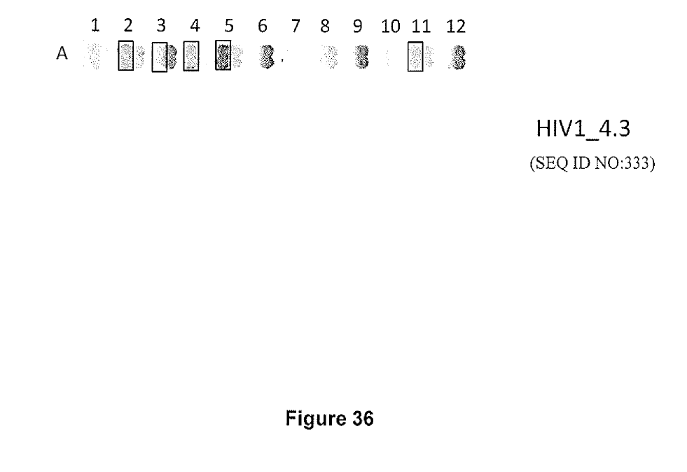

FIG. 36: Cleavage of HIV1_{—}4.3 (SEQ ID NO:333) target by combinatorial variants. The figure displays an example of screening of I-CreI combinatorial variants with the HIV1_{—}4.3 target (SEQ ID NO:333). On the filter, the positive variants correspond to: A11, SEQ ID NO:168; A5, SEQ ID NO:170; A2, SEQ ID NO:171; A4, SEQ ID NO:173; A3, SEQ ID NO:174; all described in Table XXVI. Each cluster contains 4 spots. On the spots on the left, a yeast strain harboring the HIV1_{—}4.3 (SEQ ID NO:333) target has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

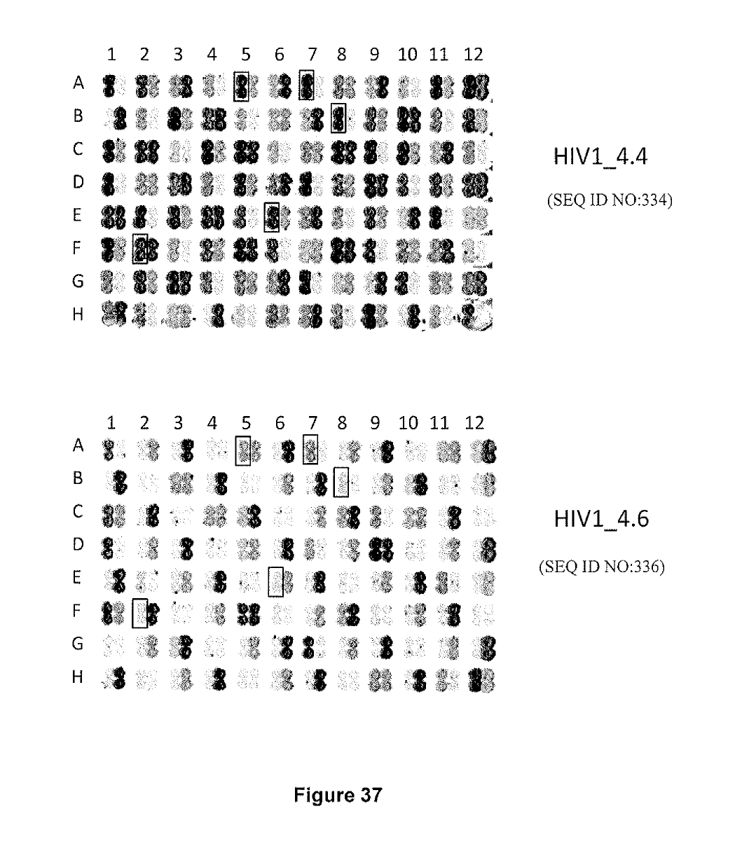

FIG. 37: Cleavage of HIV1_{—}4.4 (SEQ ID NO:334) and HIV1_{—}4.6 (SEQ ID NO:336) targets by combinatorial variants. The figure displays an example of screening of I-CreI combinatorial variants with the HIV1_{—}4.4 (SEQ ID NO:334) and HIV1_{—}4.6 (SEQ ID NO:336) targets. On the filter, the positive variants correspond to: A7, SEQ ID NO:177; A5, SEQ ID NO:178; B8, SEQ ID NO:179; E6, SEQ ID NO:180; F2, SEQ ID NO:181; all described in Table XXVIII. Each cluster contains 4 spots. On the spots on the left, a yeast strain harboring the HIV1_{—}4.4 (SEQ ID NO:334) or the HIV1_{—}4.6 (SEQ ID NO:336) targets has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

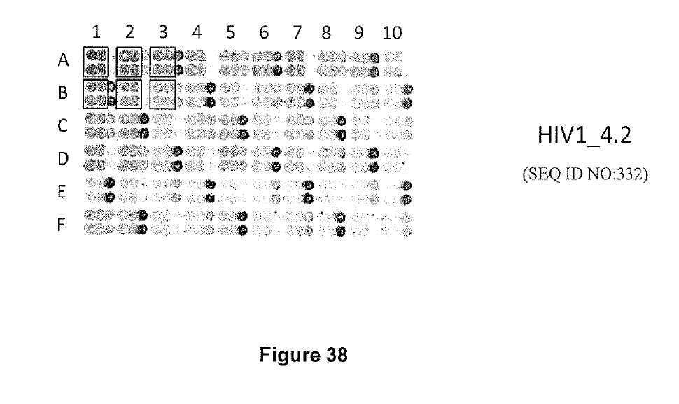

FIG. 38: Cleavage of the HIV1_{—}4.2 (SEQ ID NO:332) and HIV1_{—}4 (SEQ ID NO:331) target sequences by heterodimeric combinatorial variants. Example of screening of combinations of I-CreI variants against the HIV1_{—}4.2 target (SEQ ID NO:332). Some heterodimers resulted in cleavage of the HIV1_{—}4.2 target (SEQ ID NO:332), while no cleavage activity was detected on the HIV1_{—}4 target (SEQ ID NO:331). On the filter, the position of mutants in certain positions as an example is: line A, SEQ ID NO:170; line B, SEQ ID NO:171; column 1, SEQ ID NO:177; column 2, SEQ ID NO:178; column 3; SEQ ID NO:179. These mutants have been described in Tables XXVI and XXVIII. Each cluster contains 6 spots. On the 4 spots on the left, a yeast strain harboring the HIV1_{—}4 (SEQ ID NO:331) or HIV1_{—}4.2 target (SEQ ID NO:332) have been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

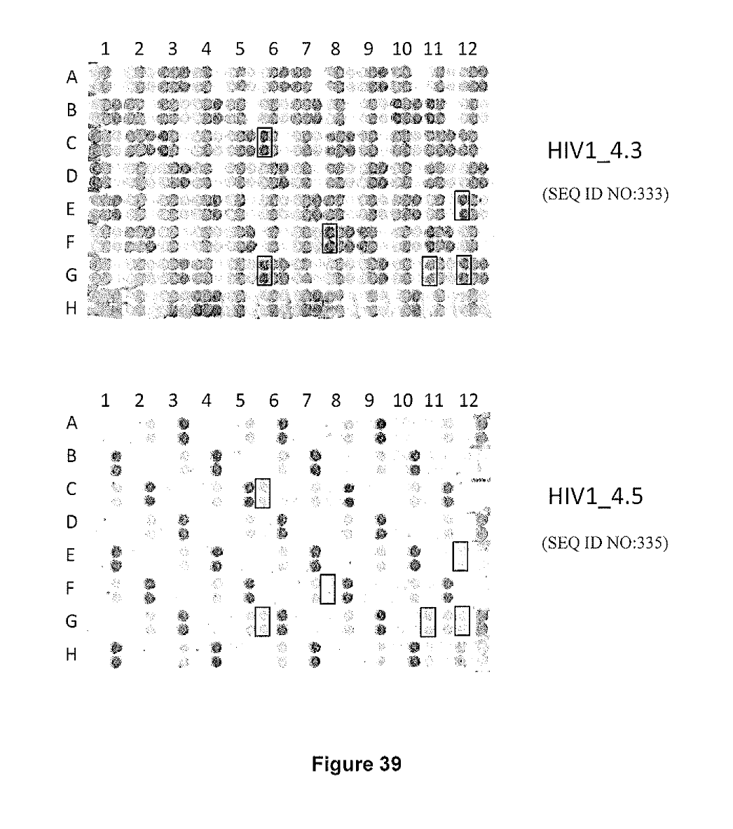

FIG. 39: Cleavage of HIV1_{—}4.3 (SEQ ID NO:333) and HIV1_{—}4.5 (SEQ ID NO:335) targets by meganuclease variants improved by random mutagenesis in example 20. The figure displays an example of screening of I-CreI meganuclease variants with the HIV1_{—}4.3 (SEQ ID NO:333) and HIV1_{—}4.5 (SEQ ID NO:335) targets. On the filter, the positive variants presented correspond to: F8, SEQ ID NO:189; C6, SEQ ID NO:190; E12, SEQ ID NO:191; G12, SEQ ID NO:192; G6, SEQ ID NO:193; G11, SEQ ID NO:194; all described in Table XXX. Each cluster contains 6 spots. On the 2 spots on the left, a yeast strain harboring the HIV1_{—}4.3 (SEQ ID NO:333) or the HIV1_{—}4.5 (SEQ ID NO:335) targets have been mated with another yeast strain containing the meganuclease variants. The two spots in the middle contain, as an internal control, a non-improved variant cleaving the HIV1_{—}4.3 target (SEQ ID NO:333). The two spots on the right contain the same negative or positive controls. These controls are serially repeated every three clusters as follows: negative control (i.e. cluster A1), positive control (i.e. cluster A2), and strong positive control (i.e. cluster A3).

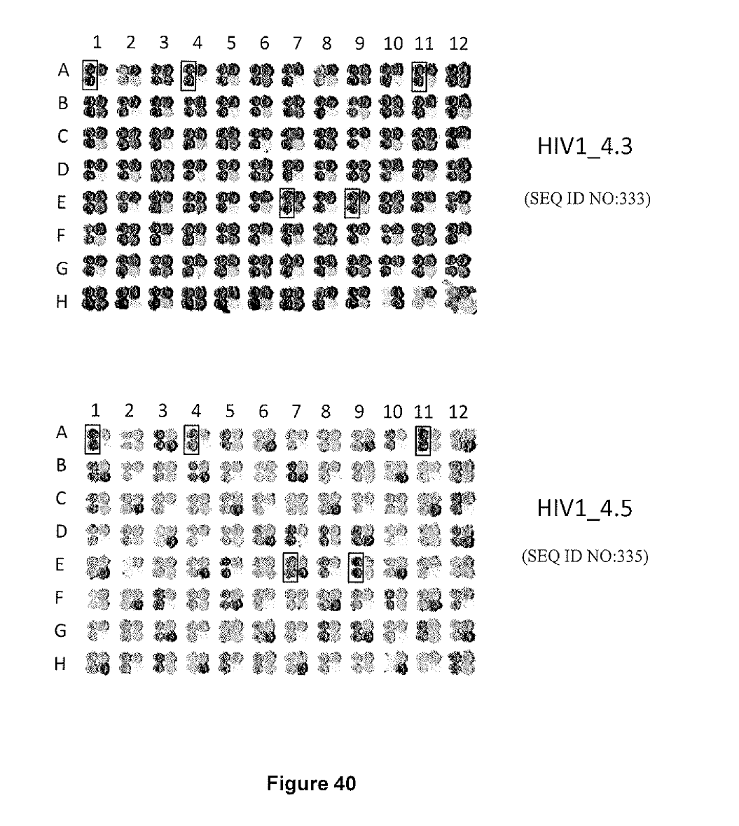

FIG. 40: Cleavage of HIV1_{—}4.3 (SEQ ID NO:333) and HIV1_{—}4.5 (SEQ ID NO:335) targets by meganuclease variants improved by a second round of random mutagenesis in example 20bis. The figure displays an example of screening of I-CreI meganuclease variants with the HIV1_{—}4.3 (SEQ ID NO:333) and HIV1_{—}4.5 (SEQ ID NO:335) targets. On the filter, the positive variants presented correspond to: E7, SEQ ID NO:200; A1, SEQ ID NO:201; E9, SEQ ID NO:202; A4, SEQ ID NO:203; A11, SEQ ID NO:204; all described in Table XXXI. Each cluster contains 4 spots. On the 2 spots on the left, a yeast strain harboring the HIV1_{—}4.3 (SEQ ID NO:333) or the HIV1_{—}4.5 (SEQ ID NO:335) targets has been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

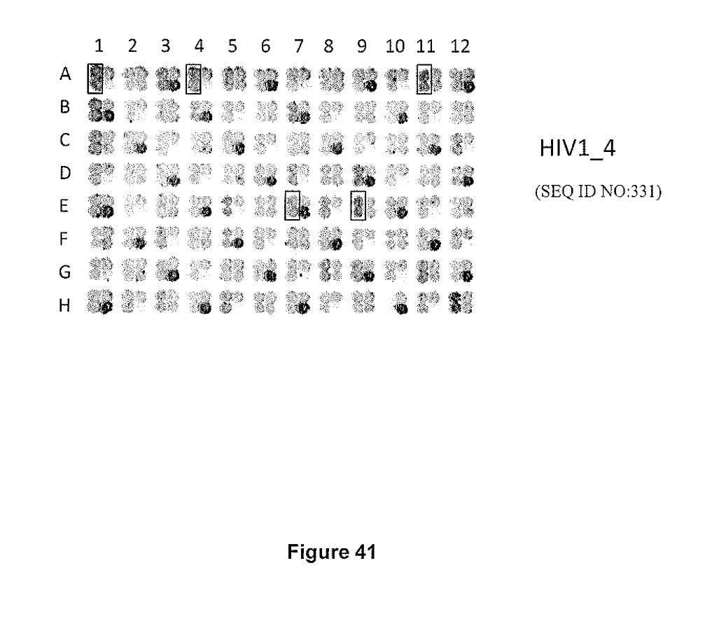

FIG. 41: Cleavage of HIV1_{—}4 (SEQ ID NO:331) target by meganuclease variants improved by a second round of random mutagenesis in example 20bis. The figure displays an example of screening of I-CreI meganuclease variants with the HIV1_{—}4 target (SEQ ID NO:331), when mated with a meganuclease (SEQ ID NO:199) cleaving the HIV1_{—}4.4 target (SEQ ID NO:334). On the filter, the positive variants presented correspond to: E7, SEQ ID NO:200; A1, SEQ ID NO:201; E9, SEQ ID NO:202; A4, SEQ ID NO:203; A11, SEQ ID NO:204; all described in Table XXXI. Each cluster contains 4 spots. On the 2 spots on the left, a yeast strain harboring the HIV1_{—}4.4 mutant (SEQ ID NO:199) and the HIV1_{—}4 target (SEQ ID NO:331) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

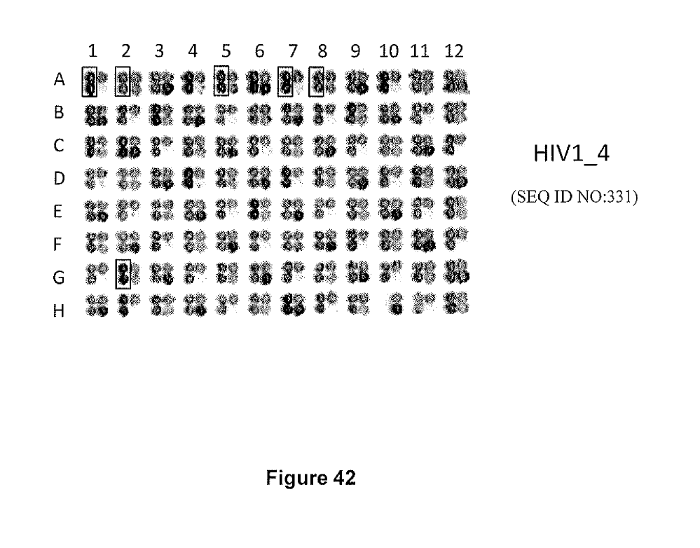

FIG. 42: Cleavage of HIV1_{—}4 (SEQ ID NO:331) target by meganuclease variants improved by site-directed mutagenesis in example 21. The figure displays an example of screening of I-CreI meganuclease variants with the HIV1_{—}4 target (SEQ ID NO:331), when mated with a meganuclease (SEQ ID NO:210) cleaving the HIV1_{—}4.4 target (SEQ ID NO:334). On the filter, the positive variants presented correspond to: A1, SEQ ID NO:211; A2, SEQ ID NO:212; A5, SEQ ID NO:213; A7, SEQ ID NO:214; A8, SEQ ID NO:215; G2, SEQ ID NO:216; described in Table XXXII. Each cluster contains 4 spots. On the 2 spots on the left, a yeast strain harboring the HIV1_{—}4.4 mutant (SEQ ID NO:210) and the HIV1_{—}4 target (SEQ ID NO:331) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

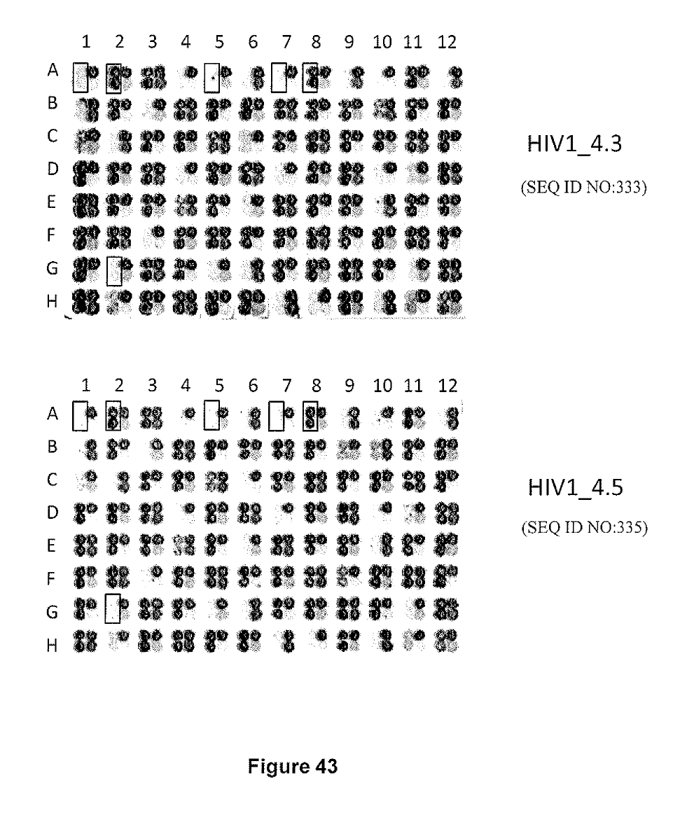

FIG. 43: Cleavage of HIV1_{—}4.3 (SEQ ID NO:333) and HIV1_{—}4.5 (SEQ ID NO:335) targets by meganuclease variants improved by site-directed mutagenesis in example 21. The figure displays an example of screening of I-CreI meganuclease variants with the HIV1_{—}4.3 (SEQ ID NO:333) and HIV1_{—}4.5 (SEQ ID NO:335) targets. On the filter, the variants presented correspond to: A1, SEQ ID NO:211; A2, SEQ ID NO:212; A5, SEQ ID NO:213; A7, SEQ ID NO:214; A8, SEQ ID NO:215; G2, SEQ ID NO:216; described in Table XXXII. Some of these variants show no cleavage activity as homodimers while they are active as heterodimers on the HIV1_{—}4 target (SEQ ID NO:331) (see FIG. 42). This is due to the presence of the G19S mutation in these variants. Each cluster contains 4 spots. On the 2 spots on the left, a yeast strain harboring the HIV1_{—}4.3 (SEQ ID NO:333) or the HIV1_{—}4.5 (SEQ ID NO:335) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

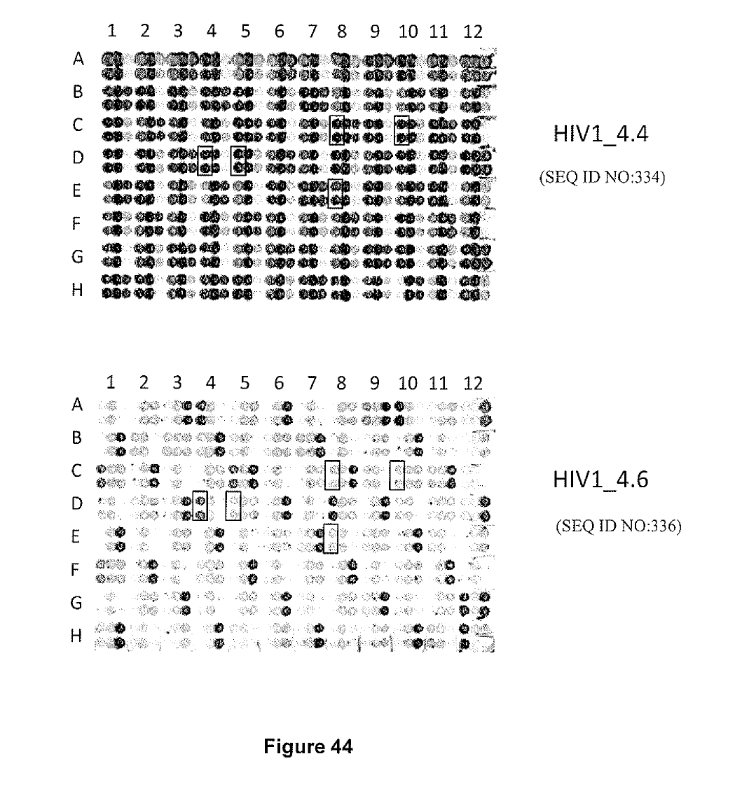

FIG. 44: Cleavage of HIV1_{—}4.4 (SEQ ID NO:334) and HIV1_{—}4.6 (SEQ ID NO:336) targets by meganuclease variants improved by random mutagenesis in example 22. The figure displays an example of screening of I-CreI meganuclease variants with the HIV1_{—}4.4 (SEQ ID NO:334) and HIV1_{—}4.6 (SEQ ID NO:336) targets. On the filter, the positive variants presented correspond to: D4, SEQ ID NO:199; D5, SEQ ID NO:210; C8, SEQ ID NO:221; C10, SEQ ID NO:222; E8, SEQ ID NO:223; all described in Table XXXIII. Each cluster contains 6 spots. On the 2 spots on the left, a yeast strain harboring the HIV1_{—}4.4 (SEQ ID NO:334) or the HIV1_{—}4.6 (SEQ ID NO:336) targets have been mated with another yeast strain containing the meganuclease variants. The two spots in the middle contain, as an internal control, a non-improved variant cleaving the HIV1_{—}4.4 target (SEQ ID NO:334). The two spots on the right contain the same negative or positive controls. These controls are serially repeated every three clusters as follows: negative control (i.e. cluster A1), positive control (i.e. cluster A2), and strong positive control (i.e. cluster A3).

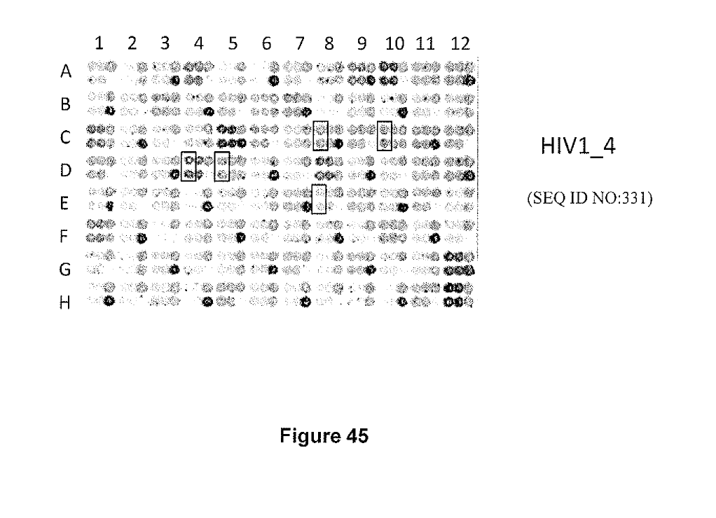

FIG. 45: Cleavage of HIV1_{—}4 (SEQ ID NO:331) target by meganuclease variants improved by random mutagenesis in example 22. The figure displays an example of screening of I-CreI meganuclease variants with the HIV1_{—}4 target (SEQ ID NO:331), when mated with a meganuclease (SEQ ID NO:190) cleaving the HIV1_{—}4.3 target (SEQ ID NO:333). On the filter, the positive variants presented correspond to: D4, SEQ ID NO:199; D5, SEQ ID NO:210; C8, SEQ ID NO:221; C10, SEQ ID NO:222; E8, SEQ ID NO:223; all described in Table XXXIII Each cluster contains 6 spots. On the 4 spots on the left, a yeast strain harboring the HIV1_{—}4.3 mutant (SEQ ID NO:190) and the HIV1_{—}4 (SEQ ID NO:331) target have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a non-improved variant.

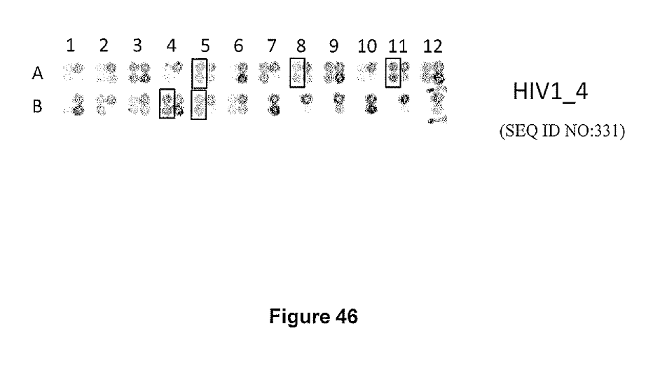

FIG. 46: Cleavage of HIV1_{—}4 (SEQ ID NO:331) target by meganuclease variants improved by site-directed mutagenesis in example 23. The figure displays an example of screening of I-CreI meganuclease variants with the HIV1_{—}4 target (SEQ ID NO:331), when mated with a meganuclease (SEQ ID NO:190) cleaving the HIV1_{—}4.3 target (SEQ ID NO:333). On the filter, the positive variants presented correspond to: B5, SEQ ID NO:229; B4, SEQ ID NO:231; A5, SEQ ID NO:235; A8, SEQ ID NO:236; A11, SEQ ID NO:237; described in Table XXXIV. Each cluster contains 4 spots. On the 2 spots on the left, a yeast strain harboring the HIV1_{—}4 target (SEQ ID NO:331) and the HIV1_{—}4.3 mutant (SEQ ID NO:190) has been mated with another yeast strain containing different meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

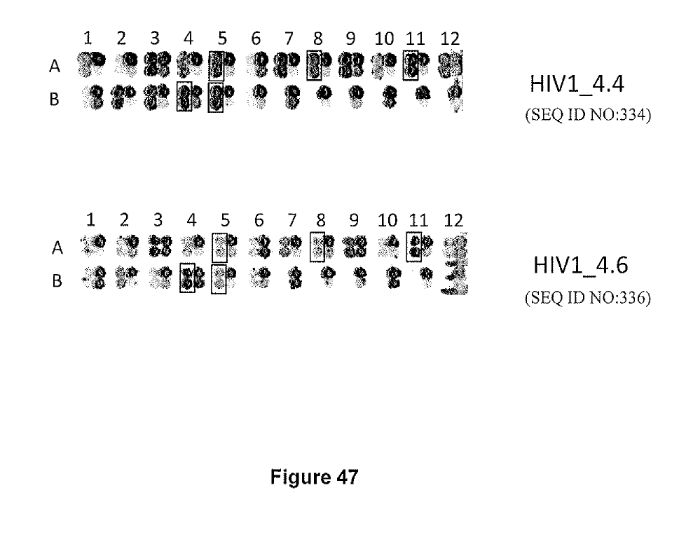

FIG. 47: Cleavage of HIV1_{—}4.4 (SEQ ID NO:334) and HIV1_{—}4.6 (SEQ ID NO:336) targets by meganuclease variants improved by site-directed mutagenesis in example 23. The figure displays an example of screening of I-CreI meganuclease variants with the HIV1_{—}4.4 (SEQ ID NO:334) and HIV1_{—}4.6 (SEQ ID NO:336) targets. On the filter, the positive variants presented correspond to: B5, SEQ ID NO:229; B4, SEQ ID NO:231; A5, SEQ ID NO:235; A8, SEQ ID NO:236; A11, SEQ ID NO:237; described in Table XXXIV. Each cluster contains 4 spots. On the 2 spots on the left, a yeast strain harboring the HIV1_{—}4.4 (SEQ ID NO:334) or the HIV1_{—}4.6 (SEQ ID NO:336) targets have been mated with another yeast strain containing different meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

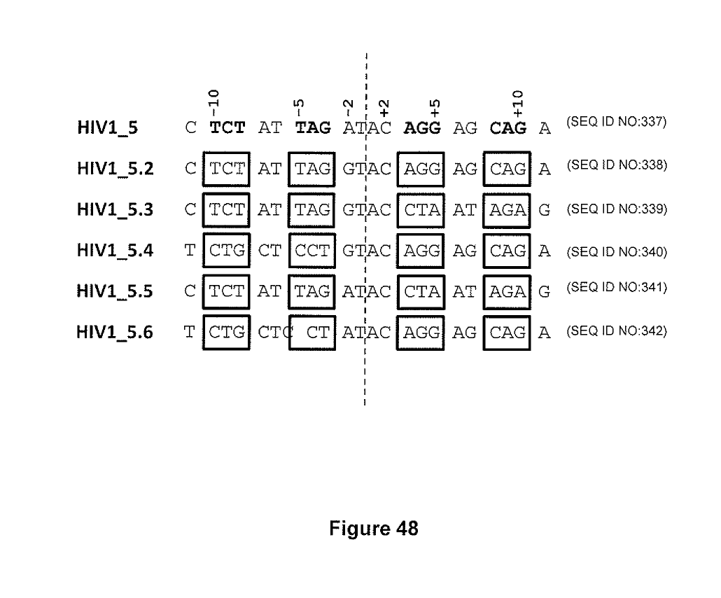

FIG. 48: The HIV1_{—}5 target sequence (SEQ ID NO:337) and its derivatives. In the HIV1_{—}5.2 target (SEQ ID NO:338), the ATAC sequence in the middle of the target is replaced with GTAC, the bases found in C1221 (SEQ ID NO:343). HIV1_{—}5.3 (SEQ ID NO:339) is the palindromic sequence derived from the left part of HIV1_{—}5.2 (SEQ ID NO:338), and HIV1_{—}5.4 (SEQ ID NO:340) is the palindromic sequence derived from the right part of HIV1_{—}5.2 (SEQ ID NO:338). HIV1_{—}5.5 (SEQ ID NO:341) and HIV1_{—}5.6 (SEQ ID NO:342) are pseudopalindromic targets derived, respectively, from HIV1_{—}5.3 (SEQ ID NO:339) and HIV1_{—}5.4 (SEQ ID NO:340), containing the natural ATAC sequence in the middle of the target. As shown in the Figure, the boxed motives from 10TCT_P (SEQ ID NO:377), 10CTG_P (SEQ ID NO:378), 5TAG_P (SEQ ID NO:386) and 5CCT_P (SEQ ID NO:384) are found in the HIV1_{—}5 series of targets (SEQ ID NO:337 to 342).

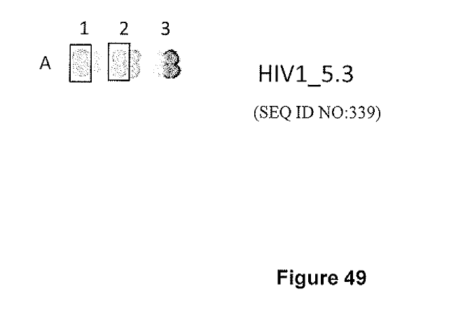

FIG. 49: Cleavage of HIV1_{—}5.3 (SEQ ID NO:339) target by combinatorial variants. The figure displays an example of screening of I-CreI combinatorial variants with the HIV1_{—}5.3 target (SEQ ID NO:339). On the filter, the two positive variants correspond to: A1, SEQ ID NO:242; A2, SEQ ID NO:241; described in Table XXXVI. Each cluster contains 4 spots. On the spots on the left, a yeast strain harboring the HIV1_{—}5.3 target (SEQ ID NO:339) has been mated with another yeast strain containing the meganuclease variants. The two spots on the right contain the same negative or positive controls. These controls are: negative control (cluster A1), positive control (cluster A2), and strong positive control (cluster A3).

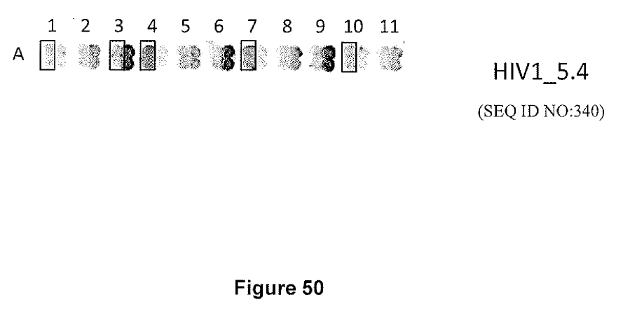

FIG. 50: Cleavage of HIV1_{—}5.4 (SEQ ID NO:340) target by combinatorial variants. The figure displays an example of screening of I-CreI combinatorial variants with the HIV1_{—}5.4 target (SEQ ID NO:340). On the filter, the positive variants correspond to: A1, SEQ ID NO:249; A3, SEQ ID NO:245; A4, SEQ ID NO:252; A7, SEQ ID NO:250; A10, SEQ ID NO:246; all described in Table XXXVIII. Each cluster contains 4 spots. On the spots on the left, a yeast strain harboring the HIV1_{—}5.4 target (SEQ ID NO:340) has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

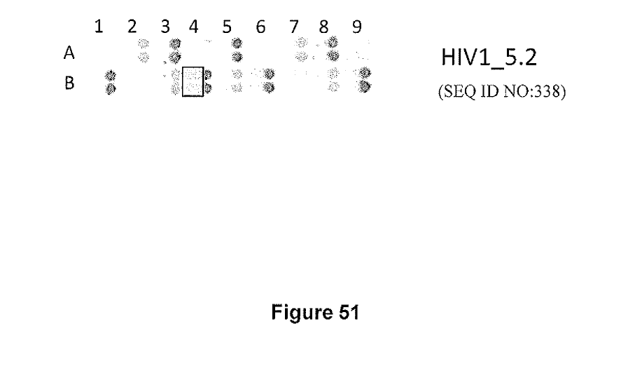

FIG. 51: Cleavage of the HIV1_{—}5.2 target sequence (SEQ ID NO:338) by heterodimeric combinatorial variants. Example of screening of combinations of I-CreI variants against the HIV1_{—}5.2 target (SEQ ID NO:338). One heterodimer resulted in cleavage of the HIV1_{—}5.2 target (SEQ ID NO:338). The heterodimer displaying a signal with HIV1_{—}5.2 target (SEQ ID NO:338) is observed at position B4. On the filter, the position of certain mutants as an example is: line A, SEQ ID NO:242; line B, SEQ ID NO:241; column 3, SEQ ID NO:245; column 4, SEQ ID NO:252; column 5; SEQ ID NO:251. These mutants have been described in Tables XXXVI and XXXVIII. Each cluster contains 6 spots. On the 4 spots on the left, a yeast strain harboring the HIV1_{—}5.2 target (SEQ ID NO:338) has been mated with another yeast strain containing the meganuclease variants. The two spots on the right contain the same negative or positive controls. These controls are serially repeated every three clusters as follows: negative control (i.e. cluster A1), positive control (i.e. cluster A2), and strong positive control (i.e. cluster A3).

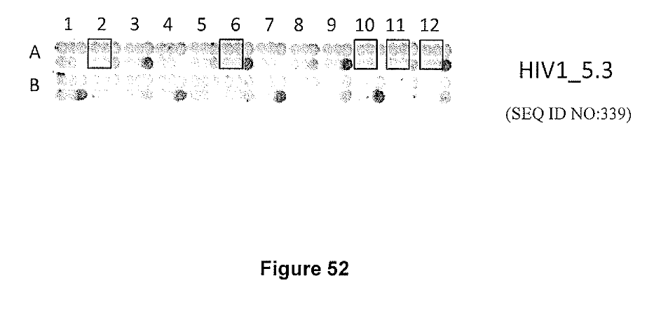

FIG. 52: Cleavage of HIV1_{—}5.3 target (SEQ ID NO:339) by meganuclease variants improved by random mutagenesis in example 28. The figure displays an example of screening of I-CreI meganuclease variants with the HIV1_{—}5.3 target (SEQ ID NO:339). On the filter, the positive variants presented correspond to: A6, SEQ ID NO:256; A12, SEQ ID NO:257; A11, SEQ ID NO:258; A10, SEQ ID NO:259; A2, SEQ ID NO:260; all described in Table XXXIX. Each cluster contains 6 spots. On the 4 spots on the left, a yeast strain harboring the HIV1_{—}5.3 target (SEQ ID NO:339) has been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a non-improved variant.

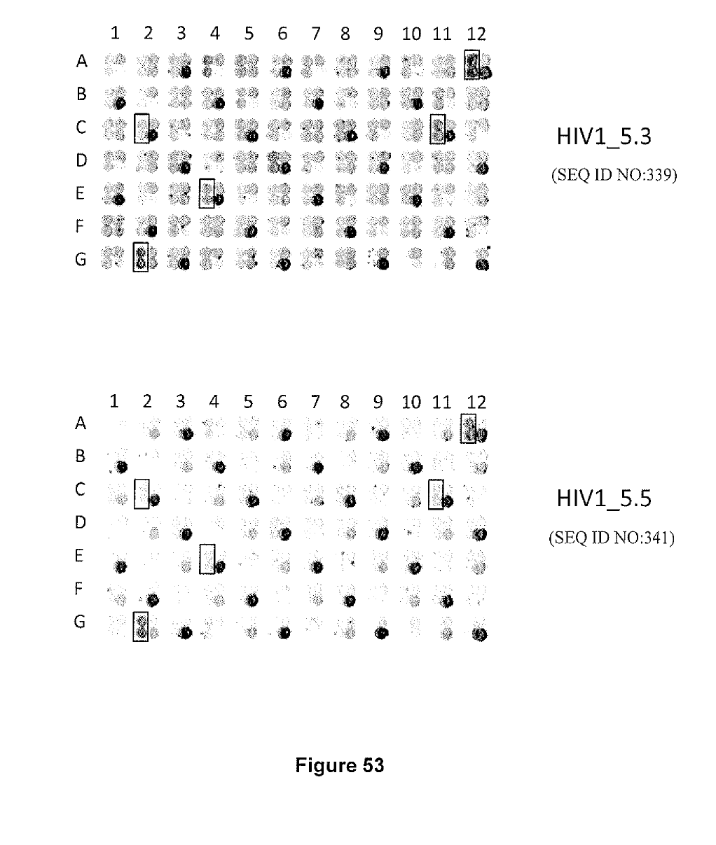

FIG. 53: Cleavage of HIV1_{—}5.3 (SEQ ID NO:339) and HIV1_{—}5.5 (SEQ ID NO:341) targets by meganuclease variants improved by a second round of random mutagenesis in example 28bis. The figure displays an example of screening of I-CreI meganuclease variants with the HIV1_{—}5.3 (SEQ ID NO:339) and HIV1_{—}5.5 (SEQ ID NO:341) targets. On the filter, the positive variants presented correspond to: G2, SEQ ID NO:266; E4, SEQ ID NO:267; C2, SEQ ID NO:268; A12, SEQ ID NO:269; C11, SEQ ID NO:270; all described in Table XL. Each cluster contains 4 spots. On the 2 spots on the left, a yeast strain harboring the HIV1_{—}5.3 (SEQ ID NO:339) or the HIV1_{—}5.5 (SEQ ID NO:341) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

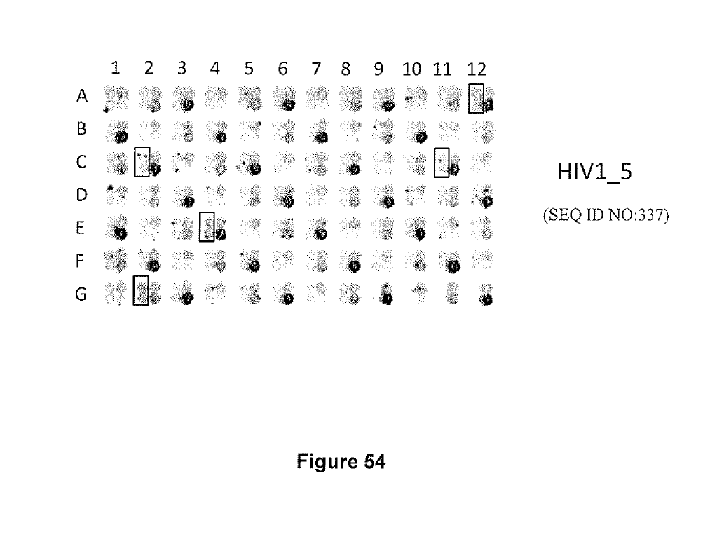

FIG. 54: Cleavage of HIV1_{—}5 target (SEQ ID NO:337) by meganuclease variants improved by a second round of random mutagenesis in example 28bis. The figure displays an example of screening of I-CreI meganuclease variants with the HIV1_{—}5 target (SEQ ID NO:337), when mated with a meganuclease (SEQ ID NO:276) cleaving the HIV1_{—}5.4 target (SEQ ID NO:340). On the filter, the positive variants presented correspond to: G2, SEQ ID NO:266; E4, SEQ ID NO:267; C2, SEQ ID NO:268; A12, SEQ ID NO:269; C11, SEQ ID NO:270; all described in Table XL. Each cluster contains 4 spots. On the 2 spots on the left, a yeast strain harboring the HIV1_{—}5.4 mutant (SEQ ID NO:276) and the HIV1_{—}5 target (SEQ ID NO:337) have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

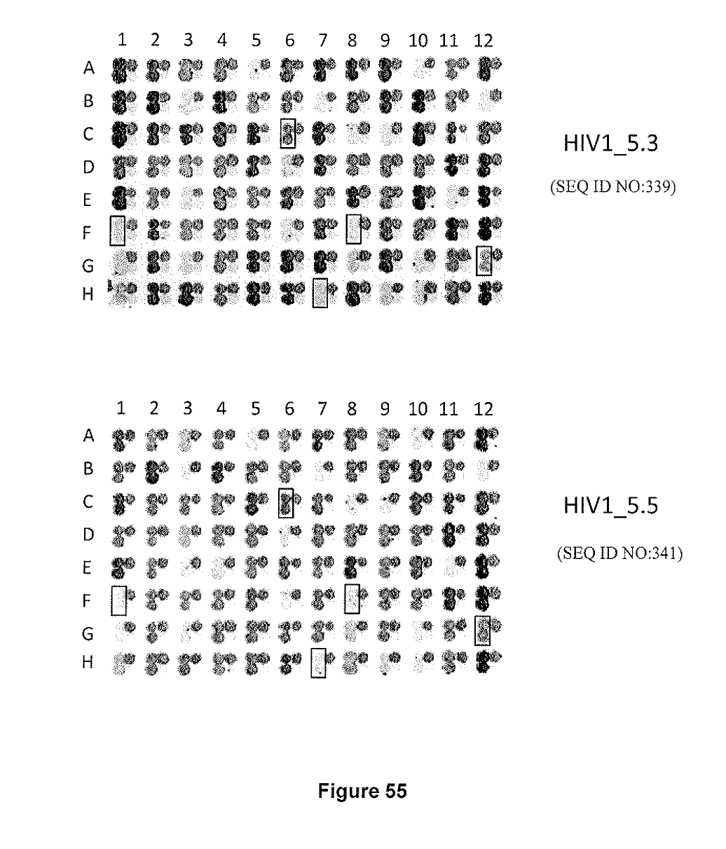

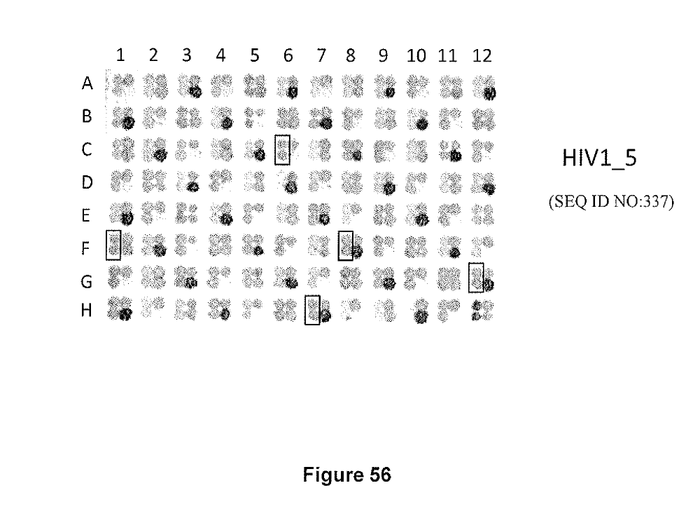

FIG. 55: Cleavage of HIV1_{—}5.3 (SEQ ID NO:339) and HIV1_{—}5.5 (SEQ ID NO:341) targets by meganuclease variants improved by site-directed mutagenesis in example 29. The figure displays an example of screening of I-CreI meganuclease variants with the HIV1_{—}5.3 (SEQ ID NO:339) and HIV1_{—}5.5 (SEQ ID NO:341) targets. On the filter, the positive variants presented correspond to: C6, SEQ ID NO:278; F8, SEQ ID NO:279; H7, SEQ ID NO:280; F1, SEQ ID NO:281; G12, SEQ ID NO:282; described in Table XLI. Some of these variants show no cleavage activity as homodimers while they are active as heterodimers on the HIV1_{—}5 target (SEQ ID NO:337) (see FIG. 56). This is due to the presence of the G19S mutation in these variants. Each cluster contains 4 spots. On the 2 spots on the left, a yeast strain harboring the HIV1_{—}5.3 (SEQ ID NO:339) or the HIV1_{—}5.5 (SEQ ID NO:341) targets has been mated with another yeast strain containing the meganuclease variants. The spot on the low-right is a negative control. The spot in the upper-right contains, as an internal control, an improved variant.

FIG. 56: Cleavage of HIV1_{—}5 target (SEQ ID NO:337) by meganuclease variants improved by site-directed mutagenesis in example 29. The figure displays an example of screening of I-CreI meganuclease variants with the HIV1_{—}5 target (SEQ ID NO:337), when mated with a meganuclease (SEQ ID NO:276) cleaving the HIV1_{—}5.4 target (SEQ ID NO:340). On the filter, the positive variants presented correspond to: C6, SEQ ID NO:278; F8, SEQ ID NO:279; H7, SEQ ID NO:280; F1, SEQ ID NO:281; G12, SEQ ID NO:282; described in Table XLI. Each cluster contains 4 spots. On the 2 spots on the left, a yeast strain harboring the HIV1_{—}5.4 mutant (SEQ ID NO:276) and the HIV1_{—}5 target (SEQ ID NO:337) has been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

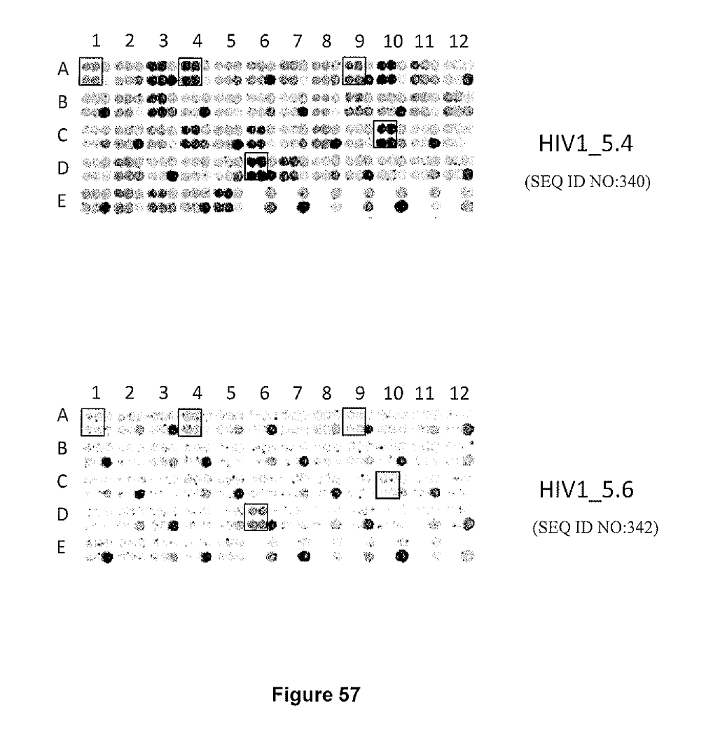

FIG. 57: Cleavage of HIV1_{—}5.4 (SEQ ID NO:340) and HIV1_{—}5.6 (SEQ ID NO:342) targets by meganuclease variants improved by random mutagenesis in example 30. The figure displays an example of screening of I-CreI meganuclease variants with the HIV1_{—}5.4 (SEQ ID NO:340) and HIV1_{—}5.6 (SEQ ID NO:342) targets. On the filter, the positive variants presented correspond to: D6, SEQ ID NO:276; A4, SEQ ID NO:288; C10, SEQ ID NO:289; A9, SEQ ID NO:290; A1, SEQ ID NO:291; all described in Table XLII. Each cluster contains 6 spots. On the 4 spots on the left, a yeast strain harboring the HIV1_{—}5.4 (SEQ ID NO:340) or the HIV1_{—}5.6 (SEQ ID NO:342) targets has been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, a non-improved variant cleaving the HIV1_{—}5.4 target (SEQ ID NO:340).

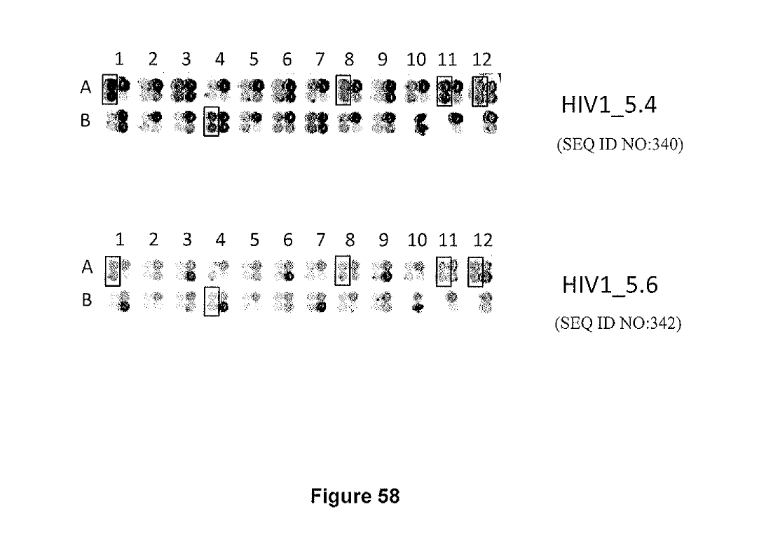

FIG. 58: Cleavage of HIV1_{—}5.4 (SEQ ID NO:340) and HIV1_{—}5.6 (SEQ ID NO:342) targets by meganuclease variants improved by a second round of random mutagenesis in example 30bis. The figure displays an example of screening of I-CreI meganuclease variants with the HIV1_{—}5.4 (SEQ ID NO:340) and HIV1_{—}5.6 (SEQ ID NO:342) targets. On the filter, the positive variants presented correspond to: A12, SEQ ID NO:297; A1, SEQ ID NO:298; A11, SEQ ID NO:299; A8, SEQ ID NO:300; B4, SEQ ID NO:301; all described in Table XLIII. Each cluster contains 4 spots. On the 2 spots on the left, a yeast strain harboring the HIV1_{—}5.4 (SEQ ID NO:340) or the HIV1_{—}5.6 (SEQ ID NO:342) targets have been mated with another yeast strain containing the meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

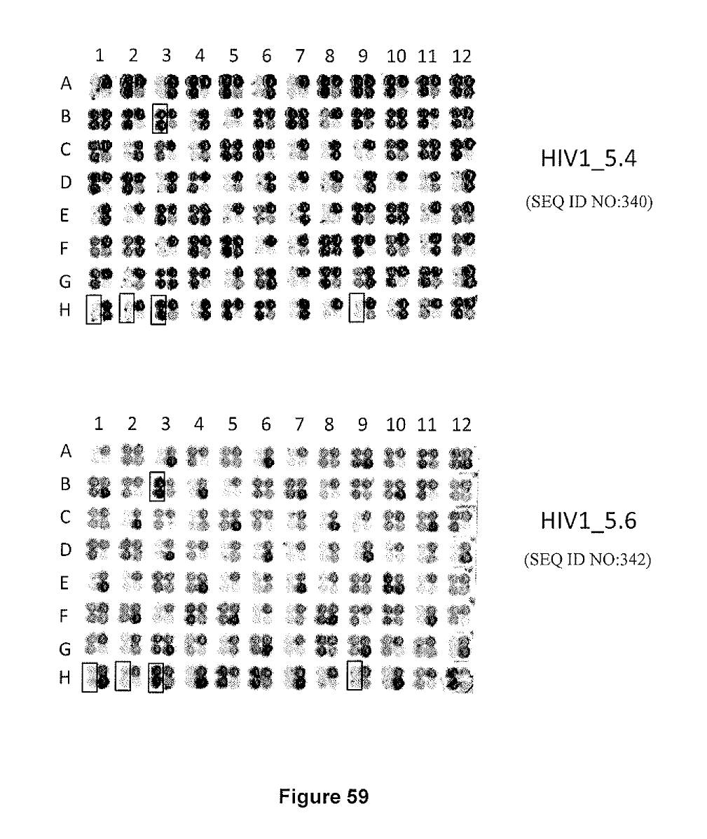

FIG. 59: Cleavage of HIV1_{—}5.4 (SEQ ID NO:340) and HIV1_{—}5.6 (SEQ ID NO:342) targets by meganuclease variants improved by site-directed mutagenesis in example 31. The figure displays an example of screening of I-CreI meganuclease variants with the HIV1_{—}5.4 (SEQ ID NO:340) and HIV1_{—}5.6 (SEQ ID NO:342) targets. On the filter, the positive variants presented correspond to: H1, SEQ ID NO:307; H2, SEQ ID NO:308; H9, SEQ ID NO:309; B3, SEQ ID NO:310; H3, SEQ ID NO:311; described in Table XLIV. Each cluster contains 4 spots. On the 2 spots on the left, a yeast strain harboring the HIV1_{—}5.4 (SEQ ID NO:340) or the HIV1_{—}5.6 (SEQ ID NO:342) targets has been mated with another yeast strain containing different meganuclease variants. The spot on the low-right contains negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

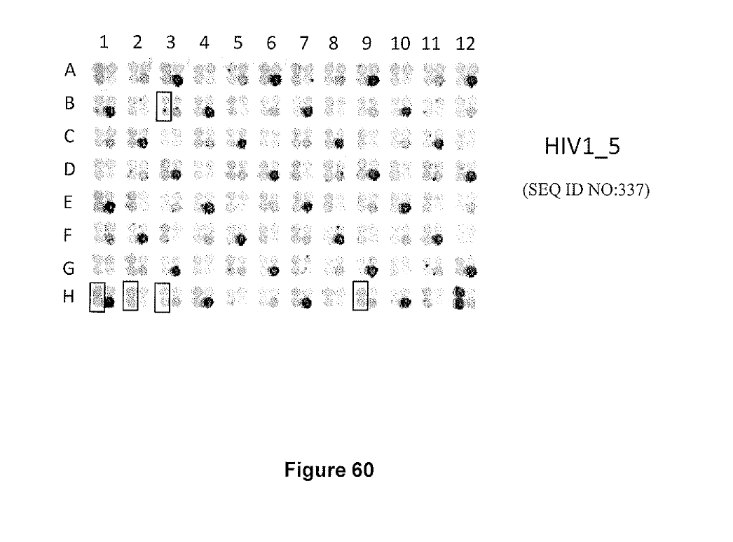

FIG. 60: Cleavage of HIV1_{—}5 (SEQ ID NO:337) target by meganuclease variants improved by site-directed mutagenesis in example 31. The figure displays an example of screening of I-CreI meganuclease variants with the HIV1_{—}5 target (SEQ ID NO:337), when mated with a meganuclease (SEQ ID NO:256) cleaving the HIV1_{—}5.3 target (SEQ ID NO:339). On the filter, the positive variants presented correspond to: H1, SEQ ID NO:307; H2, SEQ ID NO:308; H9, SEQ ID NO:309; B3, SEQ ID NO:310; H3, SEQ ID NO:311; described in Table XLIV. Each cluster contains 4 spots. On the 2 spots on the left, a yeast strain harboring the HIV1_{—}5 target (SEQ ID NO:337) and the HIV1_{—}5.3 mutant (SEQ ID NO:256) has been mated with another yeast strain containing different meganuclease variants. The spot on the low-right contain negative or positive controls. These controls are serially repeated every three clusters as follows: negative control (i.e. cluster A1), positive control (i.e. cluster A2), and strong positive control (i.e. cluster A3). The spot in the upper-right contains, as an internal control, an improved variant.

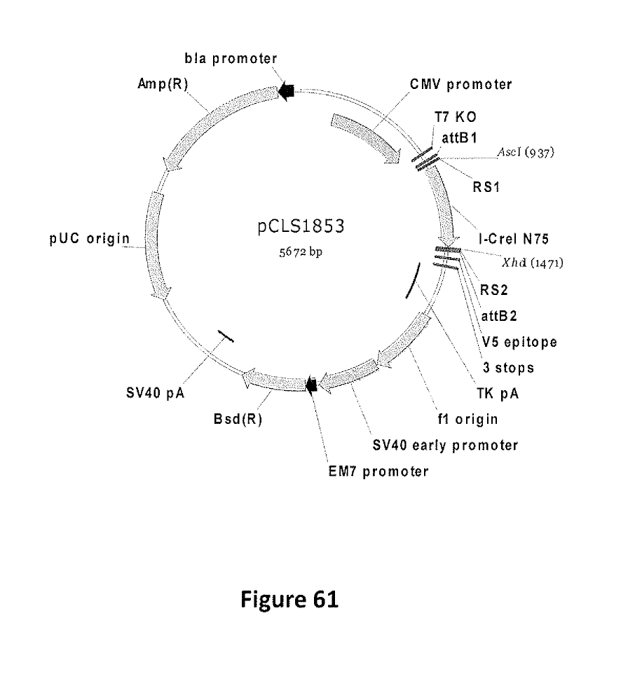

FIG. 61: pCLS1853 plasmid map.

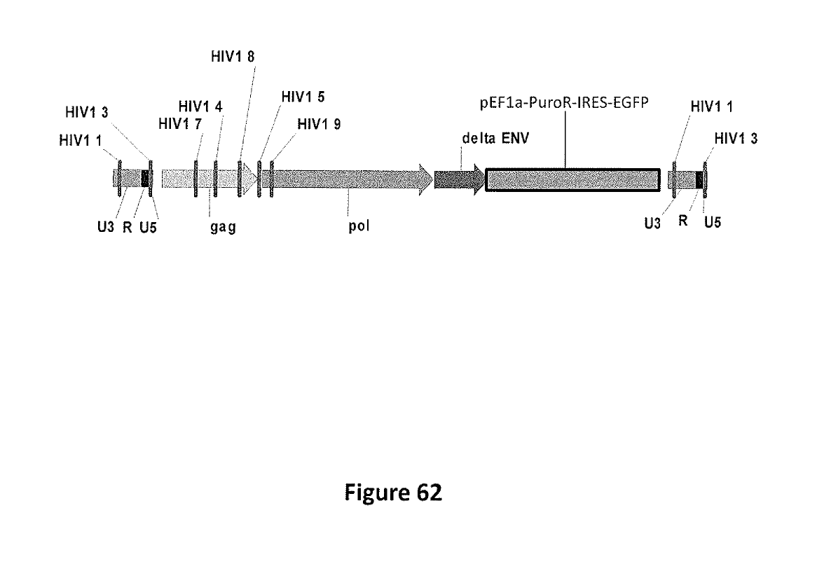

FIG. 62: Schematic representation of the pseudo-HIV provirus integrated in the HEK293-VLP-CL40 cell line used for validation of the activity of HIV meganucleases. The LTRs, encompassing the U3, R and U5 regulatory sequences are duplicated and flanking the viral genes gag and pol. The env gene has been partially deleted and a pEF1a-PuroR-IRES-EGFP cassette has been introduced between the 5′ portion of env and the 3′ LTR. The location of the meganuclease targets HIV1_{—}1 (SEQ ID NO:319), HIV1_{—}3 (SEQ ID NO:325), HIV1_{—}4 (SEQ ID NO:331), HIV1_{—}5 (SEQ ID NO:337), HIV1_{—}7 (SEQ ID NO:366), HIV1_{—}8 (SEQ ID NO:367) and HIV1_{—}9 (SEQ ID NO:368) are represented. The ORF of the TAT and REV genes have been introduced in the cellular genome using different retroviral vectors.

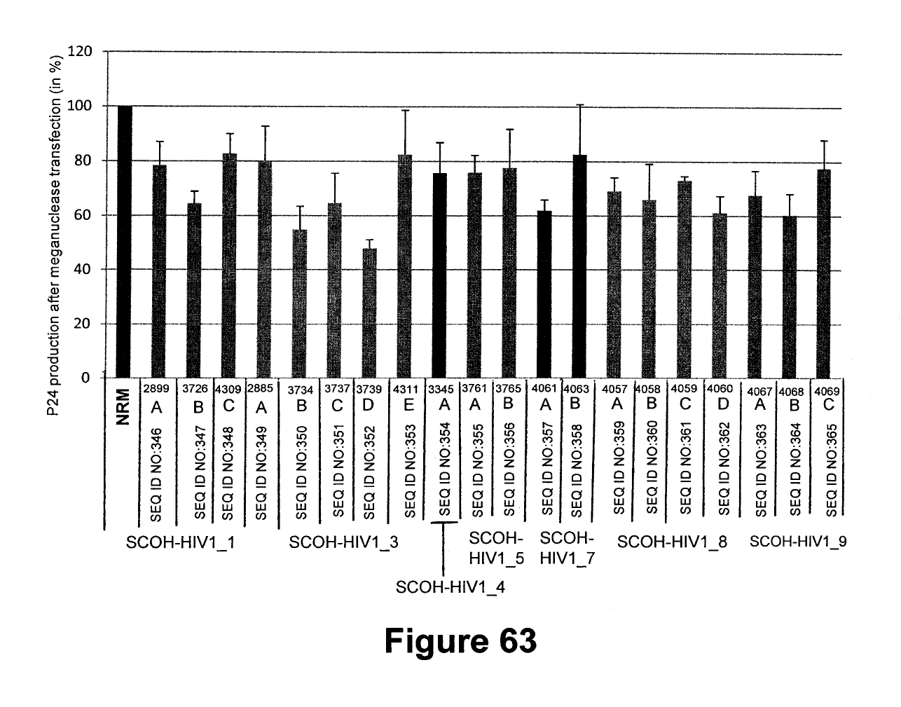

FIG. 63: Levels of p24 produced by the HEK293-VLP-CL40 cell line 48 hours after transfection with 1 μg of meganuclease expression plasmid.

The amount of p24 present in cell culture supernatants was determined by ELISA. A sample transfected by a non related meganuclease (NRM, see text) is used for normalization. In this way, the amount of p24 produced by these cells, expressed in fg/cell is considered as 100% of VLP production. The amount of p24 produced by HIV meganuclease transfected cells is represented as the percentage of VLP production respect to the amount produced by the NRM transfected cells. The values represent the data from at least 3 independent transfections.

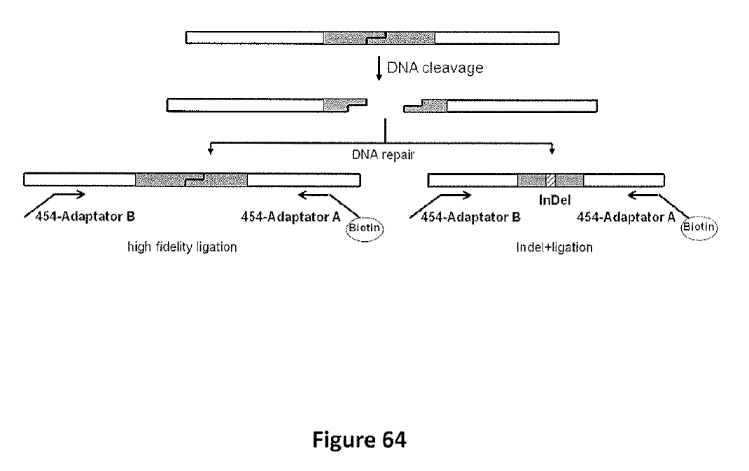

FIG. 64: represents a scheme of the mechanism leading to the generation of small deletions and insertions (InDel) during repair of double-strand break by non homologous end joining (NHEJ).

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Strategy for Engineering Meganucleases Cleaving the HIV1_{—}1 Target (SEQ ID NO:319) from the HIV1 Virus

The HIV1_{—}1 target (SEQ ID NO:319) is a 22 by (non-palindromic) target located in U3 region of the proviral LTRs (FIGS. 2 and 7). Since the LTRs are duplicated sequences flanking the viral ORFs in the integrated provirus, the HIV1_{—}1 target is present twice in the HIV_{—}1 provirus. This target is precisely located at positions 84-105 and 8159-9180 of the HIV-1 pNL4-3 vector (accession number AF324493, Adachi et al., J. Virol., 1986, 59, 284-291), this infective molecular clone was generated from the NY5 strain (Barre-Sinoussi et al., Science, 1983, 220, 868-871 and Benn et al., Science, 1985, 230, 949-951) a subtype B infectious molecular clone.

The HIV1_{—}1 sequence (SEQ ID NO:319) is partly a patchwork of the 10AGA_P (SEQ ID NO:381), 10TGG_P (SEQ ID NO:379), 5TAC_P (SEQ ID NO:389) and 5_CTG_P (SEQ ID NO:387) targets (these designations describe the 3 bp starting at the indicated nucleotide of the I-CreI target, for instance 10AGA_P (SEQ ID NO:381) indicates that nucleotides −10, −9 and −8 are A(−10) G(−9) A(−8) (FIG. 7)) which are cleaved by previously identified meganucleases. These meganucleases were obtained as described in International PCT Applications WO 2006/097784 and WO 2006/097853; Arnould et al., J. Mol. Biol., 2006, 355, 443-458; Smith et al., Nucleic Acids Res., 2006.

The 10AGA_P (SEQ ID NO:381), 10TGG_P (SEQ ID NO:379), 5TAC_P (SEQ ID NO:389) and 5_CTG_P (SEQ ID NO:387) target sequences are 24 by derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HIV1_{—}1 series of targets (SEQ ID NO:319 to 324) were defined as 22 by sequences instead of 24 bp. HIV1_{—}1 (SEQ ID NO:319) differs from C1221 (SEQ ID NO: 343) in the 4 by central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the ACAC sequence in −2 to 2 was first substituted with the GTAC sequence from C1221, resulting in target HIV1_{—}1.2 (SEQ ID NO:320) (FIG. 7). Then, two palindromic targets, HIV1_{—}1.3 (SEQ ID NO:321) and HIV1_{—}1.4 (SEQ ID NO:322), were derived from HIV1_{—}1.2 (SEQ ID NO:320) (FIG. 7). Since HIV1_{—}1.3 (SEQ ID NO:321) and HIV1_{—}1.4 (SEQ ID NO:322) are palindromic, they should be cleaved by homodimeric proteins. Two other pseudo-palindromic targets were derived from these two containing the ACAC sequence in −2 to 2 (targets HIV1_{—}1.5 (SEQ ID NO:323) and HIV1_{—}1.6 (SEQ ID NO:324), FIG. 7). Thus, proteins able to cleave HIV1_{—}1.3 (SEQ ID NO:321) and HIV1_{—}1.4 (SEQ ID NO:322) targets or, preferentially, the pseudo-palindromic targets as homodimers were first designed (examples 2 and 3) and then co-expressed to obtain heterodimers cleaving HIV1_{—}1 (SEQ ID NO:319) (example 4). Heterodimers cleaving the HIV1_{—}1.2 (SEQ ID NO:320) and HIV1_{—}1 (SEQ ID NO:319) targets could be identified. In order to improve cleavage activity for the HIV1_{—}1 target (SEQ ID NO:319), a series of variants cleaving HIV1_{—}1.3 (SEQ ID NO:321) and HIV1_{—}1.4 (SEQ ID NO:322) was chosen, and then refined. The chosen variants were subjected to random or site-directed mutagenesis, and used to form novel heterodimers that were screened against the HIV1_{—}1 target (SEQ ID NO:319) (examples 5, 6, 7 and 8). Heterodimers could be identified with an improved cleavage activity for the HIV1_{—}1 target (SEQ ID NO:319).

### EXAMPLE 2

### Identification of Meganucleases Cleaving HIV1_{—}1.3 (SEQ ID NO:321)

This example shows that I-CreI variants can cut the HIV1_{—}1.3 DNA target sequence (SEQ ID NO:321) derived from the left part of the HIV1_{—}1.2 target (SEQ ID NO:320) in a palindromic form (FIG. 7).

HIV1_{—}1.3 (SEQ ID NO:321) is similar to 10AGA_P (SEQ ID NO:381) at positions ±1, ±2, ±6, ±8, ±9, and ±10 and to 5TAC_P (SEQ ID NO:389) at positions ±1, ±2, ±3, ±4, ±5 and ±6. It was hypothesized that positions ±7 and ±11 would have little effect on the binding and cleavage activity. Variants able to cleave the 10AGA_P (SEQ ID NO:381) target were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156. Variants able to cleave 5TAC_P (SEQ ID NO:389) were obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Both sets of proteins are mutated at position 70. However, the existence of two separable functional subdomains was hypothesized. This implies that this position has little impact on the specificity at bases 10 to 8 of the target. Mutations at positions 24 found in variants cleaving the 5TAC_P target (SEQ ID NO:389) will be lost during the combinatorial process. But it was hypothesized that this will have little impact on the capacity of the combined variants to cleave the HIV1_{—}1.3 target (SEQ ID NO:321).

Therefore, to check whether combined variants could cleave the HIV1_{—}1.3 target (SEQ ID NO:321), mutations at positions 44, 68, 70, 75 and 77 from proteins cleaving 5TAC_P (SEQ ID NO:389) were combined with the 28, 30, 32, 33, 38 and 40 mutations from proteins cleaving 10AGA_P (SEQ ID NO:381).

### A) Material and Methods

### a) Construction of Target Vector

The target was cloned as follows: an oligonucleotide corresponding to the HIV1_{—}1.3 (SEQ ID NO:321) target sequence flanked by gateway cloning sequences was ordered from PROLIGO: 5′ TGGCATACAAGTTTGCAGAACTACGTACGTAGTTCTGCCAATCGTCTGTCA 3′(SEQ ID NO: 14). The same procedure was followed for cloning the HIV1_{—}1.5 target (SEQ ID NO:323), using the oligonucleotide: 5′ TGGCATACAAGTTTGCAGAACTACACACGTAGTTCTGCCAATCGTCTGTCA 3′ (SEQ ID NO: 15). Double-stranded target DNA, generated by PCR amplification of the single stranded oligonucleotide, was cloned using the Gateway protocol (INVITROGEN) into the yeast reporter vector (pCLS1055, FIG. 8). Yeast reporter vector was transformed into Saccharomyces cerevisiae strain FYBL2-7B (MATα, ura3Δ851, trp1Δ63, leu2Δ1, lys2Δ202), resulting in a reporter strain.

### b) Construction of Combinatorial Mutants

I-CreI variants cleaving 10AGA_P (SEQ ID NO:381) or 5TAC_P

(SEQ ID NO:389) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10AGA_P (SEQ ID NO:381) and 5TAC_P (SEQ ID NO:389) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′(SEQ ID NO: 17)) specific to the vector (pCLS0542, FIG. 9) and primers (assF 5′-ctannnttgaccttt-3′ (SEQ ID NO: 18) or assR 5′-aaaggtcaannntag-3′(SEQ ID NO: 19)), where nnn codes for residue 40, specific to the I-CreI coding sequence for amino acids 39-43.

The PCR fragments resulting from the amplification reaction using the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 9) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

### B) Results

I-CreI combinatorial variants were constructed by associating mutations at positions 44, 68, 70, 75 and 77 from proteins cleaving 5TAC_P (SEQ ID NO:389) with the 28, 30, 32, 33, 38 and 40 mutations from proteins cleaving 10AGA_P (SEQ ID NO:381) on the I-CreI scaffold, resulting in a library of complexity 1600. Examples of combinatorial variants are displayed in Table I. In Table I the peptide sequence of these two subdomains are provided in the first column and second row respectively.

This library was transformed into yeast and 3348 clones (2 times the diversity) were screened for cleavage against the HIV1_{—}1.3 (SEQ ID NO:321) and HIV1_{—}1.5 (SEQ ID NO:323) DNA targets. 36 positive clones were found to cleave the HIV1_{—}1.3 target (SEQ ID NO:321), which after sequencing turned out to correspond to 31 different novel endonuclease variants (Table II). Those variants showed no cleavage activity of the HIV1_{—}1.5 DNA target (SEQ ID NO:323). Examples of positives are shown in FIG. 10. Some of the variants obtained display non parental combinations at positions 28, 30, 32, 33, 38, 40 or 44, 68, 70, 75, 77. Such combinations likely result from PCR artifacts during the combinatorial process. Alternatively, the variants may be I-CreI combined variants resulting from microrecombination between two original variants during in vivo homologous recombination in yeast.

TABLE I
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, | | | | | | | | | | | |
| 70, | | | | | | | | | | | |
| 75 and | | | | | | | | | | | |
| 77 | | | | | | | | | | | |
| (ex: | | | | | | | | | | | |
| ARNNI | | | | | | | | | | | |
| stands for | | | | | | | | | | | |
| N70, R68, | | | | | | | | | | | |
| KTTYQS | | | | | | | | | | | |
| N70, | Amino acids at positions 28, 30, 32, 33, 38 and 40 | | | | | | | | | | |
| N75 and | (ex: KHSSQS stands for K28, H30, S32, 833, Q38 and S40) | | | | | | | | | | |
| I77) | KTTYQS | KQSHQS | KNSCRS | KDSRQS | KGSYHN | KTSAQS | KTSHRS | KNSGGS | KNSPRS | KNSPKS | KSSGQS |
| ARSYT | | | + | | | | | | | | |
| AYSHI | | | | | | | | | | | |
| YYSYR | | | | | | | | | | | |
| ARSRY | | | | | | | | | | | |
| AYSRV | | | | | | | | | | | |
| ARSRN | | | | | | | | | | | |
| NTSRY | | | + | | | | | | | | |
| ARRNI | | | | | | | | | | | |
| NYSRV | | | | | | | | | | | |
| NTSRV | | | | | | | | | | | |
| NRSRI | | | | | | | | | | | |
| VERNR | | | | | | | | | | | |
| NKSRT | | | | | | | | | | | |
| NYSRY | | | + | | | | | | | | |
| AYSRQ | | | | | | | | | | | |
| NTSRQ | | | | | | | | | | | |
| DNSNI | | | | | | | | | | | |
| NRSRN | | | + | | | | | | | | |
| AYSRK | | | | | | | | | | | |
| NYSRI | | | | | | | | | | | |
| AASRI | | | | | | | | | | | |
| NRRNI | | | | | | | | | | | |
| ARSRV | | | | | | | | | | | |
| NTSRI | | | | | | | | | | | |

*Only 264 out of the 1600 combinations are displayed.
+ indicates that a functional combinatorial variant cleaving the HIV1_1.3 target (SEQ ID NO: 321) was found among the identified positives.

TABLE II
I-CreI variants with additional mutations
capable of cleaving the HIV1_1.3 target
(SEQ ID NO: 321)

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and I77) | NO: |
| KGSYRS/NYSRI +93Q | 1 |
| KGSYRS/NYSRY | 2 |
| KGSYRS/VERNR +80K | 3 |
| KGSYRS/IERNR +80K | 4 |
| KGSYRS/NYSRQ | 5 |
| KNSCRS/AYSRQ +154N | 6 |

### EXAMPLE 3

### Making of Meganucleases Cleaving HIV1_{—}1.4 (SEQ ID NO:322)

This example shows that I-CreI variants can cleave the HIV1_{—}1.4 DNA target sequence (SEQ ID NO:322) derived from the right part of the HIV1_{—}1.2 target (SEQ ID NO:320) in a palindromic form (FIG. 7).

HIV1_{—}1.4 (SEQ ID NO:322) is similar to 5CTG_P (SEQ ID NO:387) at positions ±1, ±2, ±3, ±4, ±5 and ±8 and to 10TGG_P (SEQ ID NO:379) at positions +1, ±2, ±3, ±4, ±8, ±9 and ±10. It was hypothesized that positions ±6, ±7 and ±11 would have little effect on the binding and cleavage activity. Variants able to cleave 5CTG_P (SEQ ID NO:387) were obtained by mutagenesis of I-CreI N75 at positions 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10TGG_P target (SEQ ID NO:379) were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156.

Both sets of proteins are mutated at position 70. However, the existence of two separable functional subdomains was hypothesized. This implies that this position has little impact on the specificity at bases 10 to 8 of the target.

Therefore, to check whether combined variants could cleave the HIV1_{—}1.4 target (SEQ ID NO:322), mutations at positions 44, 68, 70, 75 and 77 from proteins cleaving 5TTC_P (SEQ ID NO:388) were combined with the 28, 30, 32, 33, 38 and 40 mutations from proteins cleaving 10GGA_P (SEQ ID NO:380).

### A) Material and Methods

### a) Construction of Target Vector

The experimental procedure is as described in example 2, with the exception that different oligonucleotides corresponding to the HIV1_{—}1.4 (SEQ ID NO:322) and HIV1_{—}1.6 (SEQ ID NO:324) targets. The oligonucleotide used for the HIV1_{—}1.4 target (SEQ ID NO:322) was:

(SEQ ID NO: 20)
5′TGGCATACAAGTTTCCTGGCCCTGGTACCAGGGCCAGGCAATCGTC
TGTCA 3′,
and
(SEQ ID NO: 21)
5′TGGCATACAAGTTTCCTGGCCCTGACACCAGGGCCAGGCAATCGTC
TGTCA 3′
for HIV1_1.6 target. (SEQ ID NO: 324)

### b) Construction of Combinatorial Variants

I-CreI variants cleaving 10TGG_P (SEQ ID NO:379) or 5CTG_P (SEQ ID NO:387) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TGG_P (SEQ ID NO:379) and 5CTG_P (SEQ ID NO:387) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′ (SEQ ID NO: 17)) specific to the vector (pCLS1107, FIG. 11) and primers (assF 5′-ctannnttgaccttt-3′ (SEQ ID NO: 18) or assR 5′-aaaggtcaanrmtag-3′(SEQ ID NO: 19)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. Positives resulting clones were verified by sequencing (MILLEGEN) as described in example 2.

### B) Results

I-CreI combinatorial variants were constructed by associating mutations at positions 44, 68, 70, 75 and 77 from proteins cleaving 5CTG_P (SEQ ID NO:387) with the 28, 30, 32, 33, 38 and 40 mutations from proteins cleaving 10TGG_P (SEQ ID NO:379) on the I-CreI scaffold, resulting in a library of complexity 1600. Examples of combinatorial variants are displayed in Table III. This library was transformed into yeast and 3348 clones (2 times the diversity) were screened for cleavage against the HIV1_{—}1.4 (SEQ ID NO:322) and HIV1_{—}1.6 (SEQ ID NO:324) DNA targets. A total of 32 positive clones were found to cleave HIV1_{—}1.4 (SEQ ID NO:322). Sequencing of these 32 clones allowed the identification of 25 novel endonuclease variants. One of those variants showed cleavage activity on the HIV1_{—}1.6 DNA target (SEQ ID NO:324). Examples of positives are shown in FIG. 12. The sequence of several of the variants identified display non parental combinations at positions 28, 30, 32, 33, 38, 40 or 44, 68, 70, 75, 77 as well as additional mutations (see examples Table IV). Such variants likely result from PCR artifacts during the combinatorial process. Alternatively, the variants may be I-CreI combined variants resulting from micro-recombination between two original variants during in vivo homologous recombination in yeast.

TABLE III
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, | | | | | | | | | | | |
| 70, 75 | | | | | | | | | | | |
| and 77 | | | | | | | | | | | |
| (ex: | | | | | | | | | | | |
| ARNNI | | | | | | | | | | | |
| stands | | | | | | | | | | | |
| for A44, | | | | | | | | | | | |
| R68, N70, | Amino acids at positions 28, 30, 32, 33, 38 and 40 | | | | | | | | | | |
| N75 and | (ex: KHSSQS stands for K28, H30, S32, S33, Q38 and S40) | | | | | | | | | | |
| I77) | ANSSRK | NNSSRR | QNSSRK | KNRGRS | KNSCAS | KYTCQS | KNSTGS | KNSTQT | KNSCHS | KSSSTS | KNTTQS |
| RYSDN | | | | | + | | | | | | |
| RASER | | | | | | | | | | | |
| RQSER | | | | | | | | | | | |
| RYSEI | | | | | | | | | | | |
| RYSET | | | | | + | | | | | | |
| KYSNI | | | | | | | | | | | |
| ATSNR | | | | | | | | | | | |
| RYSEY | | | | | + | | | | | | |
| EYSES | | | | | | | | | | | |
| RTSER | | | | | | | | | | | |
| RYSTI | | | | | | | | | | | |
| RYSDR | | | | | | | | | | | |
| RYSDT | | | | | + | | | | + | | |
| SRSKE | | | | | | | | | | | |
| RRSEY | | | | | | | | | | | |
| RYSEV | | | | | | | | | | | |
| RYSER | | | | | | | | | | | |
| RYSDQ | | | | | | | | | + | | |
| KYSEV | | | | | | | | | | | |
| KYSQT | | | | | | | | | | | |
| RYSNI | | | | | | | | | | | |
| RRSDY | | | | | | | | | | | |
| HYSNH | | | | | | | | | | | |
| PKSNL | | | | | | | | | | | |

*Only 264 out of the 1600 combinations are displayed.
+ indicates that a functional combinatorial variant cleaving the HIV1_1.4 target (SEQ ID NO: 322) was found among the identified positives.

TABLE IV
I-CreI variants with additional mutations
capable of cleaving the HIV1_1.4 target
(SEQ ID NO: 322).

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and 177) | NO: |
| QNSSRK/KYSES | 7 |
| KNSCAS/KYSES | 8 |
| KNSSRN/KYSES | 9 |
| KCSTQR/RYSDQ | 10 |
| KNSTQK/RYSDN | 11 |
| KNSSQS/RSSDR | 12 |

### EXAMPLE 4

### Making of Meganucleases Cleaving HIV1_{—}1.2 (SEQ ID NO:320) and HIV1_{—}1 (SEQ ID NO:319)

I-CreI variants able to cleave each of the palindromic HIV1_{—}1.2 (SEQ ID NO:320) derived targets (HIV1_{—}1.3 (SEQ ID NO:321) and HIV1_{—}1.4 (SEQ ID NO:322)) were identified in example 2 and example 3. Pairs of such variants (one cutting HIV1_{—}1.3 (SEQ ID NO:321) and one cutting HIV1_{—}1.4 (SEQ ID NO:322)) were co-expressed in yeast. Upon co-expression, there should be three active molecular species, two homodimers, and one heterodimer. It was assayed whether the heterodimers that should be formed, cut the HIV1_{—}1.2 (SEQ ID NO:320) and the non palindromic HIV1_{—}1 (SEQ ID NO:319) targets.

### A) Materials and Methods

### a) Construction of Target Vector

The experimental procedure is as described in example 2, with the exception that an oligonucleotide corresponding to the HIV1_{—}1.2 target sequence (SEQ ID NO:320): 5′TGGCATACAAGTTTGCAGAACTACGTACCAGGGCCAGGCAATCGTCTGTCA 3′ (SEQ ID NO: 22) or the HIV1_{—}1 target sequence (SEQ ID NO:319): 5′TGGCATACAAGTTTGCAGAACTACACACCAGGGCCAGGCAATCGTCTGTCA 3′(SEQ ID NO: 23) was used.

### b) Co-Expression of Variants

Yeast DNA was extracted from variants cleaving the HIV1_{—}1.4 target (SEQ ID NO:322) in the pCLS1107 expression vector using standard protocols and was used to transform E. coli. The resulting plasmid DNA was then used to transform yeast strains expressing a variant cutting the HIV1_{—}1.3 target (SEQ ID NO:321) in the pCLS0542 expression vector. Transformants were selected on synthetic medium lacking leucine and containing G418.

### c) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### B) Results

Co-expression of variants cleaving the HIV1_{—}1.4 target (SEQ ID NO:322) (6 variants chosen among those described in Table III and Table IV) and six variants cleaving the HIV1_{—}1.3 target (SEQ ID NO:321) (described in Tables I and II) resulted in cleavage of the HIV1_{—}1.2 target (SEQ ID NO:320) in most of the cases (FIG. 13). Nevertheless, only one of these combinations was able to weakly cut the HIV1_{—}1 natural target (SEQ ID NO:319) that differs from the HIV1_{—}1.2 sequence (SEQ ID NO:320) by 2 by at positions 1 and 2 (FIG. 13). Examples of functional combinations are summarized in Table V and Table VI.

TABLE V
Cleavage of the HIV1_1.2 target (SEQ ID NO: 320) by the heterodimeric variants

| | | Amino acids at positions 28, 30, | |
|---|---|---|---|
| | | 32, 33, 38, 40/44, 68, 70, 75 and | |
| | | 77 of the I-CreI variants | |
| | | cleaving the HIV1_1.4 target | |
| | | (ex: KRSRES/TYSNI stands for K28, | |
| | | R30, S32, R33, E38, S40/T44, | |
| | | Y68, S70, N75 and I77) | |
| | | KNSTQK/RYSDN | KCSTQR/RYSDQ |
| HIV1_1.2 target (SEQ ID NO: 320) | | SEQ ID NO: 11 | SEQ ID NO: 10 |
| Amino acids at positions | KGSYRS/NYSRQ | + | + |
| 28, 30, 32, 33, 38, 40/44, | SEQ ID NO: 5 | | |
| 68, 70, 75 and 77 | KGSYRS/NYSRY +162P | + | + |
| Of I-CreI variants | SEQ ID NO: 13 | | |
| cleaving the HIV1_1.3 | KGSYRS/NYSRY | + | + |
| target | SEQ ID NO: 2 | | |
| (ex: KRGYQS/RHRDI | KGSYRS/NYSRI | + | + |
| stands for K28, R30, G32, | SEQ ID NO: 1 | | |
| Y33, Q38, S40/R44, H68, | KGSYRS/VERNR | + | + |
| R70, D75 and 177) | SEQ ID NO: 3 | | |
| | KGSYRS/IERNR | + | * |
| | SEQ ID NO: 4 | | |

+ indicates a functional combination
* indicates that the combination weakly cuts the HIV1_1.2 target (SEQ ID NO: 320).

TABLE VI
Cleavage of the HIV1_1 target (SEQ ID NO: 319) by the heterodimeric variants

| | | Amino acids at positions 28, 30, | |
|---|---|---|---|
| | | 32, 33, 38, 40/44, 68, 70, 75 and 77 | |
| | | of the I-CreI variants cleaving the | |
| | | HIV1_1.4 target | |
| | | (ex: KRSRES/TYSNI stands for K28, R30, | |
| | | S32, R33, E38, S40/T44, Y68, | |
| | | S70, N75 and I77) | |
| | | KNSTQK/RYSDN | KCSTQR/RYSDQ |
| HIV1_1 target (SEQ ID NO: 319) | | SEQ ID NO: 11 | SEQ ID NO: 10 |
| Amino acids at | KGSYRS/NYSRQ | | |
| positions 28, 30, 32, | SEQ ID NO: 5 | | |
| 33, 38, 40/44, 68, 70, | KGSYRS/NYSRY +162P | | |
| 75 and 77 | SEQ ID NO: 13 | | |
| Of I-CreI variants | KGSYRS/NYSRY | * | |
| cleaving the HIV1_1.3 | SEQ ID NO: 2 | | |
| target | KGSYRS/NYSRI | | |
| (ex: KRGYQS/RHRDI | SEQ ID NO: 1 | | |
| stands for K28, R30, | KGSYRS/VERNR | | |
| G32, Y33, Q38, S40/R44, | SEQ ID NO: 3 | | |
| H68, R70, D75 and 177) | KGSYRS/IERNR | | |
| | SEQ ID NO: 4 | | |

+indicates a functional combination
*indicates that the combination weakly cuts the HIV1_1 target (SEQ ID NO: 319).

### EXAMPLE 5

### Improvement of Meganucleases Cleaving HIV1_{—}1 (SEQ ID NO:319) by Random Mutagenesis of Proteins Cleaving HIV1_{—}1.3 (SEQ ID NO:321) and Assembly with Proteins Cleaving HIV1_{—}1.4 (SEQ ID NO:322)

I-CreI variants able to cleave the HIV1_{—}1.2 (SEQ ID NO:320) and

HIV1_{—}1 (SEQ ID NO:319) target by assembly of variants cleaving the palindromic HIV1_{—}1.3 (SEQ ID NO:321) and HIV1_{—}1.4 (SEQ ID NO:322) target have been previously identified in example 4. However, these variants display stronger activity with the HIV1_{—}1.2 target (SEQ ID NO:320) compared to the HIV1_{—}1 target (SEQ ID NO:319).

Therefore six variants cleaving HIV1_{—}1.3 (SEQ ID NO:321) were mutagenized, and variants were screened for cleavage activity of HIV1_{—}1.3 (SEQ ID NO:321) and HIV1_{—}1.5 (SEQ ID NO:323) targets. Additionally the mutants with the strongest activity were screened for cleavage activity of HIV1_{—}1 (SEQ ID NO:319) when co-expressed with a variant cleaving HIV1_{—}1.4 (SEQ ID NO:322). According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, only one of the two components of the heterodimers cleaving HIV1_{—}1 (SEQ ID NO:319) was mutagenized.

Thus, in a first step, proteins cleaving HIV1_{—}1.3 (SEQ ID NO:321) were mutagenized and their homodimeric cleavage activity was determined, and in a second step, it was assessed whether they could cleave HIV1_{—}1 (SEQ ID NO:319) when co-expressed with a protein cleaving HIV1_{—}1.4 (SEQ ID NO:322).

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25), which are common to the pCLS0542 (FIG. 9) and pCLS1107 (FIG. 11) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Mating of Meganuclease Expressing Clones and Screening in Yeast

Mating was performed as previously described in example 2. Positive resulting clones were verified by sequencing (MILLEGEN) as described in example 2.

### c) Variant-Target Yeast Strains, Screening and Sequencing

The yeast strain FYBL2-7B (MATα, ura3Δ851, trp1Δ63, leu2Δ1, lys2Δ202) containing the HIV1_{—}1 target (SEQ ID NO:319) in the yeast reporter vector (pCLS1055, FIG. 8) was transformed with one variant, in the kanamycin vector (pCLS1107), cutting the HIV1_{—}1.4 (SEQ ID NO:322) target, using a high efficiency LiAc transformation protocol. Variant-target yeast strains were used as target strains for mating assays as described in example 4. Positives resulting clones were verified by sequencing (MILLEGEN) as described in example 2.

### B) Results

Six variants cleaving HIV1_{—}1.3 (SEQ ID NO:321), were pooled, randomly mutagenized and transformed into yeast. The sequences of the variants subjected to random mutagenesis are described in table VII.

2232 transformed clones were screened for cleavage against the HIV1_{—}1.3 (SEQ ID NO:321) and HIV1_{—}1.5 (SEQ ID NO:323) DNA targets. A total of 297 positive clones were found to cleave HIV1_{—}1.3 (SEQ ID NO:321), while only 6 of those cleaved the HIV1_{—}1.5 target (SEQ ID NO:323). Sequencing of the 93 clones showing the strongest activity allowed the identification of 51 novel endonuclease variants. An example of the identified variants is presented in table VIII and in FIG. 14.

TABLE VII
Sequences corresponding to the variants cleaving
the HIV1_1.3 DNA target (SEQ ID NO: 321) used
for improvement by random mutagenesis
Amino acids at positions 28, 30, 32, 33, 38,
40/44, 68, 70, 75 and 77 of I-CreI variants
cleaving the HIV1_1.3 target (SEQ ID NO: 321)
(ex: KRGYQS/RHRDI stands for K28, R30, G32, Y33,
Q38, S40/R44, H68, R70, D75 and I77)

| | |
|---|---|
| KGSYRS/NYSRQ | SEQ ID NO: 5 |
| KGSYRS/NYSRY +162P | SEQ ID NO: 13 |
| KGSYRS/NYSRY | SEQ ID NO: 2 |
| KGSYRS/NYSRI | SEQ ID NO: 1 |
| KGSYRS/VERNR | SEQ ID NO: 3 |
| KGSYRS/EERNR | SEQ ID NO: 4 |

TABLE VIII
Examples of 10 functional variants displaying strong cleavage
activity for HIV1_1.3 (SEQ ID NO: 321).
Optimized variants HIV1_1.3

| | |
|---|---|
| SEQ ID NO: 26 | I-CreI 30G 38R 44V 54L 68E 75N 77R 80K 81T 132V |
| | 163R |
| SEQ ID NO: 27 | I-CreI 30G 38R 44V 54L 68E 75N 77R 80K 99R 111H |
| SEQ ID NO: 28 | I-CreI 30G 38R 44N 68Y 70S 75R 77Y 79N |
| SEQ ID NO: 29 | I-CreI 30G 38R 44V 54L 68Y 70S 75R 77Y 100N |
| SEQ ID NO: 30 | I-CreI 30G 38R 44N 68Y 70S 75R 77Y 162P |
| SEQ ID NO: 31 | I-CreI 30G 38R 44N 68Y 70S 75R 77Y 154R |
| SEQ ID NO: 32 | I-CreI 30G 38R 44N 57R 68Y 70S 75R 77Y |
| SEQ ID NO: 33 | I-CreI 30G 38R 44N 50R 64A 68Y 70S 75R 93Q |
| SEQ ID NO: 34 | I-CreI 30G 38R 44N 68Y 70S 75R 77Y 159R |
| SEQ ID NO: 35 | I-CreI 30G 38R 44N 68Y 70S 75R 77Y 107R 162P |

* Mutations resulting from random mutagenesis are in bold.

The 93 clones showing the highest cleavage activity on target HIV1_{—}1.3 (SEQ ID NO:321) were then mated with a yeast strain that contains (i) the HIV1_{—}1 target (SEQ ID NO:319) in a reporter plasmid (ii) an expression plasmid containing a variant that cleaves the HIV1_{—}1.4 target (SEQ ID NO:322) (I-CreI 33T,40K,44R,68Y,70S,77N +132V or KNSTQK/RYSDN +132V (SEQ ID NO:46), according to the nomenclature of Table I). After mating with this yeast strain, 41 clones were found to cleave the HIV1_{—}1 target (SEQ ID NO:319) more efficiently than the original variant. Thus, 41 positives contained proteins able to form heterodimers with KNSTQK/RYSDN +132V (SEQ ID NO: 46), that showed cleavage activity on the HIV1_{—}1 target (SEQ ID NO:319). An example of positive clones is shown in FIG. 15. Sequencing of these 41 positive clones indicates that 31 distinct variants were identified. Ten of these 31 variants are presented as an example in Table VIII.

### EXAMPLE 5bis

### Improvement of Meganucleases Cleaving HIV1_{—}1 (SEQ ID NO:319) By a Second Round of Random Mutagenesis of Proteins Cleaving HIV1_{—}1.3 (SEQ ID NO:321) and Assembly with Proteins Cleaving HIV1_{—}1.4 (SEQ ID NO:322)

In order to further improve the activity of the obtained meganucleases, a second round of random mutagenesis was carried out following the same rationale of example 5. For this purpose, four variants cleaving HIV1_{—}1.3 (SEQ ID NO:321) were mutagenized, and variants were screened for cleavage activity of HIV1_{—}1.3 (SEQ ID NO:321) and HIV1_{—}1.5 (SEQ ID NO:323) targets. Additionally the mutants with the strongest activity were screened for cleavage activity of HIV1_{—}1 (SEQ ID NO:319) when co-expressed with a variant cleaving HIV1_{—}1.4 (SEQ ID NO:322).

The materials and methods have previously been described in example 5.

A) Results

Four variants cleaving HIV1_{—}1.3 (SEQ ID NO:321), were pooled, randomly mutagenized and transformed into yeast. The four variants submitted to random mutagenesis correspond to variants described in Table VIII (SEQ ID NO: 26, 27, 28 and 29).

2232 transformed clones were screened for cleavage against the HIV1_{—}1.3 (SEQ ID NO:321) and HIV1_{—}1.5 (SEQ ID NO:323) DNA targets. A total of 79 positive clones were found to cleave HIV1_{—}1.3 (SEQ ID NO:321), while 60 of those cleaved also the HIV1_{—}1.5 target (SEQ ID NO:323). Sequencing of the 79 clones allowed the identification of 47 novel endonuclease variants. An example of the identified variants is presented in table 1× and FIG. 16.

The 79 clones showing cleaving target HIV1_{—}1.3 (SEQ ID NO:321) were then mated with a yeast strain that contains (i) the HIV1_{—}1 target (SEQ ID NO:319) in a reporter plasmid (ii) an expression plasmid containing a variant that cleaves the HIV1_{—}1.4 target (SEQ ID NO:322) (I-CreI 33T,40K,44R,68Y,70S,77N,132V or KNSTQK/RYSDN +132V (SEQ ID NO:46), according to the nomenclature of Table I). After mating with this yeast strain, 76 clones were found to cleave the HIV1_{—}1 target (SEQ ID NO:319). Thus, 76 positives contained proteins able to form heterodimers with KNSTQK/RYSDN +132V (SEQ ID NO: 46) showing cleavage activity on the HIV1_{—}1 target (SEQ ID NO:319). An example of positives is shown in FIG. 17. Sequencing of these 76 positive clones indicates that 44 distinct variants were identified. Ten of these 44 variants are presented as an example in Table IX.

TABLE IX
Examples of 10 functional variants displaying strong cleavage activity
for HIV1_1.3 (SEQ ID NO: 321).
Optimized variants HIV1__1.3 (2^{nd} round)

| | |
|---|---|
| SEQ ID NO: 36 | I-CreI 30G 38R 44V 54L 68E 75N 77R 80K 85Y 99R 111H |
| SEQ ID NO: 37 | I-CreI 2S 30G 38R 44V 54L 68E 75N 77R 80K 89A 99R 111H 132V 155Q 163R |
| SEQ ID NO: 38 | I-CreI 16L 30G 31R 38R 44V 54L 57E 61G 68E 75N 77R 80K 81T 132V 162P |
| SEQ ID NO: 39 | I-CreI 30G 38R 44V 54L 68E 75N 77R 80K 99R 111H 163R |
| SEQ ID NO: 40 | I-CreI 17A 30G 38R 42A 44V 54L 64A 68E 75N 77R 80R 86D 99R 111H |
| SEQ ID NO: 41 | I-CreI 30G 38R 44V 54L 68E 75N 77R 80K 81T 121R 132V 160E |
| SEQ ID NO: 42 | I-CreI 28R 30G 38R 39I 44V 54L 68E 75N 77R 80K 81T 86S 132V 150T 162P |
| SEQ ID NO: 43 | I-CreI 30G 34R 38R 44V 54L 68E 75N 77R 80K 81T 132V 163R |
| SEQ ID NO: 44 | I-CreI 30G 38R 44N 68Y 70S 75R 77Y 79N 100E 105A |
| SEQ ID NO: 45 | I-CreI 16L 30G 38R 44V 54L 68E 75N 77R 80K 99R 111H 112Q |

* Mutations resulting from random mutagenesis are in bold.

### EXAMPLE 6

### Improvement of Meganucleases Cleaving HIV1_{—}1 (SEQ ID NO:319) By Site-Directed Mutagenesis of Proteins Cleaving HIV1_{—}1.3 (SEQ ID NO:321) and Assembly with Proteins Cleaving HIV1_{—}1.4 (SEQ ID NO:322)

The I-CreI variants cleaving HIV1_{—}1.3 (SEQ ID NO:321) described in Table IX issued from random mutagenesis in examples 5 and 5bis were also mutagenized by introducing selected amino-acid substitutions in the proteins and screening for more efficient variants cleaving HIV1_{—}1 (SEQ ID NO:319) in combination with a variant cleaving HIV1_{—}1.4 (SEQ ID NO:322).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V 105A) and Isoleucine 132 with Valine (I132V). These mutations were introduced into the coding sequence of proteins cleaving HIV1_{—}1.3 (SEQ ID NO:321), and the resulting proteins were tested for their ability to induce cleavage of the HIV1_{—}1 target (SEQ ID NO:319), upon co-expression with a variant cleaving HIV1_{—}1.4 (SEQ ID NO:322), as well as for the ability to cleave targets HIV1_{—}1.3 (SEQ ID NO:321) and HIV1_{—}1.5 (SEQ ID NO:323).

### A) Material and Methods

### a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the 1-CreI coding sequence. For both the 5′ and 3′ end, PCR amplification is carried out using a primer with homology to the vector (Gal10F 5′-gcaactttagtgctgacacatacagg-3′(SEQ ID NO: 16) or Ga110R 5′-acaaccttgattggagacttgacc-3′(SEQ ID NO: 17)) and a primer specific to the I-CreI coding sequence for amino acids 14-24 that contains the substitution mutation G19S (G19SF 5′-gccggattgtggactctgacggtagcatcatc-3′ (SEQ ID NO: 47) or G19SR 5′-gatgatgctaccgtcagagtccacaaagccggc-3′(SEQ ID NO: 48)). The same strategy is used with the following pair of oligonucleotides to introduce the mutations leading to the F54L, E80K, F87L, V105A and I132V substitutions in the coding sequences of the variants, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgcttaa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
I132VF:
5′-acctgggtggatcaggttgcagctctgaacgat-3′
and
I132VR:
5′-atcgttcagagctgcaacctgatccacccaggt-3′.

For each substitution to be introduced, the resulting PCR products contain 33 bp of homology with each other. The PCR fragments were purified. The ten PCR fragments were pooled en equimolar amounts to generate a mix containing 50 ng of PCR DNA and 75 ng of vector DNA (pCLS0542, FIG. 9), linearized by digestion with NcoI and EagI. This mix was used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

### c) Mating of Meganuclease Expressing Clones and Screening in Yeast

The experimental procedure is as described in example 5.

### d) Sequencing of Variants

The experimental procedure is as described in example 2.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of five variants cleaving HIV1_{—}1.3 (SEQ ID NO:321) (described in Table X). 558 transformed clones were screened for cleavage against the HIV1_{—}1.3 (SEQ ID NO:321) and HIV1_{—}1.5 (SEQ ID NO:323) DNA targets. A total of 395 positive clones were found to cleave HIV1_{—}1.3 (SEQ ID NO:321), while 349 of those cleaved also the HIV1_{—}1.5 target (SEQ ID NO:323). An example of positive variants is shown in FIG. 18

The 558 transformed clones were also mated with a yeast strain that contains (i) the HIV1_{—}1 target (SEQ ID NO:319) in a reporter plasmid (ii) an expression plasmid containing a variant that cleaves the HIV1_{—}1.4 target (SEQ ID NO:322) (I-CreI 33T,40K,44R,68Y,70S,77N +132V or KNSTQK/RYSDN +132V (SEQ ID NO:46), according to the nomenclature of Table I). After mating with this yeast strain, 458 clones were found to cleave the HIV1_{—}1 (SEQ ID NO:319). Thus, 458 positives contained proteins able to form heterodimers with KNSTQK/RYSDN +132V (SEQ ID NO: 46) showing cleavage activity on the HIV1_{—}1 target (SEQ ID NO:319). An example of positives is shown in FIG. 19.

Sequencing of the 186 clones with the highest cleavage activity on the HIV1_{—}1 target (SEQ ID NO:319) allowed the identification of 138 different endonuclease variants.

The sequence of the five best I-CreI variants cleaving the HIV1_{—}1 target (SEQ ID NO:319) when forming a heterodimer with the KNSTQK/RYSDN +132V variant (SEQ ID NO:46) are listed in Table XI.

TABLE X
Sequences corresponding to the variants cleaving the HIV1_1.3 DNA
target (SEQ ID NO: 321) used for improvement by site-directed mutagenesis
I-CreI variants cleaving the HIV1_1.3 target (SEQ ID NO: 321)*

| | Amino acids at | |
|---|---|---|
| | positions 28, | |
| SEQ | 30, 32, 33, 38, | |
| ID | 40/44, 68, 70, | |
| NO: | 75 and 77 | Unique mutations, compared to the I-CreI sequence |
| 26 | KGSYRS/VERNR | 30G38R44V54L68E75N77R80K81T132V163R |
| 36 | KGSYRS/VERNR | 30G38R44V54L68E75N77R80K85Y99R111H |
| 38 | KGSYRS/VERNR | 16L30G31R38R44V54L57E61G68E75N77R80K81T132V162P |
| 40 | KGSYRS/VERNR | 17A30G38R42A44V54L64A68E75N77R80R86D99R111H |
| 42 | KGSYRS/VERNR | 28R30G38R39I44V54L68E75N77R80K81T86S132V150T162P |

*The nomenclature of the mutants is the same as for Table I. (ex: KRGYQS/RHRDI stands for K28, R30, G32, Y33, Q38, S40/R44, H68, R70, D75 and 177)

TABLE XI
Functional variant combinations displaying strong cleavage activity
for HIV1_1. (SEQ ID NO: 319)

| | | Optimized* Variants HIV1_1.3 |
|---|---|---|
| | | (SEQ ID NO: 59 to 63) |
| VARIANT HIV1_1.4 | I-CreI | I-CreI 19S 30G 38R 44V 54L 68E 75N 77R 80K 81T 132V |
| (SEQ ID NO: 46) | 28K30N32S33T38Q40S44V | I-CreI 19S 30G 38R 44V 54L 68E 75N 77R 80K 81T 86D 99R 111H 132V |
| | 68E70R75N77R132V | 162P |
| | KNSTQK/RYSDN + 132V | I-CreI 19S 30G 31R 38R 44V 54L 64A 68E 75N 77R 80R 86D 105A 132V |
| | | I-CreI 19S 30G 38R 44V 54L 68E 75N 77R 80K 85Y 99R 111H 162P |
| | | I-CreI 19S 30G 38R 44V 54L 64A 68E 75N 77R 80R 86D 99R 111H |

*Mutations resulting from site-directed mutagenesis are in bold.

### EXAMPLE 7

### Improvement of Meganucleases Cleaving HIV1_{—}1 (SEQ ID NO:319) By Random Mutagenesis of Proteins Cleaving HIV1_{—}1.4 (SEQ ID NO:322) and Assembly with Proteins Cleaving HIV1_{—}1.3 (SEQ ID NO:321)

As a complement to example 4 we also decided to perform random mutagenesis with variants that cleave HIV1_{—}1.4 (SEQ ID NO:322). The mutagenized proteins cleaving HIV1_{—}1.4 (SEQ ID NO:322) were then tested to determine if they could efficiently cleave HIV1_{—}1 (SEQ ID NO:319) when co-expressed with a protein cleaving HIV1_{—}1.3 (SEQ ID NO:321).

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25). Approximately 25 ng of the PCR product and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Variant-Target Yeast Strains, Screening and Sequencing

The yeast strain FYBL2-7B (MATα, ura3Δ851, trp1Δ63, leu2Δ1, lys2Δ202) containing the HIV1_{—}1 target (SEQ ID NO:319) in the yeast reporter vector (pCLS1055, FIG. 8) was transformed with variants, in the leucine vector (pCLS0542), cutting the HIV1_{—}1.3 target (SEQ ID NO:321), using a high efficiency LiAc transformation protocol. Variant-target yeast strains were used as target strains for mating assays as described in example 4. Positives resulting clones were verified by sequencing (MILLEGEN) as described in example 2.

### B) Results

Six variants cleaving HIV1_{—}1.4 (SEQ ID NO:322) were pooled, randomly mutagenized and transformed into yeast. The sequences of the variants subjected to random mutagenesis are described in table XII.

2232 transformed clones were screened for cleavage against the HIV1_{—}1.4 (SEQ ID NO:322) and HIV1_{—}1.6 DNA targets (SEQ ID NO:324). A total of 388 positive clones were found to cleave HIV1_{—}1.4 (SEQ ID NO:322), while 88 of those also cleaved the HIV1_{—}1.6 target (SEQ ID NO:324). Sequencing of the 89 clones showing the strongest activity allowed the identification of 50 novel endonuclease variants. An example of the identified variants is presented in table XIII and in FIG. 20.

```
TABLE XII
Sequences corresponding to the
variants cleaving the HIV1_1.4 DNA target
  (SEQ ID NO: 322) used for improvement
by random mutagenesis
Amino acids at positions 28, 30, 32, 33, 38,
40/44, 68, 70, 75 and 77 of I-CreI variants
  cleaving the HIV1_1.4 target(SEQ ID NO: 322)
(ex: KRGYQS/RHRDI stands for K28, R30, G32,
Y33, Q38, S40/R44, H68, R70, D75 and 177)
```

| | |
|---|---|
| QNSSRK/KYSES | SEQ ID NO: 7 |
| KNSCAS/KYSES | SEQ ID NO: 8 |
| KNSSRN/KYSES | SEQ ID NO: 9 |
| KCSTQR/RYSDQ | SEQ ID NO: 10 |
| KNSTQK/RYSDN | SEQ ID NO: 11 |
| KNSCAS/RYSDN | SEQ ID NO: 66 |

TABLE XIII
Examples of 10 functional variants displaying strong cleavage
activity for HIV1_1.4 (SEQ ID NO: 322).
Optimized variants HIV1_1.4

| | |
|---|---|
| SEQ ID NO: 46 | I-CreI 33T 40K 44R 68Y 70S 77N 132V |
| SEQ ID NO: 67 | I-CreI 33T 40K 44R 68C 70S 77N 160R |
| SEQ ID NO: 68 | I-CreI 33T 40K 44R 68Y 70S 77N |
| SEQ ID NO: 69 | I-CreI 33T 40K 44R 68Y 70S 77N 157K |
| SEQ ID NO: 70 | I-CreI 16L 33T 40R 44R 68Y 70S 77N |
| SEQ ID NO: 71 | I-CreI 33T 40K 43L 44R 68Y 70S 77N |
| SEQ ID NO: 72 | I-CreI 6D 33T 40K 44R 68Y 70S 75E 77S 83S 154N |
| | 161P |
| SEQ ID NO: 73 | I-CreI 38R 44R 70S 75N 105A 131R |
| SEQ ID NO: 74 | I-CreI 7E 33T 40K 44K 68Y 70S 72P 75E 77S 83S |
| SEQ ID NO: 75 | I-CreI 3A 33C 40K 44R 68Y 70S 77N 129A 151G |

* Mutations resulting from random mutagenesis are in bold.

The 89 clones showing the highest cleavage activity on target HIV1_{—}1.4 (SEQ ID NO:322) were then mated with a yeast strain that contains (i) the HIV1_{—}1 target (SEQ ID NO:319) in a reporter plasmid (ii) an expression plasmid containing a variant that cleaves the HIV1_{—}1.3 target (SEQ ID NO:321) (I-CreI 30G,38R,44V,68E,75N,77R,54L,80K,81T,132V,163R or KGSYRS/VERNR +54L+80K+81T+132V+163R (SEQ ID NO:26), according to the nomenclature of Table I). After mating with this yeast strain, 88 clones were found to cleave the HIV1_{—}1 target (SEQ ID NO:319). Thus, 46 positives contained proteins able to form heterodimers with KGSYRS/VERNR +54L+80K+81T+132V+163R (SEQ ID NO: 26), that showed cleavage activity on the HIV1_{—}1 target (SEQ ID NO:319). An example of positives is shown in FIG. 21. Sequencing of these 88 positive clones indicates that 46 distinct variants were identified. Ten of these 46 variants are presented as an example in Table XIII.

### EXAMPLE 7bis

### Improvement of Meganucleases Cleaving HIV1_{—}1 (SEQ ID NO:319) By a Second Round of Random Mutagenesis of Proteins Cleaving HIV1_{—}1.4 (SEQ ID NO:322) and Assembly with Proteins Cleaving HIV1_{—}1.3 (SEQ ID NO:321)

In order to further improve the activity of the obtained meganucleases, a second round of random mutagenesis was carried out following the same rationale of example 7. For this purpose, four variants cleaving HIV1_{—}1.4 (SEQ ID NO:322) were mutagenized, and variants were screened for cleavage activity of HIV1_{—}1.4 (SEQ ID NO:322) and HIV1_{—}1.6 (SEQ ID NO:324) targets. Additionally the mutants with the strongest activity were screened for cleavage activity of HIV1_{—}1 (SEQ ID NO:319) when co-expressed with a variant cleaving HIV1_{—}1.3 (SEQ ID NO:321).

The materials and methods have previously been described in example 7.

A) Results

Four variants cleaving HIV1_{—}1.4 (SEQ ID NO:322), were pooled, randomly mutagenized and transformed into yeast. The four variants submitted to random mutagenesis correspond to variants described in Table XIII (SEQ ID NO: 46, 68, 69 and 71).

2232 transformed clones were screened for cleavage against the HIV1_{—}1.4 (SEQ ID NO:322) and HIV1_{—}1.6 DNA (SEQ ID NO:324) targets. A total of 59 positive clones were found to cleave HIV1_{—}1.4 (SEQ ID NO:322), while 16 of those cleaved also the HIV1_{—}1.6 (SEQ ID NO:324) target. Sequencing of the 49 clones allowed the identification of 35 novel endonuclease variants. An example of the identified variants is presented in table XIV and FIG. 22.

The 59 clones showing cleaving target HIV1_{—}1.4 (SEQ ID NO:322) were then mated with a yeast strain that contains (i) the HIV1_{—}1 target (SEQ ID NO:319) in a reporter plasmid (ii) an expression plasmid containing a variant that cleaves the HIV1_{—}1.3 target (SEQ ID NO:321) (I-CreI 30G,38R,44N,68Y,70S,75R,77Y +79N or KGSYRS/NYSRY +79N (SEQ ID NO:28), according to the nomenclature of Table I). After mating with this yeast strain, 42 clones were found to cleave the HIV1_{—}1 (SEQ ID NO:319). Thus, 42 positives contained proteins able to form heterodimers with KGSYRS/NYSRY +79N (SEQ ID NO: 28) showing cleavage activity on the HIV1_{—}1 target (SEQ ID NO:319). An example of positives is shown in FIG. 23. Sequencing of these 42 positive clones indicates that 35 distinct variants were identified. Ten of these 35 variants are presented as an example in Table XIV.

TABLE XIV
Examples of 10 functional variants displaying strong cleavage
activity for HIV1_1.4. (SEQ ID NO: 322)
Optimized variants HIV1_1.4 (2^{nd} round)

| | |
|---|---|
| SEQ ID NO: 76 | I-CreI 33T 40K 44R 68Y 70S 77N 157K |
| SEQ ID NO: 77 | I-CreI 33T 40K 44R 68Y 70S 77N 117G |
| SEQ ID NO: 78 | I-CreI 6D 33T 40K 44R 68Y 70S 77N |
| SEQ ID NO: 79 | I-CreI 33T 40K 44R 68Y 70S 77N |
| SEQ ID NO: 80 | I-CreI 8V 33T 40K 44R 64A 68Y 70S 77N 103K 116R |
| | 132V |
| SEQ ID NO: 81 | I-CreI 16L 33T 40K 43L 44R 68Y 70S 77N |
| SEQ ID NO: 82 | I-CreI 33T 40K 44R 57E 68N 70S 77N 121R 132V |
| SEQ ID NO: 83 | I-CreI 33T 40K 44R 64A 68Y 70T 77N 153G |
| SEQ ID NO: 84 | I-CreI 33T 40K 44R 68Y 70S 77N 105L 156N 157K |
| SEQ ID NO: 85 | I-CreI 33T 40K 43L 44R 68Y 70S 77N 105A |

* Mutations resulting from random mutagenesis are in bold.

### EXAMPLE 8

### Strategy for Engineering Meganucleases Cleaving the HIV1_{—}3 Target (SEQ ID NO:321) from the HIV1 Virus

The HIV1_{—}3 target (SEQ ID NO:321) is a 22 by (non-palindromic) target located in U5 region of the proviral LTRs. Since the LTRs are duplicated sequences flanking the viral ORFs in the integrated provirus, the HIV1_{—}3 target (SEQ ID NO:321) is present twice in the HIV1 provirus. This target is precisely located at positions 599-620 and 9674-9695 of the HIV-1 pNL4-3 vector (accession number AF324493, Adachi et al., J. Virol., 1986, 59, 284-291), a subtype B infectious molecular clone.

The HIV1_{—}3 sequence (SEQ ID NO: 325) is partly a patchwork of the 10CAG_P (SEQ ID NO:374), 10ACA_P (SEQ ID NO:375), 5CCT_P (SEQ ID NO:384) and 5_GAC_P (SEQ ID NO:385) targets (FIG. 24) which are cleaved by previously identified meganucleases, obtained as described in International PCT Applications WO 2006/097784 and WO 2006/097853; Arnould et al., J. Mol. Biol., 2006, 355, 443-458; Smith et al., Nucleic Acids Res., 2006. Thus, HIV1_{—}3 could be cleaved by combinatorial variants resulting from these previously identified meganucleases.

The 10CAG_P (SEQ ID NO:374), 10ACA_P (SEQ ID NO:375), 5CCT_P (SEQ ID NO:384) and 5_GAC_P (SEQ ID NO:385) target sequences are 24 by derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HIV1_{—}3 series of targets (SEQ ID NO:325 to 330) were defined as 22 by sequences instead of 24 bp. HIV1_{—}3 (SEQ ID NO:325) differs from C1221 (SEQ ID NO:343) in the 4 by central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the TTTA sequence in −2 to 2 was first substituted with the GTAC sequence from C1221 (SEQ ID NO:343), resulting in target HIV1_{—}3.2 (SEQ ID NO: 326, FIG. 24). Then, two palindromic targets, HIV1_{—}3.3 (SEQ ID NO: 327) and HIV1_{—}3.4 (SEQ ID NO: 328), were derived from HIV1_{—}3.2 (SEQ ID NO:326) (FIG. 24). Since HIV1_{—}3.3 (SEQ ID NO:327) and HIV1_{—}3.4 (SEQ ID NO:328) are palindromic, they should be cleaved by homodimeric proteins. Two other pseudo-palindromic targets were derived from these two, containing the TTTA sequence in −2 to 2 (targets HIV1_{—}3.5 (SEQ ID NO: 329) and HIV1_{—}3.6 (SEQ ID NO: 330), FIG. 24). Thus, proteins able to cleave HIV1_{—}3.3 (SEQ ID NO:327) and HIV1_{—}3.4 (SEQ ID NO:328) targets or, preferentially, the pseudo-palindromic targets as homodimers were first designed (examples 9 and 10) and then co-expressed to obtain heterodimers cleaving HIV1_{—}3 (SEQ ID NO:325) (example 11). Heterodimers cleaving the HIV1_{—}3.2 (SEQ ID NO:326) or HIV1_{—}3 (SEQ ID NO:325) targets could not be identified. In order to obtain cleavage activity for the HIV1_{—}3 target (SEQ ID NO:325), a series of variants cleaving HIV1_{—}3.3 (SEQ ID NO:327) and HIV1_{—}3.4 (SEQ ID NO:328) was chosen, and then refined. The chosen variants were subjected to random or site-directed mutagenesis, and used to form novel heterodimers that were screened against the HIV1_{—}3 target (SEQ ID NO:325) (examples 12, 13, 14 and 15). Heterodimers could be identified with an improved cleavage activity for the HIV1_{—}3 target (SEQ ID NO:325).

### EXAMPLE 9

### Identification of Meganucleases Cleaving HIV1_{—}3.3 (SEQ ID NO:327)

This example shows that I-CreI variants can cut the HIV1_{—}3.3 target (SEQ ID NO:327) sequence derived from the left part of the HIV1_{—}3.2 target (SEQ ID NO:326) in a palindromic form (FIG. 24).

HIV1_{—}3.3 (SEQ ID NO:327) is similar to 10CAG_P (SEQ ID NO:374) at positions ±1, ±2, ±6, ±8, ±9, and +10 and to 5CCT_P (SEQ ID NO:384) at positions ±1, ±2, ±3, ±4, ±5 and ±6. It was hypothesized that positions ±7 and ±11 would have little effect on the binding and cleavage activity. Variants able to cleave the 10CAG_P (SEQ ID NO:374) target were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156. Variants able to cleave 5CCT_P (SEQ ID NO:384) were obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Both sets of proteins are mutated at position 70. However, the existence of two separable functional subdomains was hypothesized. This implies that this position has little impact on the specificity at bases 10 to 8 of the target. Mutations at positions 24 found in variants cleaving the 5CCT_P (SEQ ID NO:384) target will be lost during the combinatorial process. But it was hypothesized that this will have little impact on the capacity of the combined variants to cleave the HIV1_{—}3.3 target (SEQ ID NO:327).

Therefore, to check whether combined variants could cleave the HIV13.3 target (SEQ ID NO:327), mutations at positions 44, 68, 70, 75 and 77 from proteins cleaving 5CCT_P (SEQ ID NO:384) were combined with the 28, 30, 32, 33, 38 and 40 mutations from proteins cleaving 10CAG_P (SEQ ID NO:374).

### A) Material and Methods

### a) Construction of Target Vector

The target was cloned as follows: an oligonucleotide corresponding to the HIV1_{—}3.3 target sequence (SEQ ID NO:327) flanked by gateway cloning sequences was ordered from PROLIGO: 5′ TGGCATACAAGTTTCTCAGACCCTGTACAGGGTCTGAGCAATCGTCTGTCA 3′ (SEQ ID NO: 86). The same procedure was followed for cloning the HIV1_{—}3.5 target (SEQ ID NO:329), using the oligonucleotide: 5′ TGGCATACAAGTTTCTCAGACCCTTTTAAGGGTCTGAGCAATCGTCTGTCA 3′ (SEQ ID NO: 87). Double-stranded target DNA, generated by PCR amplification of the single stranded oligonucleotide, was cloned using the Gateway protocol (INVITROGEN) into the yeast reporter vector (pCLS1055, FIG. 8). Yeast reporter vector was transformed into Saccharomyces cerevisiae strain FYBL2-7B (MATα, ura3Δ851, trp1Δ63, leu2Δ1, lys2Δ202), resulting in a reporter strain.

b) Construction of Combinatorial Mutants I-CreI variants cleaving 10CAG_P (SEQ ID NO:374) or 5CCT_P (SEQ ID NO:384) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10AGA_P (SEQ ID NO:381) and 5TAC_P (SEQ ID NO:389) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′(SEQ ID NO: 17)) specific to the vector (pCLS0542, FIG. 9) and primers (assF 5′-ctannnttgaccttt-3′ (SEQ ID NO: 18) or assR 5′-aaaggtcaannntag-3′(SEQ ID NO: 19), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 9) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.
c) Mating of Meganuclease Expressing Clones and Screening in Yeast Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

### B) Results

I-CreI combinatorial variants were constructed by associating mutations at positions 44, 68, 70, 75 and 77 from proteins cleaving 5CCT_P (SEQ ID NO:384) with the 28, 30, 32, 33, 38 and 40 mutations from proteins cleaving 10CAG_P (SEQ ID NO:374) on the I-CreI scaffold, resulting in a library of complexity 1600. Examples of combinatorial variants are displayed in Table XV. This library was transformed into yeast and 3348 clones (2 times the diversity) were screened for cleavage against the HIV1_{—}3.3 (SEQ ID NO:327) and HIV1_{—}3.5 (SEQ ID NO:329) DNA targets. 10 positive clones were found to cleave the HIV1_{—}3.3 target (SEQ ID NO:327), which after sequencing turned out to correspond to 7 different novel endonuclease variants (Table XVI). These variants showed no cleavage activity of the HIV1_{—}3.5 DNA target (SEQ ID NO:329). Examples of positives are shown in FIG. 25. Some of the variants obtained display non parental combinations at positions 28, 30, 32, 33, 38, 40 or 44, 68, 70, 75, 77 (SEQ ID NO: 92 to 94, Table XVI). Such combinations likely result from PCR artifacts during the combinatorial process. Alternatively, the variants may be I-CreI combined variants resulting from micro-recombination between two original variants during in vivo homologous recombination in yeast.

TABLE XV
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, 70, | | | | | | | | | | | |
| 75 and 77 | | | | | | | | | | | |
| (ex: ARNNI | | | | | | | | | | | |
| stands for | | | | | | | | | | | |
| A44, R68, | | | | | | | | | | | |
| N70, | Amino acids at positions 28, 30, 32, 33, 38 and 40 | | | | | | | | | | |
| N75 and | (ex: KHSSQS stands for K28, H30, S32, 533, Q38 and S40) | | | | | | | | | | |
| I77) | KQDYQS | KYTCQS | KCSCQS | KDSRQS | KTSCYS | KNTCQS | KDSRSS | KNSNYR | KGSYQG | KSSQQS | KTEYQS |
| KNSNI | | | | | | | | | | | |
| KTGNI | | | | | | | | | | | |
| KRTNI | | | | | | + | | | | | |
| KGTNI | | | | | | | | | | | |
| DASKR | | | | | | | | | | | |
| KASNI | | | | | | | | | | | |
| KTSDR | | | | | | | | | | | |
| KYSDY | | | | | | | | | | | |
| RYSNN | | | | | | | | | | | |
| KYSYN | | | | | | | | | | | |
| KESDR | | | | | | | | | | | |
| KASDK | | | + | | | | | | | | |
| RTSNN | | | | | | | | | | | |
| QHHNI | | | | | | | | | | | |
| TRSRT | | | | | | | | | | | |
| KSNDI | | | | | | | | + | | | |
| KDSNR | | | | | | | | | | | |
| KESDK | | | | | | | | | | | |
| PCSYT | | | | | | | | | | | |
| KESNR | | | + | | | | | | | | |
| RYSNI | | | | | | | | | | | |
| KNTNI | | | | | | | | | | | |
| KTSDI | | | | | | | | | | | |
| RRSND | | | | | | | | | | | |

*Only 264 out of the 1600 combinations are displayed.
+ indicates that a functional combinatorial variant cleaving the HIV1_3.3 target (SEQ ID NO: 327) was found among the identified positives.

TABLE XVI
I-CreI variants capable of cleaving the
HIV1_3.3 DNA target (SEQ ID NO: 327).

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and 177) | NO: |
| KNSNYR/KSNDI | 88 |
| KCSCQS/KASDK | 89 |
| KCSCQS/KESNR | 90 |
| KNTCQS/KRTNI | 91 |
| KNKCQS/QEGNL | 92 |
| KNSNYS/KYSYI | 93 |
| KSSQQS/QASET | 94 |

### EXAMPLE 10

### Making of Meganucleases Cleaving HIV1_{—}3.4 (SEQ ID NO:328)

This example shows that I-CreI variants can cleave the HIV1_{—}3.4 DNA target sequence (SEQ ID NO:328) derived from the right part of the HIV1_{—}3.2 target (SEQ ID NO:326) in a palindromic form (FIG. 24).

HIV1_{—}3.4 (SEQ ID NO:328) is similar to 5GAC_P (SEQ ID NO:385) at positions ±1, ±2, ±3, ±4, ±5 and ±8 and to 10ACA_P (SEQ ID NO:375) at positions ±1, ±2, ±3, ±4, ±8, ±9 and ±10. It was hypothesized that positions ±6, ±7 and ±11 would have little effect on the binding and cleavage activity. Variants able to cleave 5GAC_P (SEQ ID NO:385) were obtained by mutagenesis of I-CreI N75 at positions 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10ACA_P target (SEQ ID NO:375) were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156.

Both sets of proteins are mutated at position 70. However, the existence of two separable functional subdomains was hypothesized. This implies that this position has little impact on the specificity at bases 10 to 8 of the target.

Therefore, to check whether combined variants could cleave the HIV1_{—}3.4 target (SEQ ID NO:328), mutations at positions 44, 68, 70, 75 and 77 from proteins cleaving 5GAC_P (SEQ ID NO:385) were combined with the 28, 30, 32, 33, 38 and 40 mutations from proteins cleaving 10ACA_P (SEQ ID NO:375).

### A) Material and Methods

a) Construction of Target Vector The experimental procedure is as described in example 2, with the exception that different oligonucleotides corresponding to the HIV1_{—}3.4 (SEQ ID NO:328) and HIV1_{—}3.6 targets (SEQ ID NO:330). The oligonucleotide used for the HIV1_{—}3.4 target (SEQ ID NO:328) was:

(SEQ ID NO: 95)
5′ TGGCATACAAGTTTCCACACTGACGTACGTCAGTGTGGCAATCGTC
TGTCA 3′,
and
(SEQ ID NO: 96)
5′ TGGCATACAAGTTTCCACACTGACTTTAGTCAGTGTGGCAATCGTC
TGTCA 3′
for HIV1_3.6 target (SEQ ID NO: 330).

### b) Construction of Combinatorial Variants

I-CreI variants cleaving 10ACA_P (SEQ ID NO:375) or 5GAC_P (SEQ ID NO:385) were previously identified, as described in Smith et al. Nucleic Acids Res., 2006, 34, e149; International PCT Applications WO 2007/060495 and

WO 2007/049156, and Arnould et al., J. Mol. Biol., 2006, 355, 443-458; International PCT Applications WO 2006/097784 and WO 2006/097853, respectively for the 10ACA_P (SEQ ID NO:375) and 5GAC_P (SEQ ID NO:385) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′ (SEQ ID NO: 17)) specific to the vector (pCLS1107, FIG. 11) and primers (assF 5′-ctannnttgaccttt-3′ (SEQ ID NO: 18) or assR 5′-aaaggtcaannntag-3′(SEQ ID NO: 19)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. Positives resulting clones were verified by sequencing (MILLEGEN) as described in example 2.

### B) Results

I-CreI combinatorial variants were constructed by associating mutations at positions 44, 68, 70, 75 and 77 from proteins cleaving 5GAC_P (SEQ ID NO:385) with the 28, 30, 32, 33, 38 and 40 mutations from proteins cleaving 10ACA_P (SEQ ID NO:375) on the I-CreI scaffold, resulting in a library of complexity 2280. Examples of combinatorial variants are displayed in Table XVII. This library was transformed into yeast and 3348 clones (1.5 times the diversity) were screened for cleavage against the HIV1_{—}3.4 (SEQ ID NO:328) and HIV1_{—}3.6 DNA (SEQ ID NO:330) targets. A total of 305 positive clones were found to cleave HIV1_{—}3.4 (SEQ ID NO:328), and two of those variants showed cleavage activity on the HIV1_{—}3.6 (SEQ ID NO:330) target. DNA Sequencing of these 93 strongest clones allowed the identification of 64 novel endonuclease variants. Examples of positives are shown in FIG. 26. Some variants identified display non parental combinations at positions 28, 30, 32, 33, 38, 40 or 44, 68, 70, 75, 77 as well as additional mutations (see examples Table XVIII, SEQ ID NO: 102 to 104). Such variants likely result from PCR artifacts during the combinatorial process. Alternatively, the variants may be I-CreI combined variants resulting from micro-recombination between two original variants during in vivo homologous recombination in yeast.

TABLE XVII
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, 70, | | | | | | | | | | | |
| 75 and 77 | | | | | | | | | | | |
| (ex: ARNNI | | | | | | | | | | | |
| stands for | | | | | | | | | | | |
| A44, R68, | | | | | | | | | | | |
| N70, | Amino acids at positions 28, 30,32, 33, 38 and 40 | | | | | | | | | | |
| N75 and | (ex: KHSSQS stands for K28, H30, S32, S33, Q38 and S40) | | | | | | | | | | |
| I77) | KRSYER | KRSYES | KNSYYS | KSSCQS | KNSRES | KNTYSS | KNDYYS | KNSRER | KRDYQS | KNEYYS | KNTYAS |
| AYSRI | | + | + | | | | | + | + | | |
| ARSYT | | | + | | | | | | + | | |
| IRRNR | | | | | | | | | | | |
| NYSRQ | | + | + | | + | | | + | + | + | + |
| YDSRV | | | | | | | | | | | |
| YKSRQ | | | | | | | | | | | |
| NRSRV | | + | | | | | | | | | |
| YRSYN | | | | | | | | | | | |
| NYSRY | | + | | | + | | + | + | | | |
| NRSRD | | | | | | | | | + | | |
| AHRNI | | | | | | | | | | | |
| NRSRN | + | + | + | | + | | | | + | | |
| YSSRI | | + | | | | | | | + | + | |
| ARSYQ | | | | | | | | | | | |
| YSSRQ | | | | | | | | | + | | |
| YSSRV | | + | + | + | + | + | + | | + | + | |
| AYSRT | | + | | | | | | | | | |
| NASRY | | | | | | | | | | | |
| ARSYY | | | | | | | | | | | |
| NKSRN | | + | | | | | | | | | |
| NTSRQ | | + | | | | | | | + | | |
| NRSRQ | | + | | | | | | | + | | |
| NYSRD | | | | | | | | | | | |
| NRSRY | | | | | | | | | + | | |

*Only 264 out of the 2280 combinations are displayed.
+ indicates that a functional combinatorial variant cleaving the HIV1_3.4 target (SEQ ID NO: 328) was found among the identified positives.

TABLE XVIII
I-CreI variants capable of cleaving
the HIV1_3.4 DNA target (SEQ ID NO: 328).

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and 177) | NO: |
| KNSYYS/YSSRV +105A | 97 |
| KRSYER/NRSRN | 98 |
| KRSYES/YSSRQ | 99 |
| KNSYYS/YSSRV | 100 |
| KNDYYS/YSSRV | 101 |
| KRDYQS/YRSRE | 102 |
| KRDYYS/NRSRN | 103 |
| KNTYRS/YYSRT | 104 |

### EXAMPLE 11

### Making of Meganucleases Cleaving HIV1_{—}3.2 (SEQ ID NO:326) and HIV1_{—}3 (SEQ ID NO:325)

I-CreI variants able to cleave each of the palindromic HIV1_{—}3.2 (SEQ ID NO:326) derived targets (HIV1_{—}3.3 (SEQ ID NO:327) and HIV1_{—}3.4 (SEQ ID NO:328)) were identified in example 9 and example 10. Pairs of such variants (one cutting HIV1_{—}3.3 (SEQ ID NO:327) and one cutting HIV1_{—}3.4 (SEQ ID NO:328)) were co-expressed in yeast. Upon co-expression, there should be three active molecular species, two homodimers, and one heterodimer. It was assayed whether the heterodimers that should be formed, cut the HIV1_{—}3.2 (SEQ ID NO:326) and the non palindromic HIV1_{—}3 (SEQ ID NO:325) targets.

### A) Materials and Methods

a) Construction of Target Vector The experimental procedure is as described in example 9, with the exception that an oligonucleotide corresponding to the HIV1_{—}3.2 target sequence (SEQ ID NO:326): 5′ TGGCATACAAGTTTCTCAGACCCTGTACGTCAGTGTGGCAATCGTCTGTCA 3′(SEQ ID NO: 317) or the HIV1_{—}3 target sequence (SEQ ID NO:325): 5′ TGGCATACAAGTTTCTCAGACCCTTTTAGTCAGTGTGGCAATCGTCTGTCA 3′ (SEQ ID NO: 318) was used.

### b) Co-Expression of Variants

Yeast DNA was extracted from variants cleaving the HIV1_{—}3.4 (SEQ ID NO:328) target in the pCLS1107 expression vector using standard protocols and was used to transform E. coli. The resulting plasmid DNA was then used to transform yeast strains expressing a variant cutting the HIV1_{—}3.3 (SEQ ID NO:327) target in the pCLS0542 expression vector. Transformants were selected on synthetic medium lacking leucine and containing G418.

### c) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### B) Results

Co-expression of variants cleaving the HIV1_{—}3.4 target (SEQ ID NO:328) (4 variants) and five variants cleaving the HIV1_{—}3.3 target (SEQ ID NO:327) didn't result in cleavage of the HIV1_{—}3 target (SEQ ID NO:325), though most of the couples were able to cleave the HIV1_{—}3.2 target (SEQ ID NO:326).

### EXAMPLE 12

### Improvement of Meganucleases Cleaving HIV1_{—}3.3 (SEQ ID NO:327) by Random Mutagenesis of Initial Proteins Cleaving HIV1_{—}3.3 (SEQ ID NO:327)

I-CreI variants able to cleave the HIV1_{—}3.3 target (SEQ ID NO:327) have been previously identified in example 9.

These variants display, however, weak cleavage activity and where therefore mutagenized in order to improve their activity. Four mutants were selected for random mutagenesis and the variants obtained were screened for cleavage activity of HIV1_{—}3.3 (SEQ ID NO:327) and HIV1_{—}3.5 (SEQ ID NO:329) targets. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein.

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25), which are common to the pCLS0542 (FIG. 9) and pCLS1107 (FIG. 11) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Mating of Meganuclease Expressing Clones and Screening in Yeast

Experiments were performed as previously described in example 9. Positive resulting clones were verified by sequencing (MILLEGEN) as described in example 9.

### B) Results

Four variants cleaving HIV1_{—}3.3 (SEQ ID NO:327), were pooled, randomly mutagenized and transformed into yeast. The sequences of the variants subjected to random mutagenesis are described in table XVI (SEQ ID 88 to 91).

2232 transformed clones were screened for cleavage against the HIV1_{—}3.3 (SEQ ID NO:327) and HIV1_{—}3.5 (SEQ ID NO:329) DNA targets. A total of 51 positive clones were found to cleave HIV1_{—}3.3 (SEQ ID NO:327), while none of those cleaved the HIV1_{—}3.5 target (SEQ ID NO:329). Sequencing of the 51 clones allowed the identification of 35 novel endonuclease variants. An example of the identified variants is presented in table XIX and in FIG. 27.

TABLE XIX
Examples of 10 functional variants displaying strong cleavage
activity for HIV1_3.3 (SEQ ID NO: 327) after random mutagenesis.
Optimized variants HIV1_3.3

| | |
|---|---|
| SEQ ID NO: 105 | 32K 33A 44K 50R 68E 70S 75N 77R |
| SEQ ID NO: 106 | 32K 33A 44K 54I 60G 68E 70S 75N 77R 83T |
| SEQ ID NO: 107 | 32K 33A 44K 54L 68E 70S 75N 77R |
| SEQ ID NO: 108 | 32K 33A 44K 68E 70S 75N 77R 96R 105A 150S |
| SEQ ID NO: 109 | 32K 33A 44K 68E 70S 75N 77R 132N |
| SEQ ID NO: 110 | 33S 38Y 40R 44K 68S 70N 102V |
| SEQ ID NO: 111 | 33S 38Y 40R 44K 68S 70N |
| SEQ ID NO: 112 | 24V 32K 33A 35Y 44K 68E 70S 75N 77R |
| SEQ ID NO: 113 | 32K 33A 44K 68E 70S 75N 77R 81V 85R 154C |
| SEQ ID NO: 114 | 30C 33C 44K 54L 68A 70S 77K |

* Mutations resulting from random mutagenesis are in bold.

### EXAMPLE 12bis

### Improvement of Meganucleases Cleaving HIV1_{—}3.3 (SEQ ID NO:327) by a Second Round of Random Mutagenesis of Proteins Cleaving HIV1_{—}3.3 (SEQ ID NO:327)

In order to further improve the activity of the obtained meganucleases, a second round of random mutagenesis was carried out following the same rationale of example 7bis. For this purpose, ten variants cleaving HIV1_{—}3.3 (SEQ ID NO:327) were mutagenized, and variants were screened for cleavage activity of HIV1_{—}3.3 (SEQ ID NO:327) and HIV1_{—}3.5 (SEQ ID NO:329) targets. The materials and methods have previously been described in example 11.

### A) Results

Ten variants cleaving HIV1_{—}3.3 (SEQ ID NO:327), were pooled, randomly mutagenized and transformed into yeast. The variants submitted to random mutagenesis correspond to variants described in Table XIX (SEQ ID NO: 105 to 114). 2232 transformed clones were screened for cleavage against the HIV1_{—}3.3 (SEQ ID NO:327) and HIV1_{—}3.5 (SEQ ID NO:329) DNA targets. A total of 262 positive clones were found to cleave HIV1_{—}3.3 (SEQ ID NO:327), while 24 of those cleaved also, though weakly, the HIV1_{—}3.5 target (SEQ ID NO:329). Sequencing of the 93 clones showing the strongest cleavage activity in the HIV1_{—}3.3 target (SEQ ID NO:327) allowed the identification of 69 novel endonuclease variants. An example of the identified variants is presented in table XX and FIG. 28.

TABLE XX
Examples of 10 functional variants displaying strong cleavage activity
for HIV1_3.3 (SEQ ID NO: 327).
Optimized variants HIV1_3.3 (2^{nd} round)

| | |
|---|---|
| SEQ ID NO: 115 | 32K 33A 44K 68E 70S 75N 77R 96R 105A 154R |
| SEQ ID NO: 116 | 32K 33A 44K 68E 70S 72T 75N 77R 81V 85R 154C |
| SEQ ID NO: 117 | 32K 33A 43L 44K 54L 68E 70S 75N 77R |
| SEQ ID NO: 118 | 32K 33A 44K 49A 68E 70S 75N 77R 81V 85R 89A |
| | 129A 154C 158Q |
| SEQ ID NO: 119 | 30C 33C 44K 54L 68E 70S 75N 77R |
| SEQ ID NO: 120 | 16L 32K 33A 43L 44K 50R 68E 70S 75N 77R 81V |
| | 154C 155P |
| SEQ ID NO: 121 | 24V 32K 33A 44K 68E 70S 75N 77R 81V 85R |
| | 87L 154C 158E |
| SEQ ID NO: 122 | 7E 33S 38Y 40R 44K 68E 70S 75Y 77R 96R 105A |
| SEQ ID NO: 123 | 32K 33A 44K 50R 68E 70S 75N 77R |
| SEQ ID NO: 124 | 2S 32K 33A 44K 68E 70S 75N 77R 132N |

* Mutations resulting from random mutagenesis are in bold.

### EXAMPLE 13

### Improvement of Meganucleases Cleaving HIV1_{—}3 (SEQ ID NO:325) by Site-Directed Mutagenesis of Proteins Cleaving HIV1_{—}3.3 (SEQ ID NO:327) and Assembly with Proteins Cleaving HIV1_{—}3.4 (SEQ ID NO:328)

Five I-CreI variants cleaving HIV1_{—}3.3 (SEQ ID NO:327) after two cycles of random mutagenesis (examples 12 and 12bis) were mutagenized by introducing selected amino-acid substitutions in the proteins and screening for more efficient variants cleaving HIV1_{—}3 (SEQ ID NO:325) in combination with a variant cleaving HIV1_{—}3.4 (SEQ ID NO:328).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HIV1_{—}3.3 (SEQ ID NO:327), and the resulting proteins were tested for their ability to induce cleavage of the HIV1_{—}3 target (SEQ ID NO:325), upon co-expression with a variant cleaving HIV1_{—}3.4 (SEQ ID NO:328), as well as for the ability to cleave targets HIV1_{—}3.3 (SEQ ID NO:327) and HIV1_{—}3.5 (SEQ ID NO:329).

### A) Material and Methods

### a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 16) or Gal10R 5′-acaaccttgattggagacttgacc-3′(SEQ ID NO: 17) and a primer specific to the I-CreI coding sequence for amino acids 14-24 that contains the substitution mutation G19S (G19SF 5′-gccggctttgtggactctgacggtagcatcatc-3′ (SEQ ID NO: 47) or G19SR 5′-gatgatgctaccgtcagagtccacaaagccggc-3′(SEQ ID NO: 48)). The same strategy is used with the following pair of oligonucleotides to introduce the mutations leading to the F54L, E80K, F87L, V105A and I132V substitutions in the coding sequences of the variants, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgcttaa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
I132VF:
5′-acctgggtggatcaggttgcagctctgaac gat-3′
and
I132VR:
5′-atcgttcagagctgcaacctgatccacccaggt-3′.

For each substitution to be introduced, the resulting PCR products contain 33 bp of homology with each other. The PCR fragments were purified. The ten PCR fragments were pooled en equimolar amounts to generate a mix containing 50 ng of PCR DNA and 75 ng of vector DNA (pCLS0542, FIG. 9), linearized by digestion with NcoI and EagI. This mix was used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

### c) Mating of Meganuclease Expressing Clones and Screening in Yeast

The experimental procedure is as described in example 11.

### d) Sequencing of Variants

The experimental procedure is as described in example 9.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of five variants cleaving HIV1_{—}3.3 (SEQ ID NO:327) (described in Table XX, SEQ ID NO:115 to 119). 558 transformed clones were screened for cleavage against the HIV1_{—}3.3 (SEQ ID NO:327) and HIV1_{—}3.5 (SEQ ID NO:329) DNA targets. A total of 376 positive clones were found to cleave HIV1_{—}3.3 (SEQ ID NO:327), while 54 of those cleaved also the HIV1_{—}3.5 target (SEQ ID NO:329). An example of positive variants is shown in FIG. 29.

The 558 transformed clones were also mated with a yeast strain that contains (i) the HIV1_{—}3 target (SEQ ID NO:325) in a reporter plasmid (ii) an expression plasmid containing a variant that cleaves the HIV1_{—}3.4 target (SEQ ID NO:328) (38Y,44Y,68S,70S,75R,77V,43L,81V,105A,107R or KNSYYS/YSSRV +43L+81V+105A+107R (SEQ ID NO:125), according to the nomenclature of Table I). After mating with this yeast strain, 386 clones were found to cleave the HIV1_{—}3 (SEQ ID NO:325). Thus, 386 positives contained proteins able to form heterodimers with KNSYYS/YSSRV +43L+81V+105A+107R (SEQ ID NO: 125) showing cleavage activity on the HIV1_{—}3 target (SEQ ID NO:325). An example of positives is shown in FIG. 30.

Sequencing of 93 clones with the high cleavage activity on the HIV1_{—}3 (SEQ ID NO:325) and/or HIV1_{—}3.3 target (SEQ ID NO:327) allowed the identification of 62 different endonuclease variants.

As an example, ten I-CreI variants cleaving the HIV1_{—}3 target (SEQ ID NO:325) when forming a heterodimer with the KNSYYS/YSSRV variant (SEQ ID NO:125) are listed in Table XXI.

TABLE XXI
Functional variant combinations displaying strong cleavage activity
for HIV1_3 (SEQ ID NO: 325).

| | | Optimized* Variants HIV1_3.3 |
|---|---|---|
| | | (SEQ ID NO: 126 to 135, in this order) |
| VARIANT HIV1_3.4 | I-CreI | 32K 33A 44K 68E 70S 75N 77R 80K 154R |
| (SEQ ID NO: 125) | 38Y,44Y,68S,70S,75R,77V,43L,81V,105A,107R | 32K 33A 44K 68E 70S 72T 75N 77R 80K 129A 154C 158Q |
| | KNSTQK/RYSDN | 6D 19S 32K 33A 44K 49A 68E 70S 75N 77R 81V 85R 89A 129A 132V |
| | +43L+81V+105A+107R | 154R |
| | | 32K 33A 44K 68E 70S 72T 75N 77R 80K 89A 105A |
| | | 19S 32K 33A 43L 44K 49A 68E 70S 75N 77R 81V 85R 89A 129A 154C |
| | | 158Q |
| | | 32K 33A 44K 68E 70S 75N 77R 80K 96R 105A 132V 154C |
| | | 19S 32K 33A 44K 68E 70S 72T 73I 75N 77R 81V 85R 105A |
| | | 19S 30C 33C 44K 54L 68E 70S 75N 77R |
| | | 32K 33A 43L 44K 54L 68E 70S 75N 77R 80K 154C |
| | | 19S 32K 33A 44K 68E 70S 72T 75N 77R 80K 92R 96R 105A 154R |

*Mutations resulting from site-directed mutagenesis are in bold.

### EXAMPLE 14

### Improvement of Meganucleases Cleaving HIV1_{—}3.4 (SEQ ID NO:328) by Random Mutagenesis of Initial Proteins Cleaving HIV1_{—}3.4 (SEQ ID NO:328)

As a complement to example 5 we also decided to perform random mutagenesis with variants that cleave HIV1_{—}3.4 (SEQ ID NO:328). The mutagenized proteins cleaving HIV1_{—}3.4 (SEQ ID NO:328) were then tested to determine the efficiency of cleavage of the HIV1_{—}3.4 (SEQ ID NO:328) and HIV1_{—}3.6 (SEQ ID NO:330) targets.

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25). Approximately 25 ng of the PCR product and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Mating of Meganuclease Expressing Clones and Screening in Yeast

Mating was performed as previously described in example 9. Positive resulting clones were verified by sequencing (MILLEGEN) as described in example 9.

### B) Results

Five variants cleaving HIV1_{—}3.4 (SEQ ID NO:328) were pooled, randomly mutagenized and transformed into yeast. The sequences of the variants subjected to random mutagenesis are described in table XVIII (SEQ ID NO:97 to 101).

2232 transformed clones were screened for cleavage against the HIV1_{—}3.4 (SEQ ID NO:328) and HIV1_{—}3.6 (SEQ ID NO:330) DNA targets. A total of 645 positive clones were found to cleave HIV1_{—}3.4 (SEQ ID NO:328), while 156 of those also cleaved the HIV1_{—}3.6 target (SEQ ID NO:330). Sequencing of the 93 clones showing the strongest activity allowed the identification of 52 novel endonuclease variants. An example of the identified variants is presented in table XXII and in FIG. 31.

TABLE XXII
Examples of 10 functional variants displaying strong cleavage
activity for HIV1_3.4 (SEQ ID NO: 328).
Optimized variants HIV1_3.4

| | |
|---|---|
| SEQ ID NO: 136 | 38Y 43L 44Y 68S 70S 75R 77V 81V 105A 107R |
| SEQ ID NO: 137 | 38Y 44Y 68S 70S 75R 77V 105A 132V |
| SEQ ID NO: 138 | 38Y 43L 44Y 68S 70S 75R 77V 105A |
| SEQ ID NO: 139 | 30R 38E 40R 44N 70S 75R 77N 94Y 105A |
| SEQ ID NO: 140 | 30R 38E 40R 44N 70S 75R 77N 105A |
| SEQ ID NO: 141 | 38Y 44Y 54I 68S 70S 75R 77V 105A |
| SEQ ID NO: 142 | 38Y 44Y 68S 70S 75R 77V 80G 105A |
| SEQ ID NO: 143 | 30R 38E 40R 44N 66C 70S 75R 77N |
| SEQ ID NO: 144 | 38Y 44Y 46S 68S 70S 75R 77V 96R 105A |
| SEQ ID NO: 145 | 30R 38E 40R 44N 70S 75R 77N 105A |

* Mutations resulting from random mutagenesis are in bold.

### EXAMPLE 14bis

### Improvement of Meganucleases Cleaving HIV1_{—}3.4 (SEQ ID NO:328) by a Second Round of Random Mutagenesis of Proteins Cleaving HIV1_{—}3.3 (SEQ ID NO:327)

In order to further improve the activity of the obtained meganucleases, a second round of random mutagenesis was carried out following the same rationale of example 6. For this purpose, ten variants cleaving HIV1_{—}3.4 (SEQ ID NO:328) were mutagenized, and variants were screened for cleavage activity of HIV1_{—}3.4 (SEQ ID NO:328) and HIV1_{—}3.6 (SEQ ID NO:330) targets. The materials and methods have previously been described in example 11.

A) Results

Ten variants cleaving HIV1_{—}3.4 (SEQ ID NO:328), were pooled, randomly mutagenized and transformed into yeast. The variants submitted to random mutagenesis correspond to variants described in Table XXII (SEQ ID NO: 136 to 145).

2232 transformed clones were screened for cleavage against the HIV1_{—}3.4 (SEQ ID NO:328) and HIV1_{—}3.6 (SEQ ID NO:330) targets. A total of 178 positive clones were found to cleave HIV1_{—}3.4 (SEQ ID NO:328), while 63 of those cleaved also the HIV1_{—}3.6 target (SEQ ID NO:330). Sequencing of the 93 clones showing the strongest cleavage activity in the HIV1_{—}3.4 target (SEQ ID NO:328) allowed the identification of 62 novel endonuclease variants. An example of the identified variants is presented in table XXIII and FIG. 32.

TABLE XXIII
Examples of 10 functional variants displaying strong cleavage
activity for HIV1_3.4 (SEQ ID NO: 328).
Optimized variants HIV1_3.4 (2^{nd} round)

| | |
|---|---|
| SEQ ID NO: 146 | 30R 38E 40R 44N 64G 70S 75R 77N 105A 114T 153G |
| SEQ ID NO: 147 | 24V 38Y 43L 44Y 68S 70S 75R 77V 105A 153G 160R |
| SEQ ID NO: 148 | 4I 38Y 44Y 54I 68S 70S 75R 77V 105A |
| SEQ ID NO: 149 | 38Y 40C 44Y 46S 68S 70S 75R 77V 81V 92R 96R |
| | 105A |
| SEQ ID NO: 150 | 33C 38S 70S 75N 77K |
| SEQ ID NO: 151 | 30R 34R 38E 40R 44N 70S 75R 77N 94Y 105A |
| SEQ ID NO: 152 | 38Y 44Y 54I 68S 70S 75R 77V 105A 162F |
| SEQ ID NO: 153 | 7R 38Y 40C 44Y 54I 68S 69V 70S 75R 77V 105A |
| SEQ ID NO: 154 | 38Y 44Y 54I 68S 70S 75R 77V 105A 120G 160R |
| SEQ ID NO: 155 | 30R 38E 40R 44N 70S 75R 77N 105A 157K |

* Mutations resulting from random mutagenesis are in bold.

### EXAMPLE 15

### Improvement of Meganucleases Cleaving HIV1_{—}3 (SEQ ID NO:325) by Site-Directed Mutagenesis of Proteins Cleaving HIV1_{—}3.4 (SEQ ID NO:328) and Assembly with Proteins Cleaving HIV1_{—}3.3 (SEQ ID NO:327)

Four of the improved I-CreI variants cleaving HIV1_{—}3.4 (SEQ ID NO:328) described in Table XXIII and used for a second round of random mutagenesis in example 14bis were also mutagenized by introducing selected amino-acid substitutions in the proteins and screening for variants cleaving HIV1_{—}3 (SEQ ID NO:325) in combination with a variant cleaving HIV1_{—}3.3 (SEQ ID NO:327).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HIV1_{—}3.3 (SEQ ID NO:327), and the resulting proteins were tested for their ability to induce cleavage of the HIV1_{—}3 target (SEQ ID NO:325), upon co-expression with a variant cleaving HIV1_{—}3.4 (SEQ ID NO:328).

### A) Material and Methods

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 16) or Gal10R 5′-acaaccttgattggagacttgacc-3′(SEQ ID NO: 17) and a primer specific to the I-CreI coding sequence for amino acids 14-24 that contains the substitution mutation G19S (G19SF 5′-gccggctttgtggactctgacggtagcatcatc-3′ (SEQ ID NO: 47) or G19SR 5′-gatgatgctaccgtcagagtccacaaagccggc-3′(SEQ ID NO: 48)). The same strategy is used with the following pair of oligonucleotides to introduce the mutations leading to the F54L, E80K, F87L, V105A and I132V substitutions in the coding sequences of the variants, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgataa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
* I132VF:
5′-acctgggtggatcaggttgcagctctgaacgat-3′
and
I132VR:
5′-atcgttcagagctgcaacctgatccacccaggt-3′.

For each substitution to be introduced, the resulting PCR products contain 33 bp of homology with each other. The PCR fragments were purified. The ten PCR fragments were pooled en equimolar amounts to generate a mix containing 50 ng of PCR DNA and 75 ng of vector DNA (pCLS0542, FIG. 9), linearized by digestion with NcoI and EagI. This mix was used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

### c) Mating of Meganuclease Expressing Clones and Screening in Yeast

The experimental procedure is as described in example 11.

### d) Sequencing of Variants

The experimental procedure is as described in example 9.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of four variants cleaving HIV1_{—}3.4 (SEQ ID NO:328) (SEQ ID NO:136 to 139, Table XXII). 317 transformed clones were screened for cleavage against the HIV1_{—}3.4 (SEQ ID NO:328) and HIV1_{—}3.6 (SEQ ID NO:330) DNA targets. A total of 311 positive clones were found to cleave HIV1_{—}3.4 (SEQ ID NO:328), while 262 of those cleaved also the HIV1_{—}3.6 target (SEQ ID NO:330). An example of positive variants is shown in FIG. 33.

The 317 transformed clones were also mated with a yeast strain that contains (i) the HIV1_{—}3 target (SEQ ID NO:325) in a reporter plasmid (ii) an expression plasmid containing a variant that cleaves the HIV1_{—}3.3 target (SEQ ID NO:327) (I-CreI 32K,33A,44K,68E,70S,75N,77R, +132N or KNKAQS/KESNR +132N (SEQ ID NO:109), according to the nomenclature of Table I). After mating with this yeast strain, 264 clones were found to cleave the HIV1_{—}3 (SEQ ID NO:325). Thus, 264 positives contained proteins able to form heterodimers with KNKAQS/KESNR +132N (SEQ ID NO: 109, Table XIX) showing cleavage activity on the HIV1_{—}3 target (SEQ ID NO:325). An example of positive clones is shown in FIG. 34.

Sequencing of the 317 clones allowed the identification of 69 different endonuclease variants.

As an example, ten I-CreI variants cleaving the HIV1_{—}3 target (SEQ ID NO:325) when forming a heterodimer with the KNKAQS/KESNR +132N variant (SEQ ID NO:109) are listed in Table XXIV.

TABLE XXIV
Functional variant combinations
displaying cleavage activity for HIV1_3 target (SEQ ID NO: 325)

| | | Optimized* Variants HIV1_3.4 |
|---|---|---|
| | | (SEQ ID NO: 156 to 165) |
| VARIANT HIV1_3.3 | I-CreI | 38Y 44Y 68S 70S 75R 77V 80K 105A |
| | 28K30N32K33A38Q40S | 28S 40K 43L 44L 70N 75N 80K 132V |
| | 44K68E70S75N77R + 132N | 38Y 43L 44Y 68S 70S 75R 77V 80K 105A 132V |
| | (KNKAQS/KESNR + 132N) | 38Y 43L 44Y 68S 70S 75R 77V 80K 94Y 105A |
| | (SEQ ID NO: 109) | 38Y 43L 44Y 68S 70S 75R 77V 80K 105A |
| | | 30R 38E 40R 44Y 68S 70S 75R 77V 105A 132V |
| | | 38Y 43L 44Y 68S 70S 75R 77V80K 105A 107R 132V |
| | | 19S 38Y 43L 44Y 68S 70S 75R 77V 105A 132V |
| | | 38Y 43L 44Y 68S 70S 75R 77V 80K 105A 132V |
| | | 38Y 43L 44Y 68S 70S 75R 77V 105A 132V |

*Mutations resulting from site-directed mutagenesis are in bold.

### EXAMPLE 16

### Strategy for Engineering Meganucleases Cleaving the HIV1_{—}4 Target (SEQ ID NO:331) from the HIV1 Virus

The HIV1_{—}4 target (SEQ ID NO:331) is a 22 by (non-palindromic) target located in the gag gene of the HIV1 provirus. This target is precisely located at positions 1629-1650 of the HIV-1 pNL4-3 vector (accession number AF324493, Adachi et al., J. Virol., 1986, 59, 284-291), a subtype B infectious molecular clone.

The HIV1_{—}4 sequence (SEQ ID NO: 331) is partly a patchwork of the 10AGC_P (SEQ ID NO:383), 10TGT_P (SEQ ID NO:382), 5TCT_P (SEQ ID NO:390) and 5_TAT_P (SEQ ID NO:391) targets (FIG. 35) which are cleaved by previously identified meganucleases, obtained as described in International PCT Applications WO 2006/097784 and WO 2006/097853; Arnould et al., J. Mol. Biol., 2006, 355, 443-458; Smith et al., Nucleic Acids Res., 2006. Thus, HIV1_{—}4 could be cleaved by combinatorial variants resulting from these previously identified meganucleases.

The 10AGC_P (SEQ ID NO:383), 10TGT_P (SEQ ID NO:382), 5TCT_P (SEQ ID NO:390) and 5_TAT_P (SEQ ID NO:391) target sequences are 24 by derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HIV1_{—}4 series of targets (SEQ ID NO:331 to 336) were defined as 22 by sequences instead of 24 bp. HIV1_{—}4 (SEQ ID NO:331) differs from C1221 (SEQ ID NO:343) in the 4 by central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the GGAC sequence in −2 to 2 was first substituted with the GTAC sequence from C1221 (SEQ ID NO:343), resulting in target HIV1_{—}4.2 (SEQ ID NO: 332, FIG. 35). Then, two palindromic targets, HIV1_{—}4.3 (SEQ ID NO: 333) and HIV1_{—}4.4 (SEQ ID NO: 334), were derived from HIV1_{—}4.2 (SEQ ID NO:332) (FIG. 35). Since HIV1_{—}4.3 (SEQ ID NO:333) and HIV1_{—}4.4 (SEQ ID NO:334) are palindromic, they should be cleaved by homodimeric proteins. Two other pseudo-palindromic targets were derived from these two, containing the GGAC sequence in −2 to 2 (targets HIV1_{—}4.5 (SEQ ID NO: 335) and HIV1_{—}4.6 (SEQ ID NO: 336), FIG. 35). Thus, proteins able to cleave HIV1_{—}4.3 (SEQ ID NO:333) and HIV1_{—}4.4 (SEQ ID NO:334) targets or, preferentially, the pseudo-palindromic targets as homodimers were first designed (examples 17 and 18) and then co-expressed to obtain heterodimers cleaving HIV1_{—}4 (SEQ ID NO:331) (example 19). Heterodimers cleaving the HIV1_{—}4.2 (SEQ ID NO:332) and HIV1_{—}4 (SEQ ID NO:331) targets could be identified. In order to improve cleavage activity for the HIV1_{—}4 target (SEQ ID NO:331), a series of variants cleaving HIV1_{—}4.3 (SEQ ID NO:333) and HIV1_{—}4.4 (SEQ ID NO:334) was chosen, and then refined. The chosen variants were subjected to random or site-directed mutagenesis, and used to form novel heterodimers that were screened against the HIV1_{—}4 target (SEQ ID NO:331) (examples 20, 21, 22 and 23). Heterodimers could be identified with an improved cleavage activity for the HIV1_{—}4 target (SEQ ID NO:331).

### EXAMPLE 17

### Identification of Meganucleases Cleaving HIV1_{—}4.3 (SEQ ID NO:333)

This example shows that I-CreI variants can cut the HIV1_{—}4.3 DNA target sequence (SEQ ID NO:333) derived from the left part of the HIV1_{—}4.2 target (SEQ ID NO:332) in a palindromic form (FIG. 35).

HIV1_{—}4.3 (SEQ ID NO:333) is similar to 10AGC_P (SEQ ID NO:383) at positions ±1, ±2, ±6, ±8, ±9, and ±10 and to 5TCT_P (SEQ ID NO:390) at positions ±1, ±2, ±3, ±4, ±5 and ±6. It was hypothesized that positions ±7 and ±11 would have little effect on the binding and cleavage activity. Variants able to cleave the 10AGC_P (SEQ ID NO:383) target were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156. Variants able to cleave 5TCT_P (SEQ ID NO:390) were obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Both sets of proteins are mutated at position 70. However, the existence of two separable functional subdomains was hypothesized. This implies that this position has little impact on the specificity at bases 10 to 8 of the target. Mutations at positions 24 found in variants cleaving the 5TCT_P target (SEQ ID NO:390) will be lost during the combinatorial process. But it was hypothesized that this will have little impact on the capacity of the combined variants to cleave the HIV1_{—}4.3 target (SEQ ID NO:333).

Therefore, to check whether combined variants could cleave the HIV1_{—}4.3 target (SEQ ID NO:333), mutations at positions 44, 68, 70, 75 and 77 from proteins cleaving 5TCT_P (SEQ ID NO:390) were combined with the 28, 30, 32, 33, 38 and 40 mutations from proteins cleaving 10AGC_P (SEQ ID NO:383).

### A) Material and Methods

### a) Construction of Target Vector

The target was cloned as follows: an oligonucleotide corresponding to the HIV1_{—}4.3 target sequence (SEQ ID NO:333) flanked by gateway cloning sequences was ordered from PROLIGO: 5′ TGGCATACAAGTTTCCAGCATTCTGTACAGAATGCTGGCAATCGTCTGTCA 3′ (SEQ ID NO: 166). The same procedure was followed for cloning the HIV1_{—}4.5 target (SEQ ID NO:335), using the oligonucleotide: 5′TGGCATACAAGTTTCCAGCATTCTGGACAGAATGCTGGCAATCGTCTGTCA 3′(SEQ ID NO: 167). Double-stranded target DNA, generated by PCR amplification of the single stranded oligonucleotide, was cloned using the Gateway protocol (INVITROGEN) into the yeast reporter vector (pCLS1055, FIG. 8). Yeast reporter vector was transformed into Saccharomyces cerevisiae strain FYBL2-7B (MATa, ura3Δ851, trp1Δ63, leu2Δ1, lys2Δ202), resulting in a reporter strain.

### b) Construction of Combinatorial Mutants

I-CreI variants cleaving 10AGC_P (SEQ ID NO:383) or 5TCT_P (SEQ ID NO:390) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10AGC_P (SEQ ID NO:383) and 5TCT_P (SEQ ID NO:390) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′(SEQ ID NO: 17)) specific to the vector (pCLS0542, FIG. 9) and primers (assF 5′-ctannnttgaccttt-3′ (SEQ ID NO: 18) or assR 5′-aaaggtcaannntag-3′(SEQ ID NO: 19)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 9) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

### B) Results

I-CreI combinatorial variants were constructed by associating mutations at positions 44, 68, 70, 75 and 77 from proteins cleaving 5TCT_P (SEQ ID NO:390) with the 28, 30, 32, 33, 38 and 40 mutations from proteins cleaving 10AGC_P (SEQ ID NO:383) on the I-CreI scaffold, resulting in a library of complexity 3800. Examples of combinatorial variants are displayed in Table XXV. This library was transformed into yeast and 3348 clones were screened for cleavage against the HIV1_{—}4.3 (SEQ ID NO:333) and HIV1_{—}4.5 (SEQ ID NO:335) DNA targets. 7 positive clones were found to cleave the HIV1_{—}4.3 target (SEQ ID NO:333), which after sequencing turned out to correspond to 7 different novel endonuclease variants (Table XXVI). Those variants showed no cleavage activity of the HIV1_{—}4.5 DNA target (SEQ ID NO:335). Examples of positives are shown in FIG. 36. Two of the variants obtained display non parental combinations at positions 28, 30, 32, 33, 38, 40 or 44, 68, 70, 75, 77 (SEQ ID NO:168 and 174, Table XXVI). Such combinations likely result from PCR artifacts during the combinatorial process. Alternatively, the variants may be I-CreI combined variants resulting from micro-recombination between two original variants during in vivo homologous recombination in yeast.

TABLE XXV
Panel of variants* theoretically present in the combinatorial library

| Amino acids | | | | | | | | | | | |
|---|---|---|---|---|---|---|---|---|---|---|---|
| at positions | | | | | | | | | | | |
| 44, 68, 70, | | | | | | | | | | | |
| 75 and 77 | | | | | | | | | | | |
| (ex: | | | | | | | | | | | |
| ARNNI stands | | | | | | | | | | | |
| for A44, | Amino acids at positions 28, 30, 32, 33, 38 and 40 | | | | | | | | | | |
| R68, N70, | (ex: KHSSQS stands for K28, H30, S32, S33, Q38 and S40) | | | | | | | | | | |
| N75 and I77) | KDSRQS | KRSPQS | KTYYQS | QNSYRK | KNSGQQ | KNSTGS | KRDYQS | KNSGGS | KNSRQR | KTSYQR | KNSTTS |
| KQSNR | | | | | | | | | | | |
| KYSNV | | | | | | | | | | | |
| KASNI | | | | + | | | | | | | |
| KNSNI | | | | | | | | | | | |
| KYSNQ | | | | | | | | | | | |
| QGGNI | | | | | | | | | | | |
| KAANI | | | | + | | | | | | | |
| KNNNI | | | | | | | | | | | |
| KTSNV | | | | | | | | | | | |
| KRSNV | | | | | | | | | | | |
| KGGNI | | | | + | | | | | | | |
| KQSNT | | | | | | | | | | | |
| KYSNY | | | | | | | | | | | |
| KTGNI | | | | + | | | + | | | | |
| KRGNI | | | | | | | | | | | |
| KNANI | | | | | | | | | | | |
| QASNR | | | | | | | | | | | |
| QSSNR | | | | | | | | | | | |
| KKANI | | | | | | | | | | | |
| PCSYT | | | | | | | | | | | |
| KKANI | | | | | | | | | | | |
| QNSNR | | | | | | | | | | | |
| KSSNV | | | | | | | | | | | |
| KTTNI | | | | | | | | | | | |

*Only 264 out of the 3800 combinations are displayed.
+ indicates that a functional combinatorial variant cleaving the HIV1_4.3 target (SEQ ID NO: 333) was found among the identified positives.

TABLE XXVI
I-CreI variants capable of cleaving the
HIV1_4.3 DNA target (SEQ ID NO: 333).

| Amino acids at positions 28, | |
|---|---|
| 30, 32, 33, 38, 40/44, 68, | |
| 70, 75 and 77 of the I-CreI | |
| variants (ex: KRSRES/TYSNI | |
| stands for K28, R30, S32, | SEQ |
| R33, E38, S40/T44 Y68, | ID |
| S70, N75 and I77) | NO: |
| QNSYRK/KRDNI | 168 |
| KRDYQS/KTGNI | 169 |
| QNSYRK/KTGNI | 170 |
| QNSYRK/KASNI | 171 |
| QNSYRK/KAANI | 172 |
| QNSYRK/KGGNI | 173 |
| KRSYQS/QASNR | 174 |

### EXAMPLE 18

### Making of Meganucleases Cleaving HIV1_{—}4.4 (SEQ ID NO:334)

This example shows that I-CreI variants can cleave the HIV1_{—}4.4 (SEQ ID NO:334) DNA target sequence derived from the right part of the HIV1_{—}4.2 target (SEQ ID NO:332) in a palindromic form (FIG. 35).

HIV1_{—}4.4 (SEQ ID NO:334) is similar to 5TAT_P (SEQ ID NO:391) at positions ±1, ±2, ±3, ±4, ±5 and ±8 and to 10TGT_P (SEQ ID NO:382) at positions ±1, ±2, ±3, ±4, ±8, ±9 and ±10. It was hypothesized that positions ±6, ±7 and ±11 would have little effect on the binding and cleavage activity. Variants able to cleave 5TAT_P (SEQ ID NO:391) were obtained by mutagenesis of I-CreI N75 at positions 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10TGT_P target (SEQ ID NO:382) were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156.

Both sets of proteins are mutated at position 70. However, the existence of two separable functional subdomains was hypothesized. This implies that this position has little impact on the specificity at bases 10 to 8 of the target.

Therefore, to check whether combined variants could cleave the HIV1_{—}4.4 target (SEQ ID NO:334), mutations at positions 44, 68, 70, 75 and 77 from proteins cleaving 5TAT_P (SEQ ID NO:391) were combined with the 28, 30, 32, 33, 38 and 40 mutations from proteins cleaving 10TGT_P (SEQ ID NO:382).

### A) Material and Methods

### a) Construction of Target Vector

The experimental procedure is as described in example 17, with the exception that different oligonucleotides corresponding to the HIV1_{—}4.4 (SEQ ID NO:334) and HIV1_{—}4.6 (SEQ ID NO:336) targets. The oligonucleotide used for the HIV1_{—}4.4 target (SEQ ID NO:334) was: 5′TGGCATACAAGTTTCTTGTCTTATGTACATAAGACAAGCAATCGTCTGTCA3′ (SEQ ID NO: 175), and 5′TGGCATACAAGTTTCTTGTCTTATGGACATAAGACAAGCAATCGTCTGTCA3′ (SEQ ID NO: 176) for HIV1_{—}4.6 target (SEQ ID NO:336).

### b) Construction of Combinatorial Variants

I-CreI variants cleaving 10TGT_P (SEQ ID NO:382) or 5TAT_P (SEQ ID NO:391) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TGT_P (SEQ ID NO:382) and 5TAT_P (SEQ ID NO:391) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′ (SEQ ID NO: 17)) specific to the vector (pCLS1107, FIG. 11) and primers (assF 5′-ctannnttgaccttt-3′ (SEQ ID NO: 18) or assR 5′-aaaggtcaannntag-3′(SEQ ID NO: 19), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 11) linearized by digestion with Drain and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. Positives resulting clones were verified by sequencing (MILLEGEN) as described in example 2.

### B) Results

I-CreI combinatorial variants were constructed by associating mutations at positions 44, 68, 70, 75 and 77 from proteins cleaving 5TAT_P (SEQ ID NO:391) with the 28, 30, 32, 33, 38 and 40 mutations from proteins cleaving 10TGT_P (SEQ ID NO:382) on the I-CreI scaffold, resulting in a library of complexity 1406. Examples of combinatorial variants are displayed in Table XXVII. This library was transformed into yeast and 3348 clones (2.3 times the diversity) were screened for cleavage against the HIV1_{—}4.4 (SEQ ID NO:334) and HIV1_{—}4.6 (SEQ ID NO:336) DNA targets. A total of 210 positive clones were found to cleave HIV1_{—}4.4 (SEQ ID NO:334). 40 of these clones were also able to cleave the HIV1_{—}4.6 (SEQ ID NO:336) DNA target. Sequencing of these 93 clones with the strongest activity allowed the identification of 45 novel endonuclease variants. Examples of positives are shown in FIG. 37. The sequence of several of the variants identified display non parental combinations at positions 28, 30, 32, 33, 38, 40 or 44, 68, 70, 75, 77 as well as additional mutations (see examples in Table XXVIII, SEQ ID NO:178 and 184). Such variants likely result from PCR artifacts during the combinatorial process. Alternatively, the variants may be I-CreI combined variants resulting from micro-recombination between two original variants during in vivo homologous recombination in yeast.

TABLE XXVII
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, 70, | | | | | | | | | | | |
| 75 and 77 | | | | | | | | | | | |
| (ex : | | | | | | | | | | | |
| ARNNI | | | | | | | | | | | |
| stands for | | | | | | | | | | | |
| A44, R68, | | | | | | | | | | | |
| N70, | Amino acids at positions 28, 30, 32, 33, 38 and 40 | | | | | | | | | | |
| N75 and | (ex: KHSTQS stands for K28, H30, S32, S33, Q38 and S40) | | | | | | | | | | |
| I77) | ANSSRK | NNSSRK | QNSSRK | KHSCQS | KHSMAS | KHSTQS | KNDCQS | KNRAQS | KNSSRK | KNATQS | KNSSRS |
| AYSYK | | | | + | + | | + | + | | + | |
| ARSYT | + | | + | | | | | | | | |
| YRSYN | | | | | | | | | | | |
| YYSYR | + | | + | | | | | | | | |
| ATGNI | | | + | | | | | | | | |
| AKQNI | | | | | | | | | | | + |
| ANANI | | | + | | | | | | | | |
| ARSYT | | | | | | | | | | | |
| AYSNI | | + | | | | | | | | | |
| ARSNV | | | | | | | | | | | |
| NRGNI | | | | | | | | | | | + |
| AASYR | | | | | | | | | | | |
| ARSDY | | | | | | | | | | | |
| ANSYR | | | + | | | | | | | | |
| ARNNI | | | | | | | | | | | + |
| NHSYN | | | | | | | | | | | |
| ARSYV | | | | | | | | | | | |
| ARGNI | | | | | | | | | | | + |
| NYSYR | | | | | + | + | | + | + | + | |
| NRENI | | | | | | | | | | | |
| ASSYK | | | | | | | | | | | |
| NRSNT | | | | | | | | | | | + |
| YYSNQ | | | + | | | | | | | | |
| YRSYQ | | | | | | | | | | | |

*Only 264 out of the 1406 combinations are displayed.
+ indicates that a functional combinatorial variant cleaving the HIV1_4.4 target (SEQ ID NO: 334) was found among the identified positives.

TABLE XXVIII
I-CreI variants capable of cleaving the
HIV1_4.4 DNA target (SEQ ID NO: 334).

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and 177) | NO: |
| KHSMAS/NYSYR | 177 |
| KNGTQS/AYSYR | 178 |
| KHSMAS/AYSYK | 179 |
| KNATQS/NYSYR | 180 |
| KNRAQS/NYSYR | 181 |
| KNSTQA/NYSYR | 182 |
| KNSGCS/NYSYR | 183 |
| ANSSRK/NYSYK +59A | 184 |
| ANSSRK/ARSYT | 185 |
| KHSCQS/AYSYK | 186 |

### EXAMPLE 19

### Making of Meganucleases Cleaving HIV1_{—}4.2 (SEQ ID NO:332) and HIV1_{—}4 (SEQ ID NO:331)

I-CreI variants able to cleave each of the palindromic HIV1_{—}4.2 (SEQ ID NO:332) derived targets (HIV1_{—}4.3 (SEQ ID NO:333) and HIV1_{—}4.4 (SEQ ID NO:334)) were identified in example 2 and example 3. Pairs of such variants (one cutting HIV1_{—}4.3 (SEQ ID NO:333) and one cutting HIV1_{—}4.4 (SEQ ID NO:334)) were co-expressed in yeast. Upon co-expression, there should be three active molecular species, two homodimers, and one heterodimer. It was assayed whether the heterodimers that should be formed, cut the HIV1_{—}4.2 (SEQ ID NO:332) and the non palindromic HIV1_{—}4 (SEQ ID NO:331) targets.

### A) Materials and Methods

### a) Construction of Target Vector

The experimental procedure is as described in example 2, with the exception that an oligonucleotide corresponding to the HIV1_{—}4.2 target sequence (SEQ ID NO:332): 5′TGGCATACAAGTTTCCAGCATTCTGTACATAAGACAAGCAATCGTCTGTC A 3′(SEQ ID NO: 187) or the HIV1_{—}4 target sequence (SEQ ID NO:331): 5′ TGGCATACAAGTTTCCAGCATTCTGGACATAAGACAAGCAATCGTCTGTC A3′ (SEQ ID NO: 188) was used.

### b) Co-Expression of Variants

Yeast DNA was extracted from variants cleaving the HIV1_{—}4.4 (SEQ ID NO:334) target in the pCLS1107 expression vector using standard protocols and was used to transform E. coli. The resulting plasmid DNA was then used to transform yeast strains expressing a variant cutting the HIV1_{—}4.3 target (SEQ ID NO:333) in the pCLS0542 expression vector. Transformants were selected on synthetic medium lacking leucine and containing G418.

### c) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM 3-mercaptoethanol, 1% agarose, and incubated at 37° C., to monitor β-galactosidase activity. Results were analyzed by scanning and quantification was performed using appropriate software.

### B) Results

Co-expression of variants cleaving the HIV1_{—}4.4 target (SEQ ID NO:334) (10 variants corresponding to those described in Table XXVIII, SEQ ID 177 to 186) and six variants cleaving the HIV1_{—}4.3 target (SEQ ID NO:333) (Table XXVI, SEQ ID 168 and 170 to 174) resulted in cleavage of the HIV1_{—}4.2 (SEQ ID NO:332) target in most of the cases (FIG. 38). Nevertheless, none of these combinations was able to cut the HIV1_{—}4 natural target (SEQ ID NO:331) that differs from the HIV1_{—}4.2 sequence (SEQ ID NO:332) by 2 by at positions 1 and 2 (FIG. 35). Examples of functional combinations are summarized in Table XXIX.

TABLE XXIX
Cleavage of the HIV1_4.2 target (SEQ ID NO: 332) by the heterodimeric variants.

| | | Sequence of the I-CreI variants cleaving | |
|---|---|---|---|
| | | the HIV1_4.3 target (SEQ ID NO: 333)^{§} | |
| | | QNSYRK/KTGNI | QNSYRK/KASNI |
| HIV1_4.2 target (SEQ ID NO: 332) | | SEQ ID NO: 170 | SEQ ID NO: 171 |
| Sequence of the I-CreI | KHSMAS/NYSYR | + | + |
| variants cleaving the | SEQ ID NO: 177 | | |
| HIV1_4.4 target (SEQ | KNGTQS/AYSYR | + | + |
| ID NO: 334)^{§} | SEQ ID NO: 178 | | |
| | KHSMAS/AYSYK | + | + |
| | SEQ ID NO: 179 | | |
| | KNATQS/NYSYR | + | + |
| | SEQ ID NO: 180 | | |
| | KNRAQS/NYSYR | + | + |
| | SEQ ID NO: 181 | | |
| | KNSTQA/NYSYR | + | + |
| | SEQ ID NO: 182 | | |

^{§}Amino acids at positions 28, 30, 32, 33, 38, 40/44, 68, 70, 75 and 77 (ex: KRSRES/TYSNI stands for K28, R30, S32, R33, E38, S40/T44, Y68, S70, N75 and I77)
+ indicates a functional combination

### EXAMPLE 20

### Improvement of Meganucleases Cleaving HIV1_{—}4.3 (SEQ ID NO:333) by Random Mutagenesis of Proteins and Assembly with Proteins Cleaving HIV1_{—}4.4 (SEQ ID NO:334)

The assembly of I-CreI variants cleaving the palindromic HIV1_{—}4.3 (SEQ ID NO:333) and HIV1_{—}4.4 target (SEQ ID NO:334) to cleave the HIV1_{—}4.2 (SEQ ID NO:332) and HIV1_{—}4 (SEQ ID NO:331) have been previously identified in example 4. However, these variants display activity with the HIV1_{—}4.2 target (SEQ ID NO:332) and not with the HIV1_{—}4 target (SEQ ID NO:331).

Therefore seven variants cleaving HIV1_{—}4.3 (SEQ ID NO:333) were mutagenized, and variants were screened for cleavage activity of HIV1_{—}4.3 (SEQ ID NO:333) and HIV1_{—}4.5 (SEQ ID NO:335) targets. Additionally the mutants with the strongest activity were screened for cleavage activity of HIV1_{—}4 (SEQ ID NO:331) when co-expressed with a variant cleaving HIV1_{—}4.4 (SEQ ID NO:334). According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, only one of the two components of the heterodimers cleaving HIV1_{—}4 (SEQ ID NO:331) was mutagenized.

Thus, in a first step, proteins cleaving HIV1_{—}4.3 (SEQ ID NO:333) were mutagenized and their homodimeric cleavage activity was determined, and in a second step, it was assessed whether they could cleave HIV1_{—}4 (SEQ ID NO:331) when co-expressed with a protein cleaving HIV1_{—}4.4 (SEQ ID NO:334).

### A) Material and Methods

### a) Construction of Libraries by Random Mutagenesis

Random mutagenesis was performed on a pool of chosen variants, by PCR using Mn^{2±}. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25), which are common to the pCLS0542 (FIG. 9) and pCLS1107 (FIG. 11) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Mating of Meganuclease Expressing Clones and Screening in Yeast

Experiments were performed as previously described in example 17. Positive resulting clones were verified by sequencing (MILLEGEN) as described in example 17.

### c) Variant-Target Yeast Strains, Screening and Sequencing

The yeast strain FYBL2-7B (MATα, ura3Δ851, trp1Δ63, leu2Δ1, lys2Δ202) containing the HIV1_{—}4 target (SEQ ID NO:331) in the yeast reporter vector (pCLS1055, FIG. 8) was transformed with one variant, in the kanamycin vector (pCLS1107), cutting the HIV1_{—}4.4 target (SEQ ID NO:334), using a high efficiency LiAc transformation protocol. Variant-target yeast strains were used as target strains for mating assays as described in example 19. Positives resulting clones were verified by sequencing (MILLEGEN) as described in example 17.

### B) Results

Seven variants cleaving HIV1_{—}4.3 (SEQ ID NO:333), were pooled, randomly mutagenized and transformed into yeast. The sequences of the variants subjected to random mutagenesis are described in table XXVI.

2232 transformed clones were screened for cleavage against the HIV1_{—}4.3 (SEQ ID NO:333) and HIV1_{—}4.5 (SEQ ID NO:335) DNA targets. A total of 249 positive clones were found to cleave HIV1_{—}4.3 (SEQ ID NO:333), while 12 of them cleaved also the HIV1_{—}4.5 target (SEQ ID NO:335). Sequencing of the 93 clones showing the strongest activity allowed the identification of 60 novel endonuclease variants. An example of the identified variants is presented in table XXX and in FIG. 39.

TABLE XXX
Examples of 10 functional variants displaying strong cleavage
activity for HIV1_4.3 (SEQ ID NO: 333).
Optimized variants HIV1_4.3

| | |
|---|---|
| SEQ ID NO: 189 | 28Q 36N 38R 40K 44K 68T 70G 75N |
| SEQ ID NO: 190 | 28Q 38R 40K 44K 68T 70G 75N 132V |
| SEQ ID NO: 191 | 28Q 38R 40K 44K 54L 68A 70S 75N |
| SEQ ID NO: 192 | 28Q 38R 40K 44K 54L 68A 70D 75N |
| SEQ ID NO: 193 | 28Q 38R 40K 41M 44K 54L 68T 70G 75N |
| SEQ ID NO: 194 | 28Q 38R 40K 44K 68T 70G 75N 80K 114T |
| SEQ ID NO: 195 | 28Q 38R 40K 44K 68T 70G 72P 75N |
| SEQ ID NO: 196 | 28Q 38R 40K 44K 68A 70D 75N 132V 156R |
| SEQ ID NO: 197 | 28Q 38R 40K 43L 44K 68T 70G 75N |
| SEQ ID NO: 198 | 28Q 38R 40K 44K 68T 70G 75N |

* Mutations resulting from random mutagenesis are in bold.

The 93 clones showing the highest cleavage activity on target HIV1_{—}4.3 (SEQ ID NO:333) were then mated with a yeast strain that contains (i) the HIV1_{—}4 target (SEQ ID NO:331) in a reporter plasmid (ii) an expression plasmid containing a variant that cleaves the HIV1_{—}4.4 target (SEQ ID NO:334) (I-CreI 30H,33M,38A,44N,68Y,70S,75Y,77R or KHSMAS/NYSYR (SEQ ID NO:177), according to the nomenclature of Table I). After mating with this yeast strain, no clones were found to cleave the HIV1_{—}4 (SEQ ID NO:331) when forming heterodimers with KHSMAS/NYSYR (SEQ ID NO: 177, Table XXIX).

### EXAMPLE 20bis

### Improvement of Meganucleases Cleaving HIV1_{—}4.3 (SEQ ID NO:333) by a Second Round of Random Mutagenesis of Proteins and Assembly with Proteins Cleaving HIV1_{—}4.4 (SEQ ID NO:334)

In order to further improve the activity of the obtained meganucleases, a second round of random mutagenesis was carried out following the same rationale of example 20. For this purpose, four variants cleaving HIV1_{—}4.3 (SEQ ID NO:333) were mutagenized, and variants were screened for cleavage activity of HIV1_{—}4.3 (SEQ ID NO:333) and HIV1_{—}4.5 (SEQ ID NO:335) targets. Additionally the mutants with the strongest activity were screened for cleavage activity of HIV1_{—}4 (SEQ ID NO:331) when co-expressed with a variant cleaving HIV1_{—}4.4 (SEQ ID NO:334).

The materials and methods have previously been described in example 20.

### A) Results

Six variants cleaving HIV1_{—}4.3 (SEQ ID NO:333), were pooled, randomly mutagenized and transformed into yeast. The six variants submitted to random mutagenesis correspond to variants described in Table XXX (SEQ ID NO: 189 to 194).

2232 transformed clones were screened for cleavage against the HIV1_{—}4.3 (SEQ ID NO:333) and HIV1_{—}4.5 (SEQ ID NO:335) DNA targets. A total of 377 positive clones were found to cleave HIV1_{—}4.3 (SEQ ID NO:333), while 208 of those cleaved also the HIV1_{—}4.5 target (SEQ ID NO:335). Sequencing of the 93 clones with the highest activity allowed the identification of 53 novel endonuclease variants. An example of the identified variants is presented in table XXXI and FIG. 40.

The 93 clones showing cleaving target HIV1_{—}4.3 (SEQ ID NO:333) were then mated with a yeast strain that contains (i) the HIV1_{—}4 target (SEQ ID NO:331) in a reporter plasmid (ii) an expression plasmid containing a variant that cleaves the HIV1_{—}4.4 target (SEQ ID NO:334) (I-CreI 30H,33M,38A,44A,68Y,70S,75Y,77R,155R or KHSMAS/AYSYR +155R (SEQ ID NO:199), according to the nomenclature of Table I). After mating with this yeast strain, all the 93 clones were found to cleave the HIV1_{—}4 (SEQ ID NO:331). Thus, 93 positives contained proteins able to form heterodimers with KHSMAS/AYSYR +155R (SEQ ID NO: 199) showing cleavage activity on the HIV1_{—}4 target (SEQ ID NO:331). An example of positives is shown in FIG. 41. Sequencing of these 93 positive clones indicates, as mentioned before, that 53 distinct variants were identified. Ten of these 53 variants are presented as an example in Table XXXI.

TABLE XXXI
Examples of 10 functional variants displaying strong cleavage
activity for HIV1_4.3 (SEQ ID NO: 333).
Optimized variants HIV1_4.3 (2^{nd} round)

| | |
|---|---|
| SEQ ID NO: 200 | 28Q 38R 40K 44K 68T 70G 80K 100R 114T |
| SEQ ID NO: 201 | 28Q 38R 40K 41M 44K 54L 68T 70G 123M 132V |
| SEQ ID NO: 202 | 28Q 38R 40K 44K 54L 68A 70S 80K 89S 111R 132V |
| SEQ ID NO: 203 | 28Q 38R 40K 44K 68T 70G 80K 114T 162P |
| SEQ ID NO: 204 | 28Q 38R 40K 44K 54L 68T 70G 80K 114T |
| SEQ ID NO: 205 | 28Q 38R 40K 44K 68T 70G 80K 114T |
| SEQ ID NO: 206 | 28Q 38R 40K 44K 54L 57N 68T 70G 132V 159E |
| SEQ ID NO: 207 | 28Q 38R 40K 44K 54L 61G 64A 68A 70D 121R 132V |
| SEQ ID NO: 208 | 28Q 38R 40K 44K 54L 68T 70G 132V |
| SEQ ID NO: 209 | 28Q 35C 38R 40K 43Y 44K 62V 67I 68T 70G 99R |
| | 132V |

* Mutations resulting from random mutagenesis are in bold.

### EXAMPLE 21

### Improvement of Meganucleases Cleaving HIV1_{—}4 (SEQ ID NO:331) by Site-Directed Mutagenesis of Proteins Cleaving HIV1_{—}4.3 (SEQ ID NO:333) and Assembly with Proteins Cleaving HIV1_{—}4.4 (SEQ ID NO:334)

I-CreI variants cleaving HIV1_{—}4.3 (SEQ ID NO:333) were also mutagenized by introducing selected amino-acid substitutions in the proteins and screening for more efficient variants cleaving HIV1_{—}4 (SEQ ID NO:331) in combination with a variant cleaving HIV1_{—}4.4 (SEQ ID NO:334).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HIV1_{—}4.3 (SEQ ID NO:333), and the resulting proteins were tested for their ability to induce cleavage of the HIV1_{—}4 target (SEQ ID NO:331), upon co-expression with a variant cleaving HIV1_{—}4.4 (SEQ ID NO:334), as well as for the ability to cleave targets HIV1_{—}4.3 (SEQ ID NO:333) and HIV1_{—}4.5 (SEQ ID NO:335).

### A) Material and Methods

### a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′(SEQ ID NO: 16) or Gal10R 5′-acaaccttgattggagacttgacc-3′(SEQ ID NO: 17)) and a primer specific to the I-CreI coding sequence for amino acids 14-24 that contains the substitution mutation G19S (G19SF 5′-gccggctttgtggactctgacggtagcatcatc-3′ (SEQ ID NO: 47) or G19SR 5′-gatgatgctaccgtcagagtccacaaagccggc-3′(SEQ ID NO: 48)). The same strategy is used with the following pair of oligonucleotides to introduce the mutations leading to the F54L, E80K, F87L, V105A and I132V substitutions in the coding sequences of the variants, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgcttaa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
I132VF:
5′-acctgggtggatcaggttgcagctctgaacgat-3′
and
I132VR:
5′-atcgttcagagctgcaacctgatccacccaggt-3′.

For each substitution to be introduced, the resulting PCR products contain 33 bp of homology with each other. The PCR fragments were purified. The ten PCR fragments were pooled en equimolar amounts to generate a mix containing 50 ng of PCR DNA and 75 ng of vector DNA (pCLS0542, FIG. 9), linearized by digestion with NcoI and EagI. This mix was used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

### c) Mating of Meganuclease Expressing Clones and Screening in Yeast

The experimental procedure is as described in example 20.

### d) Sequencing of Variants

The experimental procedure is as described in example 17.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of six variants cleaving HIV1_{—}4.3 (SEQ ID NO:333) (described in Table XXXI, SEQ ID NO:200 to 205).

558 transformed clones were mated with a yeast strain that contains (i) the HIV1_{—}4 target (SEQ ID NO:331) in a reporter plasmid (ii) an expression plasmid containing a variant that cleaves the HIV1_{—}4.4 target (SEQ ID NO:334) (30H,33M,38A,44N,68Y,70S,75Y,77R or KHSMAS/NYSYR (SEQ ID NO:177), according to the nomenclature of Table I). After mating with this yeast strain, 486 clones were found to cleave the HIV1_{—}4 (SEQ ID NO:331). Thus, 486 positives contained proteins able to form heterodimers with KHSMAS/NYSYR (SEQ ID NO: 177) showing cleavage activity on the HIV1_{—}4 target (SEQ ID NO:331). An example of positive variants is shown in FIG. 42.

Sequencing of the 93clones with the highest cleavage activity on the HIV1_{—}4 target (SEQ ID NO:331) allowed the identification of 34 different endonuclease variants. These 93 clones were also tested for their ability to cleave the HIV1_{—}4.3 (SEQ ID NO:333) and HIV1_{—}4.5 (SEQ ID NO:335) targets. In this case, 71 clones were able to cleave the HIV1_{—}4.3 target (SEQ ID NO:333), and 69 the HIV1_{—}4.5 target (SEQ ID NO:335) (see FIG. 43 for an example). Sequence analysis of these clones showed the presence of 25 different endonuclease variants. Comparison of sequences of the positive clones in all the targets indicated the presence of a total of 40 novel endonuclease variants.

The sequence of ten I-CreI variants cleaving the HIV1_{—}4 target (SEQ ID NO:331) when forming a heterodimer with the KHSMAS/NYSYR variant are listed in Table XXXII.

TABLE XXXII
Sequences corresponding to the variants cleaving the HIV1_4
target (SEQ ID NO: 331)

| SEQ ID NO: | I-CreI variants |
|---|---|
| 211 | 19S 28Q 38R 40K 44K 68T 70G 75N 80K 114T |
| 212 | 28Q 38R 40K 44K 54L 68T 70G 75N 80K 114T |
| 213 | 19S 28Q 38R 40K 44K 54L 68T 70G 75N 114T |
| 214 | 19S 28Q 38R 40K 44K 68T 70G 75N 80K 114T 147A |
| | 162P |
| 215 | 28Q 38R 40K 44K 54L 68T 70G 75N 80K 100R 114T |
| | 132V 162P |
| 216 | 19S 28Q 38R 40K 41M 44K 54L 68T 70G 75N 80K 123M |
| | 132V |
| 217 | 19S 28Q 38R 40K 42A 44K 54L 68T 70G 75N 80K 105A |
| | 114T |
| 218 | 19S 28Q 38R 40K 42S 44K 54L 68T 70G 75N 80K 92H |
| | 94Y 105A |
| 219 | 19S 28Q 38R 40K 41M 44K 54L 68T 70G 75N 80K 114T |
| 220 | 19S 28Q 38R 40K 44K 68T 70G 75N 123M 132V |

* Mutations resulting from site-directed mutagenesis are in bold.

### EXAMPLE 22

### Improvement of Meganucleases Cleaving HIV1_{—}4.4 (SEQ ID NO:334) by Random Mutagenesis and Assembly with Proteins Cleaving HIV1_{—}4.3 (SEQ ID NO:333)

The assembly of I-CreI variants cleaving the palindromic HIV1_{—}4.3 (SEQ ID NO:333) and HIV1_{—}4.4 target (SEQ ID NO:334) to cleave the HIV1_{—}4.2 (SEQ ID NO:332) and HIV1_{—}4 (SEQ ID NO:331) have been previously described in example 19. However, these variants display activity with the HIV1_{—}4.2 target (SEQ ID NO:332) and not with the HIV1_{—}4 target (SEQ ID NO:331).

As a complement to example 4 we also decided to perform random mutagenesis with variants that cleave HIV1_{—}4.4 (SEQ ID NO:334). Therefore ten variants cleaving HIV1_{—}4.3 (SEQ ID NO:333) were mutagenized, and variants were screened for cleavage activity of HIV1_{—}4.4 (SEQ ID NO:334) and HIV1_{—}4.6 (SEQ ID NO:336) targets. Additionally the mutants with the strongest activity were screened for cleavage activity of HIV1_{—}4 (SEQ ID NO:331) when co-expressed with a variant cleaving HIV1_{—}4.3 (SEQ ID NO:333).

### A) Material and Methods

### a) Construction of Libraries by Random Mutagenesis

Random mutagenesis was performed on a pool of chosen variants, by PCR using Mn^{2±}. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25). Approximately 25 ng of the PCR product and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Variant-Target Yeast Strains, Screening and Sequencing

The yeast strain FYBL2-7B (MATα, ura3Δ851, trp1Δ63, leu2Δ1, lys2Δ202) containing the HIV1_{—}4 target (SEQ ID NO:331) in the yeast reporter vector (pCLS1055, FIG. 8) was transformed with variants, in the leucine vector (pCLS0542), cutting the HIV1_{—}4.3 target (SEQ ID NO:333), using a high efficiency LiAc transformation protocol. Variant-target yeast strains were used as target strains for mating assays as described in example 4. Positives resulting clones were verified by sequencing (MILLEGEN) as described in example 17.

### B) Results

Ten variants cleaving HIV1_{—}4.4 (SEQ ID NO:334) were pooled, randomly mutagenized and transformed into yeast. The sequences of the variants subjected to random mutagenesis are described in table XXXII.

2232 transformed clones were screened for cleavage against the HIV1_{—}4.4 (SEQ ID NO:334) and HIV1_{—}4.6 (SEQ ID NO:336) DNA targets. A total of 210 positive clones were found to cleave HIV1_{—}4.4 (SEQ ID NO:334), while 32 of those also cleaved the HIV1_{—}4.6 target (SEQ ID NO:336). Sequencing of the 93 clones showing the strongest activity allowed the identification of 65 novel endonuclease variants. An example of the identified variants is presented in table XXXIII and in FIG. 44.

TABLE XXXIII
Examples of 10 functional variants displaying strong cleavage
activity for HIV1_4.4 (SEQ ID NO: 334).
Optimized variants HIV1_4.4

| | |
|---|---|
| SEQ ID NO: 199 | 30H 33M 38A 44A 68Y 70S 75Y 77R 155R |
| SEQ ID NO: 177 | 30H 33M 38A 44N 68Y 70S 75Y 77R |
| SEQ ID NO: 221 | 30H 33M 38A 44N 68Y 70S 75Y 77R 160R |
| SEQ ID NO: 222 | 30H 33M 38A 44N 68Y 70S 75Y 77R 96R |
| SEQ ID NO: 223 | 32A 33T 44A 68Y 70S 75Y 77R 98R 129A 158M |
| SEQ ID NO: 224 | 26H 30H 33M 38A 44N 68Y 70S 75Y 77R |
| SEQ ID NO: 225 | 30H 33M 38A 44N 68Y 70S 75Y 77R 99R |
| SEQ ID NO: 226 | 32A 33T 44A 57R 68Y 70S 75Y 77R 125A 132V |
| SEQ ID NO: 227 | 30H 33M 38A 44N 68Y 70S 75Y 77R 158R |
| SEQ ID NO: 228 | 30H 33M 38A 44N 68Y 70S 75Y 77R 116R |

* Mutations resulting from random mutagenesis are in bold.

The 93 clones showing the highest cleavage activity on target HIV1_{—}4.4 (SEQ ID NO:334) were then mated with a yeast strain that contains (i) the HIV1_{—}4 target (SEQ ID NO:331) in a reporter plasmid (ii) an expression plasmid containing a variant that cleaves the HIV1_{—}4.3 target (SEQ ID NO:333) (I-CreI 28Q,38R,40K,44K,68T,70G,75N +132V or QNSYRK/KTGNI +132V (SEQ ID NO:190), according to the nomenclature of Table I). After mating with this yeast strain, 90 clones were found to cleave the HIV1_{—}4 target (SEQ ID NO:331). Thus, 90 positives contained proteins able to form heterodimers with QNSYRK/KTGNI +132V (SEQ ID NO: 190, Table XXX), that showed cleavage activity on the HIV1_{—}4 target (SEQ ID NO:331). An example of positives is shown in FIG. 45. Sequencing of these 90 positive clones indicates that 65 distinct variants were identified. Ten of these 65 variants are presented as an example in Table XXXIII.

### EXAMPLE 23

### Improvement of Meganucleases Cleaving HIV1_{—}4 (SEQ ID NO:331) by Site-Directed Mutagenesis of Proteins Cleaving HIV1_{—}4.4 (SEQ ID NO:334) and Assembly with Proteins Cleaving HIV1_{—}4.3 (SEQ ID NO:333)

Four of the I-CreI variants cleaving HIV1_{—}4.4 (SEQ ID NO:334) described in Table XXXVII were mutagenized by introducing selected amino-acid substitutions in the proteins and screening for more efficient variants cleaving HIV1_{—}4 (SEQ ID NO:331) in combination with a variant cleaving HIV1_{—}4.3 (SEQ ID NO:333).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HIV1_{—}4.4 (SEQ ID NO:334), and the resulting proteins were tested for their ability to induce cleavage of the HIV1_{—}4 target (SEQ ID NO:331), upon co-expression with a variant cleaving HIV1_{—}4.3 (SEQ ID NO:333).

### A) Material and Methods

### a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ or Gal10R 5′-acaaccttgattggagacttgacc-3′) and a primer specific to the I-CreI coding sequence for amino acids 14-24 that contains the substitution mutation G19S (G19SF 5′-gccggctttgtggactctgacggtagcatcatc-3′ (SEQ ID NO: 47) or G19SR 5′-gatgatgctaccgtcagagtccacaaagccggc-3′ (SEQ ID NO: 48)). The resulting PCR products contain 33 bp of homology with each other. The PCR fragments were purified. Approximately 25 ng of each of the two overlapping PCR fragments and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

The same strategy is used with the following pair of oligonucleotides to create other libraries containing the F54L, E80K, F87L, V105A and I132V substitutions, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgataa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
I132VF:
5′-acctgggtggatcaggttgcagctctgaacgat-3′
and
I132VR:
5′-atcgttcagagctgcaacctgatccacccaggt-3′.

### c) Mating of Meganuclease Expressing Clones and Screening in Yeast

The experimental procedure is as described in example 22.

### d) Sequencing of Variants

The experimental procedure is as described in example 17.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of four variants cleaving HIV1_{—}4.4 (SEQ ID NO:334) (see Table XXXIII, SEQ ID NO:199, 177, 221 and 228).

558 transformed clones were mated with a yeast strain that contains (i) the HIV1_{—}4 target (SEQ ID NO:331) in a reporter plasmid (ii) an expression plasmid containing a variant that cleaves the HIV1_{—}4.3 target (SEQ ID NO:333) (28Q,38R,40K,44K,68T,70G,75N or QNSYRK/KTGNI+132V (SEQ ID NO:190), according to the nomenclature of Table I). After mating with this yeast strain, 16 clones were found to cleave the HIV1_{—}4 (SEQ ID NO:331). Thus, 16 positives contained proteins able to form heterodimers with QNSYRK/KTGNI+132V (SEQ ID NO: 190, Table XXX) showing cleavage activity on the HIV1_{—}4 target (SEQ ID NO:331). An example of positive variants is shown in FIG. 46.

Sequencing of these positive clones allowed the identification of 10 different endonuclease variants. The clones cleaving the HIV1_{—}4 target (SEQ ID NO:331) were also tested for their ability to cleave the HIV1_{—}4.4 (SEQ ID NO:334) and HIV1_{—}4.6 (SEQ ID NO:336) targets (see FIG. 47 for an example). In this case, 15 of the clones were able to cleave the HIV1_{—}4.3 (SEQ ID NO:333) and the HIV1_{—}4.5 (SEQ ID NO:335) targets. Sequence analysis of these clones showed the presence of 10 different endonuclease variants. Comparison of sequences of the positive clones in all the targets indicated the presence of a total of 11 novel endonuclease variants.

The sequence of ten I-CreI variants cleaving the HIV1_{—}4 target (SEQ ID NO:331) when forming a heterodimer with the KHSMAS/NYSYR variant (SEQ ID NO:177) are listed in Table XXXIV.

TABLE XXXIV
Sequences corresponding to the variants cleaving the HIV1_4
DNA target (SEQ ID NO: 331)

| SEQ ID NO: | Unique mutations, compared to the I-CreI sequence |
|---|---|
| 229 | 30H 33M 38A 44N 68Y 70S 75Y 77R 92R |
| 230 | 13Q 19S 30H 33M 38A 44A 68Y 70S 75Y 77R |
| 231 | 30H 33M 38A 44N 68Y 70S 75Y 77R 132V |
| 232 | 30H 33M 38A 44N 68Y 70S 75Y 77R |
| 233 | 13Q 26R 30H 33M 38A 44A 68Y 70S 75Y 77K 92R 112P |
| | 132V |
| 234 | 13Q 26R 30H 33M 38A 44N 68Y 70S 75Y 77R 87L |
| 235 | 30H 33M 38A 44N 54L 68Y 70S 75Y 77R |
| 236 | 30H 33M 38A 44N 68Y 70S 75Y 77K 87L 92R 132V |
| 237 | 28Q 38R 40K 44K 54L 68T 70G 75N 80K 114T |
| 238 | 13Q 26R 30H 33M 38A 44A 68Y 70S 75Y 77R 92R 160R |

* Mutations resulting from site-directed mutagenesis are in bold.

### EXAMPLE 24

### Strategy for Engineering Meganucleases Cleaving the HIV1_{—}5 Target (SEQ ID NO:337) from the HIV1 Virus

The HIV1_{—}5 target (SEQ ID NO:337) is a 22 by (non-palindromic) target located in the pol gene of the HIV1 provirus. This target is precisely located at positions 2317-2338 of the HIV-1 pNL4-3 vector (accession number AF324493, Adachi et al., J. Virol., 1986, 59, 284-291), a subtype B infectious molecular clone.

The HIV1_{—}5 sequence (SEQ ID NO: 337) is partly a patchwork of the 10TCT_P (SEQ ID NO:377), 10CTG_P (SEQ ID NO:378), 5TAG_P (SEQ ID NO:386) and 5_CCT_P (SEQ ID NO:384) targets (FIG. 48) which are cleaved by previously identified meganucleases, obtained as described in International PCT Applications WO 2006/097784 and WO 2006/097853; Arnould et al., J. Mol. Biol., 2006, 355, 443-458; Smith et al., Nucleic Acids Res., 2006. Thus, HIV1_{—}5 could be cleaved by combinatorial variants resulting from these previously identified meganucleases.

The 10TCT_P (SEQ ID NO:377), 10CTG_P (SEQ ID NO:378), 5TAG_P (SEQ ID NO:386) and 5_CCT_P (SEQ ID NO:384) target sequences are 24 by derivatives of C1221 (SEQ ID NO:343), a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HIV1_{—}5 series of targets (SEQ ID NO:337 to 342) were defined as 22 by sequences instead of 24 bp. HIV1_{—}5 (SEQ ID NO:337) differs from C1221 (SEQ ID NO:343) in the 4 by central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the ATAC sequence in −2 to 2 was first substituted with the GTAC sequence from C1221 (SEQ NO:343), resulting in target HIV1_{—}5.2 (SEQ ID NO: 338, FIG. 48). Then, two palindromic targets, HIV1_{—}5.3 (SEQ ID NO: 339) and HIV1_{—}5.4 (SEQ ID NO: 340), were derived from HIV1_{—}5.2 (SEQ ID NO:338) (FIG. 48). Since HIV1_{—}5.3 (SEQ ID NO:339) and HIV1_{—}5.4 (SEQ ID NO:340) are palindromic, they should be cleaved by homodimeric proteins. Two other quasi-palindromic targets were derived from these two, containing the ATAC sequence in −2 to 2 (targets HIV1_{—}5.5 (SEQ ID NO: 341) and HIV1_{—}5.6 (SEQ ID NO: 342), FIG. 48). Thus, proteins able to cleave HIV1_{—}5.3 (SEQ ID NO:339) and HIV1_{—}5.4 (SEQ ID NO:340) targets or, preferentially, the quasipalindromic targets as homodimers were first designed (examples 25 and 26) and then co-expressed to obtain heterodimers cleaving HIV1_{—}5 (SEQ ID NO:337) (example 27). Heterodimers cleaving the HIV1_{—}5.2 (SEQ ID NO:338) and HIV1_{—}5 (SEQ ID NO:337) targets could be identified. In order to improve cleavage activity for the HIV1_{—}5 target (SEQ ID NO:337), a series of variants cleaving HIV1_{—}5.3 (SEQ ID NO:339) and HIV1_{—}5.4 (SEQ ID NO:340) was chosen, and then refined. The chosen variants were subjected to random or site-directed mutagenesis, and used to form novel heterodimers that were screened against the HIV1_{—}5 target (SEQ ID NO:337) (examples 28, 29, 30 and 31). Heterodimers could be identified with an improved cleavage activity for the HIV1_{—}5 target (SEQ ID NO:337).

### EXAMPLE 25

### Identification of Meganucleases Cleaving HIV1_{—}5.3 (SEQ ID NO:339)

This example shows that I-CreI variants can cut the HIV1_{—}5.3 (SEQ ID NO:339) DNA target sequence derived from the left part of the HIV1_{—}5.2 target (SEQ ID NO:338) in a palindromic form (FIG. 48).

HIV1_{—}5.3 (SEQ ID NO:339) is similar to 10TCT_P (SEQ ID NO:377) at positions ±1, ±2, ±6, ±8, ±9, and ±10 and to 5TAG_P (SEQ ID NO:386) at positions ±1, ±2, ±3, ±4, ±5 and ±6. It was hypothesized that positions ±7 and ±11 would have little effect on the binding and cleavage activity. Variants able to cleave the 10TCT_P target (SEQ ID NO:377) were obtained by mutagenesis of I-CreI N75 or D75, at positions

28, 30, 32, 33, 38, 40 and 70, as described previously in Smith et al. Nucleic Acids Res., 2006, 34, e149; International PCT Applications WO 2007/060495 and WO 2007/049156. Variants able to cleave 5TAG_P (SEQ ID NO:386) were obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Both sets of proteins are mutated at position 70. However, the existence of two separable functional subdomains was hypothesized. This implies that this position has little impact on the specificity at bases 10 to 8 of the target. Mutations at positions 24 found in variants cleaving the 5TAG_P target (SEQ ID NO:386) will be lost during the combinatorial process. But it was hypothesized that this will have little impact on the capacity of the combined variants to cleave the HIV1_{—}5.3 target (SEQ ID NO:339).

Therefore, to check whether combined variants could cleave the HIV1_{—}5.3 target (SEQ ID NO:339), mutations at positions 44, 68, 70, 75 and 77 from proteins cleaving 5TAG_P (SEQ ID NO:386) were combined with the 28, 30, 32, 33, 38 and 40 mutations from proteins cleaving 10TCT_P (SEQ ID NO:377).

### A) Material and Methods

### a) Construction of Target Vector

The target was cloned as follows: an oligonucleotide corresponding to the HIV1_{—}5.3 target sequence (SEQ ID NO:339) flanked by gateway cloning sequences was ordered from PROLIGO: 5′ TGGCATACAAGTTTGCTCTATTAGGTACCTAATAGAGCCAATCGTCTGTCA 3′ (SEQ ID NO: 52). The same procedure was followed for cloning the HIV1_{—}5.5 target (SEQ ID NO:341), using the oligonucleotide: 5′ TGGCATACAAGTTTGCTCTATTAGATACCTAATAGAGCCAATCGTCTGTCA 3′ (SEQ ID NO: 53). Double-stranded target DNA, generated by PCR amplification of the single stranded oligonucleotide, was cloned using the Gateway protocol (INVITROGEN) into the yeast reporter vector (pCLS1055, FIG. 8). Yeast reporter vector was transformed into Saccharomyces cerevisiae strain FYBL2-7B (MATα, ura3Δ851, trp1Δ63, leu2Δ1, lys2Δ202), resulting in a reporter strain.

### b) Construction of Combinatorial Mutants

I-CreI variants cleaving 10TCT_P (SEQ ID NO:377) or 5TAG_P (SEQ ID NO:386) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TCT_P (SEQ ID NO:377) and 5TAG_P (SEQ ID NO:386) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Gal10R 5′-acaaccttgattggagacttgacc-3′(SEQ ID NO: 17)) specific to the vector (pCLS0542, FIG. 9) and primers (assF 5′-ctannnttgaccttt-3′ (SEQ ID NO: 18) or assR 5′-aaaggtcaannntag-3′(SEQ ID NO: 19)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 9) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

### B) Results

I-CreI combinatorial variants were constructed by associating mutations at positions 44, 68, 70, 75 and 77 from proteins cleaving 5TAG_P (SEQ ID NO:386) with the 28, 30, 32, 33, 38 and 40 mutations from proteins cleaving 10TCT_P (SEQ ID NO:377) on the I-CreI scaffold, resulting in a library of complexity 1920. Examples of combinatorial variants are displayed in Table XXXV, none of the variants tested from the combinatorial library produced a positive result. This library was transformed into yeast and 3348 clones (1.7 times the diversity) were screened for cleavage against the HIV15.3 (SEQ ID NO:339) and HIV15.5 (SEQ ID NO:341) DNA targets. Two positive clones were found (though having weak cleavage activity), which after sequencing turned out to correspond to 2 different novel endonuclease variants (Table XXXVI). These two positives are shown in FIG. 49. These two variants display non parental combinations at positions 28, 30, 32, 33, 38, 40 or 44, 68, 70, 75, 77. Such combinations likely result from PCR artifacts during the combinatorial process. Alternatively, the variants may be I-CreI combined variants resulting from micro-recombination between two original variants during in vivo homologous recombination in yeast.

TABLE XXXV
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, 70, | | | | | | | | | | | |
| 75 and 77 | | | | | | | | | | | |
| (ex : | | | | | | | | | | | |
| ARNNI | | | | | | | | | | | |
| stands for | | | | | | | | | | | |
| A44, R68, | | | | | | | | | | | |
| N70, | Amino acids at positions 28, 30, 32, 33, 38 and 40 | | | | | | | | | | |
| N75 and | (ex: KHSSQS stands for K28, H30, S32, S33, Q38 and S40) | | | | | | | | | | |
| I77) | KASTQS | KCSGQS | KHSCQS | KTSAQS | KNSGTS | KNSTES | KSSSTS | KNSAWS | KQSGQS | KNTCQS | KKSTQS |
| AYSYK | | | | | | | | | | | |
| ARSNI | | | | | | | | | | | |
| SRSYT | | | | | | | | | | | |
| VERNR | | | | | | | | | | | |
| ARSYT | | | | | | | | | | | |
| TRSYV | | | | | | | | | | | |
| AANNI | | | | | | | | | | | |
| AASYR | | | | | | | | | | | |
| AGNNI | | | | | | | | | | | |
| AHQNI | | | | | | | | | | | |
| NHSYN | | | | | | | | | | | |
| NYSYK | | | | | | | | | | | |
| NYSYV | | | | | | | | | | | |
| NRSYN | | | | | | | | | | | |
| SRSYS | | | | | | | | | | | |
| YRSNV | | | | | | | | | | | |
| ARDNI | | | | | | | | | | | |
| ARSYI | | | | | | | | | | | |
| NRSYI | | | | | | | | | | | |
| ANANI | | | | | | | | | | | |
| ARHDI | | | | | | | | | | | |
| DNSNI | | | | | | | | | | | |
| TYSYK | | | | | | | | | | | |
| ARSYT | | | | | | | | | | | |

*Only 264 out of the 1920 combinations are displayed. None of them were identified in the positive clones.

TABLE XXXVI
I-CreI variants with additional mutations
capable of cleaving the HIV1_5.3 DNA
target (SEQ ID NO: 339).

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and I77) | NO: |
| KNSCYS/AYQNI | 241 |
| KNSCAS/NHSYN +80K | 242 |

### EXAMPLE 26

### Making of Meganucleases Cleaving HIV1_{—}5.4 (SEQ ID NO:340)

This example shows that I-CreI variants can cleave the HIV1_{—}5.4 DNA target sequence (SEQ ID NO:340) derived from the right part of the HIV1_{—}5.2 target (SEQ ID NO:338) in a palindromic form (FIG. 4).

HIV1_{—}5.4 (SEQ ID NO:340) is similar to 5CCT_P (SEQ ID NO:384) at positions ±1, ±2, ±3, ±4, ±5 and ±8 and to 10CTG_P (SEQ ID NO:378) at positions ±1, ±2, ±3, ±4, ±8, ±9 and ±10. It was hypothesized that positions ±6, ±7 and +11 would have little effect on the binding and cleavage activity. Variants able to cleave 5CCT_P (SEQ ID NO:384) were obtained by mutagenesis of I-CreI N75 at positions 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10TGG_P target (SEQ ID NO:379) were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156.

Both sets of proteins are mutated at position 70. However, the existence of two separable functional subdomains was hypothesized. This implies that this position has little impact on the specificity at bases 10 to 8 of the target.

Therefore, to check whether combined variants could cleave the HIV1_{—}5.4 target (SEQ ID NO:340), mutations at positions 44, 68, 70, 75 and 77 from proteins cleaving 5CCT_P (SEQ ID NO:384) were combined with the 28, 30, 32, 33, 38 and 40 mutations from proteins cleaving 10CTG_P (SEQ ID NO:378).

### A) Material and Methods

### a) Construction of Target Vector

The experimental procedure is as described in example 2, with the exception that different oligonucleotides corresponding to the HIV1_{—}5.4 (SEQ ID NO:340) and HIV1_{—}5.6 (SEQ ID NO:342) targets. The oligonucleotide used for the HIV1_{—}5.4 target (SEQ ID NO:340) was: 5′TGGCATACAAGTTTATCTGCTCCTGTACAGGAGCAGATCAATCGTCTGTCA 3′ (SEQ ID NO: 243), and 5′ TGGCATACAAGTTTATCTGCTCCTATACAGGAGCAGATCAATCGTCTGTCA 3′ (SEQ ID NO: 244) for HIV1_{—}5.6 target (SEQ ID NO:342).

### b) Construction of Combinatorial Variants

I-CreI variants cleaving 10CTG_P (SEQ ID NO:378) or 5CCT_P (SEQ ID NO:384) were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10CTG_P (SEQ ID NO:378) and 5CCT_P (SEQ ID NO:384) targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 16) or Ga110R 5′-acaaccttgattggagacttgacc-3′ (SEQ ID NO: 17)) specific to the vector (pCLS1107,

FIG. 11) and primers (assF 5′-ctannnttgaccttt-3′ (SEQ ID NO: 18) or assR 5′-aaaggtcaannntag-3′(SEQ ID NO: 19), where mm codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. Positives resulting clones were verified by sequencing (MILLEGEN) as described in example 2.

### B) Results

I-CreI combinatorial variants were constructed by associating mutations at positions 44, 68, 70, 75 and 77 from proteins cleaving 5CCT_P (SEQ ID NO:384) with the 28, 30, 32, 33, 38 and 40 mutations from proteins cleaving 10CTG_P (SEQ ID NO:378) on the I-CreI scaffold, resulting in a library of complexity 1600. Examples of combinatorial variants are displayed in Table XXXXI. This library was transformed into yeast and 3348 clones (2 times the diversity) were screened for cleavage against the HIV1_{—}5.4 (SEQ ID NO:340) and HIV1_{—}5.6 (SEQ ID NO:342) DNA targets. A total of 10 positive clones were found to cleave HIV1_{—}5.4 (SEQ ID NO:340). Sequencing of these 10 clones allowed the identification of 9 novel endonuclease variants, which are represented in Table XXXVII. Examples of positives are shown in FIG. 50. The sequence of several of the variants identified display non parental combinations at positions 28, 30, 32, 33, 38, 40 or 44, 68, 70, 75, 77 as well as additional mutations (Table)(XXVIII, SEQ ID 246, 247, 251, 252 and 253). Such variants likely result from PCR artifacts during the combinatorial process. Alternatively, the variants may be I-CreI combined variants resulting from microrecombination between two original variants during in vivo homologous recombination in yeast.

TABLE XXXVII
Panel of variants* theoretically present in the combinatorial library

| Amino | | | | | | | | | | | |
|---|---|---|---|---|---|---|---|---|---|---|---|
| acids at | | | | | | | | | | | |
| positions | | | | | | | | | | | |
| 44, 68, 70, | | | | | | | | | | | |
| 75 and 77 | | | | | | | | | | | |
| (ex : | | | | | | | | | | | |
| ARNNI | | | | | | | | | | | |
| stands for | | | | | | | | | | | |
| A44, R68, | | | | | | | | | | | |
| N70, | Amino acids at positions 28, 30, 32, 33, 38 and 40 | | | | | | | | | | |
| N75 and | (ex: KHSSQS stands for K28, H30, S32, S33, Q38 and S40) | | | | | | | | | | |
| I77) | KDSRSS | KWSTQS | KASSQS | KPSGQS | KDSRSS | KQSGQS | KQSTQS | KNTCQS | KSSNQS | KSSTQS | KTSGQS |
| KASNI | | | | | | | | | | | |
| QASET | | | | | | | | | | | |
| KESDK | | | | | | | | | | | |
| KTGNI | | | | | | | | | | | |
| KRSDA | | | | | | | | | | | |
| KYSNI | | | | | | | | | | | |
| KASDK | | | | | | | | | | | |
| KGTNI | | | | | | | | | | | |
| KTSDI | | | | | | | | | | | |
| KTSDR | | | | | | | | | + | | |
| DASKR | | | | | | | | | | | |
| KESDR | | | | | | | | | | | |
| KYSYQ | | | | | | | | | | | |
| RASNN | | | | | | | | | | | |
| RYSNN | | | + | | | | | | + | | |
| KNTNI | | | | | | | | | | | |
| KRGNI | | | | | | | | | | | |
| KDSNR | | | | | | | | | | | |
| RASNI | | | | | | | | | | | |
| KYSYI | | | | | | | | | | | |
| RYSNI | | | | | | | | | | | |
| KESNR | | | | | | | | | | | |
| RRSND | | | | | | | | | | | |
| KNSNI | | | | | | | | | | | |

*Only 264 out of the 1600 combinations are displayed.
+ indicates that a functional combinatorial variant cleaving the HIV1_5.4 target (SEQ ID NO: 340) was found among the identified positives.

TABLE XXXVIII
I-CreI variants capable of cleaving the
HIV1_5.4 target (SEQ ID NO: 340).

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32 , R33, E38, S40/T44, | ID |
| Y68, S70, N75 and 177) | NO: |
| KASSQS/RYSNN | 245 |
| KQSGQS/KYSNT | 246 |
| KQSTQS/KYSNQ | 247 |
| KSSNQS/KTSDR | 248 |
| KSSNQS/KTSDR | 249 |
| KSSNQS/KTSDR +132V | 250 |
| KSSTQS/KYSNQ | 251 |
| KTSGQS/KYSDR +151A | 252 |
| KNSSQS/KYSNI | 253 |

### EXAMPLE 27

### Making of Meganucleases Cleaving HIV1_{—}5.2 (SEQ ID NO:338) and HIV1_{—}5 (SEQ ID NO:337)

I-CreI variants able to cleave each of the palindromic HIV1_{—}5.2 (SEQ ID NO:338) derived targets (HIV1_{—}5.3 (SEQ ID NO:339) and HIV1_{—}5.4 (SEQ ID NO:340)) were identified in example 25 and example 26. Pairs of such variants (one cutting HIV1_{—}5.3 (SEQ ID NO:339) and one cutting HIV1_{—}5.4 (SEQ ID NO:340)) were co-expressed in yeast. Upon co-expression, there should be three active molecular species, two homodimers, and one heterodimer. It was assayed whether the heterodimers that should be formed, cut the HIV1_{—}5.2 (SEQ ID NO:338) and the non palindromic HIV1_{—}5 targets (SEQ ID NO:337).

### A) Materials and Methods

### a) Construction of Target Vector

The experimental procedure is as described in example 2, with the exception that an oligonucleotide corresponding to the HIV1_{—}5.2 target sequence: 5′TGGCATACAAGTTTGCTCTATTAGGTACAGGAGCAGATCAATCGTCTGTC A3′ (SEQ ID NO: 254) or the HIV1_{—}5 target sequence: 5′TGGCATACAAGTTTGCTCTATTAGATACAGGAGCAGATCAATCGTCTGTC A 3′ (SEQ ID NO: 255) was used.

### b) Co-Expression of Variants

Yeast DNA was extracted from variants cleaving the HIV1_{—}5.4 (SEQ ID NO:340) target in the pCLS1107 expression vector using standard protocols and was used to transform E. coli. The resulting plasmid DNA was then used to transform yeast strains expressing a variant cutting the HIV1_{—}5.3 (SEQ ID NO:339) target in the pCLS0542 expression vector. Transformants were selected on synthetic medium lacking leucine and containing G418.

### c) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### B1 Results

Co-expression of variants cleaving the HIV1_{—}5.4 target (SEQ ID NO:340) (9 variants chosen among those described in Table)(XXVIII) and the two variants cleaving the HIV1_{—}5.3 target (SEQ ID NO:339) (described in Table XXXVI) resulted in cleavage of the HIV1_{—}5.2 target (SEQ ID NO:338) in one of the cases (FIG. 51). Nevertheless, this combination was not able to cut the HIV1_{—}5 natural target (SEQ ID NO:337), that differs from the HIV1_{—}5.2 sequence (SEQ ID NO:338) by 2 by at positions 1 and 2 (FIG. 48). The functional combination cleaving the HIV1_{—}5.2 target (SEQ ID NO:338) correspond to mutants KNSCYS/AYQNI (SEQ ID 241, cleaving HIV1_{—}5.3 (SEQ ID NO:339)) and KTSGQS/KYSDR +151A (SEQ ID 252, cleaving HIV1_{—}5.4 (SEQ ID NO:340))

### EXAMPLE 28

### Improvement of Meganucleases Cleaving HIV1_{—}5.3 (SEQ ID NO:339) by Random Mutagenesis and Assembly with Proteins Cleaving HIV1_{—}5.4 (SEQ ID NO:340)

I-CreI variants able to cleave the HIV1_{—}5.3 (SEQ ID NO:339) have been identified in example 25. Since these two variants show a weak activity, and only one of them is able to cleave the HIV1_{—}5.2 target (SEQ ID NO:338) when assembled with a meganuclease cleaving the HIV1_{—}5.4 (SEQ ID NO:340), these two variants were mutagenized, and the clones generated were screened for cleavage activity of HIV1_{—}5.3 (SEQ ID NO:339) and HIV1_{—}5.5 (SEQ ID NO:341) targets. Additionally the mutants with the strongest activity were screened for cleavage activity of HIV1_{—}5 (SEQ ID NO:337) when co-expressed with a variant cleaving HIV1_{—}5.4 (SEQ ID NO:340). According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, only one of the two components of the heterodimers cleaving HIV1_{—}5 (SEQ ID NO:337) was mutagenized.

Thus, in a first step, proteins cleaving HIV1_{—}5.3 (SEQ ID NO:339) were mutagenized and their homodimeric cleavage activity was determined, and in a second step, it was assessed whether they could cleave HIV1_{—}5 (SEQ ID NO:337) when co-expressed with a protein cleaving HIV1_{—}5.4 (SEQ ID NO:340).

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25), which are common to the pCLS0542 (FIG. 9) and pCLS1107 (FIG. 11) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Mating of Meganuclease Expressing Clones and Screening in Yeast

Experiment were performed as previously described in example 25. Positive resulting clones were verified by sequencing (MILLEGEN) as described in example 25.

### c) Variant-Target Yeast Strains, Screening and Sequencing

The yeast strain FYBL2-7B (MATα, ura3Δ851, trp1Δ63, leu2Δ1, lys2Δ202) containing the HIV1_{—}5 target (SEQ ID NO:337) in the yeast reporter vector (pCLS1055, FIG. 8) was transformed with one variant, in the kanamycin vector (pCLS1107), cutting the HIV1_{—}5.4 target (SEQ ID NO:340), using a high efficiency LiAc transformation protocol. Variant-target yeast strains were used as target strains for mating assays as described in example 27. Positives resulting clones were verified by sequencing (MILLEGEN) as described in example 25.

### B) Results

Two variants cleaving HIV1_{—}5.3 (SEQ ID NO:339), were pooled, randomly mutagenized and transformed into yeast. The sequences of the variants subjected to random mutagenesis are described in table XXXVI.

2232 transformed clones were screened for cleavage against the HIV1_{—}5.3 (SEQ ID NO:339) and HIV1_{—}5.5 (SEQ ID NO:341) DNA targets. A total of 20 positive clones were found to cleave HIV1_{—}5.3 (SEQ ID NO:339), while none of those cleaved the HIV1_{—}5.5 target (SEQ ID NO:341). Sequencing of the 20 clones allowed the identification of 13 novel endonuclease variants. An example of these variants is presented in table XXXIX and in FIG. 52.

TABLE XXXIX
Examples of 10 functional improved variants displaying cleavage
activity for HIV1_5.3 (SEQ ID NO: 339).
Optimized variants HIV1_5.3

| | |
|---|---|
| SEQ ID NO: 256 | 33C 38Y 44A 68Y 70Q 75N 89A |
| SEQ ID NO: 257 | 33C 38A 44N 68H 70S 75Y 77N 80K 103S |
| SEQ ID NO: 258 | 33C 38Y 44A 60E 68Y 70Q 75N 103D |
| SEQ ID NO: 259 | 33C 38A 44N 68H 70S 75Y 77N 80K |
| SEQ ID NO: 260 | 33C 38A 44N 59A 68H 70S 75Y 77N 80K |
| SEQ ID NO: 261 | 33C 38Y 43L 44A 68Y 70Q 75N |
| SEQ ID NO: 262 | 33C 38Y 44A 54L 68Y 70Q 75N 117G |
| SEQ ID NO: 263 | 33C 38Y 44A 68Y 70Q 75N |
| SEQ ID NO: 264 | 6S 30S 33C 38Y 44A 68H 70S 75Y 77N 80K |
| SEQ ID NO: 265 | 33C 38Y 44A 68Y 70Q 72Y 75N 107R |

* Mutations resulting from random mutagenesis are in bold.

The 20 clones showing cleavage activity on target HIV1_{—}5.3 (SEQ ID NO:339) were also mated with a yeast strain that contains (i) the HIV1_{—}5 target (SEQ ID NO:337) in a reporter plasmid (ii) an expression plasmid containing a variant that cleaves the HIV1_{—}5.4 target (SEQ ID NO:340) (SEQ ID 252; I-CreI 30T,33G,44K,68Y,70S,77R +151A or KTSGQS/KYSDR +151A, according to the nomenclature of Table I). After mating with this yeast strain, no clones were found to cleave the HIV1_{—}5 target (SEQ ID NO:337).

### EXAMPLE 28bis

### Improvement of Meganucleases Cleaving HIV1_{—}5.3 (SEQ ID NO:339) by a Second Round of Random Mutagenesis and Assembly with Proteins Cleaving HIV1_{—}5.4 (SEQ ID NO:340)

In order to further improve the activity of the obtained meganucleases, a second round of random mutagenesis was carried out following the same rationale of example 28. For this purpose, ten variants cleaving HIV1_{—}5.3 (SEQ ID NO:339) were mutagenized, and variants were screened for cleavage activity of HIV1_{—}5.3 (SEQ ID NO:339) and HIV15.5 (SEQ ID NO:341) targets. Additionally, the mutants with the strongest activity were screened for cleavage activity of HIV1_{—}5 (SEQ ID NO:337) when co-expressed with a variant cleaving HIV1_{—}5.4 (SEQ ID NO:340).

The materials and methods have previously been described in example 28.

### A) Results

Ten variants cleaving HIV1_{—}5.3 (SEQ ID NO:339), were pooled, randomly mutagenized and transformed into yeast. The variants submitted to random mutagenesis correspond to variants described in Table XXXIX (SEQ ID NO: 256 to 265).

2232 transformed clones were screened for cleavage against the HIV1_{—}5.3 (SEQ ID NO:339) and HIV1_{—}5.5 (SEQ ID NO:341) DNA targets. A total of 80 positive clones were found to cleave HIV1_{—}5.3 (SEQ ID NO:339), while 25 of those cleaved also the HIV1_{—}5.5 target (SEQ ID NO:341). Sequencing of the 80 clones allowed the identification of 39 novel endonuclease variants. An example of the identified variants is presented in table XXXX and FIG. 53.

The 80 clones showing cleavage activity on target HIV1_{—}5.3 (SEQ ID NO:339) were then mated with a yeast strain that contains (i) the HIV1_{—}5 target (SEQ ID NO:337) in a reporter plasmid (ii) an expression plasmid containing a variant that cleaves the HIV1_{—}5.4 target (SEQ ID NO:340) (I-CreI 30S,33N,44K,68Y,70S,77R +103T or KSSNQS/KYSDR +103T (SEQ ID NO:276), according to the nomenclature of Table I). After mating with this yeast strain, 4 clones were found to cleave the HIV1_{—}5 (SEQ ID NO:337). Thus, 4 positives contained proteins able to form heterodimers with KSSNQS/KYSDR +103T (SEQ ID NO: 276) showing cleavage activity on the HIV1_{—}5 target (SEQ ID NO:337). An example of positives is shown in FIG. 54. These 4 variants are presented as an example in Table XXXX (SEQ ID NO:266 to 269).

TABLE XL
Examples of 10 functional variants displaying strong cleavage activity
for HIV1_5.3 (SEQ ID NO: 339).
Optimized variants HIV1_5.3 (2nd round)

| | |
|---|---|
| SEQ ID NO: 266 | 33C 38Y 44A 50R 60E 68Y 70Q 75N 79T 85R 103D |
| SEQ ID NO: 267 | 33C 38A 44N 68H 70S 75Y 77N 80K 161P |
| SEQ ID NO: 268 | 24F 33C 38Y 44A 68Y 70Q 72Y 75N 107R 153Y |
| | 163G 164G |
| SEQ ID NO: 269 | 33C 38Y 44A 66H 68Y 70Q 72Y 75N 108V |
| SEQ ID NO: 270 | 6S 11Q 30S 33C 38Y 44A 68Y 70Q 75N 89A |
| SEQ ID NO: 271 | 33C 38A 44N 68H 70S 75Y 77N 80K 103S 132V |
| SEQ ID NO: 272 | 33C 38Y 44A 68Y 70Q 75N 89A |
| SEQ ID NO: 273 | 33C 38Y 44A 68Y 70Q 72Y 75N |
| SEQ ID NO: 274 | 33C 38Y 44A 60E 68Y 70Q 75N 103D 157V |
| SEQ ID NO: 275 | 33C 38Y 44A 68Y 70Q 75N 89A 114T 151A |

* Mutations resulting from random mutagenesis are in bold.

### EXAMPLE 29

### Improvement of Meganucleases Cleaving HIV1_{—}5 (SEQ ID NO:337) by Site-Directed Mutagenesis of Proteins Cleaving HIV1_{—}5.3 (SEQ ID NO:339) and Assembly with Proteins Cleaving HIV1_{—}5.4 (SEQ ID NO:340)

Three of the I-CreI variants cleaving HIV1_{—}5.3 (SEQ ID NO:339) described in Table XL were mutagenized by introducing selected amino-acid substitutions in the proteins and screening for more efficient variants cleaving HIV1_{—}5 (SEQ ID NO:337) in combination with a variant cleaving HIV1_{—}5.4 (SEQ ID NO:340).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HIV1_{—}5.3 (SEQ ID NO:339), and the resulting proteins were tested for their ability to induce cleavage of the HIV1_{—}5 target (SEQ ID NO:337), upon co-expression with a variant cleaving HIV1_{—}5.4 (SEQ ID NO:340), as well as for the ability to cleave targets HIV1_{—}5.3 (SEQ ID NO:339) and HIV1_{—}5.5 (SEQ ID NO:341).

### A) Material and Methods

### a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 16) or Ga110R 5′-acaaccttgattggagacttgacc-3′(SEQ ID NO: 17)) and a primer specific to the I—CreI coding sequence for amino acids 14-24 that contains the substitution mutation G19S (G19SF 5′-gccggctttgtggactctgacggtagcatcatc-3′ (SEQ ID NO: 47) or G19SR 5′-gatgatgctaccgtcagagtccacaaagccggc-3′(SEQ ID NO: 48)). The same strategy is used with the following pair of oligonucleotides to introduce the mutations leading to the F54L, E80K, F87L, V105A and I132V substitutions in the coding sequences of the variants, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgcttaa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
I132VF:
5′-acctgggtggatcaggttgcagctctgaacgat-3′
and
I132VR:
5′-atcgttcagagctgcaacctgatccacccaggt-3′.

For each substitution to be introduced, the resulting PCR products contain 33 bp of homology with each other. The PCR fragments were purified. The ten PCR fragments were pooled en equimolar amounts to generate a mix containing 50 ng of PCR DNA and 75 ng of vector DNA (pCLS0542, FIG. 9), linearized by digestion with NcoI and EagI. This mix was used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

### c) Mating of Meganuclease Expressing Clones and Screening in Yeast

The experimental procedure is as described in example 28.

### d) Sequencing of Variants

The experimental procedure is as described in example 25.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of three variants cleaving HIV1_{—}5.3 (SEQ ID NO:339) (SEQ ID NO: 266, 269 and 270; described in Table XL). 558 transformed clones were screened for cleavage against the HIV1_{—}5.3 (SEQ ID NO:339) and HIV1_{—}5.5 (SEQ ID NO:341) DNA targets. A total of 450 positive clones were found to cleave HIV1_{—}5.3 (SEQ ID NO:339), while 435 of those cleaved also the HIV1_{—}5.5 target (SEQ ID NO:341). An example of positive variants is shown in FIG. 55.

The 558 transformed clones were also mated with a yeast strain that contains (i) the HIV1_{—}5 target (SEQ ID NO:337) in a reporter plasmid (ii) an expression plasmid containing a variant that cleaves the HIV1_{—}5.4 target (SEQ ID NO:340) (I-CreI 30S,33N,44K,68Y,70S,77R +103T or KSSNQS/KYSDR +103T (SEQ ID NO:276), according to the nomenclature of Table I). After mating with this yeast strain, 444 clones were found to cleave the HIV1_{—}5 (SEQ ID NO:337). Thus, 444 positives contained proteins able to form heterodimers with KSSNQS/KYSDR +103T (SEQ ID NO: 276) showing cleavage activity on the HIV1_{—}5 target (SEQ ID NO:337). An example of positive clones is shown in FIG. 56.

Sequencing of the 93 clones with the highest cleavage activity on the HIV1_{—}5 target (SEQ ID NO:337) allowed the identification of 50 different endonuclease variants.

The sequence of ten I-CreI variants cleaving the HIV1_{—}5 target (SEQ ID NO:337) when forming a heterodimer with the KSSNQS/KYSDR +103T variant (SEQ ID NO:276) are listed in Table XLI.

TABLE XLI
Examples of 10 functional variants displaying strong cleavage
activity for HIV1_5(SEQ ID NO: 337)
Optimized variants HIV1_5.3

| | |
|---|---|
| SEQ ID NO: 277 | 6S 30S 33C 38Y 44A 50R 60E 68Y 70Q 75N 79T 85R |
| | 108V |
| SEQ ID NO: 278 | 33C 38Y 44A 50R 60E 68Y 70Q 75N 79T 85R 103D |
| SEQ ID NO: 279 | 19S 33C 38Y 44A 50R 60E 68Y 70Q 75N 79T 85R |
| | 108V |
| SEQ ID NO: 280 | 19S 33C 38Y 44A 68Y 70Q 75N 79T 85R 105A |
| SEQ ID NO: 281 | 6S 11Q 19S 30S 33C 38Y 44A 68Y 70Q 75N 89A |
| | 105A 132V |
| SEQ ID NO: 282 | 33C 38Y 44A 50R 60E 68Y 70Q 75N 79T 85R 105A |
| SEQ ID NO: 283 | 33C 38Y 44A 66H 68Y 70Q 73G 75N 89A 105A |
| SEQ ID NO: 284 | 19S 33C 38Y 44A 50R 60E 68Y 70Q 75N 79T 85R |
| | 103D |
| SEQ ID NO: 285 | 6S 11Q 19S 33C 38Y 44A 66H 68Y 70Q 72Y 75N |
| | 103D 108V |
| SEQ ID NO: 286 | 33C 38Y 44A 68Y 70Q 75N 79T 85R 105A |

* Mutations resulting from site directed mutagenesis are in bold.

### EXAMPLE 30

### Improvement of Meganucleases Cleaving HIV1_{—}5.4 (SEQ ID NO:340) by Random Mutagenesis and Assembly with Proteins Cleaving HIV1_{—}5.3 (SEQ ID NO:339)

As a complement to example 29 we also decided to perform random mutagenesis with variants that cleave HIV1_{—}5.4 (SEQ ID NO:340). The variants generated were screened for their cleavage activity on targets HIV1_{—}5.4 (SEQ ID NO:340) and HIV1_{—}5.6 (SEQ ID NO:342); and the mutagenized proteins cleaving HIV1_{—}5.4 (SEQ ID NO:340) were then tested to determine if they could efficiently cleave HIV1_{—}5 (SEQ ID NO:337) when co-expressed with a protein cleaving HIV1_{—}5.3 (SEQ ID NO:339).

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 24) and ICreIpostRev (5′-ggctcgaggagctcgtctagaggatcgctcgagttatcagtcggccgc-3′; SEQ ID NO: 25). Approximately 25 ng of the PCR product and 75 ng of vector DNA (pCLS1107, FIG. 11) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Variant-Target Yeast Strains, Screening and Sequencing

The yeast strain FYBL2-7B (MATα, ura3Δ851, trp1Δ63, leu2Δ1, lys2Δ202) containing the HIV1_{—}5 target (SEQ ID NO:337) in the yeast reporter vector (pCLS1055, FIG. 8) was transformed with variants, in the leucine vector (pCLS0542), cutting the HIV1_{—}5.3 target (SEQ ID NO:339), using a high efficiency LiAc transformation protocol. Variant-target yeast strains were used as target strains for mating assays as described in example 27. Positives resulting clones were verified by sequencing (MILLEGEN) as described in example 25.

### B) Results

Nine variants cleaving HIV1_{—}5.4 (SEQ ID NO:340) were pooled, randomly mutagenized and transformed into yeast. The sequences of the variants subjected to random mutagenesis are described in Table XXXVIII.

2232 transformed clones were screened for cleavage against the HIV1_{—}5.4 (SEQ ID NO:340) and HIV1_{—}5.6 (SEQ ID NO:342) DNA targets. A total of 53 positive clones were found to cleave HIV1_{—}5.4 (SEQ ID NO:340), while 6 of those also cleaved the HIV1_{—}5.6 target (SEQ ID NO:342). Sequencing of the 53 clones showing the strongest activity allowed the identification of 42 novel endonuclease variants. An example of the identified variants is presented in Table XLII and in FIG. 57.

TABLE XLII
Examples of 10 functional variants displaying strong cleavage
activity for HIV1_5.4 (SEQ ID NO: 340).
Optimized variants HIV1_5.4

| | |
|---|---|
| SEQ ID NO: 276 | 30S 33N 44K 68Y 70S 77R 103T |
| SEQ ID NO: 288 | 30S 33N 44R 54I 68Y 70S 75N 77Q 124V |
| SEQ ID NO: 289 | 30A 33S 44R 66H 68Y 70S 75N 77N 89S |
| SEQ ID NO: 290 | 33S 43L 44K 54L 68Y 70S 75Y 92R |
| SEQ ID NO: 291 | 30S 33N 44R 56N 68Y 70S 75N 77N 132V |
| SEQ ID NO: 292 | 33S 43L 44K 54L 68Y 70S 75Y 82R 132V |
| SEQ ID NO: 293 | 33S 44K 68Y 70S 75N 77V |
| SEQ ID NO: 294 | 30Q 33T 44K 68Y 70S 77R 83S 151A 159R |
| SEQ ID NO: 295 | 30T 33G 44K 66F 68Y 70S 77R 151A |
| SEQ ID NO: 296 | 33S 44K 54L 68Y 70S 75N |

* Mutations resulting from random mutagenesis are in bold.

The 53 positive clones showing the highest cleavage activity on target

HIV1_{—}5.4 (SEQ ID NO:340) were then mated with a yeast strain that contains (i) the HIV1_{—}5 target (SEQ ID NO:337) in a reporter plasmid (ii) an expression plasmid containing a variant that cleaves the HIV1_{—}5.3 target (SEQ ID NO:339) (I-CreI 33C,38Y,44A,68Y,70Q,75N +89A or KNSCYS/AYQNI +89A, according to the nomenclature of Table I; SEQ ID NO:256). After mating with this yeast strain, no clones were found to cleave the HIV1_{—}5 target (SEQ ID NO:337).

### EXAMPLE 30bis

### Improvement of Meganucleases Cleaving HIV1_{—}5 (SEQ ID NO:337) by a Second Round of Random Mutagenesis of Proteins Cleaving HIV1_{—}5.4 (SEQ ID NO:340) and Assembly with Proteins Cleaving HIV1_{—}5.3 (SEQ ID NO:339)

In order to further improve the activity of the obtained meganucleases, a second round of random mutagenesis was carried out following the same rationale of example 30. For this purpose, six variants cleaving HIV1_{—}5.4 (SEQ ID NO:340) were mutagenized, and variants were screened for cleavage activity of HIV1_{—}5.4 (SEQ ID NO:340) and HIV1_{—}5.6 (SEQ ID NO:342) targets. Additionally the mutants were screened for cleavage activity of HIV1_{—}5 (SEQ ID NO:337) when co-expressed with a variant cleaving HIV1_{—}5.3 (SEQ ID NO:339).

The materials and methods have previously been described in example 30.

A) Results

Six variants cleaving HIV1_{—}5.4 (SEQ ID NO:340), were pooled, randomly mutagenized and transformed into yeast. The six variants submitted to random mutagenesis correspond to variants described in Table XLII (SEQ ID NO: 276 and 288 to 292).

2232 transformed clones were screened for cleavage against the HIV1_{—}5.4 (SEQ ID NO:340) and HIV1_{—}5.6 (SEQ ID NO:342) DNA targets. A total of 21 positive clones were found to cleave HIV1_{—}5.4 (SEQ ID NO:340), while 9 of those cleaved also the HIV1_{—}5.6 target (SEQ ID NO:342). Sequencing of the 21 clones allowed the identification of 16 novel endonuclease variants. An example of the identified variants is presented in Table XLIII and FIG. 58.

The 21 positive clones showing cleavage activity on target HIV1_{—}5.4 (SEQ ID NO:340) were then mated with a yeast strain that contains (i) the HIV1_{—}5 target (SEQ ID NO:337) in a reporter plasmid (ii) an expression plasmid containing a variant that cleaves the HIV1_{—}5.3 target (SEQ ID NO:339) (I-CreI 33C,38Y,44A,68Y,70Q,75N +89A or KNSCYS/AYQNI +89A, according to the nomenclature of Table I; SEQ ID NO:256). After mating with this yeast strain, no clones were found to cleave the HIV1_{—}5 target (SEQ ID NO:337).

TABLE XLIII
Examples of 10 functional variants displaying strong cleavage
activity for HIV1_5.4 (SEQ ID NO: 340).
Optimized variants HIV1_5.4 (2^{nd} round)

| | |
|---|---|
| SEQ ID NO: 297 | 6S 33S 44R 54I 68Y 70S 75N 77Q 124V 158R 163T |
| SEQ ID NO: 298 | 30S 33N 44K 68Y 70S 77R 103T |
| SEQ ID NO: 299 | 30S 33N 44K 68Y 70S 77R 103T 142R 160E |
| SEQ ID NO: 300 | 30S 33N 44R 54I 68Y 70S 75N 77Q 124V |
| SEQ ID NO: 301 | 33S 43L 44K 45M 54L 66H 68Y 70S 75N 77N 89S |
| SEQ ID NO: 302 | 2Y 16L 30S 33S 44R 66H 68Y 70S 75N 77N 82E 89S 103S 147A |
| SEQ ID NO: 303 | 33S 43L 44K 54L 68Y 70S 75Y 92R 114Y |
| SEQ ID NO: 304 | 33S 43L 44K 54L 68Y 70S 75Y 92R |
| SEQ ID NO: 305 | 33S 43L 44K 54L 68Y 70S 75Y 92R 153Y |
| SEQ ID NO: 306 | 30A 33S 44R 64A 66H 68Y 70S 75N 77N 89S 103D 128R 146V 151A |

* Mutations resulting from random mutagenesis are in bold.

### EXAMPLE 31

### Improvement of Meganucleases Cleaving HIV1_{—}5 (SEQ ID NO:337) by Site-Directed Mutagenesis of Proteins Cleaving HIV1_{—}5.4 (SEQ ID NO:340) and assembly with proteins cleaving HIV1_{—}5.3 (SEQ ID NO:339)

Two of the I-CreI variants cleaving HIV1_{—}5.4 (SEQ ID NO:340) described in Table XLIII were mutagenized by introducing selected aminoacid substitutions in the proteins and screening for more efficient variants cleaving HIV1_{—}5.4 (SEQ ID NO:340) and HIV1_{—}5.6 (SEQ ID NO:342), as well as for cleavage of the HIV1_{—}5 (SEQ ID NO:337) target when in combination with a variant cleaving HIV1_{—}5.3 (SEQ ID NO:339).

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V 105A) and Isoleucine 132 with Valine (I132V).

### A) Material and Methods

### a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 16) or Gal10R 5′-acaaccttgattggagacttgacc-3′(SEQ ID NO: 17)) and a primer specific to the I-CreI coding sequence for amino acids 14-24 that contains the substitution mutation G19S (G19SF 5′-gccggctttgtggactctgacggtagcatcatc-3′ (SEQ ID NO: 47) or G19SR 5′-gatgatgctaccgtcagagtccacaaagccggc-3′(SEQ ID NO: 48)). The same strategy is used with the following pair of oligonucleotides to introduce the mutations leading to the F54L, E80K, F87L, V105A and I132V substitutions in the coding sequences of the variants, respectively:

(SEQ ID NO: 49 and 50)
F54LF:
5′-acccagcgccgttggctgctggacaaactagtg-3′
and
F54LR:
5′-cactagtttgtccagcagccaacggcgctgggt-3′;
SEQ ID NO: 51 and 52)
E80KF:
5′-ttaagcaaaatcaagccgctgcacaacttcctg-3′
and
E80KR:
5′-caggaagttgtgcagcggcttgattttgcttaa-3′;
SEQ ID NO: 53 and 54)
F87LF:
5′-aagccgctgcacaacctgctgactcaactgcag-3′
and
F87LR:
5′-ctgcagttgagtcagcaggttgtgcagcggctt-3′;
SEQ ID NO: 55 and 56)
V105AF:
5′-aaacaggcaaacctggctctgaaaattatcgaa-3′
and
V105AR:
5′-ttcgataattttcagagccaggtttgcctgttt-3′;
SEQ ID NO: 57 and 58)
I132VF:
5′-acctgggtggatcaggttgcagctctgaacgat-3′
and
I132VR:
5′-atcgttcagagctgcaacctgatccacccaggt-3′.

For each substitution to be introduced, the resulting PCR products contain 33 bp of homology with each other. The PCR fragments were purified. The ten

PCR fragments were pooled en equimolar amounts to generate a mix containing 50 ng of PCR DNA and 75 ng of vector DNA (pCLS0542, FIG. 9), linearized by digestion with NcoI and EagI. This mix was used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the substitutions are generated in vivo by homologous recombination in yeast.

### c) Mating of Meganuclease Expressing Clones and Screening in Yeast

The experimental procedure is as described in example 28.

### d) Sequencing of Variants

The experimental procedure is as described in example 25.

### B) Results

A library containing a population harboring the six amino-acid substitutions (Glycine 19 with Serine, Phenylalanine 54 with Leucine, Glutamic acid 80 with Lysine, Phenylalanine 87 with Leucine, Valine 105 with Alanine and Isoleucine 132 with Valine) was constructed on a pool of two variants cleaving HIV1_{—}5.4 (SEQ ID NO:340) (SEQ ID NO: 297 and 299; described in Table XLIII). 558 transformed clones were screened for cleavage against the HIV1_{—}5.4 (SEQ ID NO:340) and HIV1_{—}5.6 (SEQ ID NO:342) DNA targets. A total of 378 positive clones were found to cleave HIV1_{—}5.4 (SEQ ID NO:340), while 321 of those cleaved also the HIV1_{—}5.6 target (SEQ ID NO:342). An example of positive variants is shown in FIG. 59.

The 558 transformed clones were also mated with a yeast strain that contains (i) the HIV1_{—}5 target (SEQ ID NO:337) in a reporter plasmid (ii) an expression plasmid containing a variant that cleaves the HIV1_{—}5.3 target (SEQ ID NO:339) (I-CreI 33C,38Y,44A,68Y,70Q,75N +89A or KNSCYS/AYQNI +89A (SEQ ID NO:256), according to the nomenclature of Table I). After mating with this yeast strain, 137 clones were found to cleave the HIV1_{—}5 (SEQ ID NO:337). Thus, 137 positives contained proteins able to form heterodimers with KNSCYS/AYQNI +89A (SEQ ID NO: 256) showing cleavage activity on the HIV1_{—}5 target (SEQ ID NO:337). An example of positives is shown in FIG. 60.

Sequencing of the 93 clones with the highest cleavage activity on the HIV1_{—}5 target (SEQ ID NO:337) allowed the identification of 48 different endonuclease variants.

The sequence of ten I-CreI variants cleaving the HIV1_{—}5 target (SEQ ID NO:337) when forming a heterodimer with the KNSCYS/AYQNI +89A (SEQ ID NO:256) variant are listed in Table XXXXIV.

TABLE XLIV
Examples of 10 functional variants displaying strong cleavage
activity for HIV1_5 (SEQ ID NO: 337)
Optimized variants HIV1_5.4

| | |
|---|---|
| SEQ ID NO: 307 | 19S 33S 44R 54I 68Y 70S 75N 77Q 124V 158R 163T |
| SEQ ID NO: 308 | 6S 19S 30S 33N 44K 68Y 70S 77R 103T 142R 160E |
| SEQ ID NO: 309 | 19S 30S 33N 44K 68Y 70S 77R 103T 132V 142R 160E |
| SEQ ID NO: 310 | 30S 31R 33S 44R 54I 68Y 70S 75N 77Q 124V 158R 163T |
| SEQ ID NO: 311 | 30S 33N 44K 54I 68Y 70S 75N 77Q 158R 163T 164T |
| SEQ ID NO: 312 | 6S 19S 30S 33N 44K 68Y 70S 77R 80K 89A 103T 124V 158R 163T |
| SEQ ID NO: 313 | 6S 19S 30S 33N 44K 68Y 70S 77R 103T 158R 163T |
| SEQ ID NO: 314 | 30S 44K 68Y 70S 77R 103T 160E |
| SEQ ID NO: 315 | 19S 30S 33N 44K 68Y 70S 77R 103T 142R 160E |
| SEQ ID NO: 316 | 6S 19S 33S 44R 54I 68Y 70S 75N 77Q 105A 124V 131R 158R 163T |

* Mutations resulting from site directed mutagenesis are in bold.

### EXAMPLE 32

### Covalent Assembly as Single Chain and Improvement of Meganucleases Cleaving Different HIV1 Targets by Site-Directed Mutagenesis

Coexpression of the variants cleaving the non-palindromic targets used during the custom meganuclease development process described in previous examples leads to cleavage of the corresponding DNA target in yeast. Different mutants were selected, either showing a high cleavage activity as heterodimers in the corresponding non-palindromic targets, or a high cleavage activity as homodimers in the HIV1_N.5 and in the HIV1_N.6 pseudo-palindromic targets (N standing for any of the targets described in the present patent application: 1, 3, 4, 5, 7, 8 and 9). In all cases the mutant cleaving the HIV1_N.5 target and the mutant cleaving the HIV1_N.6 target will be called Ma and Mb. This nomenclature is not related to the identity of the HIV1_N.5 or HIV1_N.6 cutter, but to the position in the single chain molecule (Ma being the N-terminal mutant and Mb being the C-terminal mutant).

Single chain constructs were engineered using the linker RM2 (AAGGSDKYNQALSKYNQALSKYNQALSGGGGS) (SEQ ID NO: 345) resulting in the production of the canonical single chain molecule: Ma-RM2-Mb. During this design step, the G19S mutation was introduced in the C-terminal (Mb) mutant. In addition, mutations K7E and K96E were introduced into the Ma mutant, while mutations E8K and E61R were introduced into the Mb mutant. This leads to the generation of the single chain molecule: Ma(K7E K96E)-RM2-Mb(E8K E61R) that is called SCOH-HIV1-MaMb.

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Certain combinations of these mutations were introduced into the coding sequence of N-terminal and C-terminal protein fragment (if these mutations were not present in the original mutants). The coding sequences of the single chain proteins were cloned into a mammalian expression vector, and their activity on the corresponding target in the HIV1 genome was tested in a cellular model developed for this purpose. Table XLV shows an example of the single chain molecules that have been generated for the different HIV1 targets.

TABLE XLV
Single Chain I-Cre I variants targeting the HIV1 provirus.

| | | Mutations on | Mutations on | | |
|---|---|---|---|---|---|
| | | N-terminal | C-terminal | Mutations in Single | SEQ ID |
| Construct | Single chain | segment | segment | Chain | NO |
| pCLS2899 | SCOH-HIV1_1-A | 7E33T40K44R6 | 8K19S30G38 | 7E33T40K44R68Y70S | 346 |
| | | 8Y70S77N96E1 | R44V54L61R | 77N96E117G132V_8 | |
| | | 17G132V | 68E75N77R8 | K19S30G38R44V54L6 | |
| | | | 0K81T132V | 1R68E75N77R80K81T | |
| | | | | 132V | |
| pCLS3726 | SCOH-HIV1_1-B | 7E17A30G38R4 | 8K19S33T40 | 7E17A30G38R42A44 | 347 |
| | | 2A44V54L64A6 | K44R61R68Y | V54L64A68E75N77R8 | |
| | | 8E75N77R80R8 | 70S77N117G | 0R86D96E99R111H1 | |
| | | 6D96E99R111H | 132V | 32V_8K19S33T40K44 | |
| | | 132V | | R61R68Y70S77N117 | |
| | | | | G132V | |
| pCLS4309 | SCOH-HIV1_1-C | 7E17A30G38R4 | 8K19S33T40 | 7E17A30G38R42A44 | 348 |
| | | 2A44V54L64A6 | K44R61R68Y | V54L64A68E77R80R8 | |
| | | 8E77R80R86D9 | 70S77N117G | 6D96E99R111H132V_ | |
| | | 6E99R111H132 | 132V | 8K19S33T40K44R61 | |
| | | V | | R68Y70S77N117G13 | |
| | | | | 2V | |
| pCLS2885 | SCOH-HIV1_3-A | 7E32K33A44K6 | 8K19S38Y43 | 7E32K33A44K68E70S | 349 |
| | | 8E70S72T75N7 | L44Y61R68S | 72T75N77R80K96E12 | |
| | | 7R80K96E129A | 70S75R77V1 | 9A132V154C158Q_8 | |
| | | 132V154C158Q | 05A132V | K19S38Y43L44Y61R6 | |
| | | | | 8S70S75R77V105A13 | |
| | | | | 2V | |
| pCLS3734 | SCOH-HIV1_3-B | 7E32K33A44K6 | 8K19S38Y43 | 7E32K33A44K68E70S | 350 |
| | | 8E70S75N77R8 | L44Y61R68S | 75N77R80K96E132V | |
| | | 0K96E132V154 | 70S75R77V8 | 154R_8K19S38Y43L4 | |
| | | R | 0K105A132V | 4Y61R68S70S75R77V | |
| | | | | 80K105A132V | |
| pCLS3737 | SCOH-HIV1_3-C | 7E32K33A44K6 | 8K19S38Y43 | 7E32K33A44K68E70S | 351 |
| | | 8E70S72T75N7 | L44Y61R68S | 72T75N77R80K96E12 | |
| | | 7R80K96E129A | 70S75R77V8 | 9A132V154C158Q_8 | |
| | | 132V154C158Q | 7L94Y105A1 | K19S38Y43L44Y61R6 | |
| | | | 32V | 8S70S75R77V87L94Y | |
| | | | | 105A132V | |
| pCLS3739 | SCOH-HIV1_3-D | 7E32K33A43L4 | 8K19S38Y43 | 7E32K33A43L44K54L | 352 |
| | | 4K54L68E70S75 | L44Y61R68S | 68E70S75N77R80K96 | |
| | | N77R80K96E13 | 70S75R77V8 | E132V_8K19S38Y43L | |
| | | 2V | 7L94Y105A1 | 44Y61R68S70S75R77 | |
| | | | 32V | V87L94Y105A132V | |
| pCLS4311 | SCOH-HIV1_3-E | 7E32K33A44K6 | 8K19S38Y43 | 7E32K33A44K68E70S | 353 |
| | | 8E70S75N77R8 | L44Y61R68S | 75N77R80K96E132V | |
| | | 0K96E132V154 | 77V80K105A | 154R_8K19S38Y43L4 | |
| | | R | 132V | 4Y61R68S77V80K105 | |
| | | | | A132V | |
| pCLS3345 | SCOH-HIV1_4-A | 7E30H33M38A | 8K19S28Q38 | 7E30H33M38A44A68 | 354 |
| | | 44A68Y70S75Y | R40K44K61R | Y70S75Y77R96E132V_ | |
| | | 77R96E132V | 68T70G75N | 8K19S28Q38R40K44 | |
| | | | 123M132V | K61R68T70G75N123 | |
| | | | | M132V | |
| pCLS3761 | SCOH-HIV1_5-A | 6S7E30S33C38 | 8K19S30S31 | 6S7E30S33C38Y44A5 | 355 |
| | | Y44A50R60E68 | R33S44R54I | 0R60E68Y70Q75N79 | |
| | | Y70Q75N79T85 | 61R68Y70S7 | T85R96E108V132V_8 | |
| | | R96E108V132V | 5N77Q124V | K19S30S31R33S44R5 | |
| | | | 132V158R16 | 4I61R68Y70S75N77Q | |
| | | | 3T | 124V132V158R163T | |
| pCLS3765 | SCOH-HIV1_5-B | 7E30S33C38Y4 | 8K19S30S33 | 7E30S33C38Y44A68Y | 356 |
| | | 4A68Y70Q75N7 | N44K54I61R | 70Q75N79T96E108V | |
| | | 9T96E108V132 | 68Y70S75N7 | 132V_8K19S30S33N4 | |
| | | V | 7Q132V158 | 4K54I61R68Y70S75N | |
| | | | R163T^{~}T | 77Q132V158R163T1 | |
| | | | | 64T | |
| pCLS4061 | SCOH-HIV1_7-A | 7E24V30R44K6 | 8K12H19S33 | 7E24V30R44K68Y69G | 357 |
| | | 8Y69G70S75Y7 | C40Q44R48 | 70S75Y77S96E100E1 | |
| | | 7S96E100E132 | R61R68Y70S | 32V_8K12H19S33C40 | |
| | | V | 75Y77N80G | Q44R48R61R68Y70S7 | |
| | | | 105A132V | 5Y77N80G105A132V | |
| pCLS4063 | SCOH-HIV1_7-B | 7E24V30R32T4 | 8K12H19S33 | 7E24V30R32T44K68Y | 358 |
| | | 4K68Y70S75Y7 | C40Q44R61 | 70575Y77S89196E132 | |
| | | 7S89I96E132V | R68Y70S75Y | V_8K12H19S33C40Q | |
| | | | 77N105A132 | 44R61R68Y70S75Y77 | |
| | | | V | N105A132V | |
| pCLS4057 | SCOH-HIV1_8-A | 7E26R30R44R4 | 8K19S28Q38 | 7E26R30R44R46G68 | 359 |
| | | 6G68N70S73M | R40K44T50R | N70S73M75Q77C96E | |
| | | 75Q77C96E103 | 61R70S75Y1 | 103S132V_8K19S28Q | |
| | | S132V | 32V153G | 38R40K44T50R61R70 | |
| | | | | S75Y132V153G | |
| pCLS4058 | SCOH-HIV1_8-B | 2S7E28Q38R40 | 8K19S30R44 | 2S7E28Q38R40K68H | 360 |
| | | K68H70S75Y77 | K46G61R68 | 70S75Y77N80K96E13 | |
| | | N80K96E132V | N70S73M75 | 2V_8K19530R44K46G | |
| | | | Q77C132V | 61R68N70S73M75Q7 | |
| | | | | 7C132V | |
| pCLS4059 | SCOH-HIV1_8-C | 7E28Q38R40K4 | 8K19S30R44 | 7E28Q38R40K44T68T | 361 |
| | | 4T68T70S75Y7 | K46G61R68 | 70S75Y77R96E132V_ | |
| | | 7R96E132V | N70S73M75 | 8K19S30R44K46G61R | |
| | | | Q77C132V | 68N70S73M75Q77C1 | |
| | | | | 32V | |
| pCLS4060 | SCOH-HIV1_8-D | 7E28Q38R40K4 | 8K19S30R44 | 7E28Q38R40K44T50R | 362 |
| | | 4T50R70S75Y9 | K46G61R68 | 70S75Y96E132V153G_ | |
| | | 6E132V153G | N70S73M75 | 8K19S30R44K46G61 | |
| | | | Q77C103S13 | R68N70S73M75Q77C | |
| | | | 2V | 103S132V | |
| pCLS4067 | SCOH-HIV1_9-A | 7E31R33H40Q4 | 8K19S24V44 | 7E31R33H40Q44K68 | 363 |
| | | 4K68Y70S75E7 | Y54L61R70S | Y70S75E77V96E132V | |
| | | 7V96E132V139 | 75Q77V132 | 139R154N_8K19S24V | |
| | | R154N | V | 44Y54L61R70S75Q77 | |
| | | | | V132V | |
| pCLS4068 | SCOH-HIV1_9-B | 7E31R33H40Q4 | 8K19S24V44 | 7E31R33H40Q44K68 | 364 |
| | | 4K68Y70S75E7 | Y61R70S75Q | Y70S75E77V96E132V | |
| | | 7V96E132V139 | 77V80V87L1 | 139R154N_8K19S24V | |
| | | R154N | 00R132V | 44Y61R70S75Q77V80 | |
| | | | | V87L100R132V | |
| pCLS4069 | SCOH-HIV1_9-C | 7E31R33H40Q4 | 8K19S24V44 | 7E31R33H40Q44K68 | 365 |
| | | 4K68Y70S75E7 | Y61R70S75Q | Y70S75E77V96E117G | |
| | | 7V96E117G132 | 77V80V87L1 | 132V154N_8K19S24V | |
| | | V154N | 00R132V | 44Y61R70S75Q77V80 | |
| | | | | V87L100R132V | |

### 1) Material and Methods

### a) Cloning of the SC_OH Single Chain Molecules

A series of synthetic gene assemblies were ordered to MWG-EUROFINS. Synthetic genes coding for the different single chain variants targeting the HIV1 provirus were cloned in pCLS1853 (FIG. 61) using AscI and XhoI restriction sites.

### EXAMPLE 33

### Determination of Antiviral Effect of HIV1 Meganuclease Variants Derived from I-CreI

The efficacy of HIV meganucleases to cleave the corresponding proviral DNA target was assessed in a cellular system containing a defective integrated provirus. This cellular model produces viral-like particles (VLPs) containing all the essential HIV1 proteins with the exception of the viral envelope glycoproteins. Nevertheless, the produced VLPs are not able to infect the cells due to the absence of entry-mediating proteins in the viral envelope. Production of VLPs can be measured in the supernatants of cultured cells using an HIV1-p24 ELISA kit. The VLP-producing cells were transfected with the plasmids coding for the different versions of the SCOH-HIV1 meganucleases (SEQ ID NO:346 to 365) and the antiviral effect was measured by the reduction in the titres of p24 present in the supernatants of transfected cells respect to a “control” sample in which the cells were transfected by a non-related meganuclease (NRM), which has no cleavage activity on the HIV1 proviral DNA.

1) Material and Methods

### a) Generation of a Cellular System Allowing to Test the Antiviral Activity of HIV1 Meganucleases (SEQ ID NO:346 to 365)

A cell line capable of producing non-replicative VLPs was generated in order to dispose of a model allowing to determine the efficacy of antiviral meganucleases. With the aim of introducing an HIV provirus in the cells, a lentiviral vector pseudotyped by the VSV envelope protein was used to transduce the HEK-293 human cell line. In order to avoid viral replication on the cellular model, the integrated provirus harbours deletion of the HIV1 accessory proteins (Vif, Vpr, Vpu and Nef) as well as of the viral envelope glycoprotein (env). A cassette conferring puromycin resistance to the cell line was introduced, as well as the EGFP coding sequence (EF1alfa.p-PuroR-IRES-EGFP) to replace the env coding sequence.

For safety reasons, two other HIV1 essential proteins have been deleted from the proviral sequence, those of the Tat and the Rev proteins, which are essential for the production of viral progeny.

To produce the cellular system, two retroviral vectors were generated harbouring either the tat or the rev coding sequences. These two vectors were used to sequentially transduce HEK-293 cells, leading to the generation of a cell line able to produce the tat and rev proteins after integration of the retroviral vectors in the cellular genome. The generated cell line was then transduced by a lentiviral expression vector that, after integration of the dsDNA resulting from reverse transcription, would generate the pseudo-HIV1 provirus containing the meganuclease target hits. The structure of the integrated provirus correspond to the sequence elements U3RU5(HIV)-PsiGAGPOL(HIV)-EF1a:Puro:IRES:GFP-U3RU5 (HIV) and is schematically represented in FIG. 62.

The cells were tested for their ability to produce VLPs by determining the presence of the HIV1 p24 protein in the culture supernatants using the Alliance® HIV1-p24 ELISA Kit (Perkin Elmer Inc, Waltham, Mass., USA). In a next step, the VLP producing cells were subjected to clonal dilutions in order to characterize the number of pseudo HIV1 integrated provirus in different clones. A cellular clone (HEK293-VLP-CL40) containing between 1 and 2 copies of the pseudo HIV1 provirus (as determined by qPCR) was used for assessing the antiviral activity of meganucleases.

HEK293-VLP-CL40 cells were cultured in DMEM media supplemented with 2 mM L-glutamine, penicillin (100 IU/ml), streptomycin (100 mg/ml), amphotericin B (Fongizone: 0.25 mg/ml, Invitrogen-Life Science) and 10% of foetal bovine serum (FBS).

### b) Transfection of HEK293-VLP-CL40 Cells

The day before transfection, HEK293-VLP-CL40 cells were seeded in 12-well culture plates (Falcon, Becton Dickinson, Le Pont De Claix, France) at 10⁵ cells per well and incubated overnight at 37° C. in 1 ml of complete growth medium. The cultures were about 70% confluent on the day of transfection. Transfection with 1 μg of plasmid expressing I-CreI variants cleaving different HIV1 target sequences was done using FuGENE® HD Transfection Reagent (Roche Diagnostics, Indianapolis, Ind., USA) according to manufacturer's instruction. Transfection media was replaced 24 h after transfection and cells were kept at 37° C. in complete growth medium for other 24 hours.

### c) Cell Harvesting and p24 Determination

Cell supernatants were harvested 48 h post-transfection and p24 titres were either measured immediately or the supernatants were kept at −20° C. for ulterior quantification of the p24. HEK-293-CL40 transfected cells were then recovered and counted, prior to centrifugation at 1500 rpm for 5 minutes and storage of the dry cellular pellet at −20° C. for ulterior extraction of the genomic DNA.

The amount of p24 present in cellular supernatants was determined using the Alliance® HIV1-p24 ELISA Kit (Perkin Elmer Inc, Waltham, Mass., USA) according to the manufacturer's instructions. Results were expressed as p24 in pg/ml (or as pg/well, according to the cell culture conditions). The production of p24 was normalized by the number of cells present in the well at the moment of media harvesting, and expressed as p24 levels in fg/cell.

### 2) Results

The single chain molecules described in Table XLV (SEQ ID NO: 346 to 365) were tested for their ability to target the HIV1 provirus and reduce the amount of VLPs produced in the HEK293-VLP-CL40 cellular model. Cells were transfected with 1 μg of plasmid expressing the meganuclease variants and the level of p24 present in the culture supernatants was determined 48 h after transfection, as previously described. As a control, a non related meganuclease (NRM) was transfected. This NRM is not active against the HIV1 provirus and should have no effect on the level of p24 produced by NRM transfected cells. The p24 levels of NRM transfected cells, expressed in fg/cell, was considered as 100% of VLP production, and the p24 levels present in samples transfected with HIV meganucleases were compared to the NRM value, in order to determine the percentage of VLP production in these samples.

### a) Sequences Targeted in the HIV1 Provirus by the HIV1 Meganucleases

The meganuclease target sites have already been described except for the HIV1_{—}7 (SEQ ID NO:366), HIV1_{—}8 (SEQ ID NO:367) and HIV1_{—}9 (SEQ ID NO:368) targets.

The HIV1_{—}1 target (SEQ ID NO:319), described in example 1, is located in the U3 region of the proviral LTRs; while the HIV1_{—}3 target (SEQ ID NO:325), described in example 8, is located in the U5 region of the proviral LTRs. Since the LTRs are duplicated sequences flanking the viral ORFs in the integrated provirus, each of these two targets are present twice in the HIV1 provirus.

The HIV1_{—}4 target (SEQ ID NO:331) has been described in example 16, and is located in the gag gene of the HIV1 provirus, more precisely in the coding sequence of the p24 (CApsid) protein. The HIV1_{—}7 target (G GAG CC ACC CCAC AAG AT TTA A, SEQ ID NO: 366) also cleaves the coding sequence of the p24 protein, though at a different position. The HIV1_{—}7 target (SEQ ID NO:366) is also a 22 by (non-palindromic) target precisely located at positions 1321-1342 of the HIV-1 pNL4-3 vector (accession number AF324493, Adachi et al., J. Virol., 1986, 59, 284-291), a subtype B infectious molecular clone.

The HIV1_{—}5 target (SEQ ID NO:337) has been described in example 24, and is located in the pol gene of the HIV1 provirus, more precisely in the sequence coding for the PRotease protein. The HIV1_{—}9 target (SEQ ID NO:368) also cleaves the coding sequence of the protease, though at a different position. The HIV1_{—}9 target (A GAA AT CTG TTGA CTC AG ATT G, SEQ ID NO: 368) is also a 22 by (non-palindromic) target located at positions 2511-2532 of the HIV-1 pNL4-3 vector.

The HIV1_{—}8 target (G GGC CC CTA GGAA AAA GG GCT G, SEQ ID NO: 367) is a 22 by (non-palindromic) target located in the gag gene of the HIV1 provirus. This target is precisely located at positions 2006-2027 of the HIV-1 pNL4-3 vector, on the coding sequence of the p7 (NC, NucleoCapsid) protein.

Over again, it should be noted that two cleavage sites are present in the HIV1 proviral DNA for targets HIV1_{—}1 (SEQ ID NO:319) and HIV1_{—}3 (SEQ ID NO:325), while the remaining targets present only one cleavage site in the integrated provirus.

The presence of the HIV1 meganuclease cleavage sites in the HEK293-VLP-CL40 cells was confirmed by sequencing and their position is represented in FIG. 62.

### b) I-CreI Variants Targeting the HIV1 Genome Induce a Decrease in p24 Titres in a Cellular Model Harbouring an HIV1 Provirus

p24 titres were determined 48 hours after transfection with the HIV1 meganucleases as previously described. The values, expressed as p24 in fg/cell, were normalized respect to the amount of p24 released in a well transfected by a NRM, which was considered to be 100% for VLP production.

FIG. 63 shows the levels of p24 (in %) produced by the cells transfected with the different meganuclease plasmids. A reduction of p24 production is observed in samples transfected with HIV meganucleases. The meganucleases showing a higher reduction in p24 titers correspond to variants SCOH-HIV1_{—}3-B and SCOH-HIV1_{—}3-D (SEQ ID NO: 350 and 352), leading to nearly a 50% reduction of p24 levels compared to cells transfected with the NRM.

A significant reduction of p24 titers, ranging from 35-40%, is observed also for other I-CreI variants cleaving different targets in the HIV1 provirus (SCOH-HIV1_{—}1-B, SEQ ID NO: 347; SCOH-HIV1_{—}7-A, SEQ ID NO: 357; SCOH-HIV1_{—}8-D, SEQ ID NO: 362; and SCOH-HIV1_{—}9-B, SEQ ID NO: 364).

### EXAMPLE 34

### Detection of Cleavage Activity at the HIV1_{—}8 Locus in a Human Cell Line Harbouring an Integrated HIV1 Provirus

I-CreI variants targeting the HIV1_{—}8 target (SEQ ID NO:367), as well as their activity have been described in Examples 32 and 33. The efficiency of two of the HIV1_{—}8 meganucleases (SEQ ID NO:359 to 362) to cleave their endogenous DNA target sequence was next tested. This example will demonstrate that meganucleases engineered to cleave the HIV1_{—}8 target sequence (SEQ ID NO:367) cleave their cognate endogenous site in human cells harboring an integrated HIV1 provirus (HEK293-VLP-CL40 cells).

Repair of double-strand break by non homologous end joining (NHEJ) can generate small deletions and insertions (InDel) (FIG. 64). In nature, this error-prone mechanism can be deleterious for the cells survival but provides a rapid indicator of meganucleases activity at endogenous loci.

### EXAMPLE 34.1

### Detection of Induced Mutagenesis at the Endogenous Site

Two Single Chain I-CreI variants targeting the HIV1_{—}8 target (SEQ ID NO:367) cloned in the pCLS1853 plasmid were used for this experiment. The day previous to the experiment, cells derived from the human embryonic kidney cell line, 293-H (HEK293-VLP-CL40) were seeded in a 10 cm dish at density of 10⁶ cells/dish.

The following day, cells were transfected with 3 μg of an empty plasmid or a meganuclease-expressing plasmid using FuGene® HD Transfection Reagent (Roche Diagnostics, Indianapolis, Ind., USA) according to manufacturer's instruction. 72 hours after transfection, cells were collected and diluted (dilution 1/20) in fresh culture medium. After 7 days of culture, cells were collected and genomic DNA extracted. 200 ng of genomic DNA were used to amplify the endogenous locus surrounding the meganuclease cleavage site by PCR amplification. A 325 by fragment corresponding to the HIV1_{—}8 locus was amplified using specific PCR primers HI8f (SEQ ID NO 369; 5′-GACCCGGCCATAAAGCAAGAGTTTTGGCTG-3′) and HI8r (SEQ ID NO 370; 5′-AAGCTCTCTTCTGGTGGGGCTGTTGGCTCT-3′). PCR amplification was performed to obtain a fragment flanked by specific adaptator sequences (SEQ ID NO 371; 5′-CCATCTCATCCCTGCGTGTCTCCGACTCAG-3′ and 25 SEQ ID NO: 372 5′-CCTATCCCCTGTGTGCCTTGGCAGTCTCAG-3′) provided by the company offering sequencing service (GATC Biotech AG, Germany) on the 454 sequencing system (454 Life Sciences).

An average of 3,000 sequences was obtained from pools of the amplicons (500 ng). After sequencing, different samples were identified based on barcode sequences introduced in the first of the above adaptators. 15 sequences showed the presence of insertions or deletions in the cleavage site of HIV1_{—}8 meganucleases (SEQ ID NO:359 to 362).

### EXAMPLE 34.2

### Results

Table XLVI summarizes the results that were obtained.

| | Total sequence | InDel containing | |
|---|---|---|---|
| Vector expressing: | number: | sequences | % of InDel events |
| SCOH-HIV1_8-B | 833 | 8 | 0.96 |
| (SEQ ID NO: 360) | | | |
| (pCLS4058) | | | |
| SCOH-HIV1_8-D | 748 | 7 | 0.936 |
| (SEQ ID NO: 362) | | | |
| (pCLS4060) | | | |
| Empty | 1625 | 0 | 0 |

The analysis of the genomic DNA extracted from cells transfected with the meganucleases targeting the HIV1_{—}8 locus showed that around 1% of the analyzed sequences contained InDel events within the recognition site of HIV1_{—}8 meganucleases (SEQ ID NO:359 to 362) (Table XLVI). Since small deletions or insertions could be related to PCR or sequencing artefacts, the same locus was analyzed after transfection with a plasmid that does not express the meganuclease. The analysis of the HIV1_{—}8 locus revealed that no InDel events could be detected. These data demonstrate that meganucleases engineered to target the HIV1_{—}8 locus are active in human cells and can cleave their cognate endogenous sequence. Moreover, it shows that meganucleases have the ability to generate small InDel events within a sequence which would disrupt a gene ORF and thus inactivate the corresponding gene expression product.

1. An I-CreI variant, which cleaves at least one DNA target in a provirus of a pathogenic retrovirus, suitable for treating an infection of the retrovirus.

2. The variant of claim 1, wherein the pathogenic retrovirus is from at least one genus selected from the group consisting of Alpharetrovirus, Betaretrovirus, Gammaretrovirus, Deltaretrovirus, Epsilonretrovirus, Lentivirus, and Spumavirus.

3. The variant of claim 1, wherein the retrovirus is selected from the group consisting of Human T-lymphotrophic virus, Rous Sarcoma, and Human Immunodeficiency Virus.

4. The variant of claim 3, wherein the Human Immunodeficiency Virus is present and is at least one selected from the group consisting of Human Immunodeficiency Virus Type 1 (HIV1) and Human Immunodeficiency Virus Type 2 (HIV2).

5. The variant of claim 1, wherein the DNA target is at least one of SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 366, SEQ ID NO: 367, and SEQ ID NO: 368.

6. The variant of claim 1, comprising at least one sequence selected from the group consisting of SEQ ID NO: 350; SEQ ID NO: 352; SEQ ID NO: 1-13; SEQ ID NO: 26-46; SEQ ID NO: 59-85; SEQ ID NO: 88-94; SEQ ID NO: 97-165; SEQ ID NO: 168-174; SEQ ID NO: 177-186; SEQ ID NO: 189-238; SEQ ID NO: 241-242; SEQ ID NO: 245-253; SEQ ID NO: 256-316; SEQ ID NO: 346-349; SEQ ID NO: 351; and SEQ ID NO: 353-365.

7. The variant of claim 1, wherein at least one of the two I-CreI monomers has at least two substitutions, one in each of the two functional subdomains of the LAGLIDADG core domain, comprises at least one substitution in at least one position selected from the group consisting of 26, 28, 30, 32, 33, 38, and 40, and in the second functional subdomain comprises at least one substitution in at least one position selected from the group consisting of 44, 68, 70, 75, and 77,
wherein the variant is obtained by a method comprising:
(a) constructing a first series of I-CreI variants having at least one substitution in a first functional subdomain of the LAGLIDADG core domain comprising at least one substitution at at least one position selected from the group consisting of 26, 28, 30, 32, 33, 38, and 40 of I-CreI;
(b) constructing a second series of I-CreI variants having at least one substitution in a second functional subdomain of the LAGLIDADG core domain comprising at least one substitution at at least one position selected from the group consisting of 44, 68, 70, 75, and/or and 77 of I-CreI;
(c) selecting, screening, or selecting and screening the variants from the first series of the constructing (a) which are able to cleave at least one DNA target sequence selected from the group of SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 366, SEQ ID NO: 367, and SEQ ID NO: 368, wherein at least one of (i) the nucleotide triplet in positions −10 to −8 of the I-CreI site has been replaced with the nucleotide triplet which is present in positions −10 to −8 of the selected DNA target sequence from the provirus and (ii) the nucleotide triplet in positions +8 to +10 has been replaced with the reverse complementary sequence of the nucleotide triplet which is present in position −10 to −8 of the selected DNA target sequence from the provirus;
(d) selecting, screening, or selecting and screening the variants from the second series of the constructing (b) which are able to cleave at least one DNA target sequence selected from the group of SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 366, SEQ ID NO: 367, and SEQ ID NO: 368, wherein at least one of (i) the nucleotide triplet in positions −5 to −3 of the I-CreI site has been replaced with the nucleotide triplet which is present in positions −5 to −3 of the selected DNA target sequence from the provirus and (ii) the nucleotide triplet in positions +3 to +5 has been replaced with the reverse complementary sequence of the nucleotide triplet which is present in position −5 to −3 of the selected DNA target sequence from the provirus;
(e) selecting, screening, or selecting and screening the variants from the first series of the constructing (a) which are able to cleave at least one DNA target sequence selected from the group of SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 366, SEQ ID NO: 367, and SEQ ID NO: 368, wherein at least one of (i) the nucleotide triplet in positions +8 to +10 of the I-CreI site has been replaced with the nucleotide triplet which is present in positions +8 to +10 of the selected DNA target sequence from the provirus and (ii) the nucleotide triplet in positions −10 to −8 has been replaced with the reverse complementary sequence of the nucleotide triplet which is present in positions +8 to +10 of the selected DNA target sequence from the provirus;
(f) selecting, screening, or selecting and screening the variants from the second series of (b) which are able to cleave at least one DNA target sequence selected from the group of SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 366, SEQ ID NO: 367, and SEQ ID NO: 368, wherein at least one of (i) the nucleotide triplet in positions +3 to +5 of the I-CreI site has been replaced with the nucleotide triplet which is present in positions +3 to +5 of the selected DNA target sequence from the provirus and (ii) the nucleotide triplet in positions −5 to −3 has been replaced with the reverse complementary sequence of the nucleotide triplet which is present in positions +3 to +5 of the selected DNA target sequence from the provirus;
(g) combining in a single variant, the at least one mutation in at least one of positions 26, 28, 30, 32, 33, 38, 44, 68, 70, 75, and 77 of two variants from (c) and (d), to obtain a novel homodimeric I-CreI variant which cleaves a sequence wherein (i) the nucleotide triplet in positions −10 to −8 is identical to the nucleotide triplet which is present in positions −10 to −8 of the selected DNA target sequence from the provirus, (ii) the nucleotide triplet in positions +8 to +10 is identical to the reverse complementary sequence of the nucleotide triplet which is present in positions −10 to −8 of the selected DNA target sequence from the provirus, (iii) the nucleotide triplet in positions −5 to −3 is identical to the nucleotide triplet which is present in positions −5 to −3 of the selected DNA target sequence from the provirus and (iv) the nucleotide triplet in positions +3 to +5 is identical to the reverse complementary sequence of the nucleotide triplet which is present in positions −5 to −3 of the selected DNA target sequence from said provirus; and/or
(h) combining in a single variant, the at least one mutation in at least one of positions 26, 28, 30, 32, 33, 38, 40, 44, 68, 70, 75, and 77 of two variants from (e) and (f), to obtain a novel homodimeric I-CreI variant which cleaves a sequence wherein (i) the nucleotide triplet in positions +8 to +10 of the I-CreI site has been replaced with the nucleotide triplet which is present in positions +8 to +10 of the selected DNA target sequence from the provirus and (ii) the nucleotide triplet in positions −10 to −8 is identical to the reverse complementary sequence of the nucleotide triplet in positions +8 to +10 of the selected DNA target sequence from the provirus, (iii) the nucleotide triplet in positions +3 to +5 is identical to the nucleotide triplet which is present in positions +3 to +5 of the selected DNA target sequence from the provirus, (iv) the nucleotide triplet in positions −5 to −3 is identical to the reverse complementary sequence of the nucleotide triplet which is present in positions +3 to +5 of the selected DNA target sequence from the provirus;
(i) combining the variants obtained in (g) and (h) to form heterodimers; and
(j) selecting, screening, or selecting and screening the heterodimers from (i) which are able to cleave said DNA target sequence from the provirus.

8. A combination comprising:
the variant of claim 1; and
another anti-retroviral medicament.

9. A polynucleotide fragment, encoding the variant of claim 1.

10. An expression vector, comprising at least one polynucleotide fragment of claim 9.

11. A host cell which is modified by the polynucleotide fragment of claim 9 or an expression vector comprising the polynucleotide fragment.

12. A non-human transgenic animal, modified by a polynucleotide fragment of claim 9 or an expression comprising the polynucleotide fragment vector.

13. A method of non-therapeutic genome engineering, the method comprising:
contacting at least one variant of claim 1 with a DNA target with a provirus of a pathogenic retrovirus.

14. The variant of claim 2, wherein the pathogenic retrovirus is from the genus Alpharetrovirus.

15. The variant of claim 2, wherein the pathogenic retrovirus is from the genus Betaretrovirus.

16. The variant of claim 2, wherein the pathogenic retrovirus is from the genus Gammaretrovirus.

17. The variant of claim 2, wherein the pathogenic retrovirus is from the genus Deltaretrovirus.

18. The variant of claim 2, wherein the pathogenic retrovirus is from the genus Epsilonretrovirus.

19. The variant of claim 2, wherein the pathogenic retrovirus is from the genus Lentivirus.

20. A method of treating a retroviral infection in a subject, the method comprising:
administering to a subject in need thereof, an effective amount of the I-CreI variant of claim 1.
